(19) United States
(12) Patent Application Publication
(10) Pub. No.: US 2009/0075989 A1
(43) Pub. Date: Mar. 19, 2009

(54) NON-STEROIDAL PROGESTERONE RECEPTOR MODULATORS

(76) Inventors: Wolfgang Schwede, Glienicke (DE); Carsten Moeller, Berlin (DE); Anja Schmidt, Berlin (DE); Ulrike Fuhrmann, Berlin (DE); Andrea Rotgeri, Berlin (DE); Thomas Andrew Kirkland, Canyon, CA (US); Ralf Wyrwa, Rothenstein (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/961,314

(22) Filed: Dec. 20, 2007

Related U.S. Application Data
(60) Provisional application No. 60/880,707, filed on Jan. 17, 2007.
(60) Provisional application No. 60/979,208, filed on Oct. 11, 2007.

(30) Foreign Application Priority Data
Dec. 21, 2006 (DE) 10 2006 061 912.9

(51) Int. Cl. A61K 31/536; C07D 265/02; C07D 307/77; A61K 31/343; C07C 233/00; A61K 31/164; C07D 213/02; A61K 31/44; C07D 295/00; A61K 31/5375; A61P 15/00; A61P 35/00; A61P 15/08

(52) U.S. Cl. 514/230.5; 544/63; 549/466; 514/470; 564/169; 514/621; 564/174; 514/622; 546/337; 514/357; 544/168; 514/237.5

(57) ABSTRACT

The present invention relates to non-steroidal progesterone receptor modulators of the general formula 1,

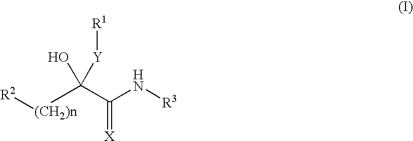

the use of the progesterone receptor modulators for the manufacture of medicaments, and pharmaceutical compositions which comprise these compounds.

The compounds according to the invention are suitable for the therapy and prophylaxis of gynaecological disorders such as endometriosis, leiomyomas of the uterus, dysfunctional bleeding and dysmenorrhoea, and for the therapy and prophylaxis of hormone-dependent tumours and for use for female fertility control and for hormone replacement therapy.

This application claims the benefit of the filing date of U.S. Provisional Application Ser. No. 60/880707 filed Jan. 17, 2007 and U.S. Provisional Application Ser. No. 60/979,208 filed Oct. 11, 2007.

The present invention relates to non-steroidal progesterone receptor modulators, a method for their preparation, the use of the progesterone receptor modulators for the manufacture of medicaments, and pharmaceutical compositions which comprise these compounds.

The steroid hormone progesterone controls in a decisive manner the reproductive process in the female body. Progesterone is secreted in large quantities during the cycle and pregnancy respectively by the ovary and the placenta. Progesterone in cooperation with oestrogens brings about cyclic changes in the uterine mucosa (endometrium) during the menstrual cycle. Elevated progesterone levels after ovulation influence the uterine mucosa to convert it into a state permitting nidation of an embryo (blastocyst). During pregnancy, progesterone controls the relaxation of the myometrium and maintains the function of the decidual tissue.

It is further known that progesterone inhibits endometrial proliferation by suppressing oestrogen-mediated mitosis in uterine tissue (K. Chwalisz, R. M. Brenner, U. Fuhrmann, H. Hess-Stumpp, W. Elger, Steroids 65, 2000, 741-751).

Progesterone and progesterone receptors are also known to play a significant part in pathophysiological processes. Progesterone receptors have been detected in the foci of endometriosis, but also in tumours of the uterus, of the breast and of the CNS. It is further known that uterine leiomyomas grow progesterone-dependently.

The effects of progesterone in the tissues of the genital organs and in other tissues occur through interactions with progesterone receptors which are responsible for the cellular effects.

Progesterone receptor modulators are either pure agonists or inhibit the effect of progesterone partly or completely. Accordingly, substances are defined as pure agonists, partial agonists (selective progesterone receptor modulators=SPRMs) and pure antagonists.

In accordance with the ability of progesterone receptor modulators to display their effect via the progesterone receptor, these compounds have a considerable potential as therapeutic agents for gynaecological and oncological indications and for obstetrics and fertility control.

Pure progesterone receptor antagonists completely inhibit the effect of progesterone on the progesterone receptor. They have anti-ovulatory properties and the ability to inhibit oestrogen effects in the endometrium, as far as complete atrophy. They are therefore particularly suitable for intervening in the female reproductive process, e.g. post-ovulation, in order to prevent nidation of a fertilized egg cell, during pregnancy in order to increase the reactivity of the uterus to prostaglandins or oxytocin, or in order to achieve opening and softening (“ripening”) of the cervix, and to induce a great readiness of the myometrium to contract.

A beneficial effect on the pathological event is expected in foci of endometriosis and in tumour tissues which are equipped with progesterone receptors after administration of pure progesterone receptor antagonists. There might be particular advantages for influencing pathological states such as endometriosis or uterine leiomyomas if ovulation inhibition can additionally be achieved by the progesterone receptor antagonists. Ovulation inhibition also dispenses with some of the ovarian hormone production and thus the stimulating effect, deriving from this proportion, on the pathologically altered tissue.

The first progesterone receptor antagonist described, RU 486 (also mifepristone), was followed by the synthesis and characterization of a large number of analogues with progesterone receptor-antagonistic activity of varying strength. Whereas RU 486 also shows an antiglucocorticoid effect in addition to the progesterone receptor-antagonistic effect, compounds synthesized later are notable in particular for a more selective effect as progesterone receptor antagonists.

Besides steroidal compounds such as onapristone or lilopristone, which are notable by comparison with RU 486 for a better dissociation of the progesterone receptor-antagonistic effect and the antiglucocorticoid effect, also known from the literature are various non-steroidal structures whose antagonistic effect on the progesterone receptor is being investigated [see, for example, S. A. Leonhardt and D. P. Edwards, Exp. Biol. Med. 227: 969-980 (2002) and R. Winneker, A. Fensome, J. E. Wrobel, Z. Zhang, P. Zhang, Seminars in Reproductive Medicine, Volume 23: 46-57 (2005)]. However, non-steroidal compounds disclosed to date have only moderate antagonistic activity compared with the activity of known steroidal structures. The most effective non-steroidal compounds are reported to have in vitro activities which are 10% of the activity of RU 486.

The antiglucocorticoid activity is disadvantageous for therapeutic use, where the inhibition of progesterone receptors is at the forefront of the therapy. An antiglucocorticoid activity causes unwanted side effects at the dosages necessary for therapy. This may prevent administration of a therapeutically worthwhile dose or lead to discontinuation of the treatment.

Partial or complete reduction of the antiglucocorticoid properties is therefore an important precondition for therapy with progesterone receptor antagonists, especially for those indications requiring treatment lasting weeks or months.

In contrast to the pure antagonists, partial progesterone receptor agonists (SPRMs) show a residual agonistic property which may vary in strength. This leads to these substances showing agonistic effects on the progesterone receptor in certain organ systems (D. DeManno, W. Elger, R. Garg, R. Lee, B. Schneider, H. Hess-Stumpp, G. Schuber, K. Chwalisz, Steroids 68, 2003, 1019-1032). Such an organ-specific and dissociated effect may be of therapeutic benefit for the described indications.

It is therefore an object of the present invention to provide further non-steroidal progesterone receptor modulators. These compounds are intended to have a reduced antiglucocorticoid effect and therefore be suitable for the therapy and prophylaxis of gynaecological disorders such as endometriosis, leiomyomas of the uterus, dysfunctional bleeding and dysmenorrhoea. The compounds according to the invention are additionally intended to be suitable for the therapy and prophylaxis of hormone-dependent tumours, for example of breast, endometrial, ovarian and prostate carcinomas. The compounds are intended furthermore to be suitable for use in female fertility control and for female hormone replacement therapy.

This object is achieved according to the present invention by the provision of non-steroidal compounds of the general formula I

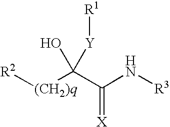

in which
q is 0 or 1,
R¹ is a mono- or bicyclic C₆-C₁₂-aryl, 5-12-membered heteroaryl, C₃-C₁₀-cycloalkyl or 3-10-membered heterocycloalkyl radical which may in each case be unsubstituted or optionally substituted by up to 3 radicals, where the substituents each independently of one another have the following meaning:
   C₁-C₈-alkyl, C₂-C₈-alkenyl, C₂-C₈-alkynyl, partly or fully fluorinated C₁-C₆-alkyl, partly or fully fluorinated C₁-C₆-alkoxy, C₁-C₆-alkoxy-C₁-C₆-alkyl, C₁-C₆-alkoxy-C₁-C₆-alkoxy, (CH₂)ₚ-C₃-C₁₀-cycloalkyl, (CH₂)ₚ-heterocycloalkyl, (CH₂)ₚCN, (CH₂)ₚHal, (CH₂)ₚNO₂, an optionally Z-substituted (CH₂)ₚ-C₆-C₁₂-aryl, an optionally Z-substituted (CH₂)ₚ-heteroaryl,
   —(CH₂)ₚPO₃(R^{b})₂, —(CH₂)ₚNR^{c}R^{d}, —(CH₂)ₚNR^{e}COR^{b},
   —(CH₂)ₚNR^{e}CSR^{b}, —(CH₂)ₚNR^{e}S(O)R^{b}, —(CH₂)ₚNR^{e}S(O)₂R^{b},
   —(CH₂)ₚNR^{e}CONR^{c}R^{d}, —(CH₂)ₚNR^{e}COOR^{b},
   —(CH₂)ₚNR^{e}C(NH)NR^{c}R^{d}, —(CH₂)ₚNR^{e}CSNR^{c}R^{d},
   —(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚCSR^{b}, —(CH₂)ₚ S(O)R^{b}, —(CH₂)ₚS(O)(NH)R^{b}, —(CH₂)ₚS(O)₂R^{b},
   —(CH₂)ₚS(O)₂NR^{c}R^{d}, —(CH₂)ₚSO₂OR^{b}, —(CH₂)ₚCO₂R^{b},
   —(CH₂)ₚCONR^{c}R^{d}, —(CH₂)ₚCSNR^{c}R^{d}, —(CH₂)ₚOR^{b}, —(CH₂)ₚSR^{b},
   —(CH₂)ₚCR^{b}(OH)—R^{b}, —(CH₂)ₚ—C═NOR^{b},
   —O—(CH₂)ₙ—O—, —O—(CH₂)ₙ—CH₂—, —O—CH═CH— or —(CH₂)ₙ₊₂—,
   where n is 1 or 2 and the terminal oxygen atoms and/or carbon atoms are linked to directly adjacent ring carbon atoms, and p is 0, 1, 2, 3, 4, 5 or 6, and
Z is a cyano, halogen, nitro, —(CH₂)ₚOR^{b}, —(CH₂)ₚS(O)₂R^{b}, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}—NR^{c}R^{d}, partly or fully fluorinated C₁-C₆-alkyl or partly or fully fluorinated C₁-C₆-alkoxy,
   R^{b} is a hydrogen or a C₁-C₆-alkyl, C₂-C₈-alkenyl, C₂-C₈-alkynyl, C₃-C₁₀-cycloalkyl, C₆-C₁₂-aryl or a partly or completely fluorinated C₁-C₃-alkyl and
   R^{c} and R^{d} are independently of one another a hydrogen, an optionally W-substituted C₁-C₆-alkyl, C₂-C₈-alkenyl, C₂-C₈-alkynyl, C₃-C₁₀-cycloalkyl, C₆-C₁₂-aryl, a 5- to 12-membered heteroaryl radical, C(O)R^{b} or a hydroxy group and
   W is —NR^{f}R^{g} where
   R^{f} is hydrogen or C₁-C₃-alkyl and
   R^{g} is hydrogen or C₁-C₃-alkyl or
   R^{f} and R^{g}, with inclusion of the nitrogen, form a 3- to 7-membered ring which is optionally extended by O, S or NR^{h} where
   R^{h} is hydrogen, C₁-C₃-alkyl, C₁-C₃-alkanoyl, C₁-C₃-alkyl-sulphonyl or C₁-C₃-alkoxycarbonyl,
where if
   R^{c} is a hydroxy group, R^{d} can only be a hydrogen, a C₁-C₆-alkyl, C₂-C₈-alkenyl, C₂-C₈-alkynyl, C₃-C₁₀-cycloalkyl or C₆-C₁₂-aryl and vice versa, and
   R^{e} is a hydrogen, C₁-C₆-alkyl, C₂-C₈-alkenyl, C₂-C₈-alkynyl, C₃-C₁₀-cycloalkyl or C₆-C₁₂-aryl,
R² is a mono- or bicyclic C₆-C₁₂-aryl or 5-12-membered heteroaryl radical which may be unsubstituted or optionally substituted by up to 3 of the radicals mentioned under R¹,
R³ has the meaning indicated for R² or is one of the following groups mentioned under A or B:

### A: 6-Membered/6-Membered Ring Systems

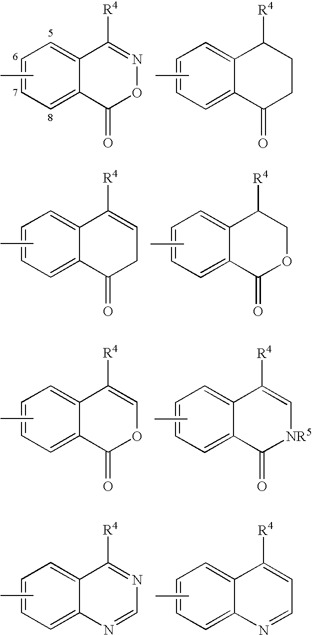

### Linkage to the Basic Structure at Position 5, 6, 7 or 8

### B: 6-Membered/5-Membered Ring Systems

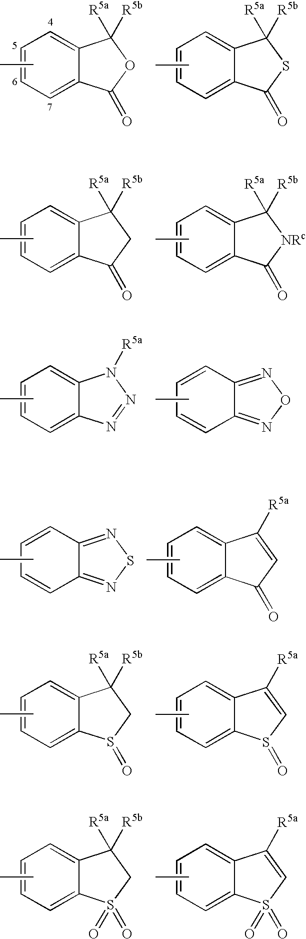

### Linkage to the Basic Structure at Position 4, 5, 6 or 7

where
R⁴ is hydrogen or C₁-C₄-alkyl or partly or completely fluorinated C₁-C₄-alkyl,
R^{5a} and R^{5b} are independently of one another hydrogen, C₁-C₄-alkyl or partly or fully fluorinated C₁-C₄-alkyl, or together with the ring carbon atom form a 3- to 6-membered ring,

and
X is oxygen or two hydrogen atoms,
Y is —(CH₂)ₘ—, —C≡C— or —CH═CH—, and
m is 0 or 1,

and the pharmaceutically acceptable salts thereof.

The compounds according to the invention of the general formula I may, owing to the presence of centres of asymmetry, exist as different stereoisomers. Both the racemates and the separate stereoisomers belong to the subject matter of the present invention.

The present invention further includes the novel compounds as active pharmaceutical ingredients, their therapeutic use and pharmaceutical dosage forms which comprise the novel substances.

The compounds according to the invention of the general formula (I) or their pharmaceutically acceptable salts can be used to produce a medicament, in particular for the treatment and prophylaxis of gynaecological disorders such as endometriosis, leiomyomas of the uterus, dysfunctional bleeding and dysmenorrhoea. The compounds according to the invention may further be used for the treatment and prophylaxis of hormone-dependent tumours such as, for example, for breast, prostate and endometrial carcinoma.

The compounds according to the invention of the general formula (I) or their pharmaceutically acceptable salts are also suitable for use for female fertility control or for female hormone replacement therapy.

The non-steroidal compounds according to the invention of the general formula I have strong antagonistic or strong partial agonistic effects on the progesterone receptor with high potency. They show a strong dissociation of effects in relation to their strength of binding to the progesterone receptor and to the glucocorticoid receptor. Whereas known progesterone receptor antagonists such as mifepristone (RU 486) show, besides the desired high binding affinity for the progesterone receptor, likewise a high affinity for the glucocorticoid receptor, the compounds according to the invention are notable for a very low glucocorticoid receptor binding with simultaneously a high progesterone receptor affinity.

The substituents, defined as groups, of the compounds according to the invention of the general formula I may in each case have the following meanings:

C₁-C₄-, C₁-C₆- and C₁-C₈-alkyl group means unbranched or optionally branched alkyl radicals. Examples thereof are a methyl, ethyl, n-propyl, isopropyl, n-, iso-, tert-butyl, hexyl, heptyl or octyl group.

Preferred in the meaning of R⁴ are methyl or ethyl.

Alkenyl means unbranched or optionally branched alkenyl radicals. Examples of the meaning of a C₂-C₈-alkenyl group in the context of the invention are the following: vinyl, allyl, 3-buten-1-yl or 2,3-dimethyl-2-propenyl.

Alkynyl means unbranched or optionally branched alkynyl radicals. A C₂-C₈-alkynyl radical is intended to be for example an ethynyl, propynyl, butynyl, pentynyl, hexynyl and octynyl group, but preferably an ethynyl or propynyl group.

Possible examples of C₁-C₆-alkoxyl-C₁-C₆-alkoxy group are methoxymethoxy, ethoxymethoxy or 2-methoxyethoxy.

A radical OR^{b} in the context of the invention is a hydroxy, methoxy, ethoxy, n-propoxy, isopropoxy, n-, iso-, tert-butoxy or n-pentoxy, phenoxy, 2,2-dimethylpropoxy or 3-methylbutoxy group. Hydroxy, methoxy, ethoxy and phenoxy are preferred.

Suitable for a partly or completely fluorinated C₁-C₄-alkyl group are in particular the trifluoromethyl or pentafluoroethyl group.

A halogen atom may be a fluorine, chlorine, bromine or iodine atom. Fluorine, chlorine or bromine is preferred here.

Examples which may be mentioned of monocyclic C₃-C₁₀-cycloalkyl in the meaning of R¹ are cyclopropane, cyclobutane, cyclopentane and cyclohexane. Cyclopropyl, cyclopentyl and cyclohexyl are preferred.

Examples of monocyclic 3-10-membered heterocyclic radicals in the meaning of R¹ are morpholine, tetrahydrofuran, piperidine, pyrrolidine oxirane, oxetane, aziridine, dioxolane, dioxane, thiophene, furan, pyran, pyrrole, imidazole, pyrazole, pyridine, pyrazine, pyrimidine, pyridazine, piperazine, thiazole, oxazole, furazane, pyrroline, thiazoline, triazole, tetrazole, using any of the chemically possible isomers in relation to the positions of the heteroatoms.

Examples which may be mentioned of bicyclic 3-10-membered heterocycles are quinoline, quinazoline and naphthyridine.

Examples of the aromatic mono- or bicyclic system in the meaning of R¹, R² or R³, which may optionally be substituted by up to 3 radicals, are a phenyl or naphthyl radical, preferably a phenyl radical.

In the meaning of R², preference is given to a substituted or unsubstituted phenyl or naphthyl ring. If the phenyl ring is substituted, preference is given in turn to the following substituents: nitro, cyano, trifluoromethyl, phenyl, tert-butyl, methoxy, dimethylamino, methylsulphonyl, phenoxy, acetyl, hydroxy, acetoxy, thiomethyl, hydroxymethyl, fluorine, chlorine or bromine.

For R¹, R² or R³, preference is also given to substituted or unsubstituted mono- or bicyclic heteroaryl radicals. Particular preference is given to heteroaryls having 1 to 3 nitrogen atoms and/or one sulphur atom and/or one oxygen atom.

The following substituents are preferred here: nitro, cyano, trifluoromethyl, phenyl, tert-butyl, methoxy, dimethylamino, methylsulphonyl, phenoxy, acetyl, hydroxy, acetoxy, thiomethyl, hydroxymethyl, fluorine, chlorine or bromine.

Examples of a heteroaromatic radical in the meaning of R¹, R² or R³, which may optionally be substituted by up to 3 radicals, are the 2-, 3- or 4-pyridinyl, the 2- or 3-furyl, the 2- or 3-thienyl, the 2- or 3-pyrrolyl, the 2-, 4- or 5-imidazolyl, the pyrazinyl, the 2-, 4- or 5-pyrimidinyl or 3- or 4-pyridazinyl radical or quinolinyl and quinazolyl.

The number p for the (CH₂)ₚ radical may be a number 0, 1, 2, 3, 4, 5 or 6, preferably 0, 1 or 2. “Radical” means according to the invention all functional groups which are mentioned under R¹ in connection with (CH₂)ₚ.

In the case where the compounds of the general formula I are in the form of salts, this is possible for example in the form of the hydrochloride, sulphate, nitrate, tartrate, citrate, fumarate, succinate or benzoate.

If the compounds according to the invention are in the form of racemic mixtures, they can be fractionated by methods of racemate resolution familiar to the skilled person into the pure optically active forms. For example, the racemic mixtures can be separated into the pure isomers by chromatography on a support material which is itself optically active (CHIRALPAK AD®). It is also possible to esterify the free hydroxy group in a racemic compound of the general formula I with an optically active acid, and to separate the resulting diastereoisomeric esters by fractional crystallization or chromatography and to hydrolyse the separated esters in each case to the optically pure isomers. It is possible to use as optically active acid for example mandelic acid, camphorsulphonic acid or tartaric acid.

Compounds of the general formula (I) preferred according to the present invention are those in which:
R¹ is a mono- or bicyclic C₆-C₁₂-aryl or 5-12-membered heteroaryl radical, each of which may be unsubstituted or optionally substituted by up to 2 radicals, where the substituents each independently of one another have the meanings mentioned under R¹ hereinbefore,
R² is a mono- or bicyclic C₆-C₁₂-aryl or 5-12-membered heteroaryl radical which may be unsubstituted or optionally substituted by up to 2 of the radicals mentioned under R¹, and
R³ is a mono- or bicyclic C₆-C₁₂-aryl or 5-12-membered heteroaryl radical which may be unsubstituted or optionally substituted by up to 3 of the radicals mentioned under R¹, or a radical from the group A mentioned under R³, where the linkage to the NH group which is adjacent according to general formula (I) takes place at position 5 or 6 of the ring systems mentioned under A, or a radical from the group B mentioned under R³, where the linkage to the NH group which is adjacent according to general formula (I) takes place at position 4 or 5 of the ring systems mentioned under B.

Particularly preferred compounds of the general formula (I) are furthermore those in which:
R¹ is a monocyclic C₆-C₁₂-aryl or 5-12-membered heteroaryl radical which is unsubstituted or substituted by up to 2 of the groups appropriately mentioned under R¹, in particular a 6-membered aromatic ring or a 5-6-membered heteroaryl radical, particularly preferably an aromatic ring, very particularly preferably a phenyl radical, and
R³ is a phenyl ring substituted by up to 2 of the radicals mentioned under R¹.
   Particularly preferred substituents on R³ are chlorine, cyano and/or a trifluoromethyl radical.
   Very particular preference is given to the following combinations:

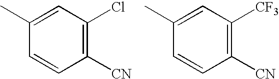

Further preferred compounds of the general formula (I) are those in which:
R³ has one of the following meanings:

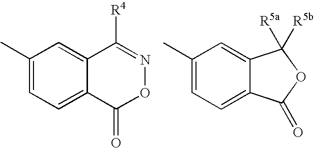

Further preferences are
R⁴ a methyl or ethyl radical,
R^{5a} and R^{5b} both simultaneously hydrogen and
q 0 and
p 0, 1 or 2.

The substituents of the mono- or bicyclic systems mentioned under R¹, R² and R³ preferably have the following meaning:
C₁-C₈-alkyl, C₂-C₈-alkenyl, C₂-C₈-alkynyl, partly or fully fluorinated C₁-C₆-alkyl, (CH₂)ₚ—heterocycloalkyl,
—(CH₂)ₚCN, (CH₂)ₚHal, (CH₂)ₚNO₂, (CH₂)ₚ—C₆-C₁₂-aryl, —(CH₂)ₚ-heteroaryl, —(CH₂)ₚNR^{c}R^{d},
—(CH₂)ₚNR^{e}COR^{b}, —(CH₂)ₚNR^{e}S(O)R^{b}, —(CH₂)ₚNR^{e}S(O)₂R^{b}, —(CH₂)ₚNR^{e}CONR^{c}R^{d},
—(CH₂)ₚNR^{e}S(O)NR^{c}R^{d}, —(CH₂)ₚNR^{e}S(O)₂NR^{c}R^{d}, —(CH₂)ₚCOR^{b}, —(CH₂)ₚS(O)R^{b},
—(CH₂)ₚS(O)(NH)R^{b}, —(CH₂)ₚS(O)(NH)R^{b}, —(CH₂)ₚS(O)₂R^{b}, —(CH₂)ₚS(O)₂NR^{c}R^{d},
—(CH₂)ₚCO₂R^{b},—(CH₂)ₚCONR^{c}R^{d}, —(CH₂)ₚOR^{b}, —(CH₂)ₚCR^{b}(OH)—R^{b}, —O—(CH₂)ₙ—O—.

Particularly preferred for R¹, R² and R³ are C₁-C₄-alkyl, C₂-C₄-alkenyl, C₂-C₄-alkynyl, partly or fully fluorinated C₁-C₂-alkyl, and C₁-C₆-alkoxy, C₁-C₆-acyl, halogen, NO₂, CN, (CH₃)₂N, CH₃SO₂— and C₁-C₆-aryl.

The compounds mentioned below, and the use thereof, are preferred according to the invention:

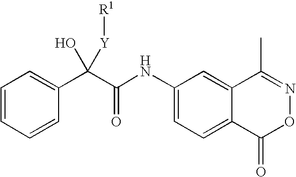

| | | Racemic or | |
|---|---|---|---|
| No. | Example | enantiomer | —Y—R¹ |
| 1 2 3 | 1 | rac+− | 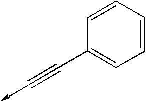 |
| 4 5 6 | 2 | rac+− | 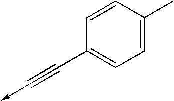 |
| 7 8 9 | 4 | rac+− | 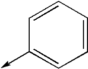 |
| 101112 | 5 6a 6b | rac+− | 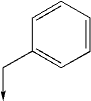 |
| 131415 | 7 | rac+− | 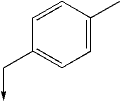 |
| 161718 | 8 | rac+− | 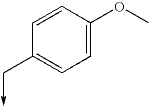 |
| 192021 | 9 | rac+− | 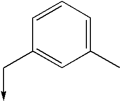 |
| 222324 | 10 | rac+− | 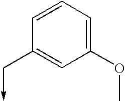 |

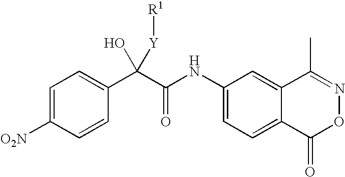

| | | Racemic or | |
|---|---|---|---|
| No. | Example | enantiomer | —Y—R¹ |
| 252627 | | rac+− | 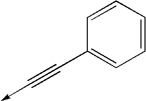 |
| 282930 | | rac+− | 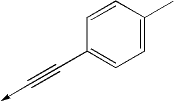 |
| 313233 | | rac+− | 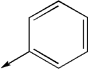 |
| 343536 | 11 | rac+− | 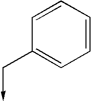 |
| 373839 | 12 | rac+− | 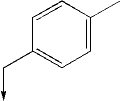 |
| 404142 | 13 | rac+− | 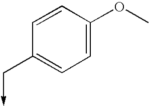 |
| 434445 | 14 | rac+− | 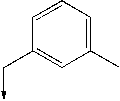 |
| 464748 | 15 | rac+− | 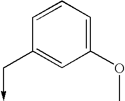 |

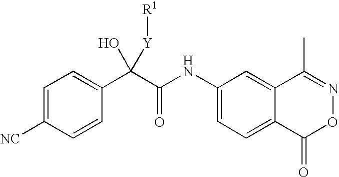

| | | Racemic or | |
|---|---|---|---|
| No. | Example | enantiomer | —Y—R¹ |
| 495051 | | rac+− | 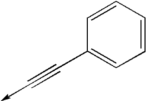 |
| 525354 | | rac+− | 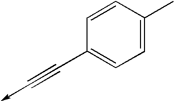 |
| 555657 | 16 | rac+− | 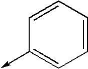 |
| 585960 | 17 | rac+− | 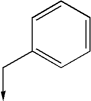 |
| 616263 | 19 | rac+− | 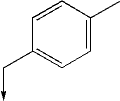 |
| 646566 | | rac+− | 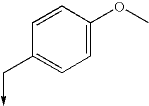 |
| 676869 | 18 | rac+− | 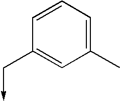 |
| 707172 | | rac+− | 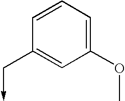 |

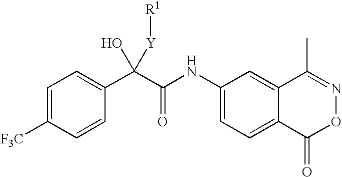

| | | Racemic or | |
|---|---|---|---|
| No. | Example | enantiomer | —Y—R¹ |
| 737475 | | rac+− | 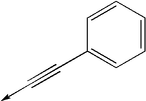 |
| 767778 | | rac+− | 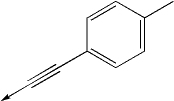 |
| 798081 | | rac+− | 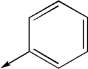 |
| 828384 | 20 | rac+− | 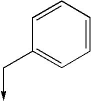 |
| 858687 | 21 | rac+− | 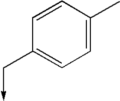 |
| 888990 | | rac+− | 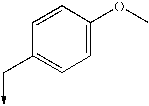 |
| 919293 | | rac+− | 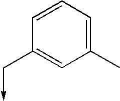 |
| 949596 | | rac+− | 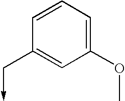 |

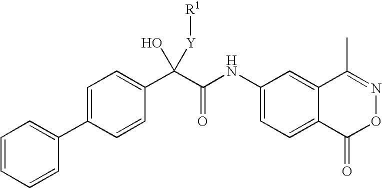

| | | Racemic or | |
|---|---|---|---|
| No. | Example | enantiomer | —Y—R¹ |
| 97 98 99 | | rac+− | 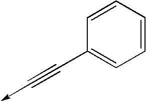 |
| 100101102 | | rac+− | 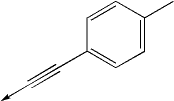 |
| 103104105 | | rac+− | 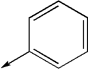 |
| 106107108 | 22 | rac+− | 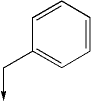 |
| 109110111 | | rac+− | 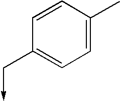 |
| 112113114 | | rac+− | 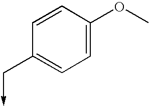 |
| 115116117 | | rac+− | 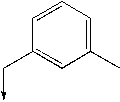 |
| 118119120 | | rac+− | 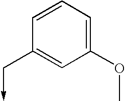 |

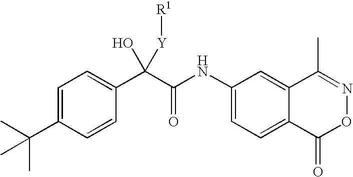

| | | Racemic or | |
|---|---|---|---|
| No. | Example | enantiomer | —Y—R¹ |
| 121122123 | | rac+− | 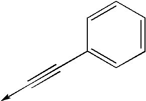 |
| 124125126 | | rac+− | 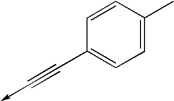 |
| 127128129 | | rac+− | 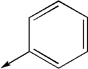 |
| 130131132 | | rac+− | 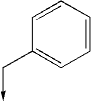 |
| 133134135 | | rac+− | 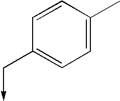 |
| 136137138 | | rac+− | 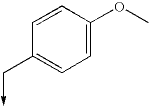 |
| 139140141 | | rac+− | 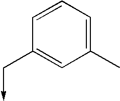 |
| 142143144 | | rac+− | 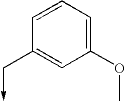 |

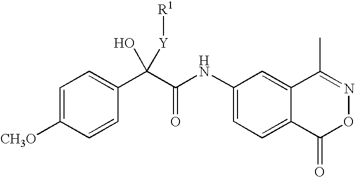

| | | Racemic or | |
|---|---|---|---|
| No. | Example | enantiomer | —Y—R¹ |
| 145146147 | | rac+− | 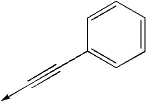 |
| 148149150 | | rac+− | 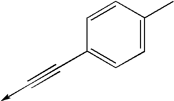 |
| 151152153 | | rac+− | 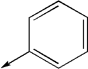 |
| 154155156 | | rac+− | 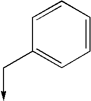 |
| 157158159 | | rac+− | 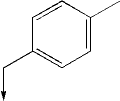 |
| 160161162 | | rac+− | 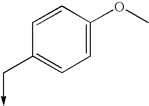 |
| 163164165 | | rac+− | 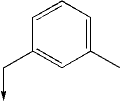 |
| 166167168 | | rac+− | 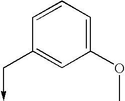 |

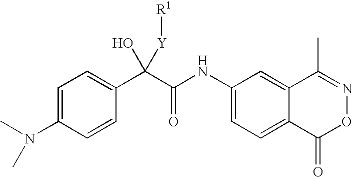

| | | Racemic or | |
|---|---|---|---|
| No. | Example | enantiomer | —Y —R¹ |
| 169170171 | | rac+− | 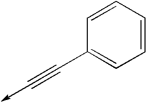 |
| 172173174 | | rac+− | 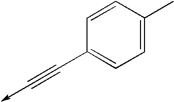 |
| 175176177 | | rac+− | 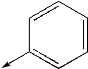 |
| 178179180 | | rac+− | 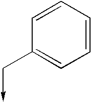 |
| 181182183 | | rac+− | 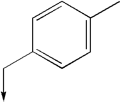 |
| 184185186 | | rac+− | 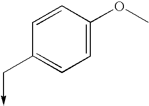 |
| 187188189 | | rac+− | 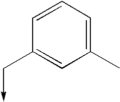 |
| 190191192 | | rac+− | 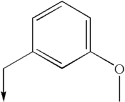 |

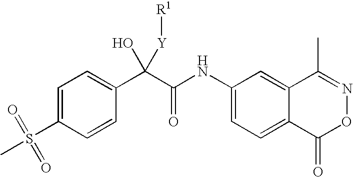

| | | Racemic or | |
|---|---|---|---|
| No. | Example | enantiomer | —Y —R¹ |
| 193194195 | | rac+− | 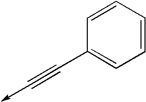 |
| 196197198 | | rac+− | 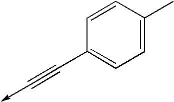 |
| 199200201 | | rac+− | 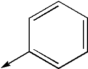 |
| 202203204 | | rac+− | 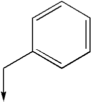 |
| 205206207 | | rac+− | 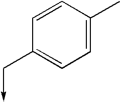 |
| 208209210 | | rac+− | 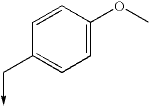 |
| 211212213 | | rac+− | 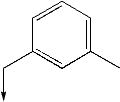 |
| 214215216 | | rac+− | 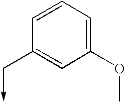 |

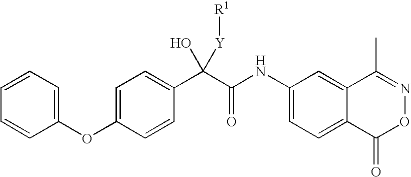

| | | Racemic or | |
|---|---|---|---|
| No. | Example | enantiomer | —Y —R¹ |
| 217218219 | | rac+− | 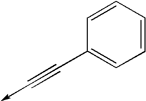 |
| 220221222 | | rac+− | 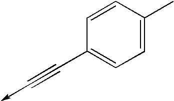 |
| 223224225 | | rac+− | 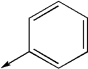 |
| 226227228 | | rac+− | 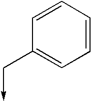 |
| 229230231 | | rac+− | 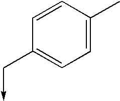 |
| 232233234 | | rac+− | 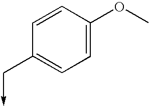 |
| 235236237 | | rac+− | 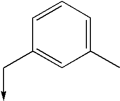 |
| 238239240 | | rac+− | 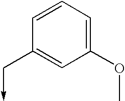 |

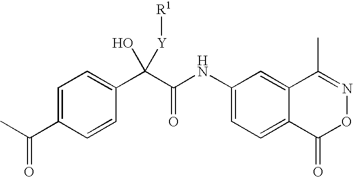

| | | Racemic or | |
|---|---|---|---|
| No. | Example | enantiomer | —Y —R¹ |
| 241 | | rac | |
| 242 | | + | |
| 243 | | − | |
| 244245246 | | rac+− | 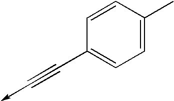 |
| 247248249 | | rac+− | 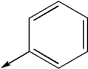 |
| 250251252 | | rac+− | 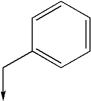 |
| 253254255 | | rac+− | 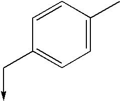 |
| 256257258 | | rac+− | 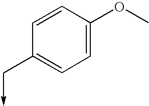 |
| 259260261 | | rac+− | 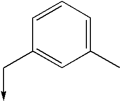 |
| 262263264 | | rac+− | 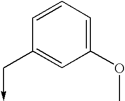 |

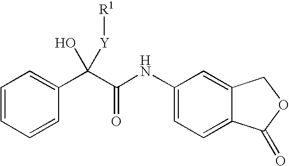

| | | Racemic or | |
|---|---|---|---|
| No. | Example | enantiomer | —Y —R¹ |
| 265266267 | 23 | rac+− | 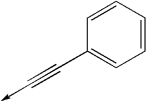 |
| 268269270 | | rac+− | 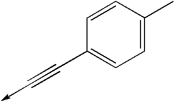 |
| 271272273 | 24 | rac+− | 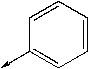 |
| 274275276 | 25 | rac+− | 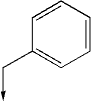 |
| 277278279 | | rac+− | 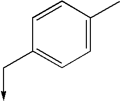 |
| 280281282 | | rac+− | 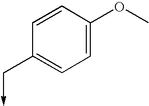 |
| 283284285 | | rac+− | 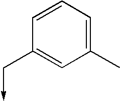 |
| 286287288 | | rac+− | 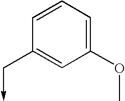 |

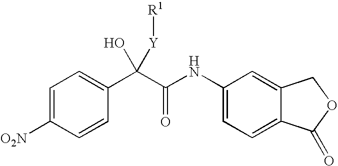

| | | Racemic or | |
|---|---|---|---|
| No. | Example | enantiomer | —Y —R¹ |
| 289 | 26 | rac | |
| 290 | | + | |
| 291 | | − | |
| 292293294 | | rac+− | 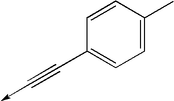 |
| 295296297 | 27 | rac+− | 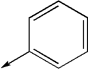 |
| 298299300 | 28 | rac+− | 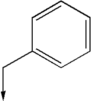 |
| 301302303 | | rac+− | 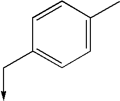 |
| 304305306 | | rac+− | 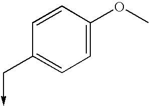 |
| 307308309 | | rac+− | 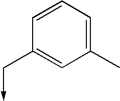 |
| 310311312 | | rac+− | 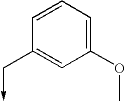 |

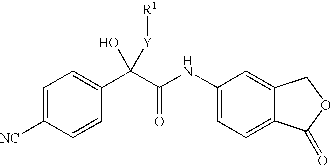

| | | Racemic or | |
|---|---|---|---|
| No. | Example | enantiomer | —Y —R¹ |
| 313 | | rac | |
| 314 | | + | |
| 315 | | − | |
| 316317318 | | rac+− | 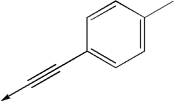 |
| 319320321 | | rac+− | 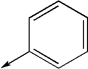 |
| 322323324 | 29 | rac+− | 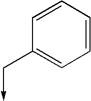 |
| 325326327 | | rac+− | 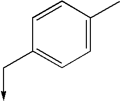 |
| 328329330 | | rac+− | 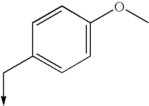 |
| 331332333 | | rac+− | 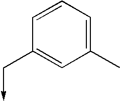 |
| 334335336 | | rac+− | 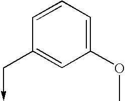 |

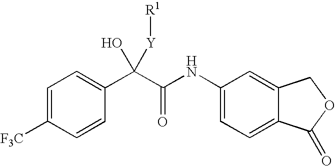

| | | Racemic or | |
|---|---|---|---|
| No. | Example | enantiomer | —Y —R¹ |
| 337338339 | | rac+− | 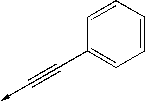 |
| 340341342 | | rac+− | 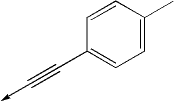 |
| 343344345 | | rac+− | 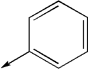 |
| 346347348 | | rac+− | 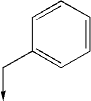 |
| 349350351 | | rac+− | 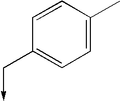 |
| 352353354 | | rac+− | 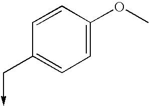 |
| 355356357 | | rac+− | 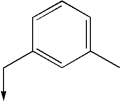 |
| 358359360 | | rac+− | 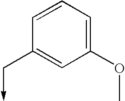 |

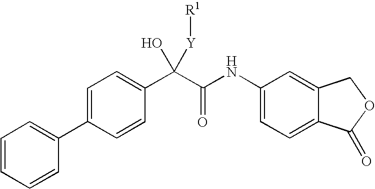

| | | Racemic or | |
|---|---|---|---|
| No. | Example | enantiomer | —Y —R¹ |
| 361 | | rac | |
| 362 | | + | |
| 363 | | − | |
| 364365366 | | rac+− | 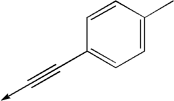 |
| 367368369 | | rac+− | 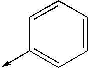 |
| 370371372 | | rac+− | 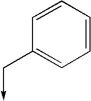 |
| 373374375 | | rac+− | 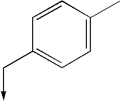 |
| 376377378 | | rac+− | 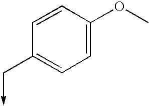 |
| 379380381 | | rac+− | 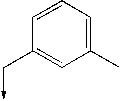 |
| 382383384 | | rac+− | 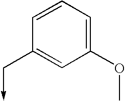 |

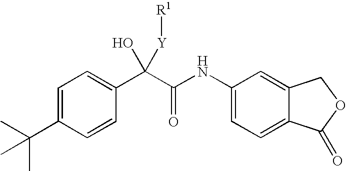

| | | Racemic or | |
|---|---|---|---|
| No. | Example | enantiomer | —Y —R¹ |
| 385 | | rac | |
| 386 | | + | |
| 387 | | − | |
| 388389390 | | rac+− | 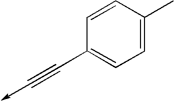 |
| 391392393 | | rac+− | 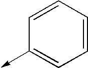 |
| 394395396 | | rac+− | 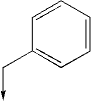 |
| 397398399 | | rac+− | 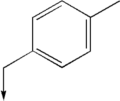 |
| 400401402 | | rac+− | 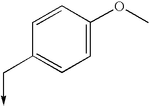 |
| 403404405 | | rac+− | 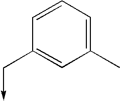 |
| 406407408 | | rac+− | 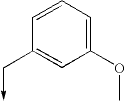 |

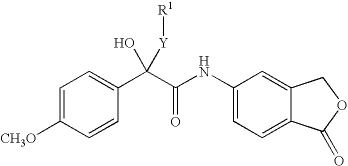

| | | Racemic or | |
|---|---|---|---|
| No. | Example | enantiomer | —Y —R¹ |
| 409410411 | | rac+− | 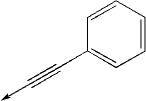 |
| 412413414 | | rac+− | 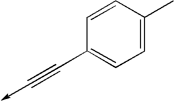 |
| 415416417 | | rac+− | 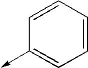 |
| 418419420 | | rac+− | 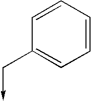 |
| 421422423 | | rac+− | 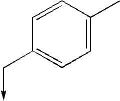 |
| 424425426 | | rac+− | 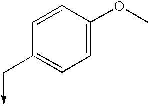 |
| 427428429 | | rac+− | 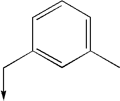 |
| 430431432 | | rac+− | 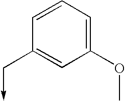 |

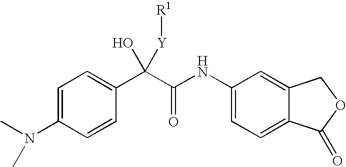

| | | Racemic or | |
|---|---|---|---|
| No. | Example | enantiomer | —Y —R¹ |
| 433434435 | | rac+− | 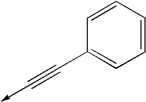 |
| 436437438 | | rac+− | 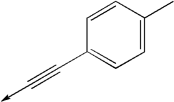 |
| 439440441 | | rac+− | 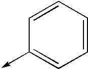 |
| 442443444 | | rac+− | 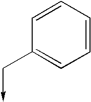 |
| 445446447 | | rac+− | 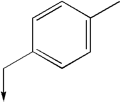 |
| 448449450 | | rac+− | 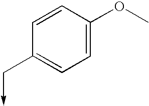 |
| 451452453 | | rac+− | 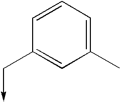 |
| 454455456 | | rac+− | 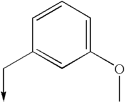 |

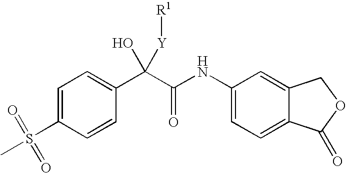

| | | Racemic or | |
|---|---|---|---|
| No. | Example | enantiomer | —Y —R¹ |
| 457 | | rac | |
| 458 | | + | |
| 459 | | − | |
| 460461462 | | rac+− | 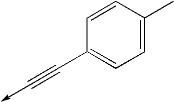 |
| 463464465 | | rac+− | 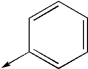 |
| 466467468 | | rac+− | 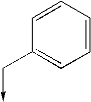 |
| 469470471 | | rac+− | 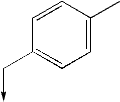 |
| 472473474 | | rac+− | 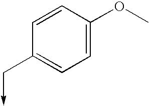 |
| 475476477 | | rac+− | 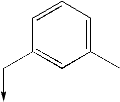 |
| 478479480 | | rac+− | 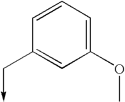 |

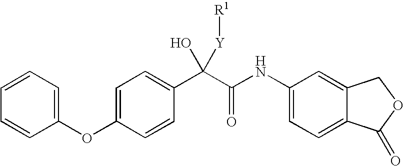

| | | Racemic or | |
|---|---|---|---|
| No. | Example | enantiomer | —Y —R¹ |
| 481 | | rac | |
| 482 | | + | |
| 483 | | − | |
| 484485486 | | rac+− | 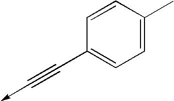 |
| 487488489 | | rac+− | 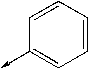 |
| 490491492 | | rac+− | 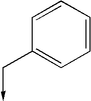 |
| 493494495 | | rac+− | 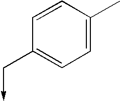 |
| 496497498 | | rac+− | 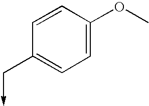 |
| 499599501 | | rac+− | 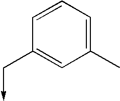 |
| 502503504 | | rac+− | 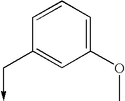 |

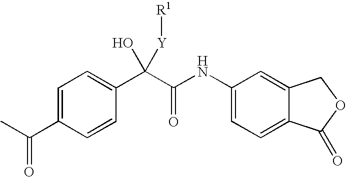

| | | Racemic or | |
|---|---|---|---|
| No. | Example | enantiomer | —Y —R¹ |
| 505 | | rac | |
| 506 | | + | |
| 507 | | − | |
| 508509510 | | rac+− | 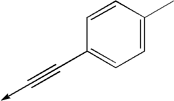 |
| 511512513 | | rac+− | 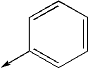 |
| 514515516 | | rac+− | 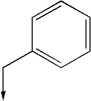 |
| 517518519 | | rac+− | 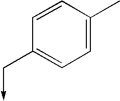 |
| 520521522 | | rac+− | 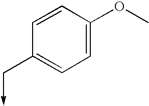 |
| 523524525 | | rac+− | 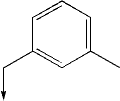 |
| 526527528 | | rac+− | 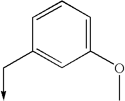 |

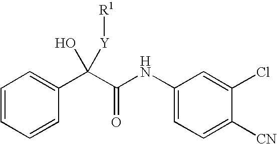

| | | Racemic or | |
|---|---|---|---|
| No. | Example | enantiomer | —Y —R¹ |
| 529530531 | | rac+− | 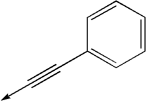 |
| 532533534 | | rac+− | 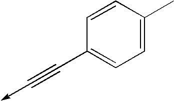 |
| 535536537 | | rac+− | 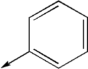 |
| 538539540 | 30 | rac+− | 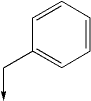 |
| 541542543 | 31 | rac+− | 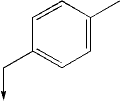 |
| 544545546 | 32 | rac+− | 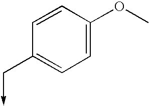 |
| 547548549 | 33 | rac+− | 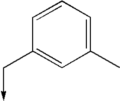 |
| 550551552 | 34 | rac+− | 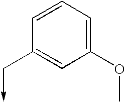 |

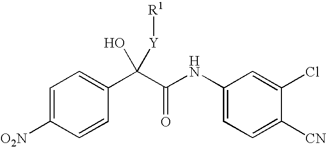

| | | Racemic or | |
|---|---|---|---|
| No. | Example | enantiomer | —Y —R¹ |
| 553 | | rac | |
| 554 | | + | |
| 555 | | − | |
| 556557558 | | rac+− | 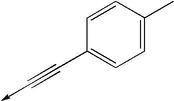 |
| 559560561 | | rac+− | 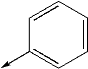 |
| 562563564 | | rac+− | 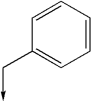 |
| 565566567 | | rac+− | 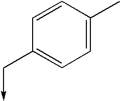 |
| 568569570 | | rac+− | 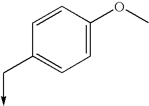 |
| 571572573 | | rac+− | 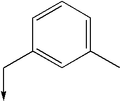 |
| 574575576 | | rac+− | 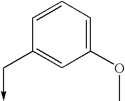 |

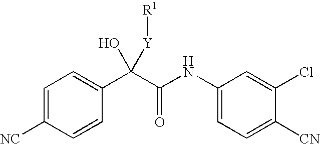

| | | Racemic or | |
|---|---|---|---|
| No. | Example | enantiomer | —Y —R¹ |
| 577578579 | | rac+− | 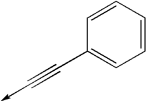 |
| 580581582 | | rac+− | 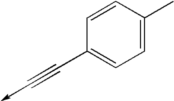 |
| 583584585 | | rac+− | 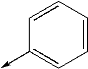 |
| 586587588 | | rac+− | 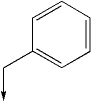 |
| 589590591 | | rac+− | 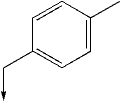 |
| 592593594 | | rac+− | 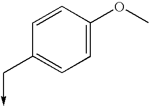 |
| 595596597 | | rac+− | 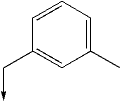 |
| 598599600 | | rac+− | 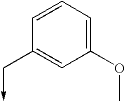 |

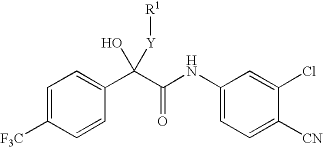

| | | Racemic or | |
|---|---|---|---|
| No. | Example | enantiomer | —Y—R¹ |
| 601 | | rac | |
| 602 | | + | |
| 603 | | − | |
| 604605606 | | rac+− | 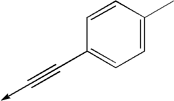 |
| 607608609 | | rac+− | 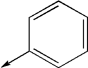 |
| 610611612 | | rac+− | 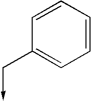 |
| 613614615 | | rac+− | 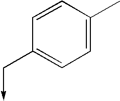 |
| 616617618 | | rac+− | 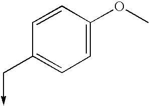 |
| 619620621 | | rac+− | 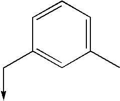 |
| 622623624 | | rac+− | 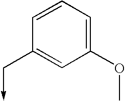 |

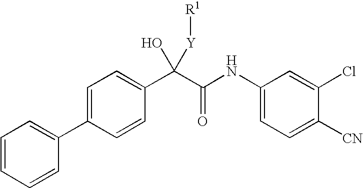

| | | Racemic or | |
|---|---|---|---|
| No. | Example | enantiomer | —Y—R¹ |
| 625626627 | | rac+− | 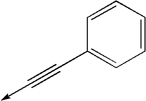 |
| 628629630 | | rac+− | 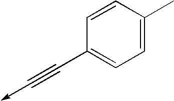 |
| 631632633 | | rac+− | 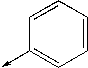 |
| 634635636 | | rac+− | 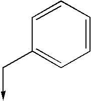 |
| 637638639 | | rac+− | 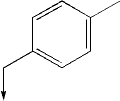 |
| 640641642 | | rac+− | 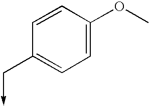 |
| 643644645 | | rac+− | 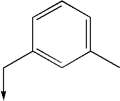 |
| 646647648 | | rac+− | 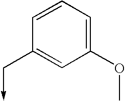 |

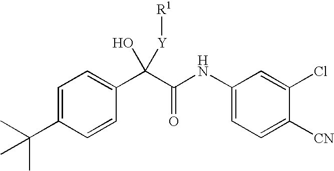

| | | Racemic or | |
|---|---|---|---|
| No. | Example | enantiomer | —Y—R¹ |
| 649 | | rac | |
| 650 | | + | |
| 651 | | − | |
| 652653654 | | rac+− | 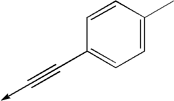 |
| 655656657 | | rac+− | 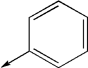 |
| 658659660 | | rac+− | 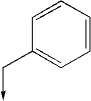 |
| 661662663 | | rac+− | 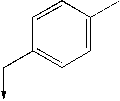 |
| 664665666 | | rac+− | 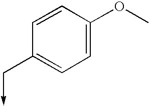 |
| 667668669 | | rac+− | 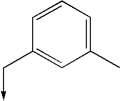 |
| 670671672 | | rac+− | 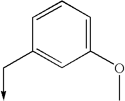 |

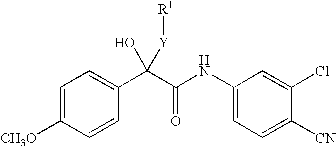

| | | Racemic or | |
|---|---|---|---|
| No. | Example | enantiomer | —Y—R¹ |
| 673674675 | | rac+− | 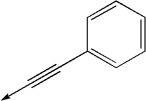 |
| 676677678 | | rac+− | 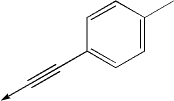 |
| 679680681 | | rac+− | 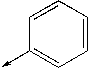 |
| 682683684 | | rac+− | 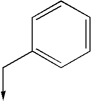 |
| 685686687 | | rac+− | 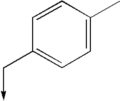 |
| 688689690 | | rac+− | 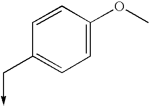 |
| 691692693 | | rac+− | 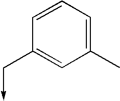 |
| 694695696 | | rac+− | 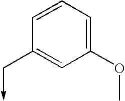 |

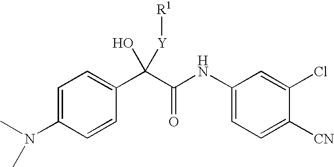

| | | Racemic or | |
|---|---|---|---|
| No. | Example | enantiomer | —Y—R¹ |
| 697698699 | | rac+− | 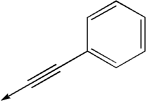 |
| 700701702 | | rac+− | 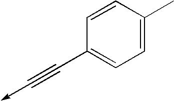 |
| 703704705 | | rac+− | 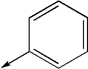 |
| 706707708 | | rac+− | 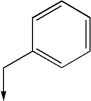 |
| 709710711 | | rac+− | 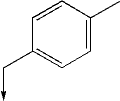 |
| 712713714 | | rac+− | 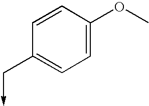 |
| 715716717 | | rac+− | 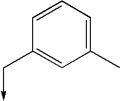 |
| 718719720 | | rac+− | 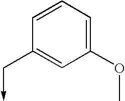 |

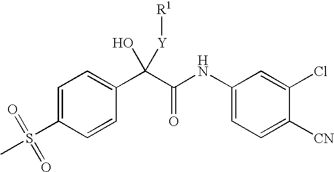

| | | Racemic or | |
|---|---|---|---|
| No. | Example | enantiomer | —Y—R¹ |
| 721 | | rac | |
| 722 | | + | |
| 723 | | − | |
| 724725726 | | rac+− | 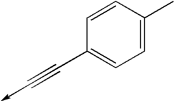 |
| 727728729 | | rac+− | 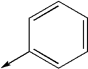 |
| 730731732 | | rac+− | 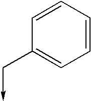 |
| 733734735 | | rac+− | 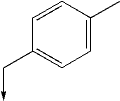 |
| 736737738 | | rac+− | 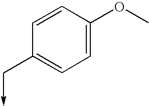 |
| 739740741 | | rac+− | 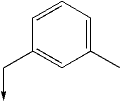 |
| 742743744 | | rac+− | 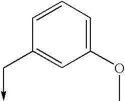 |

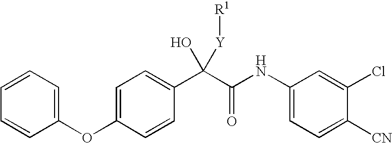

| | | Racemic or | |
|---|---|---|---|
| No. | Example | enantiomer | —Y—R¹ |
| 745746747 | | rac+− | 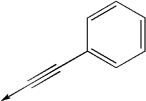 |
| 748749750 | | rac+− | 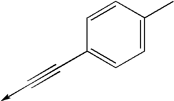 |
| 751752753 | | rac+− | 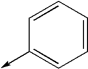 |
| 754755756 | | rac+− | 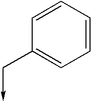 |
| 757758759 | | rac+− | 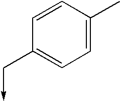 |
| 760761762 | | rac+− | 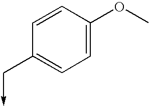 |
| 763764765 | | rac+− | 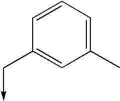 |
| 766767768 | | rac+− | 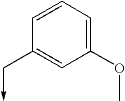 |

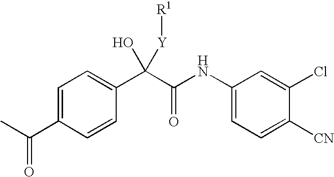

| | | Racemic or | |
|---|---|---|---|
| No. | Example | enantiomer | —Y—R¹ |
| 769 | | rac | |
| 770 | | + | |
| 771 | | − | |
| 772773774 | | rac+− | 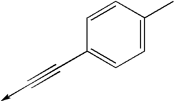 |
| 775776777 | | rac+− | 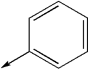 |
| 778779780 | | rac+− | 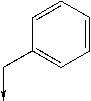 |
| 781782783 | | rac+− | 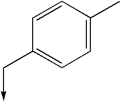 |
| 784785786 | | rac+− | 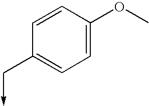 |
| 787788789 | | rac+− | 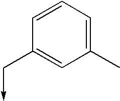 |
| 790791792 | | rac+− | 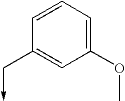 |

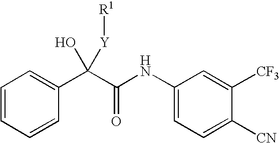

| | | Racemic or | |
|---|---|---|---|
| No. | Example | enantiomer | —Y—R¹ |
| 793794795 | | rac+− | 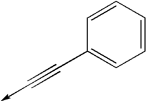 |
| 796797798 | | rac+− | 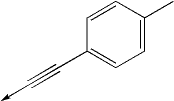 |
| 799800801 | | rac+− | 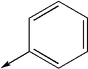 |
| 802803804 | | rac+− | 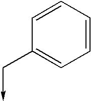 |
| 805806807 | | rac+− | 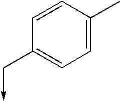 |
| 808809810 | | rac+− | 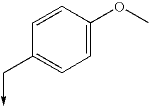 |
| 811812813 | | rac+− | 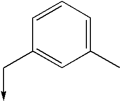 |
| 814815816 | | rac+− | 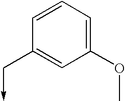 |

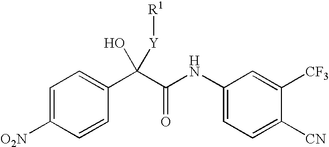

| | | Racemic or | |
|---|---|---|---|
| No. | Example | enantiomer | —Y—R¹ |
| 817818819 | | rac+− | 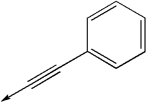 |
| 820821822 | | rac+− | 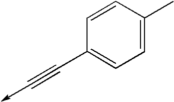 |
| 823824825 | | rac+− | 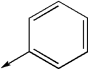 |
| 826827828 | | rac+− | 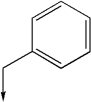 |
| 829830831 | | rac+− | 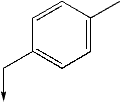 |
| 832833834 | | rac+− | 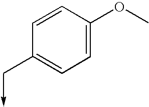 |
| 835836837 | | rac+− | 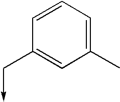 |
| 838839840 | | rac+− | 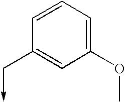 |

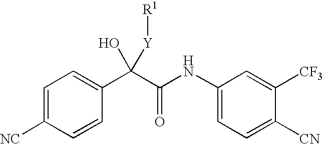

| | | Racemic or | |
|---|---|---|---|
| No. | Example | enantiomer | —Y—R¹ |
| 841842843 | | rac+− | 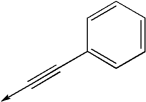 |
| 844845846 | | rac+− | 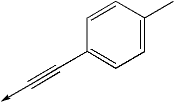 |
| 847848849 | | rac+− | 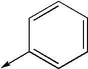 |
| 850851852 | | rac+− | 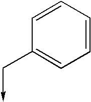 |
| 853854855 | | rac+− | 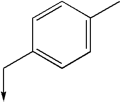 |
| 856857858 | | rac+− | 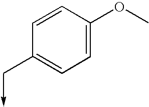 |
| 859860861 | | rac+− | 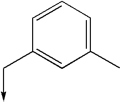 |
| 862863864 | | rac+− | 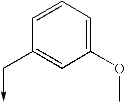 |

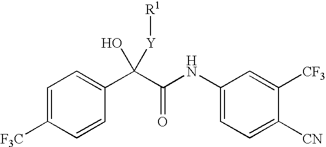

| | | Racemic or | |
|---|---|---|---|
| No. | Example | enantiomer | —Y—R¹ |
| 865 | | rac | |
| 866 | | + | |
| 867 | | − | |
| 868869870 | | rac+− | 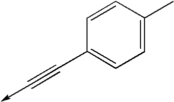 |
| 871872873 | | rac+− | 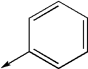 |
| 874875876 | | rac+− | 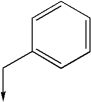 |
| 877878879 | | rac+− | 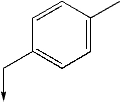 |
| 880881882 | | rac+− | 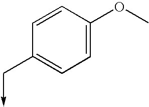 |
| 883884885 | | rac+− | 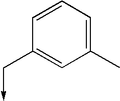 |
| 886887888 | | rac+− | 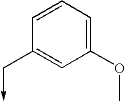 |

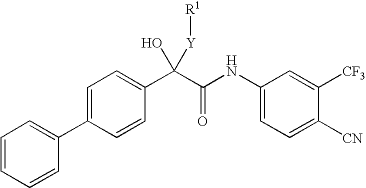

| | | Racemic or | |
|---|---|---|---|
| No. | Example | enantiomer | —Y—R¹ |
| 889890891 | | rac+− | 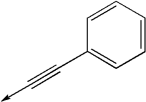 |
| 892893894 | | rac+− | 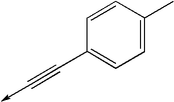 |
| 895896897 | | rac+− | 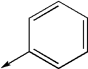 |
| 898899900 | | rac+− | 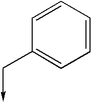 |
| 901902903 | | rac+− | 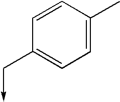 |
| 904905906 | | rac+− | 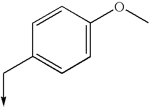 |
| 907908909 | | rac+− | 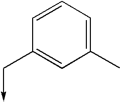 |
| 910911912 | | rac+− | 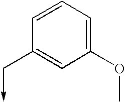 |

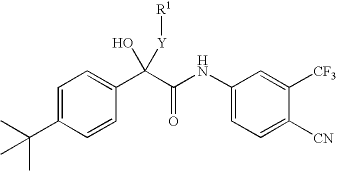

| | | Racemic or | |
|---|---|---|---|
| No. | Example | enantiomer | —Y—R¹ |
| 913914915 | | rac+− | 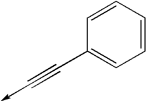 |
| 916917918 | | rac+− | 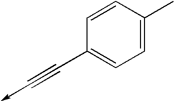 |
| 919920921 | | rac+− | 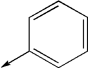 |
| 922923924 | | rac+− | 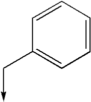 |
| 925926927 | | rac+− | 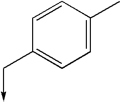 |
| 928929930 | | rac+− | 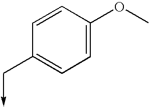 |
| 931932933 | | rac+− | 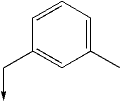 |
| 934935936 | | rac+− | 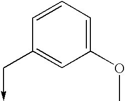 |

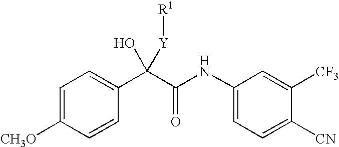

| | | Racemic or | |
|---|---|---|---|
| No. | Example | enantiomer | —Y—R¹ |
| 937 | | rac | |
| 938 | | + | |
| 939 | | − | |
| 940941942 | | rac+− | 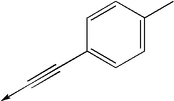 |
| 943944945 | | rac+− | 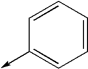 |
| 946947948 | | rac+− | 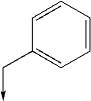 |
| 949950951 | | rac+− | 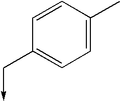 |
| 952953954 | | rac+− | 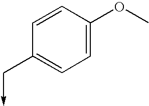 |
| 955956957 | | rac+− | 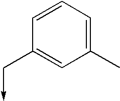 |
| 958959960 | | rac+− | 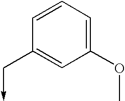 |

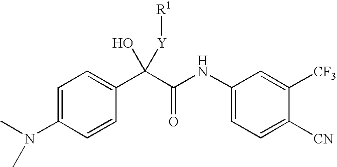

| | | Racemic or | |
|---|---|---|---|
| No. | Example | enantiomer | —Y—R¹ |
| 961962963 | | rac+− | 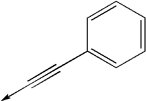 |
| 964965966 | | rac+− | 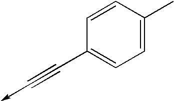 |
| 967968969 | | rac+− | 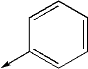 |
| 970971972 | | rac+− | 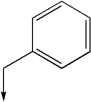 |
| 973974975 | | rac+− | 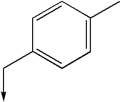 |
| 976977978 | | rac+− | 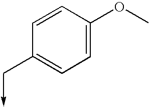 |
| 979980981 | | rac+− | 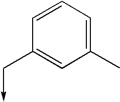 |
| 982983984 | | rac+− | 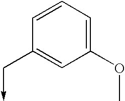 |

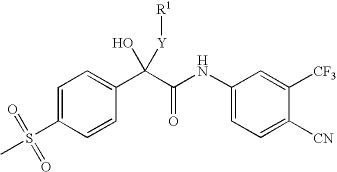

| | | Racemic or | |
|---|---|---|---|
| No. | Example | enantiomer | —Y—R¹ |
| 985 | | rac | |
| 986 | | + | |
| 987 | | − | |
| 988 989 990 | | rac+− | 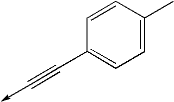 |
| 991 992 993 | | rac+− | 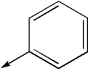 |
| 994 995 996 | | rac+− | 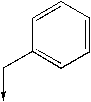 |
| 997 998 999 | | rac+− | 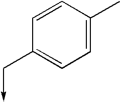 |
| 100010011002 | | rac+− | 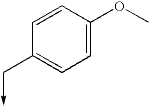 |
| 100310041005 | | rac+− | 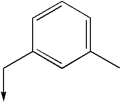 |
| 100610071008 | | rac+− | 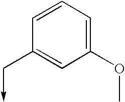 |

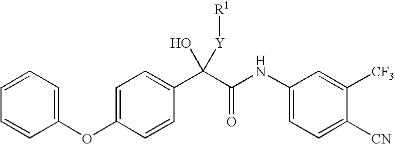

| | | Racemic or | |
|---|---|---|---|
| No. | Example | enantiomer | —Y—R¹ |
| 100910101011 | | rac+− | 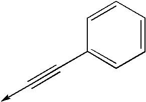 |
| 101210131014 | | rac+− | 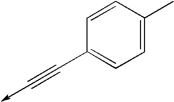 |
| 101510161017 | | rac+− | 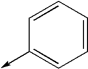 |
| 101810191020 | | rac+− | 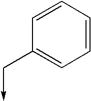 |
| 102110221023 | | rac+− | 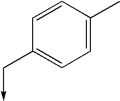 |
| 102410251026 | | rac+− | 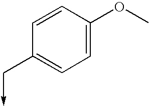 |
| 102710281029 | | rac+− | 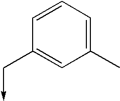 |
| 103010311032 | | rac+− | 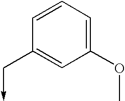 |

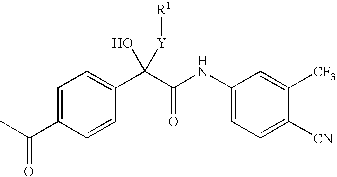

| | | Racemic or | |
|---|---|---|---|
| No. | Example | enantiomer | —Y—R¹ |
| 103310341035 | | rac+− | 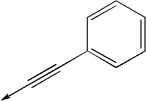 |
| 103610371038 | | rac+− | 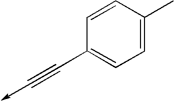 |
| 103910401041 | | rac+− | 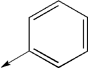 |
| 104210431044 | | rac+− | 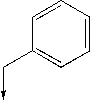 |
| 104510461047 | | rac+− | 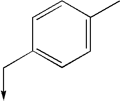 |
| 104810491050 | | rac+− | 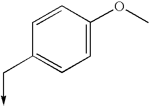 |
| 105110521053 | | rac+− | 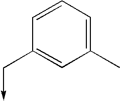 |
| 105410551056 | | rac+− | 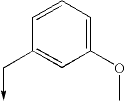 |

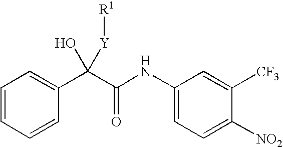

| | | Racemic or | |
|---|---|---|---|
| No. | Example | enantiomer | —Y—R¹ |
| 105710581059 | | rac+− | 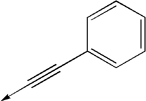 |
| 106010611062 | | rac+− | 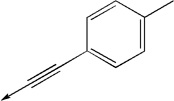 |
| 106310641065 | | rac+− | 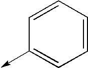 |
| 106610671068 | | rac+− | 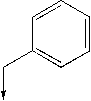 |
| 106910701071 | | rac+− | 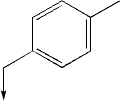 |
| 107210731074 | | rac+− | 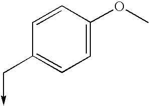 |
| 107510761077 | | rac+− | 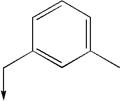 |
| 107810791080 | | rac+− | 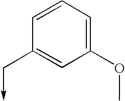 |

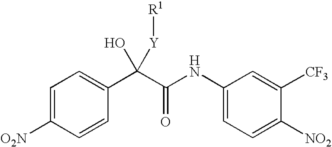

| | | Racemic or | |
|---|---|---|---|
| No. | Example | enantiomer | —Y—R¹ |
| 1081 | | rac | |
| 1082 | | + | |
| 1083 | | − | |
| 108410851086 | | rac+− | 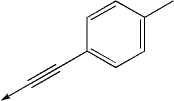 |
| 108710881089 | | rac+− | 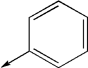 |
| 109010911092 | | rac+− | 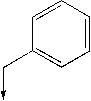 |
| 109310941095 | | rac+− | 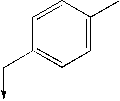 |
| 109610971098 | | rac+− | 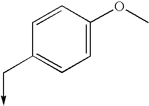 |
| 109911001101 | | rac+− | 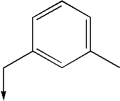 |
| 110211031104 | | rac+− | 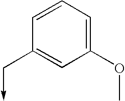 |

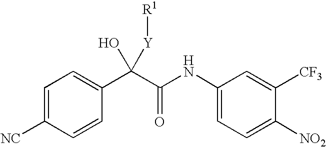

| | | Racemic or | |
|---|---|---|---|
| No. | Example | enantiomer | —Y—R¹ |
| 1105 | | rac | |
| 1106 | | + | |
| 1107 | | − | |
| 110811091110 | | rac+− | 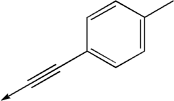 |
| 111111121113 | | rac+− | 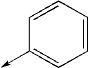 |
| 111411151116 | | rac+− | 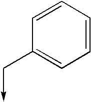 |
| 111711181119 | | rac+− | 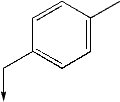 |
| 112011211122 | | rac+− | 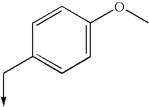 |
| 112311241125 | | rac+− | 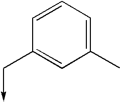 |
| 112611271128 | | rac+− | 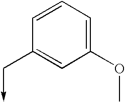 |

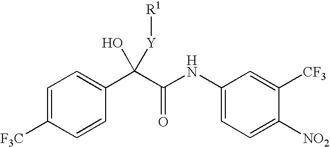

| | | Racemic or | |
|---|---|---|---|
| No. | Example | enantiomer | —Y—R¹ |
| 112911301131 | | rac+− | 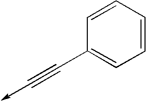 |
| 113211331134 | | rac+− | 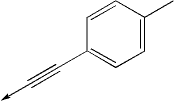 |
| 113511361137 | | rac+− | 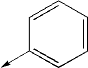 |
| 113811391140 | | rac+− | 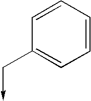 |
| 114111421143 | | rac+− | 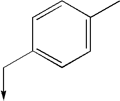 |
| 114411451146 | | rac+− | 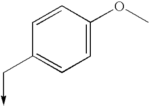 |
| 114711481149 | | rac+− | 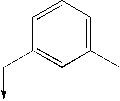 |
| 115011511152 | | rac+− | 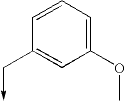 |

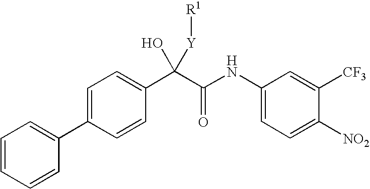

| | | Racemic or | |
|---|---|---|---|
| No. | Example | enantiomer | —Y—R¹ |
| 115311541155 | | rac+− | 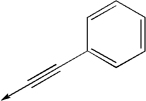 |
| 115611571158 | | rac+− | 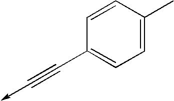 |
| 115911601161 | | rac+− | 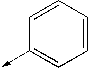 |
| 116211631164 | | rac+− | 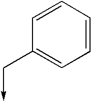 |
| 116511661167 | | rac+− | 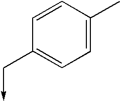 |
| 116811691170 | | rac+− | 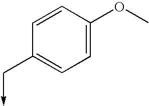 |
| 117111721173 | | rac+− | 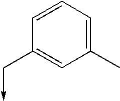 |
| 117411751176 | | rac+− | 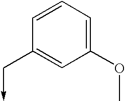 |

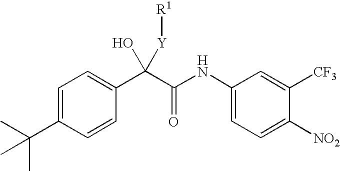

| | | Racemic or | |
|---|---|---|---|
| No. | Example | enantiomer | —Y—R¹ |
| 1177 | | rac | |
| 1178 | | + | |
| 1179 | | − | |
| 118011811182 | | rac+− | 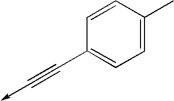 |
| 118311841185 | | rac+− | 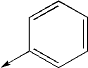 |
| 118611871188 | | rac+− | 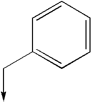 |
| 118911901191 | | rac+− | 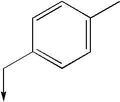 |
| 119211931194 | | rac+− | 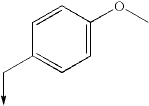 |
| 119511961197 | | rac+− | 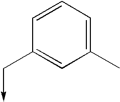 |
| 119811991200 | | rac+− | 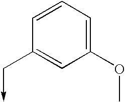 |

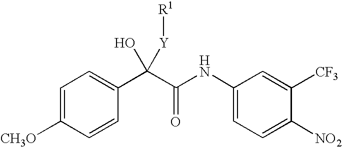

| | | Racemic or | |
|---|---|---|---|
| No. | Example | enantiomer | —Y—R¹ |
| 1201 | | rac | |
| 1202 | | + | |
| 1203 | | − | |
| 120412051206 | | rac+− | 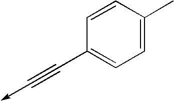 |
| 120712081209 | | rac+− | 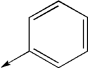 |
| 121012111212 | | rac+− | 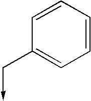 |
| 121312141215 | | rac+− | 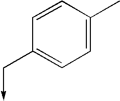 |
| 121612171218 | | rac+− | 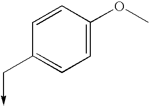 |
| 121912201221 | | rac+− | 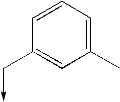 |
| 122212231224 | | rac+− | 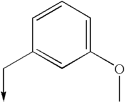 |

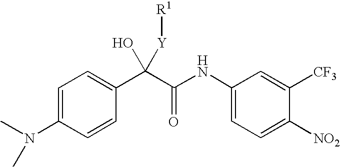

| | | Racemic or | |
|---|---|---|---|
| No. | Example | enantiomer | —Y—R¹ |
| 122512261227 | | rac+− | 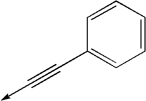 |
| 122812291230 | | rac+− | 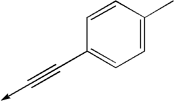 |
| 123112321233 | | rac+− | 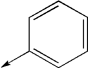 |
| 123412351236 | | rac+− | 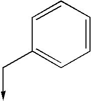 |
| 123712381239 | | rac+− | 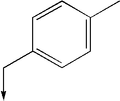 |
| 124012411242 | | rac+− | 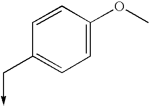 |
| 124312441245 | | rac+− | 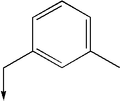 |
| 124612471248 | | rac+− | 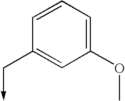 |

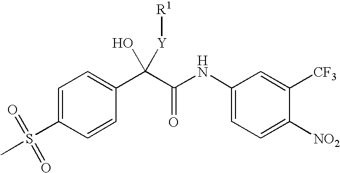

| | | Racemic or | |
|---|---|---|---|
| No. | Example | enantiomer | —Y—R¹ |
| 1249 | | rac | |
| 1250 | | + | |
| 1251 | | − | |
| 125212531254 | | rac+− | 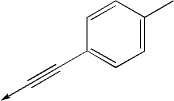 |
| 125512561257 | | rac+− | 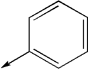 |
| 125812591260 | | rac+− | 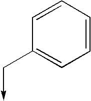 |
| 126112621263 | | rac+− | 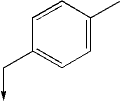 |
| 126412651266 | | rac+− | 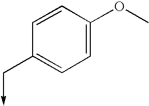 |
| 126712681269 | | rac+− | 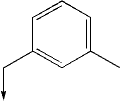 |
| 127012711272 | | rac+− | 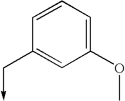 |

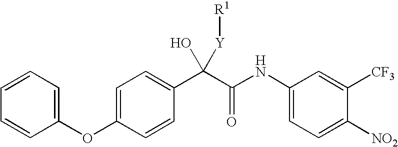

| | | Racemic or | |
|---|---|---|---|
| No. | Example | enantiomer | —Y—R¹ |
| 127312741275 | | rac+− | 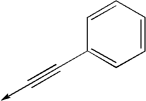 |
| 127612771278 | | rac+− | 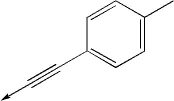 |
| 127912801281 | | rac+− | 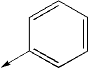 |
| 128212831284 | | rac+− | 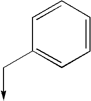 |
| 128512861287 | | rac+− | 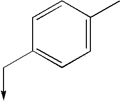 |
| 128812891290 | | rac+− | 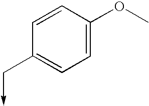 |
| 129112921293 | | rac+− | 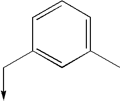 |
| 129412951296 | | rac+− | 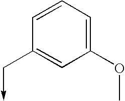 |

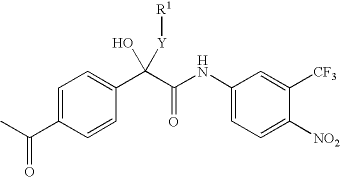

| | | Racemic or | |
|---|---|---|---|
| No. | Example | enantiomer | —Y—R¹ |
| 1297 | | rac | |
| 1298 | | + | |
| 1299 | | − | |
| 130013011302 | | rac+− | 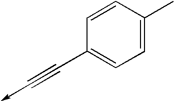 |
| 130313041305 | | rac+− | 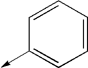 |
| 130613071308 | | rac+− | 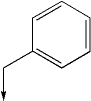 |
| 130913101311 | | rac+− | 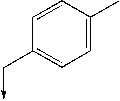 |
| 131213131314 | | rac+− | 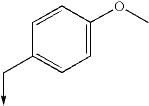 |
| 131513161317 | | rac+− | 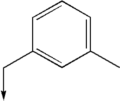 |
| 131813191320 | | rac+− | 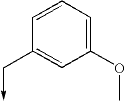 |

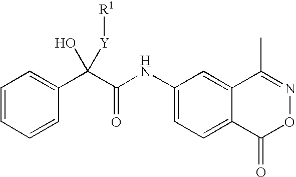

| | | Racemic or | |
|---|---|---|---|
| No. | Example | enantiomer | —Y—R¹ |
| 132113221323 | | rac+− | 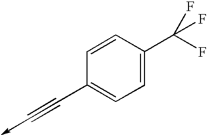 |
| 132413251326 | | rac+− | 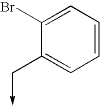 |
| 132713281329 | | rac+− | 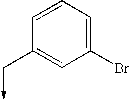 |
| 133013311332 | | rac+− | 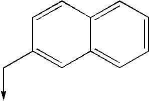 |
| 133313341335 | | rac+− | 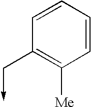 |
| 133613371338 | | rac+− | 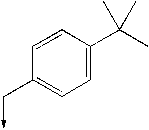 |
| 133913401341 | | rac+− | 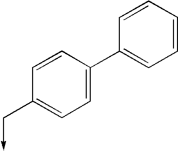 |

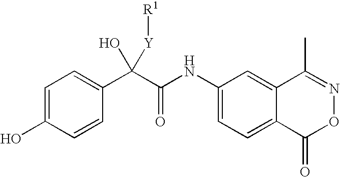

| | | Racemic or | |
|---|---|---|---|
| No. | Example | enantiomer | —Y—R¹ |
| 134213431344 | | rac+− | 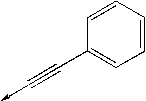 |
| 134513461347 | | rac+− | 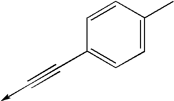 |
| 134813491350 | | rac+− | 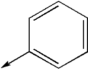 |
| 135113521353 | | rac+− | 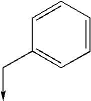 |
| 135413551356 | | rac+− | 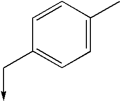 |
| 135713581359 | | rac+− | 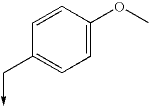 |
| 136013611362 | | rac+− | 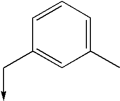 |
| 136313641365 | | rac+− | 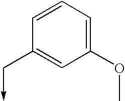 |

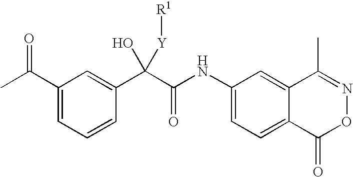

| | | Racemic or | |
|---|---|---|---|
| No. | Example | enantiomer | —Y—R¹ |
| 136613671368 | | rac+− | 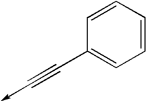 |
| 136913701371 | | rac+− | 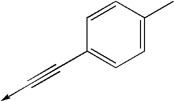 |
| 137213731374 | | rac+− | 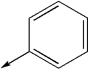 |
| 137513761377 | | rac+− | 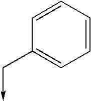 |
| 137813791380 | | rac+− | 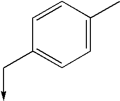 |
| 138113821383 | | rac+− | 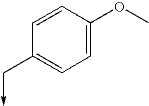 |
| 138413851386 | | rac+− | 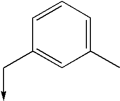 |
| 138713881389 | | rac+− | 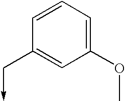 |

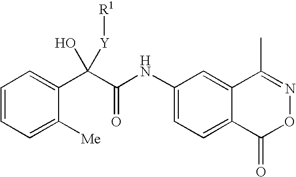

| | | Racemic or | |
|---|---|---|---|
| No. | Example | enantiomer | —Y—R¹ |
| 139013911392 | | rac+− | 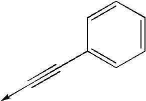 |
| 139313941395 | | rac+− | 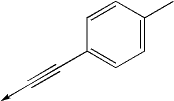 |
| 139613971398 | | rac+− | 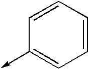 |
| 139914001401 | | rac+− | 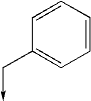 |
| 140214031404 | | rac+− | 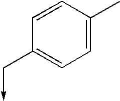 |
| 140514061407 | | rac+− | 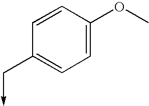 |
| 140814091410 | | rac+− | 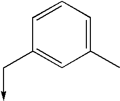 |
| 141114121413 | | rac+− | 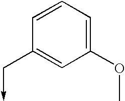 |

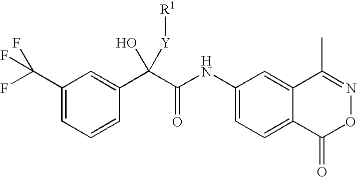

| | | Racemic or | |
|---|---|---|---|
| No. | Example | enantiomer | —Y—R¹ |
| 141414151416 | | rac+− | 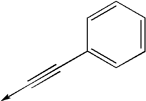 |
| 141714181419 | | rac+− | 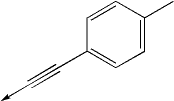 |
| 142014211422 | | rac+− | 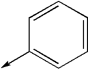 |
| 142314241425 | | rac+− | 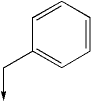 |
| 142614271428 | | rac+− | 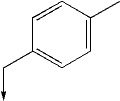 |
| 142914301431 | | rac+− | 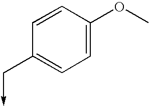 |
| 143214331434 | | rac+− | 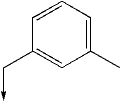 |
| 143514361437 | | rac+− | 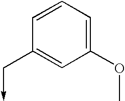 |

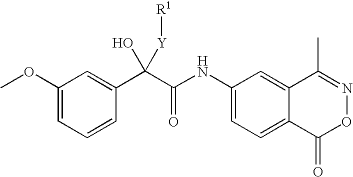

| | | Racemic or | |
|---|---|---|---|
| No. | Example | enantiomer | —Y—R¹ |
| 143814391440 | | rac+− | 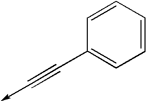 |
| 144114421443 | | rac+− | 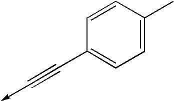 |
| 144414451446 | | rac+− | 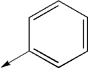 |
| 144714481449 | | rac+− | 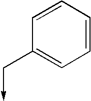 |
| 145014511452 | | rac+− | 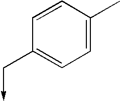 |
| 145314541455 | | rac+− | 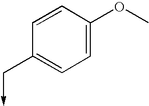 |
| 145614571458 | | rac+− | 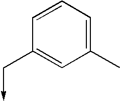 |
| 145914601461 | | rac+− | 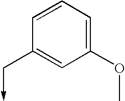 |

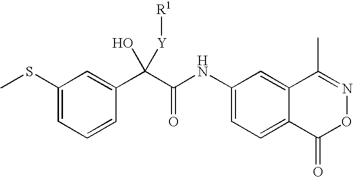

| | | Racemic or | |
|---|---|---|---|
| No. | Example | enantiomer | —Y—R¹ |
| 146214631464 | | rac+− | 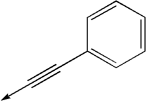 |
| 146514661467 | | rac+− | 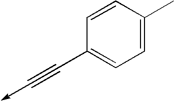 |
| 146814691470 | | rac+− | 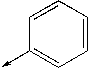 |
| 147114721473 | | rac+− | 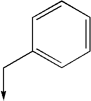 |
| 147414751476 | | rac+− | 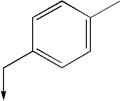 |
| 147714781479 | | rac+− | 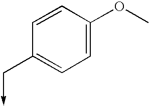 |
| 148014811482 | | rac+− | 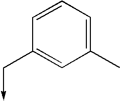 |
| 148314841485 | | rac+− | 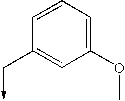 |

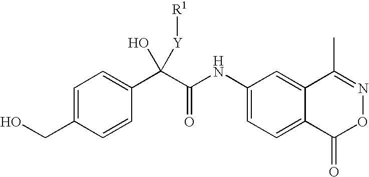

| | | Racemic or | |
|---|---|---|---|
| No. | Example | enantiomer | —Y—R¹ |
| 148614871488 | | rac+− | 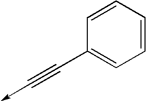 |
| 148914901491 | | rac+− | 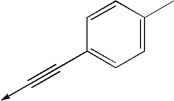 |
| 149214931494 | | rac+− | 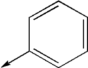 |
| 149514961497 | | rac+− | 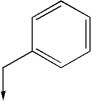 |
| 149814991500 | | rac+− | 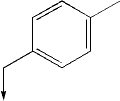 |
| 150115021503 | | rac+− | 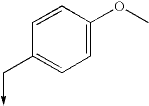 |
| 150415051506 | | rac+− | 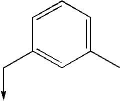 |
| 150715081509 | | rac+− | 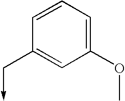 |

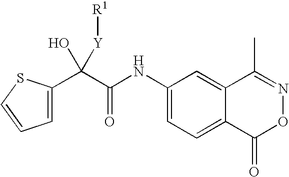

| | | Racemic or | |
|---|---|---|---|
| No. | Example | enantiomer | —Y—R¹ |
| 151015111512 | | rac+− | 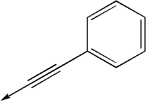 |
| 151315141515 | | rac+− | 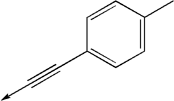 |
| 151615171518 | | rac+− | 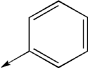 |
| 151915201521 | | rac+− | 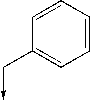 |
| 152215231524 | | rac+− | 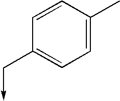 |
| 152515261527 | | rac+− | 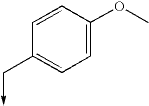 |
| 152815291530 | | rac+− | 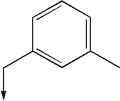 |
| 153115321533 | | rac+− | 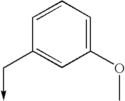 |

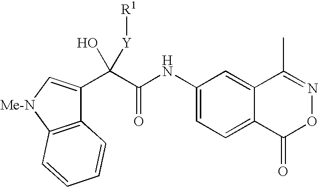

| | | Racemic or | |
|---|---|---|---|
| No. | Example | enantiomer | —Y—R¹ |
| 153415351536 | | rac+− | 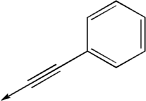 |
| 153715381539 | | rac+− | 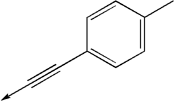 |
| 154015411542 | | rac+− | 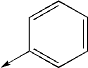 |
| 154315441545 | | rac+− | 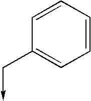 |
| 154615471548 | | rac+− | 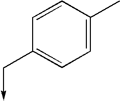 |
| 154915501551 | | rac+− | 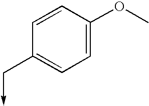 |
| 155215531554 | | rac+− | 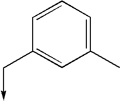 |
| 155515561557 | | rac+− | 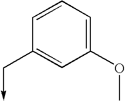 |

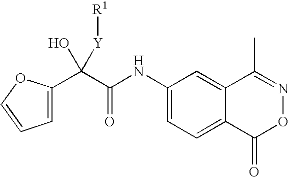

| | | Racemic or | |
|---|---|---|---|
| No. | Example | enantiomer | —Y—R¹ |
| 155815591560 | | rac+− | 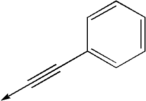 |
| 156115621563 | | rac+− | 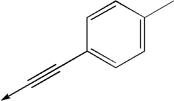 |
| 156415651566 | | rac+− | 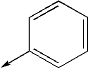 |
| 156715681569 | | rac+− | 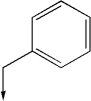 |
| 157015711572 | | rac+− | 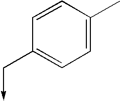 |
| 157315741575 | | rac+− | 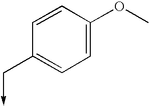 |
| 157615771578 | | rac+− | 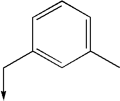 |
| 157915801581 | | rac+− | 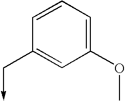 |

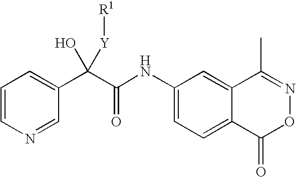

| | | Racemic or | |
|---|---|---|---|
| No. | Example | enantiomer | —Y—R¹ |
| 158215831584 | | rac+− | 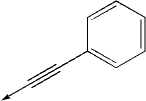 |
| 158515861587 | | rac+− | 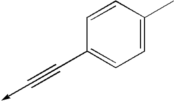 |
| 158815891590 | | rac+− | 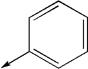 |
| 159115921593 | | rac+− | 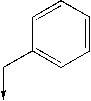 |
| 159415951596 | | rac+− | 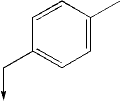 |
| 159715981599 | | rac+− | 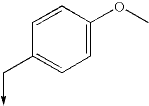 |
| 160016011602 | | rac+− | 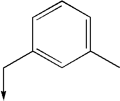 |
| 160316041605 | | rac+− | 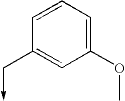 |

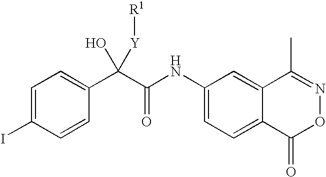

| | | Racemic or | |
|---|---|---|---|
| No. | Example | enantiomer | —Y—R¹ |
| 160616071608 | | rac+− | 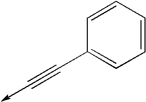 |
| 160916101611 | | rac+− | 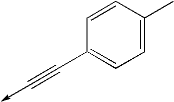 |
| 161216131614 | | rac+− | 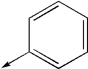 |
| 161516161617 | | rac+− | 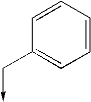 |
| 161816191620 | | rac+− | 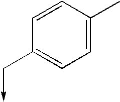 |
| 162116221623 | | rac+− | 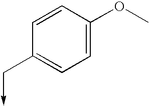 |
| 162416251626 | | rac+− | 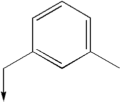 |
| 162716281629 | | rac+− | 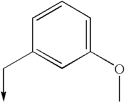 |

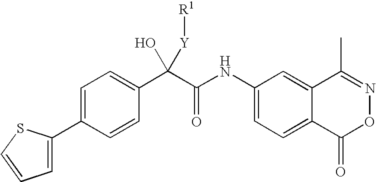

| | | Racemic or | |
|---|---|---|---|
| No. | Example | enantiomer | —Y—R¹ |
| 163016311632 | | rac+− | 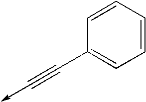 |
| 163316341635 | | rac+− | 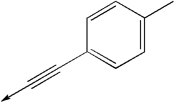 |
| 163616371638 | | rac+− | 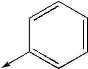 |
| 163916401641 | | rac+− | 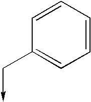 |
| 164216431644 | | rac+− | 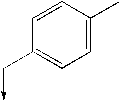 |
| 164516461647 | | rac+− | 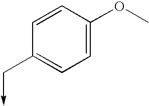 |
| 164816491650 | | rac+− | 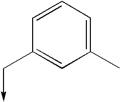 |
| 165116521653 | | rac+− | 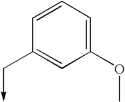 |

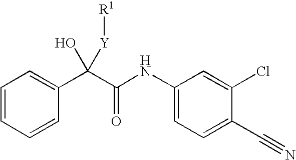

| | | Racemic or | |
|---|---|---|---|
| No. | Example | enantiomer | —Y—R¹ |
| 165416551656 | | rac+− | 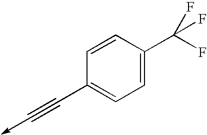 |
| 165716581659 | | rac+− | 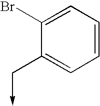 |
| 166016611662 | | rac+− | 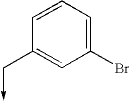 |
| 166316641665 | | rac+− | 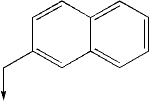 |
| 166616671668 | | rac+− | 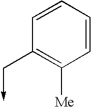 |
| 166916701671 | | rac+− | 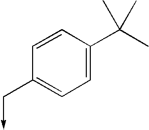 |
| 167216731674 | | rac+− | 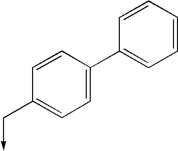 |
| 167516761677 | | rac+− | 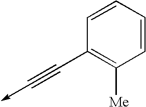 |
| 167816791680 | | rac+− | 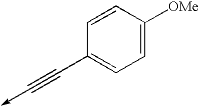 |
| 168116821683 | | rac+− | 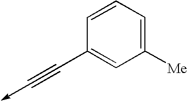 |
| 168416851686 | | rac+− | 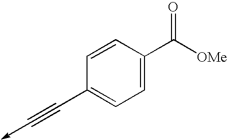 |
| 168716881689 | | rac+− | 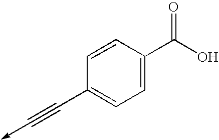 |

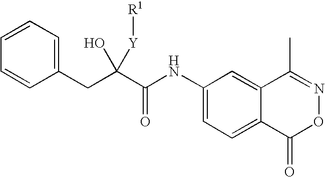

| | | Racemic or | |
|---|---|---|---|
| No. | Example | enantiomer | —Y—R¹ |
| 169016911692 | | rac+− | 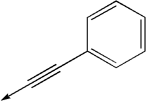 |
| 169316941695 | | rac+− | 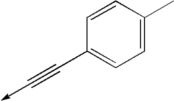 |
| 169616971698 | | rac+− | 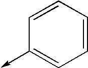 |
| 169917001701 | | rac+− | 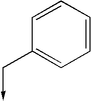 |
| 170217031704 | | rac+− | 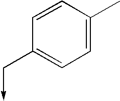 |
| 170517061707 | | rac+− | 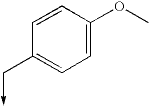 |
| 170817091710 | | rac+− | 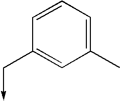 |
| 171117121713 | | rac+− | 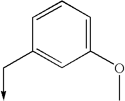 |

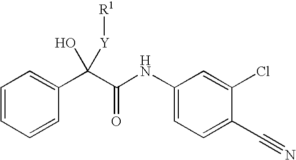

| | Ex- | | |
|---|---|---|---|
| | am- | Racemic or | |
| No. | ple | enantiomer | —Y—R¹ |
| 171417151716 | 86 | rac+− | 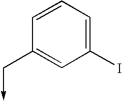 |
| 171717181719 | 97 | rac+− | 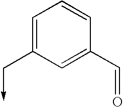 |
| 172017211722 | 90 | rac+− | 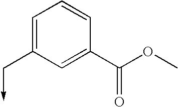 |
| 172317241725 | 92 | rac+− | 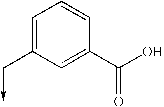 |
| 172617271728 | 94 | rac+− | 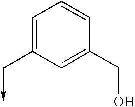 |
| 172917301731 | 87 | rac+− | 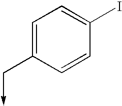 |
| 173217331734 | 88 | rac+− | 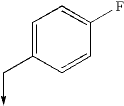 |
| 173517361737 | 93 | rac+− | 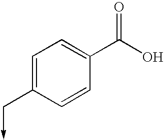 |
| 173817391740 | 98 | rac+− | 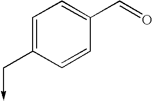 |
| 174117421743 | 91 | rac+− | 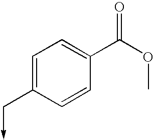 |
| 174417451746 | 100 | rac+− | 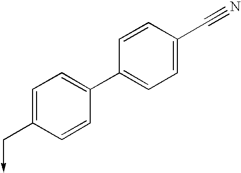 |
| 174717481749 | 103 | rac+− | 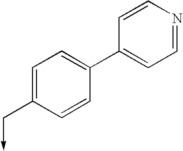 |
| 175017511753 | 109 | rac+− | 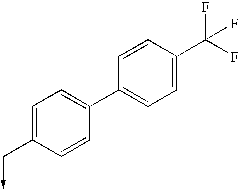 |
| 175417551756 | 112 | rac+− | 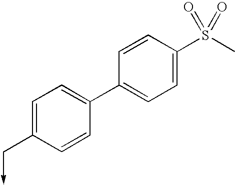 |
| 175717581759 | 115 | rac+− | 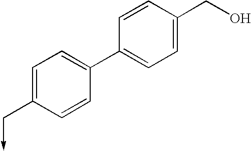 |
| 176017611762 | 118 | rac+− | 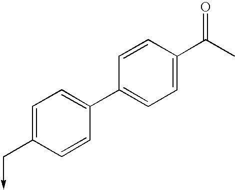 |
| 176317641765 | 106 | rac+− | 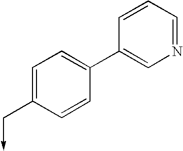 |
| 176617671768 | 121 | rac+− | 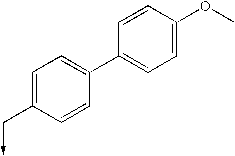 |
| 176917701771 | 123 | rac+− | 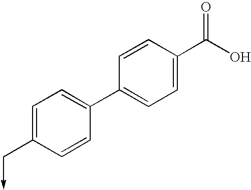 |
| 177217731774 | 99 | rac+− | 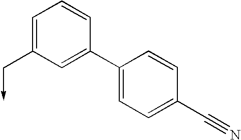 |
| 177517761777 | 120 | rac+− | 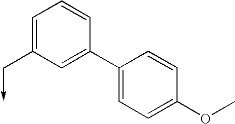 |
| 177817791780 | 105 | rac+− | 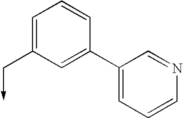 |
| 178117821783 | 111 | rac+− | 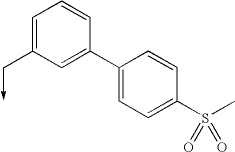 |
| 178417851787 | 114 | rac+− | 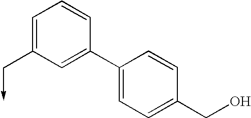 |
| 178817891790 | 102 | rac+− | 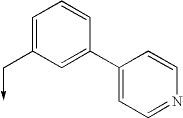 |
| 179117921793 | 117 | rac+− | 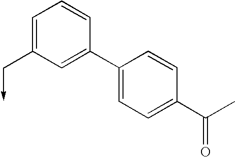 |
| 179417951796 | 108 | rac+− | 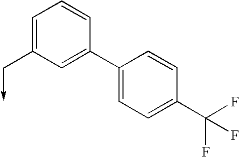 |
| 179717981799 | 101 | rac+− | 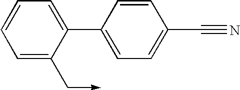 |
| 180018011802 | 122 | rac+− | 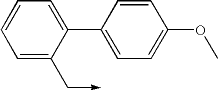 |
| 180318041805 | 119 | rac+− | 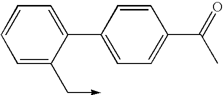 |
| 180618071808 | 124 | rac+− | 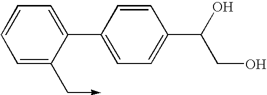 |
| 180918101811 | 107 | rac+− | 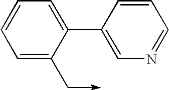 |
| 181218131814 | 110 | rac+− | 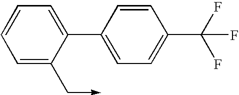 |
| 181518161817 | 113 | rac+− | 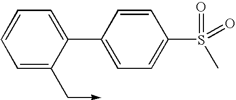 |
| 181818191820 | 116 | rac+− | 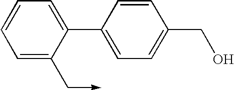 |
| 182118221823 | 104 | rac+− | 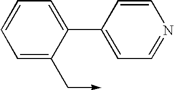 |
| 182418251826 | 125 | rac+− | 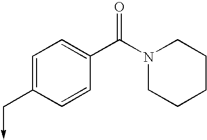 |
| 182718281829 | 127 | rac+− | 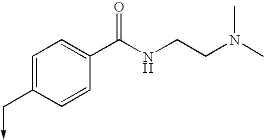 |
| 183018311832 | 128 | rac+− | 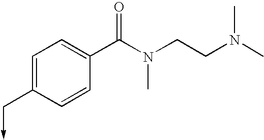 |
| 183318341835 | 126 | rac+− | 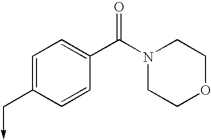 |
| 183618371838 | 134 | rac+− | 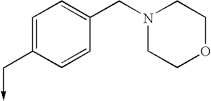 |
| 183918401841 | 131 | rac+− | 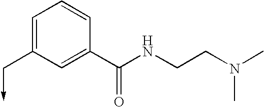 |
| 184218431844 | 133 | rac+− | 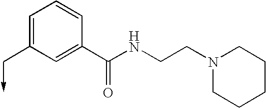 |
| 184518461847 | 132 | rac+− | 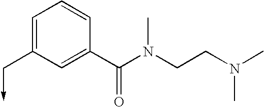 |
| 184818491850 | 130 | rac+− | 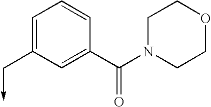 |
| 185118521853 | 129 | rac+− | 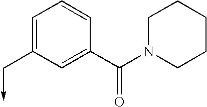 |
| 185418551856 | 135 | rac+− | 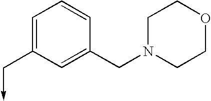 |
| 185718581859 | 137 | rac+− | 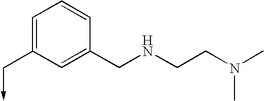 |
| 186018611862 | 136 | rac+− | 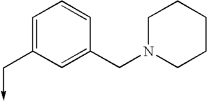 |
| 186318641865 | 138 | rac+− | 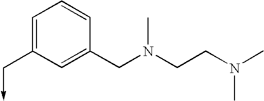 |
| 186618671868 | 139 | rac+− | 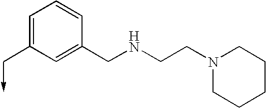 |
| 186918701871 | 95 | rac+− | 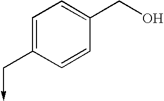 |
| 187218731874 | 96 | rac+− | 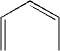 |
| 187518761877 | 89 | rac+− | 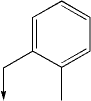 |

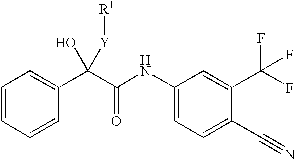

| | | Racemic or | |
|---|---|---|---|
| No. | Example | enantiomer | —Y—R1 |
| 187818791880 | 140 | rac+− | 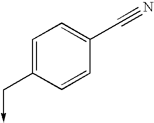 |
| 188118821883 | 141 | rac+− | 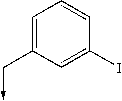 |
| 188418851886 | 142 | rac+− | 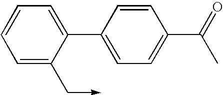 |

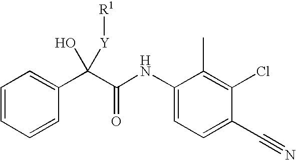

| | | Racemic or | |
|---|---|---|---|
| No. | Example | enantiomer | —Y—R1 |
| 188718881889 | 144 | rac+− | 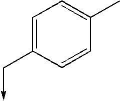 |
| 189018911892 | 145 | rac+− | 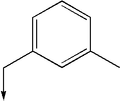 |
| 189318941895 | 146 | rac+− | 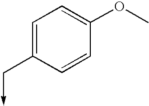 |
| 189618971898 | 143 | rac+− | 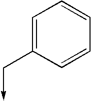 |
| 189919001901 | 147 | rac+− | 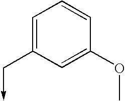 |

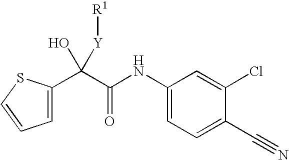

| | | Racemic or | |
|---|---|---|---|
| No. | Example | enantiomer | —Y—R1 |
| 190219031904 | 148 | rac+− | 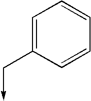 |

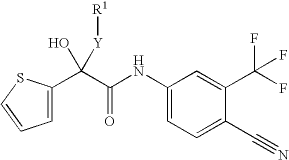

| | | Racemic or | |
|---|---|---|---|
| No. | Example | enantiomer | —Y—R1 |
| 190519061907 | 150 | rac+− | 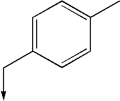 |
| 190819091910 | 149 | rac+− | 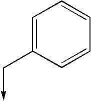 |

Biological characterization of the compounds according to the invention

Progesterone receptor modulators can be identified with the aid of simple methods, test programmes known to the skilled person. It is possible for this purpose for example to incubate a compound to be tested together with a progestogen in a test system for progesterone receptor ligands and to check whether the effect mediated by progesterone is altered in the presence of the modulator in this test system. The substances according to the invention of the general formula I were tested in the following models:

Progesterone receptor-binding assay

Measurement of the receptor binding affinity:

The receptor binding affinity was determined by competitive binding of a specifically binding ³H-labelled hormone (tracer) and of the compound to be tested on receptors in the cytosol from animal target organs. The aim in this case was receptor saturation and reaction equilibrium.

The tracer and increasing concentrations of the compound to be tested (competitor) were coincubated at 0-4° C. for 18 h with the receptor-containing cytosol fraction. After removal of unbound tracer with carbon-dextran suspension, the receptor-bound tracer content was measured for each concentration, and the IC₅₀ was determined from the concentration series. The relative molar binding affinity (RBA) was calculated as ratio of the IC₅₀ values for reference substance and compound to be tested (×100%) (RBA of the reference substance=100%).

The following incubation conditions were chosen for the receptor types:

Progesterone receptor:

Uterus cytosol of the estradiol-primed rabbit, homogenized in TED buffer (20 mMTris/HCl, pH 7.4; 1 mM ethylenediamine tetraacetate, 2 mM dithiothreitol) with 250 mM sucrose; stored at −30° C. Tracer: ³H-ORG 2058, 5 nM; reference substance: progesterone.

Glucocorticoid receptor:

Thymus cytosol from the adrenalectomized rat, thymi stored at −300° C.; buffer: TED.

Tracer: ³H-dexamethasone, 20 nM; reference substance: dexamethasone.

The competition factors (CF values) for the compounds according to the invention of the general formula (I) on the progesterone receptor are between 0.2 and 35 relative to progesterone. The CF values on the glucocorticoid receptor are in the range from 3 to 35 relative to dexamethasone.

The compounds according to the invention accordingly have a high affinity for the progesterone receptor, but only a low affinity for the glucocorticoid receptor.

Antagonism on the progesterone receptor PR

The transactivation assay is carried out as described in WO 02/054064.

The IC₅₀ values are in the range from 0.1 to 150 nM.

The table below shows, by way of example, results from the transactivation test for antagonistic activity on (PR-B).

| Example | |
|---|---|
| No. | IC₅₀ [nM] |
| 1b | 30 |
| 5 | 5 |
| 10 | 5 |
| 27 | 3 |
| 29b | 40 |
| 34b | 71 |
| 45c | 3.2 |
| 48d | 50 |
| 51b | 32 |
| 55f | 32 |
| 64b | 8.9 |
| 75 | 39.8 |
| 80 | 40 |

Agonism on the progesterone receptor PR

The transactivation assay is carried out as described in Fuhrmann et al. (Fuhrmann U., Hess-Stump H., Cleve A., Neef G., Schwede W., Hoffmann J., Fritzemeier K.-H., Chwalisz K., Journal of Medicinal Chemistry, 43, 26, 2000, 5010-5016). The EC₅₀ values are in the range from 0.01 to 150 nM.

Dosage

The progesterone receptor modulators can be administered orally, enterally, parenterally or transdermally for the use according to the invention.

Satisfactory results are generally to be expected in the treatment of the indications mentioned hereinbefore when the daily doses cover a range from 1 μg to 1000 mg of the compound according to the invention for gynaecological indications such as the treatment of endometriosis, leiomyomas of the uterus and dysfunctional bleeding, and for use in fertility control and for hormone replacement therapy. For oncological indications, daily dosages in the range from 1 μg to 2000 mg of the compound according to the invention are to be administered.

Suitable dosages of the compounds according to the invention in humans for the treatment of endometriosis, of leiomyomas of the uterus and dysfunctional bleeding and for use in fertility control and for hormone replacement therapy are from 50 μg to 500 mg per day, depending on the age and constitution of the patient, it being possible to administer the necessary daily dose by single or multiple administration.

The dosage range for the compounds according to the invention for the treatment of breast carcinomas is 10 mg to 2000 mg per day.

The pharmaceutical products based on the novel compounds are formulated in a manner known per se by processing the active ingredient with the carrier substances, fillers, substances influencing disintegration, binders, humectants, lubricants, absorbents, diluents, masking flavours, colorants, etc. which are used in pharmaceutical technology, and converting into the desired administration form. Reference should be made in this connection to Remington's Pharmaceutical Science, 15^{th} ed. Mack Publishing Company, East Pennsylvania (1980).

Suitable for oral administration are in particular tablets, film-coated tablets, sugar-coated tablets, capsules, pills, powders, granules, pastilles, suspensions, emulsions or solutions.

Preparations for injection and infusion are possible for parenteral administration.

Appropriately prepared crystal suspensions can be used for intraarticular injection.

Aqueous and oily solutions for injection or suspensions and corresponding depot preparations can be used for intramuscular injection.

For rectal administration, the novel compounds can be used in the form of suppositories, capsules, solutions (e.g. in the form of enemas) and ointments, both for systemic and for local therapy.

Furthermore, compositions for vaginal use may also be mentioned as preparation.

For pulmonary administration of the novel compounds, they can be used in the form of aerosols and inhalants.

Patches are possible for transdermal administration, and formulations in gels, ointments, fatty ointments, creams, pastes, dusting powders, milk and tinctures are possible for topical application. The dosage of the compounds of the general formula I in these preparations should be 0.01% -20% in order to achieve an adequate pharmacological effect.

Corresponding tablets can be obtained for example by mixing the active ingredient with known excipients, for example inert diluents such as dextrose, sugar, sorbitol, mannitol, polyvinylpyrrolidone, disintegrants such as maize starch or alginic acid, binders such as starch or gelatin, lubricants such as magnesium stearate or talc and/or means to achieve a depot effect such as carboxypolymethylene, carboxymethylcellulose, cellulose acetate phthalate or polyvinyl acetate. The tablets may also consist of a plurality of layers.

Correspondingly, coated tablets can be produced by coating cores produced in analogy to the tablets with compositions normally used in tablet coatings, for example polyvinylpyrrolidone or shellac, gum arabic, talc, titanium oxide or sugar. The tablet covering may in this case also consist of a plurality of layers, it being possible to use the excipients mentioned above for tablets.

Solutions or suspensions of the compounds according to the invention of the general formula I may additionally comprise taste-improving agents such as saccharin, cyclamate or sugar, and, for example, flavourings such as vanillin or orange extract. They may additionally comprise suspending excipients such as sodium carboxymethylcellulose or preservatives such as p-hydroxybenzoates.

Capsules comprising the compounds of the general formula I can be produced for example by mixing the compound(s) of the general formula I with an inert carrier such as lactose or sorbitol and encapsulating it in gelatin capsules.

Suitable suppositories can be produced for example by mixing with carriers intended for this purpose, such as neutral fats or polyethylene glycol or derivatives thereof.

The compounds according to the invention of the general formula (I) or their pharmaceutically acceptable salts can be used, because of their antagonistic or partial agonistic activity, for the manufacture of a medicament, in particular for the treatment and prophylaxis of gynaecological disorders such as endometriosis, leiomyomas of the uterus, dysfunctional bleeding and dysmenorrhoea. They can furthermore be employed to counteract hormonal irregularities, for inducing menstruation and alone or in combination with prostaglandins and/or oxytocin to induce labour.

The compounds according to the invention of the general formula (I) or their pharmaceutically acceptable salts are furthermore suitable for the manufacture of products for female contraception (see also WO 93/23020, WO 93/21927).

The compounds according to the invention or their pharmaceutically acceptable salts can additionally be employed alone or in combination with a selective estrogen receptor modulator (SERM) for female hormone replacement therapy.

In addition, the said compounds have an antiproliferative effect in hormone-dependent tumours. They are therefore suitable for the therapy of hormone-dependent carcinomas such as, for example, for breast, prostate and endometrial carcinomas.

The compounds according to the invention or their pharmaceutically acceptable salts can be employed for the treatment of hormone-dependent carcinomas both in first-line therapy and in second-line therapy, especially after tamoxifen failure.

The compounds according to the invention, having antagonistic or partially agonistic activity, of the general formula (I) or their pharmaceutically acceptable salts can also be used in combination with compounds having antiestrogenic activity (estrogen receptor antagonists or aromatase inhibitors) or selective estrogen receptor modulators (SERM) for producing pharmaceutical products for the treatment of hormone-dependent tumours. The compounds according to the invention can likewise be used in combination with SERMs or an antiestrogen (estrogen receptor antagonist or aromatase inhibitor) for the treatment of endometriosis or of leiomyomas of the uterus.

Suitable for combination with the non-steroidal progesterone receptor modulators according to the invention in this connection are for example the following antiestrogens (estrogen receptor antagonists or aromatase inhibitors) or SERMs: tamoxifen, 5-(4-{5-[(RS)-(4,4,5,5,5-pentafluoropentyl)sulphinyl]pentyloxy}phenyl)-6-phenyl-8,9-dihydro-7H-benzocyclohepten-2-ol (WO 00/03979), ICI 182 780 (7alpha-[9-(4,4,5,5-pentafluoropentylsulphinyl)nonyl]estra-1,3,5(10)-triene-3,17beta-diol), 11beta-fluoro-7alpha-[5-(methyl{3-[(4,4,5,5,5-pentafluoropentyl)sulphanyl]propyl}amino)pentyl]-estra-1,3,5(10)-triene-3,17beta-diol (WO98/07740), 11beta-fluoro-7alpha-{5-[methyl(7,7,8,8,9,9,10,10,10-nonafluorodecyl)amino]pentyl}estra-1,3,5(10)-triene-3,17-beta-diol (WO 99/33855), 11beta-fluoro-17alpha-methyl-7alpha-{5-[methyl(8,8,9,9,9-pentafluorononyl)amino]pentyl}estra-1,3,5(10)-triene-3,17beta-diol (WO 03/045972), clomifene, raloxifene, and further compounds having antiestrogenic activity, and aromatase inhibitors such as, for example, fadrozole, formestane, letrozole, anastrozole or atamestane.

Finally, the present invention also relates to the use of the compounds of the general formula I, where appropriate together with an antiestrogen or SERM, for the manufacture of a medicament.

The present invention further relates to pharmaceutical compositions which comprise at least one compound according to the invention, where appropriate in the form of a pharmaceutically/pharmacologically acceptable salt.

These pharmaceutical compositions and medicaments may be intended for oral, rectal, vaginal, subcutaneous, percutaneous, intravenous or intramuscular administration. Besides conventional carriers and/or diluents, they comprise at least one compound according to the invention.

The medicaments of the invention are manufactured with the conventional solid or liquid carriers or diluents and the excipients normally used in pharmaceutical technology appropriate for the desired mode of administration with a suitable dosage in a known manner. The preferred preparations consist of a dosage form suitable for oral administration. Examples of such dosage forms are tablets, film-coated tablets, sugar-coated tablets, capsules, pills, powders, solutions or suspensions, where appropriate as depot form.

The pharmaceutical compositions comprising at least one of the compounds according to the invention are preferably administered orally.

Also suitable are parenteral preparations such as solutions for injection. Further preparations which may also be mentioned are for example suppositories and compositions for vaginal use.

The following examples serve to explain the subject-matter of the invention in more detail without intending to restrict it thereto.

General procedures for preparing compounds of the general formula (I)

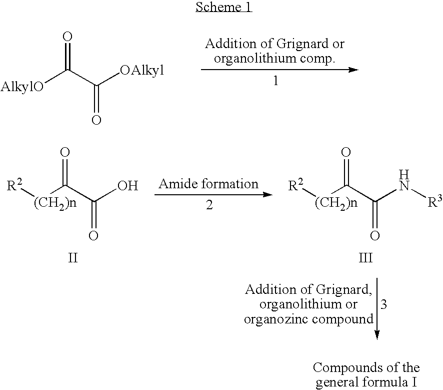

The compounds of the general formula (I) can be synthesized as shown in Scheme 1. Monoaddition of Grignard or organolithium compounds onto, for example, an oxalic bisester and subsequent hydrolysis affords carboxylic acids of the general formula II. The amides of the general formula III are preferably prepared via formation of the acid chlorides and subsequent reaction with the appropriate amines. As alternative to this, however, it is also possible to use other methods for amide formation depending on the amine to be introduced. The compounds of the general formula I are then prepared from the amides of the general formula III by renewed addition of Grignard, organolithium or organozinc compounds. Steps 1, 2 and 3 can, however, also be carried out in the reverse sequence.

Compounds of the general formula I in which X is 2 hydrogen atoms are prepared by reductive amination of the appropriate aldehydes.

The substituents R¹, R² and R³ may where appropriate also be modified further after introduction has taken place. Suitable for this purpose are for example oxidation, reduction, alkylations, acylations, nucleophilic additions or especially also transition metal-catalysed coupling reactions.

Functional groups in compounds of the general formulae II and III are provided where appropriate with temporary protective groups which are then eliminated again at a suitable stage.

The preparation of 6-amino-4-methyl-2,3-benzoxazin-1-one has been described for example in WO 199854159.

### SYNTHESIS EXAMPLES

### Example 1

### rac-6-[2,4-diphenyl-2-hydroxybut-3-ynoylamino]-4-methyl-2,3-benzoxazin-1-one

### a) 6-[2-phenyl-2-oxoacetoylamino]-4-methyl-2,3-benzoxazin-1-one

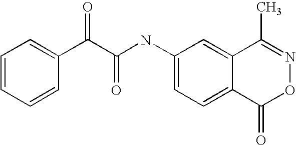

Phenylglyoxylic acid (3 g) was dissolved in 50 ml of N,N-dimethylacetamide. At −10° C., 1.75 ml of thionyl chloride were added, and the mixture was stirred at −10° C. for one hour. Then 4.9 g of 6-amino-4-methyl-2,3-benzoxazin-1-one were added in portions. This was followed by stirring for 3 hours (−10° C. to 0° C.). The reaction mixture was then poured into ice-water. The mixture was stirred for 2 hours and filtered with suction. The resulting solid was purified by column chromatography on silica gel with a hexane/ethyl acetate mixture. 4.42 g of product were obtained.

¹H NMR (ppm, DMSO-D₆, 400 MHz): 2.50 (3H); 7.59 (2H); 7.75 (1H); 8.07 (2H); 8.20-8.32 (3H).

### b) rac-6-[2,4-diphenyl-2-hydroxybut-3-ynoylamino]-4-methyl-2,3-benzoxazin-1-one

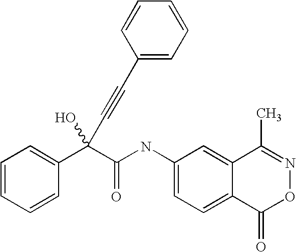

n-Butyllithium (810 μl, 1.6 M in hexane) was added to a solution of 145 μl of phenylacetylene in tetrahydrofuran at −78° C. The mixture was stirred at this temperature for 30 minutes and then a solution of the substance (200 mg) described under 1a in 10 ml of tetrahydrofuran was added dropwise. The mixture was then allowed to reach 23° C. over about 3 h and was subsequently stirred for 10 h. The reaction mixture was then poured into ice-cold saturated ammonium chloride solution. This was extracted with ethyl acetate. The combined organic phases were washed with saturated sodium chloride solution and dried over sodium sulphate. The crude product was chromatographed on silica gel. 135 mg of product were obtained.

¹H NMR (ppm, CDCl₃, 400 MHz): 2.53 (3H); 4.06 (1H); 7.30-7.50 (6H); 7.53 (2H); 7.69 (1H); 7.83 (2H); 8.33 (2H); 8.91 (1H).

Compounds 2) and 3) were prepared in analogy to Example 1 from the substance described under 1a) and the respective lithium arylacetylide.

### Example 2

### rac-6-[2-hydroxy-2-phenyl-4-(4-methylphenyl)but-3-ynoylamino]-4-methyl-2,3-benzoxazin-1-one

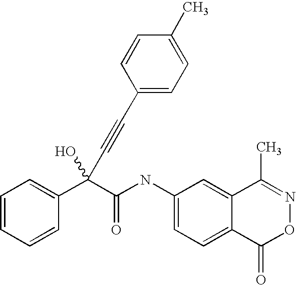

¹H NMR (ppm, CDCl₃, 400 MHz): 2.38 (3H); 2.54 (3H); 4.00 (1H); 7.15 (2H); 7.38-7.52 (5H); 7.66 (1H); 7.82 (2H); 8.32 (2H); 8.91 (1H).

### Example 3

### rac-6-[2-hydroxy-2-phenyl-4-(4-(trifluoromethyl)phenyl)but-3-ynoylamino]-4-methyl-2,3-benzoxazin-1-one

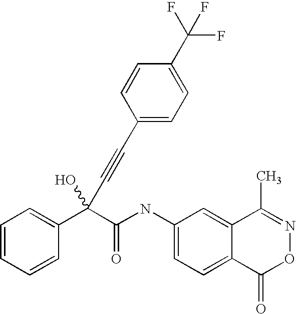

¹H NMR (ppm, CDCl₃, 300 MHz): 2.52 (3H); 4.05 (1H); 7.42-7.53 (3H); 7.58-7.74 (5H) 7.82 (2H); 8.33 (2H); 8.97 (1H).

### Example 4

### rac-6-[2-hydroxy-2,2-diphenylacetoylamino]-4-methyl-2,3-benzoxazin-1-one

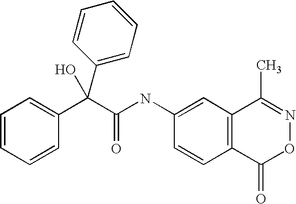

A 2M solution of phenylmagnesium bromide in tetrahydrofuran (1.23 ml) was diluted with 2 ml of tetrahydrofuran. It was cooled to −78° C., and a solution of 180 mg of the substance described under 1a) in tetrahydrofuran was added dropwise. The mixture was stirred at −78° C. for 2 hours and then worked up in analogy to 1b). 123 mg of product were isolated after column chromatography.

¹H NMR (ppm, DMSO-D₆, 300 MHz): 7.22-7.38 (6H); 7.42 (4H); 8.16 (1H); 8.40 (2H) 10.73 (1H).

### Example 5

### rac-6-[2-hydroxy-2,3-diphenylpropionylamino]-4-methyl-2,3-benzoxazin-1-one

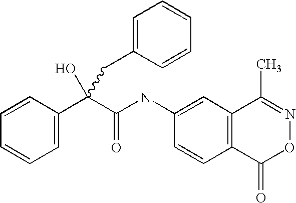

A 2M solution of benzylmagnesium chloride in tetrahydrofuran (0.6 ml) was diluted with 2 ml of tetrahydrofuran. It was cooled to −78° C., and a solution of 180 mg of the substance described under 1a) in tetrahydrofuran was added dropwise. The mixture was stirred at −78° C. for 2 hours and then worked up in analogy to 1b). 142 mg of product were isolated after column chromatography.

¹H NMR (ppm, CDCl₃, 400 MHz): 2.56 (3H); 2.90 (1H); 3.29 (1H); 3.93 (1H); 7.18 (2H); 7.25-7.45 (6H); 7.62 (1H); 7.74 (2H); 8.26 (2H); 9.00 (1H).

### Example 5a and 5b

### (+)-6-[2-hydroxy-2,3-diphenylpropionylamino]-4-methyl-2,3-benzoxazin-1-one 5a and

### (−)-6-[2-hydroxy-2,3-diphenylpropionylamino]-4-methyl-2,3-benzoxazin-1-one 5b

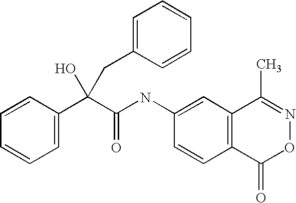

The racemic mixture (350 mg) obtained in Example 5 was separated by preparative chiral HPLC (Chiralpak AD column 250×10 mm) into the enantiomers 5a (165 mg) and 5b (182 mg).

5a and 5b:

6a: [α]^{D}₂₀: +27.0° (CHCl₃, 10.1 mg/1 ml; λ=589 nM)

6b: [α]^{D}₂₀: −26.5° (CHCl₃, 10.1 mg/1 ml; λ=589 nM)

Compounds 6-9 were prepared in analogy to Example 5 from the substance described under 1a) and the respective benzyl Grignard reagent.

### Example 6

### rac-6-[2-hydroxy-2-phenyl-3-(4-methylphenyl)propionylamino]-4-methyl-2,3-benzoxazin-1-one

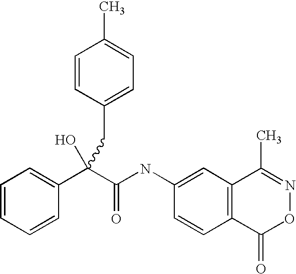

¹H NMR (ppm, CDCl₃, 400 MHz): 2.30 (3H); 2.57 (3H); 2.86 (1H); 3.22 (1H); 3.91 (1H); 7.02 (2H); 7.10 (2H); 7.34 (1H); 7.41 (2H); 7.63 (1H); 7.74 (2H); 8.28 (2H); 9.00 (1H).

### Example 6a and 6b

### (+)-6-[2-hydroxy-2-phenyl-3-(4-methylphenyl)propionylamino]-4-methyl-2,3-benzoxazin-1-one 6a and

### (−)-6-[2-hydroxy-2-phenyl-3-(4-methylphenyl)propionylamino]-4-methyl-2,3-benzoxazin-1-one 6b

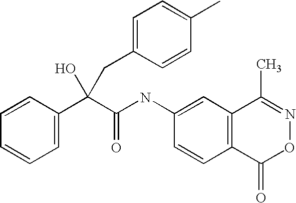

The racemic mixture obtained under Example 6 was separated by preparative chiral HPLC (column: Chiralpak AD 250×10 mm) into enantiomers 6a and 6b.

6a and 6b:

6a: [α]^{D}₂₀: +32.7° (CHCl₃, 4.6 mg/1 ml; λ=589 nM)

6b: [α]^{D}₂₀: −34.5° (CHCl₃, 4.3 mg/1 ml; λ=589 nM)

### Example 7

### rac-6-[2-Hydroxy-2-phenyl-3-(4-methoxyphenyl)propionylamino]-4-methyl-2,3-benzoxazin-1-one

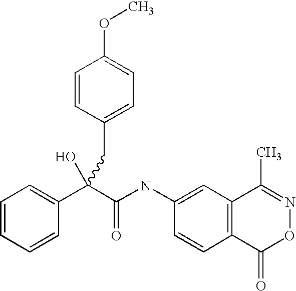

¹H NMR (ppm, CDCl₃, 400 MHz): 2.56 (3H); 2.81 (1H); 3.20 (1H); 3.76 (3H); 3.89 (1H); 6.81 (2H); 7.07 (2H); 7.34 (1H); 7.41 (2H); 7.65 (1H); 7.73 (2H); 3.28 (2H); 9.00 (1H).

### Example 7a and 7b

### (+)-6-[2-hydroxy-2-phenyl-3-(4-methoxyphenyl)propionylamino]-4-methyl-2,3-benzoxazin-1-one 7a and

### (−)-6-[2-hydroxy-2-phenyl-3-(4-methoxyphenyl)propionylamino]-4-methyl-2,3-benzoxazin-1-one 7b

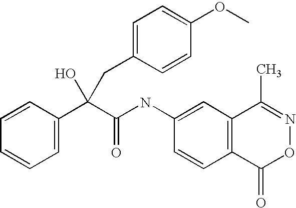

The racemic mixture obtained under Example 7 was separated by preparative chiral HPLC (column: Chiralpak AD 250×10 mm) into enantiomers 7a and 7b.

7a and 7b:

7a: [α]^{D}₂₀: +31.2° (CHCl₃, 3.3 mg/1 ml; λ=589 nM)

7b: [α]^{D}₂₀: −32.6° (CHCl₃, 3.2 mg/1 ml; λ=589 nM)

### Example 8

### rac-6-[2-hydroxy-2-phenyl-3-(3-methylphenyl)propionylamino]-4-methyl-2,3-benzoxazin-1-one

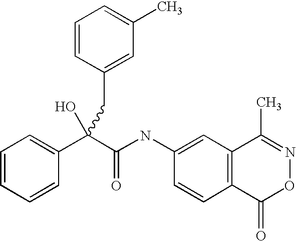

¹H NMR (ppm, CDCl₃, 300 MHz): 2.26 (3H); 2.57 (3H); 2.89 (1H); 3.22 (1H); 3.91 (1H); 6.97 (2H); 7.09 (1H); 7.18 (1H); 7.30-7.48 (3H); 7.63 (1H); 7.73 (2H); 8.26 (2H); 9.00 (1H).

### Example 8a and 8b

### (+)-6-[2-hydroxy-2-phenyl-3-(3-methylphenyl)propionylamino]-4-methyl-2,3-benzoxazin-1-one 8a and

### (−)-6-[2-hydroxy-2-phenyl-3-(3-methylphenyl)propionylamino]-4-methyl-2,3-benzoxazin-1-one 8b

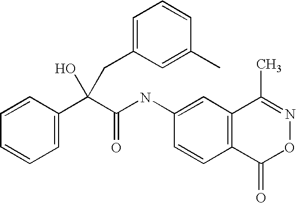

The racemic mixture obtained under Example 8 was separated by preparative chiral HPLC (column: Chiralpak AD 250×10 mm) into enantiomers 8a and 8b.

8a and 8b:

8a: [α]^{D}₂₀: +36.5° (CHCl₃, 5.7 mg/1 ml; λ=589 nM)

8b: [α]^{D}₂₀: −39.0+ (CHCl₃, 5.0 mg/1 ml; λ=589 nM)

### Example 9

### rac-6-[2-hydroxy-2-phenyl-3-(3-methoxyphenyl)propionylamino]-4-methyl-2,3-benzoxazin-1-one

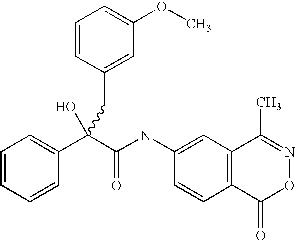

¹H NMR (ppm, CDCl₃, 300 MHz): 2.54 (3H); 2.97 (1H); 3.27 (1H); 3.67 (3H); 3.90 (1H); 6.67 (1H); 6.76 (2H); 7.20 (1H); 7.30-7.48 (3H); 7.67 (1H); 7.73 (2H); 8.27 (2H); 9.02 (1H).

### Example 9a and 9b

### (+)-6-[2-hydroxy-2-phenyl-3-(3-methoxyphenyl)propionylamino]-4-methyl-2,3-benzoxazin-1-one 9a and

### (−)-6-[2-hydroxy-2-phenyl-3-(3-methoxyphenyl)propionylamino]-4-methyl-2,3-benzoxazin-1-one 9b

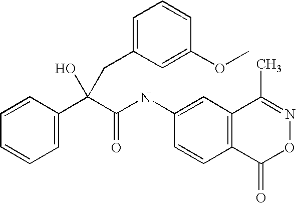

The racemic mixture obtained under Example 9 was separated by preparative chiral HPLC (column: Chiralpak AD 250×10 mm) into enantiomers 9a and 9b.

9a and 9b:

9a: [α]^{D}₂₀: +40.5° (CHCl₃, 9.7 mg/1 ml; λ=589 nM)

9b: [α]^{D}₂₀: −42.1° (CHCl₃, 6.0 mg/1 ml; λ=589 nM)

### Example 10

### rac-6-[2-hydroxy-2-(4-nitrophenyl)-3-phenylpropionylamino]-4-methyl-2,3-benzoxazin-1-one

### a) 6-[2-(4-nitrophenyl)-2-oxoacetoylamino]-4-methyl-2,3-benzoxazin-1-one

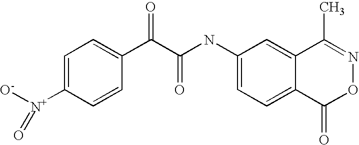

Compound 10a) was synthesized in analogy to Example 1a) from 4-nitrophenylglyoxylic acid, thionyl chloride and 6-amino-4-methyl-2,3-benzoxazin-1-one in N,N-dimethylacetamide.

¹H NMR (ppm, DMSO-D₆, 400 MHz): 2.49 (3H); 7.60 (2H); 7.77 (1H); 8.06 (2H); 8.20-8.32 (3H).

Compounds 10b) and 11-14 were prepared in analogy to Example 5 from the substance described under 10a) and the respective benzyl Grignard reagent.

### b) rac-6-[2-hydroxy-2-(4-nitrophenyl)-3-phenylpropionylamino]-4-methyl-2,3-benzoxazin-1-one

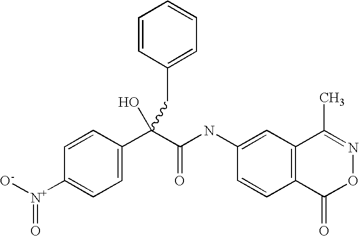

¹H NMR (ppm, CDCl₃, 300 MHz): 2.58 (3H); 3.02 (1H); 3.28 (1H); 3.99 (1H); 7.15 (2H): 7.32 (3H); 7.67 (1H); 7.98 (2H); 8.21-8.32 (4H); 9.00 (1H).

### Example 11

### rac-6-[2-Hydroxy-2-(4-nitrophenyl)-3-(4-methylphenyl)propionylamino]-4-methyl-2,3-benzoxazin-1-one

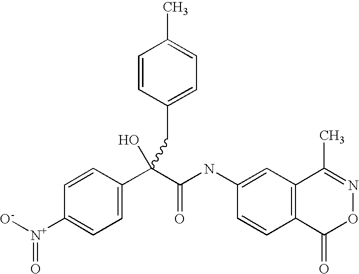

¹H NMR (ppm, CDCl₃, 400 MHz): 2.30 (3H); 2.58 (3H); 3.05 (1H); 3.20 (1H); 3.94 (1H): 7.00 (2H); 7.12 (2H); 7.66 (1H); 7.98 (2H); 8.20-8.30 (4H); 9.01 (1H).

### Example 11a and 11b

### (+)-6-[2-hydroxy-2-(4-nitrophenyl)-3-(4-methylphenyl)propionylamino]-4-methyl-2,3-benzoxazin-1-one 11a and

### (−)-6-[2-hydroxy-2-(4-nitrophenyl)-3-(4-methylphenyl)propionylamino]-4-methyl-2,3-benzoxazin-1-one 11b

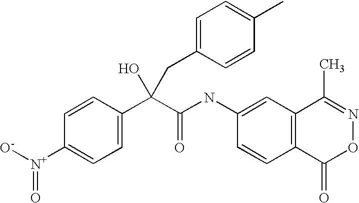

The racemic mixture obtained under Example 11 was separated by preparartive chiral HPLC (column: Chiralpak AD 250×10 mm) into enantiomers 11a and 11b. 11a and 11b:

11a: [α]^{D}₂₀: +13.2° (CHCl₃, 3.5 mg/1 ml; λ=589 nM)

11b: [α]^{D}₂₀: −12.4° (CHCl₃, 3.5 mg/1 ml; λ=589 nM)

### Example 12

### rac-6-[2-hydroxy-2-(4-nitrophenyl)-3-(4-methoxyphenyl)propionylamino]-4-methyl-2,3-benzoxazin-1-one

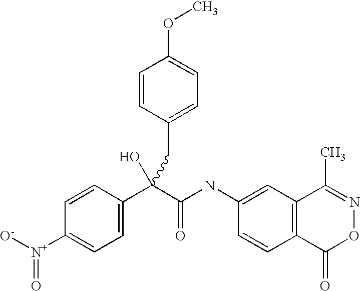

¹H NMR (ppm, CDCl₃, 400 MHz): 2.58 (3H); 3.05 (1H); 2.19 (1H); 3.76 (3H); 3.92 (1H); 6.83 (2H); 7.06 (2H); 7.67 (1H); 7.98 (2H); 8.22-8.32 (4H); 9.01 (1H).

### Example 12a and 12b

### (+)-6-[2-hydroxy-2-(4-nitrophenyl)-3-(4-methoxyphenyl)propionylamino]-4-methyl-2,3-benzoxazin-1-one 12a and

### (−)-6-[2-hydroxy-2-(4-nitrophenyl)-3-(4-methoxyphenyl)propionylamino]-4-methyl-2,3-benzoxazin-1-one 12b

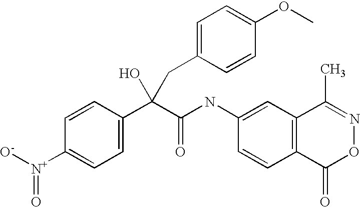

The racemic mixture obtained under Example 12 was separated by preparative chiral HPLC (column: Chiralpak AD 250×10 mm) into enantiomers 12a and 12b.

12a and 12b:

12a: [α]^{D}₂₀: +11.4° (CHCl₃, 3.1 mg/1 ml; λ=589 nM)

12b: [α]^{D}₂₀: −12.8° (CHCl₃, 3.3 mg/1 ml; λ=589 nM)

### Example 13

### rac-6-[2-hydroxy-2-(4-nitrophenyl)-3-(3-methylphenyl)propionylamino]-4-methyl-2,3-benzoxazin-1-one

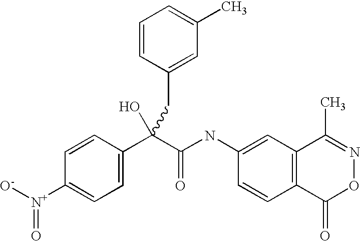

¹H NMR (ppm, CDCl₃, 400 MHz): 2.28 (3H); 2.58 (3H); 3.07 (1H); 3.19 (1H); 3.96 (1H); 6.93 (2H); 7.11 (1H); 7.20 (1H); 7.66 (1H); 7.99 (2H); 8.21-8.31 (4H); 9.00 (1H).

### Example 13a and 13b

### (+)-6-[2-hydroxy-2-(4-nitrophenyl)-3-(3-methylphenyl)propionylamino]-4-methyl-2,3-benzoxazin-1-one 13a and

### (−)-6-[2-hydroxy-2-(4-nitrophenyl)-3-(3-methylphenyl)propionylamino]-4-methyl-2,3-benzoxazin-1-one 13b

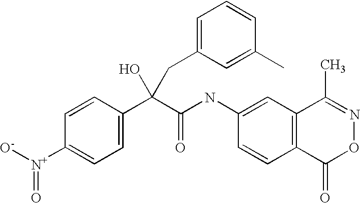

The racemic mixture obtained under Example 13 was separated by preparative chiral HPLC (column: Chiralpak AD 250×10 mm) into enantiomers 13a and 13b.

13a and 13b:

13a: [α]^{D}₂₀: +1.9° (CHCl₃, 5.0 mg/1 ml; λ=589 nM)

13b: [α]^{D}₂₀: −2.5° (CHCl₃, 6.1 mg/1 ml; λ=589 nM)

### Example 14

### rac-6-[2-hydroxy-2-(4-nitrophenyl)-3-(3-methoxyphenyl)propionylamino]-4-methyl-2,3-benzoxazin-1-one

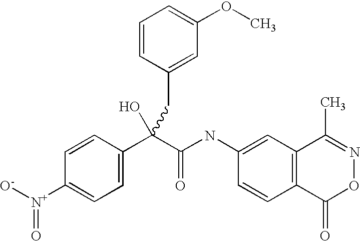

¹H NMR (ppm, CDCl₃, 300 MHz): 2.57 (3H); 3.12 (1H); 3.20 (1H); 3.70 (3H); 3.95 (1H); 6.68 (1H); 6.71 (1H); 6.83 (1H); 7.26 (1H); 7.68 (1H); 7.98 (2H); 8.20-8.35 (4H); 9.02 (1H).

### Example 15

### rac-6-[2-hydroxy-2-(4-cyanophenyl)-2-phenylacetoylamino]-4-methyl-2,3-benzoxazin-1-one

### a) 6-[2-(4-cyanophenyl)-2-oxoacetoylamino]-4-methyl-2,3-benzoxazin-1-one

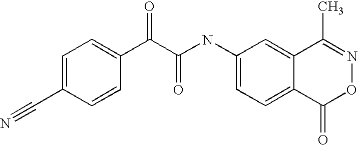

Compound 15a) was synthesized in analogy to Example 1a) from 4-cyanophenylglyoxylic acid, thionyl chloride and 6-amino-4-methyl-2,3-benzoxazin-1-one in N,N-dimethylacetamide.

Compounds 15b) and 16-18 were prepared in analogy to Example 5) from the substance described under 15a) and the respective phenyl or benzyl Grignard reagent.

### b) rac-6-[2-hydroxy-2-(4-cyanophenyl)-2-phenylacetoylamino]-4-methyl-2,3-benzoxazin-1-one

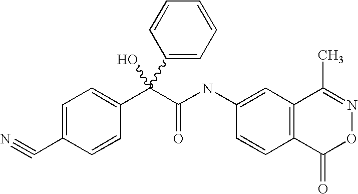

¹H NMR (ppm, CDCl₃, 300 MHz): 2.52 (3H); 3.80 (1H); 7.49 (5H); 7.67 (2H); 7.76 (3H); 8.28 (1H); 8.39 (1H); 9.56 (1H).

### Example 16

### rac-6-[2-hydroxy-2-(4-cyanophenyl)-3-phenylpropionylamino]-4-methyl-2,3-benzoxazin-1-one

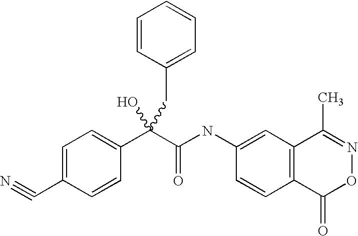

¹H NMR (ppm, CDCl₃, 300 MHz): 2.57 (3H); 3.02 (1H); 3.21 (1H); 3.93 (1H); 7.12 (2H); 7.30 (3H); 7.65-7.75 (3H); 7.90 (2H); 8.20-8.30 (2H); 8.99 (1H).

### Example 16a and 16b

### (+)-6-[2-hydroxy-2-(4-cyanophenyl)-3-phenylpropionylamino]-4-methyl-2,3-benzoxazin-1-one 16a and

### (−)-6-[2-hydroxy-2-(4-cyanophenyl)-3-phenylpropionylamino]-4-methyl-2,3-benzoxazin-1-one 16b

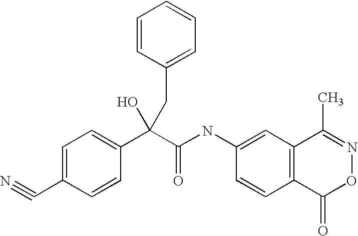

The racemic mixture obtained under Example 16 was separated by preparative chiral HPLC (column: Chiralpak AD 250×10 mm) into enantiomers 16a and 16b.

16a and 16b:

16a: [α]^{D}₂₀: +15.5° (CHCl₃, 10.8 mg/1 ml; λ=589 nM)

16b: [α]^{D}₂₀: −17.0° (CHCl₃, 10.2 mg/1 ml; λ=589 nM)

### Example 17

### rac-6-[2-hydroxy-2-(4-cyanophenyl)-3-(3-methylphenyl)propionylamino]-4-methyl-2,3-benzoxazin-1-one

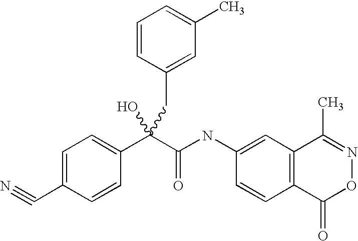

¹H NMR (ppm, CDCl₃, 300 MHz): 2.28 (3H); 2.57 (3H); 3.09 (1H); 3.18 (1H); 3.91 (1H); 6.92 (2H); 7.10 (1H); 7.20 (1H); 7.62-7.75 (3H); 7.91 (2H); 8.20-8.30 (2H); 7.99 (1H).

### Example 17a and 17b

### (+)-6-[2-hydroxy-2-(4-cyanophenyl)-3-(3-methylphenyl)propionylamino]-4-methyl-2,3-benzoxazin-1-one 17a and

### (−)-6-[2-hydroxy-2-(4-cyanophenyl)-3-(3-methylphenyl)propionylamino]-4-methyl-2,3-benzoxazin-1-one 17b

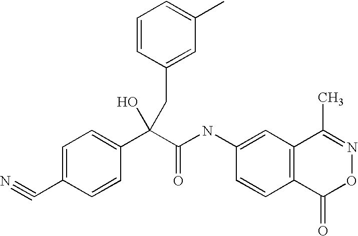

The racemic mixture obtained under Example 17 was separated by preparative chiral HPLC (column: Chiralpak AD 250×10 mm) into enantiomers 17a and 17b.

17a and 17b:

17a: [α]^{D}₂₀: +1.7° (CHCl₃, 7.2 mg/1 ml; λ=589 nM)

17b: [α]^{D}₂₀: −3.4° (CHCl₃, 8.4 mg/1 ml; λ=589 nM)

### Example 18

### rac-6-[2-hydroxy-2-(4-cyanophenyl)-3-(4-methylphenyl)propionylamino]-(4-methyl-2,3-benzoxazin-1-one

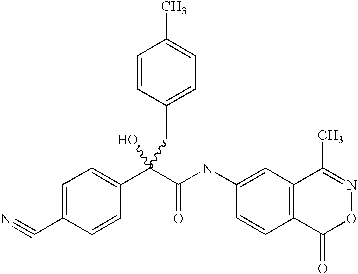

¹H NMR (ppm, CDCl₃, 400 MHz): 2.30 (3H); 2.55 (3H); 3.02 (1H); 3.18 (1H); 3.90 (1H); 7.00 (2H); 7.11 (2H); 7.63-7.35 (3H); 7.90 (2H); 8.23-8.32 (2H); 9.00 (1H).

### Example 19

### rac-6-[2-hydroxy-2-(4-trifluoromethylphenyl)-3-phenylpropionylamino]-4-methyl-2,3-benzoxazin-1-one

### a) 6-[2-(4-trifluorophenyl)-2-oxoacetoylamino]-4-methyl-2,3-benzoxazin-1-one

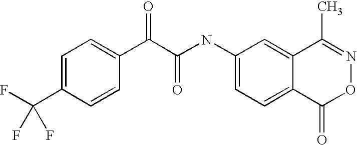

Compound 19a) was synthesized in analogy to Example 1a) from 4-trifluoro-phenylglyoxylic acid, thionyl chloride and 6-amino-4-methyl-2,3-benzoxazin-1-one in N,N-dimethylacetamide.

Compounds 19b) and 20) were prepared in analogy to Example 5) from the substance described under 19a) and the respective benzyl Grignard reagent.

### b) rac-6-[2-hydroxy-2-(4-trifluoromethylphenyl)-3-phenylpropionylamino]-4-methyl-2,3-benzoxazin-1-one

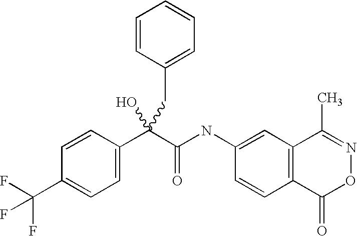

¹H NMR (ppm, CDCl₃, 400 MHz): 2.56 (3H); 3.05 (1H); 3.24 (1H); 3.98 (1H); 7.17 (2H); 7.30 (3H); 7.62-7.72 (3H); 7.90 (2H); 8.26 (2H); 9.00 (1H).

### Example 20

### rac-[2-hydroxy-2-(4-trifluoromethylphenyl)-3-(4-methylphenyl)propionylamino]-4-methyl-2,3-benzoxazin-1-one

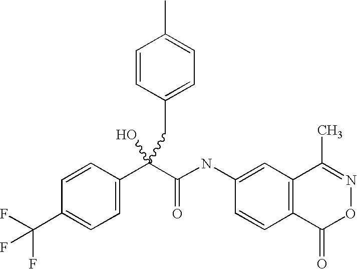

¹H NMR (ppm, CDCl₃, 300 MHz): 2.30 (3H); 2.57 (3H); 2.98 (1H); 3.20 (1H); 3.93 (1H); 7.02 (2H); 7.12 (2H); 7.60-7.72 (3H); 7.90 (2H); 8.27 (2H); 9.00 (1H).

### Example 20a and 20b

### (+)-6-[2-hydroxy-2-(4-trifluoromethylphenyl)-3-(4-methylphenyl)propionylamino]-4-methyl-2,3-benzoxazin-1-one 20a and

### (−)-6-[2-hydroxy-2-(4-trifluoromethylphenyl)-3-(4-methylphenyl)propionylamino]-4-methyl-2,3-benzoxazin-1-one 20b

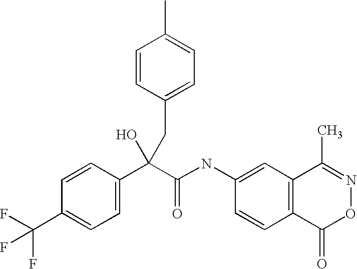

The racemic mixture obtained under Example 20 was separated by preparative chiral HPLC (column: Chiralpak AD 250×10 mm) into enantiomers 20a and 20b.

20a and 20b:

20a: [α]^{D}₂₀: +23.8° (CHCl₃, 10.2 mg/1 ml; λ=589 nM)

20b: [α]^{D}₂₀: −23.3° (CHCl₃, 9.8 mg/1 ml; λ=589 nM)

### Example 21

### rac-[2-hydroxy-2-(4-phenylphenyl)-3-phenylpropionylamino]-4-methyl-2,3-benzoxazin-1-one

### a) 6-[2-(4-phenylphenyl)-2-oxoacetoylamino]-4-methyl-2,3-benzoxazin-1-one

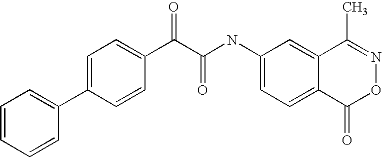

Compound 21a) was synthesized in analogy to Example 1a) from 4-diphenylglyoxylic acid, thionyl chloride and 6-amino-4-methyl-2,3-benzoxazin-1-one in N,N-dimethylacetamide.

¹H NMR (ppm, DMSO-D₆, 300 MHz): 2.50 (H); 7.40-7.55 (3H); 7.75 (2H); 7.90 (2H); 8.16 (2H); 8.26 (2H); 8.32 (1H); 11.64 (1H).

### b) rac-[2-hydroxy-2-(4-phenylphenyl)-3-phenylpropionylamino]-4-methyl-2,3-benzoxazin-1-one

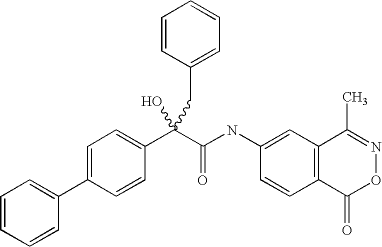

Compound 21b) was prepared in analogy to Example 5) from the substance described under 21a) and benzylmagnesium chloride.

¹H NMR (ppm, CDCl₃, 400 MHz): 2.57 (3H); 2.88 (1H); 3.31 (1H); 3.99 (1H); 7.20 (2H); 7.27-7.40 (4H); 7.44 (2H); 7.60 (2H); 7.63 (3H); 7.80 (2H); 8.28 (2H); 9.01 (1H).

### Example 22

### rac-5-[2-hydroxy-2,4-diphenylbut-3-ynoylamino]phthalide

### a) 5-[2-phenyl-2-oxoacetoylamino]phthalide

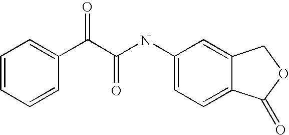

Compound 22a) was synthesized in analogy to Example 1a) from phenylglyoxylic acid, thionyl chloride and 5-aminophthalide in N,N-dimethylacetamide.

¹H NMR (ppm, DMSO-D₆, 400 MHz): 5.38 (2H); 7.58 (2H); 7.70-7.88 (3H); 8.02 (2H); 8.12 (1H); 11.43 (1H).

### b) rac-5-[2-hydroxy-2,4-diphenylbut-3-ynoylamino]phthalide

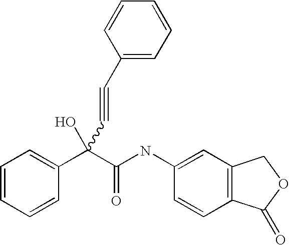

Compound 22b) was prepared in analogy to Example 1b) from the substance described under 22a), phenylacetylene and n-butyllithium.

¹H NMR (ppm, CDCl₃, 400 MHz): 5.25 (2H); 7.30-7.50 (7H); 7.53 (2H); 7.80-7.90 (3H); 8.10 (1H); 8.64 (1H).

### Example 23

### 5-[2-hydroxy-2,2-diphenylacetoylamino]phthalide

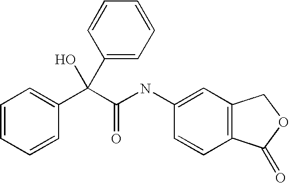

Compound 23 was prepared in analogy to Example 4 from the substance described under 22a) and phenylmagnesium bromide.

¹H NMR (ppm, DMSO-D₆, 400 MHz): 5.31 (2H); 7.20-7.35 (7H); 7.40 (4H); 7.72 (1H); 7.85 (1H); 8.20 (1H); 10.48 (1H).

### Example 24

### rac-5-[2-hydroxy-2,3-diphenylpropionylamino]phthalide 24

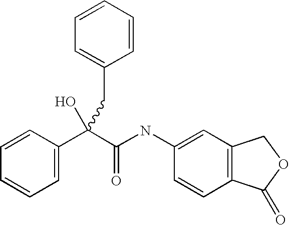

Compound 24 was prepared in analogy to Example 5) from the substance described under 22a) and benzylmagnesium chloride.

¹H NMR (ppm, CDCl₃, 400 MHz): 2.83 (1H); 3.23 (1H); 3.96 (1H); 5.24 (2H); 7.18 (2H); 7.22-7.38 (5H); 7.40 (2H); 7.74 (2H); 7.79 (1H); 8.10 (1H); 8.86 (1H).

### Example 24a and 24b

### (+)-5-[2-hydroxy-2,3-diphenylpropionylamino]phthalide 24a and

### (−)-5-[2-hydroxy-2,3-diphenylpropionylamino]phthalide 24b

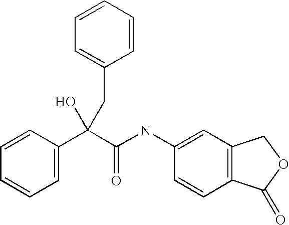

The racemic mixture obtained under Example 24 was separated by preparative chiral HPLC (column: Chiralpak AD 250×10 mm) into enantiomers 24a and 24b.

24a and 24b:

24a: [α]^{D}₂₀: +27.0° (CHCl₃, 10.1 mg/1 ml; λ=589 nM)

24b: [α]^{D}₂₀: −27.0° (CHCl₃, 10.5 mg/1 ml; λ=589 nM)

### Example 25

### rac-5-[2-hydroxy-2-(4-nitrophenyl)-4-phenyl but-3-ynoylamino]phthalide 25

### a) 5-[2-(4-nitrophenyl)-2-oxoacetoylamino]phthalide

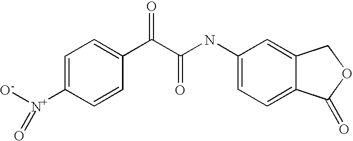

Compound 25a) was synthesized in analogy to Example 1a) from (4-nitro-phenyl)glyoxylic acid, thionyl chloride and 5-aminophthalide in N,N-dimethylacetamide.

¹H NMR (ppm, DMSO-D₆, 400 MHz): 5.39 (2H); 7.60 (2H); 7.70-7.90 (3H); 8.02 (2H); 8.13 (1H).

### b) rac-5-[2-hydroxy-2-(4-nitrophenyl)-4-phenyl but-3-ynoylamino]phthalide

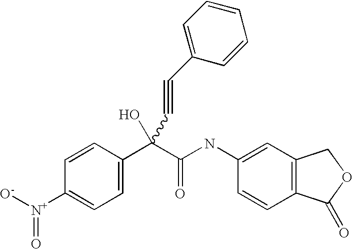

Compound 25b) was prepared in analogy to Example 1b) from the substance described under 25a), phenylacetylene and n-butyllithium.

¹H NMR (ppm, CDCl₃, 400 MHz): 4.46 (1H); 5.24 (2H); 7.32-7.48 (4H); 7.51 (2H); 7.84 (1H); 8.02 (2H); 8.09 (1H); 8.29 (2H); 8.96 (1H).

### Example 26

### rac-5-[2-hydroxy-2-(4-nitrophenyl)-2-phenylacetoylamino]phthalide

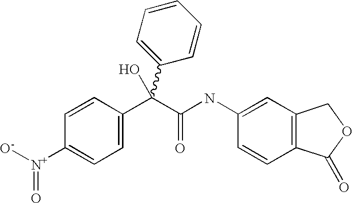

Compound 26 was prepared in analogy to Example 4 from the substance described under 25a) and phenylmagnesium bromide.

¹H NMR (ppm, CDCl₃, 400 MHz): 3.51 (1H); 5.26 (2H); 7.33-7.46 (6H); 7.84 (3H); 8.21 (3H); 9.31 (1H).

### Example 27

### rac-6-[2-hydroxy-2-(4-nitrophenyl)-3-phenylpropionylamino]phthalide

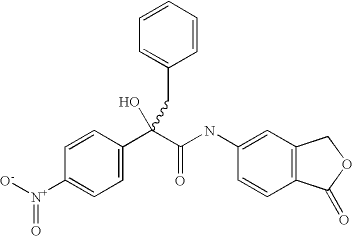

Compound 27 was prepared in analogy to Example 5) from the substance described under 25a) and benzylmagnesium chloride.

¹H NMR (ppm, CDCl₃, 300 MHz): 3.00 (1H); 3.20 (1H); 3.99 (1H); 5.26 (2H); 7.17 (2H);

7.25-7.38 (4H); 7.81 (1H); 7.97 (2H); 8.10 (1H); 8.25 (2H); 8.86 (1H).

### Example 27a and 27b

### (+)-6-[2-hydroxy-2-(4-nitrophenyl)-3-phenylpropionylamino]phthalide 27a and

### (−)-6-[2-hydroxy-2-(4-nitrophenyl)-3-phenylpropionylamino]phthalide 27b

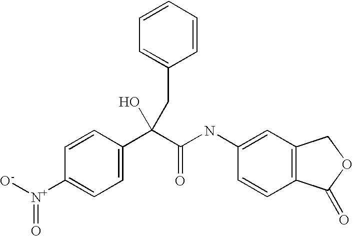

The racemic mixture obtained under Example 27 was separated by preparative chiral HPLC (column: Chiralpak AD 250×10 mm) into enantiomers 27a and 27b.

27a and 27b:

27a: [α]^{D}₂₀: +18.9° (CHCl₃, 6.8 mg/1 ml; λ=589 nM)

27b: [α]^{D}₂₀: −18.8° (CHCl₃, 5.2 mg/1 ml; λ=589 nM)

### Example 28

### rac-6-[2-hydroxy-2-(4-cyanophenyl)-3-phenylpropionylamino]phthalide

### a) 5-[2-(4-cyanophenyl)-2-oxoacetoylamino]phthalide

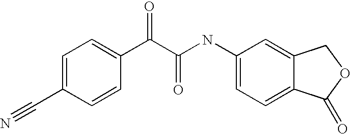

Compound 28a) was synthesized in analogy to Example 1a) from (4-cyano-phenyl)glyoxylic acid, thionyl chloride and 5-aminophthalide in N,N-dimethylacetamide.

¹H NMR (ppm, DMSO-D₆, 300 MHz): 5.38 (2H); 7.82 (2H); 8.02 (2H); 8.12 (1H); 8.18 (2H); 11.42 (1H).

### b) rac-6-[2-hydroxy-2-(4-cyanophenyl)-3-phenylpropionylamino]phthalide

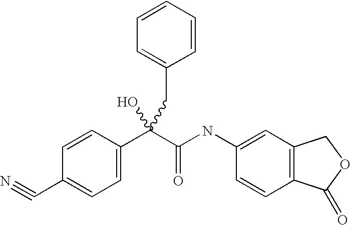

Compounds 28b) was prepared in analogy to Example 5) from the substance described under 28a) and benzylmagnesium chloride.

¹H NMR (ppm, CDCl₃, 400 MHz): 2.98 (1H); 3.18 (1H); 3.96 (1H); 5.25 (2H); 7.15 (2H); 7.22-7.40 (4H); 7.69 (2H); 7.80 (1H); 7.90 (2H); 8.09 (1H); 8.85 (1H).

### Example 29

### rac-N-(3-chloro-4-cyanophenyl)-2-hydroxy-2,3-diphenylpropionamide

### a) N-(3-chloro-4-cyanophenyl)-2-oxo-2-phenylacetamide

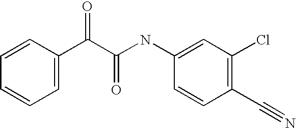

Compound 29a) was synthesized in analogy to Example 1a) from phenylglyoxylic acid, thionyl chloride and 4-amino-2-chlorobenzonitrile in N,N-dimethylacetamide.

¹H NMR (ppm, DMSO-D₆, 400 MHz): 7.58 (2H); 7.74 (1H); 7.80 (1H); 7.96 (1H); 8.04 (2); 8.15 (1H); 11.50 (1H).

Compounds 29b) and 30-33 were prepared in analogy to Example 5) from the substance described under 29a) and the respective benzyl Grignard reagent.

### b) rac-N-(3-chloro-4-cyanophenyl)-2-hydroxy-2,3-diphenylpropionamide

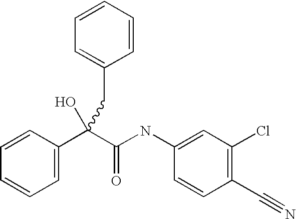

¹H NMR (ppm, CDCl₃, 400 MHz): 2.74 (1H); 3.21 (1H); 3.92 (1H); 7.16 (2H); 7.22-7.46 (7H); 7.53 (1H); 7.70 (2H); 7.90 (1H); 8.72 (2H).

### Example 29c and 29d

### (+)-N-(3-chloro-4-cyanophenyl)-2-hydroxy-2,3-diphenylpropionamide 29c and

### (−)-N-(3-chloro-4-cyanophenyl)-2-hydroxy-2,3-diphenylpropionamide 29d

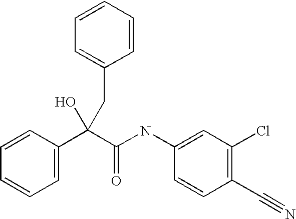

The racemic mixture obtained under Example 29b was separated by preparative chiral HPLC (column: Chiralpak AD 250×10 mm) into enantiomers 29c and 29d.

29c and 29d:

29c: [α]^{D}₂₀: +37.4° (CHCl₃, 10.5 mg/1 ml; λ=589 nM)

29d: [α]^{D}₂₀: −37.8° (CHCl₃, 10.4 mg/1 ml; λ=589 nM)

### Example 30

### rac-N-(3-chloro-4-cyanophenyl)-2-hydroxy-2-phenyl-3-(4-methylphenyl)propion-amide

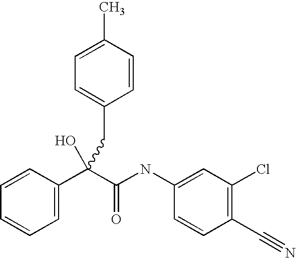

¹H NMR (ppm, CDCl₃, 400 MHz): 2.30 (3H); 2.78 (1H); 3.16 (1H); 3.90 (1H); 7.02 (2H); 7.11 (2H); 7.30-7.50 (4H); 7.55 (1H), 7.70 (2H); 7.90 (1H); 8.76 (1H).

### Example 31

### rac-N-(3-chloro-4-cyanophenyl)-2-hydroxy-3-(4-methoxyphenyl)-2-phenylpropion-amide

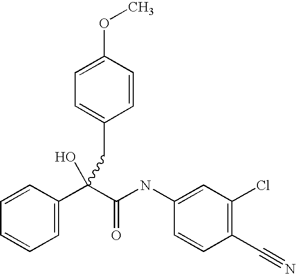

¹H NMR (ppm, CDCl₃, 400 MHz): 2.73 (1H); 3.13 (1H); 3.76 (3H); 3.88 (1H); 6.82 (2H); 7.08 (2H); 7.30-7.45 (4H); 7.55 (1H); 7.70 (2H); 7.90 (1H); 8.76 (1H).

### Example 32

### rac-N-(3-chloro-4-cyano-

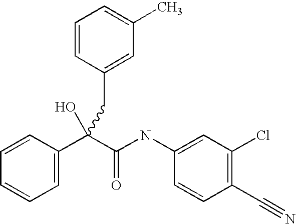

¹H NMR (ppm, CDCl₃, 400 MHz): 2.29 (3H); 2.79 (1H); 3.16 (1H); 3.90 (1H); 6.94 (2H); 7.09 (1H); 7.19 (1H); 7.30-7.46 (4H); 7.55 (1H); 7.70 (2H); 7.90 (1H); 8.75 (1H).

### Example 33

### rac-N-(3-chloro-4-cyanophenyl)-2-hydroxy-3-(3-methoxyphenyl)-2-phenyl-propionamide

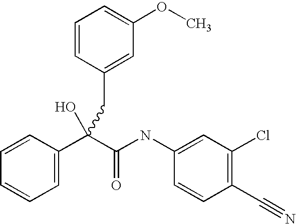

¹H NMR (ppm, CDCl₃, 400 MHz): 2.81 (1H); 3.19 (1H); 3.68 (3H); 3.89 (1H); 6.65 (1H); 6.72 (1H); 6.81 (1H); 7.21 (1H); 7.30-7.48 (4H); 7.54 (1H); 7.70 (2H); 7.90 (1H); 8.79 (1H).

### Example 34

### rac-N-(4-cyano-3-trifluoromethylphenyl)-2-hydroxy-2,3-diphenylpropionamide

### a) N-(4-cyano-3-trifluoromethylphenyl)-2-oxo-2-phenylacetamide

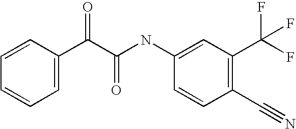

Compound 34a) was synthesized in analogy to Example 1a) from phenylglyoxylic acid, thionyl chloride and 4-4-amino-2-trifluoromethylbenzonitrile in N,N-dimethylacetamide.

¹H NMR (ppm, CDCl₃, 300MHz): 7.54 (2H); 7.70 (1H); 7.88 (1H); 8.02 (1H); 8.21 (1H); 8.42 (2H); 9.34 (1H).

Compounds 34b) and 35-38 were prepared in analogy to Example 5) from the substance described under 34a) and the respective benzyl Grignard reagent.

### b) rac-N-(4-cyano-3-trifluoromethylphenyl)-2-hydroxy-2,3-diphenylpropionamide

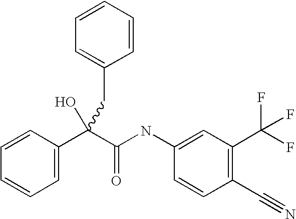

¹H NMR (ppm, CDCl₃, 400 MHz): 2.78 (1H); 3.24 (1H); 3.92 (1H); 7.15 (2H); 7.22-7.46 (6H); 7.71 (3H); 7.37 (1H); 7.98 (1H); 8.89 (1H).

### Example 34c and 34d

### (+)-N-(4-cyano-3-trifluoromethylphenyl)-2-hydroxy-2,3-diphenylpropionamide 34c and

### (−)-N-(4-cyano-3-trifluoromethylphenyl)-2-hydroxy-2,3-diphenylpropionamide 34d

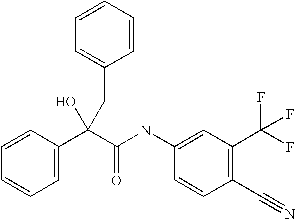

The racemic mixture obtained under Example 34b was separated by preparative chiral HPLC (column: Chiralpak AD 250×10 mm) into enantiomers 34c and 34d.

34c and 34d:

34c: [α]^{D}₂₀: +36.5° (CHCl₃, 10.4 mg/1 ml; λ=589 nM)

34d: [α]^{D}₂₀: −36.2° (CHCl₃, 10.4 mg/1 ml; λ=589 nM)

### Example 35

### rac-N-(4-cyano-3-trifluoromethylphenyl)-2-hydroxy-2-phenyl-3-(4-methylphenyl)-propionamide

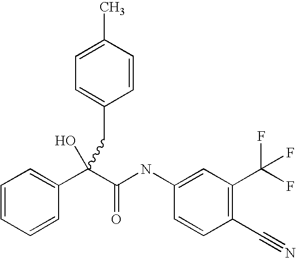

¹H NMR (ppm, CDCl₃, 300 MHz): 2.30 (3H); 2.80 (1H); 3.19 (1H); 3.90 (1H); 7.02 (2H); 7.10 (2H); 7.30-7.48 (3H); 7.72 (3H); 7.88 (1H); 7.98 (1H); 8.90 (1H).

### Example 36

### rac-N-(4-cyano-3-trifluoromethylphenyl)-2-hydroxy-3-(4-methoxyphenyl)-2-phenylpropionamide

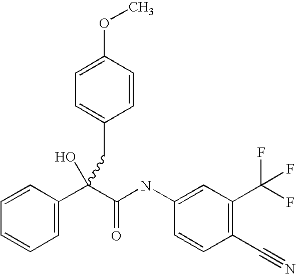

¹H NMR (ppm, CDCl₃, 300 MHz): 2.78 (1H); 3.17 (1H); 3.77 (3H); 3.89 (1H); 6.81 (2H); 7.08 (2H); 7.30-7.48 (3H); 7.71 (3H); 7.87 (1H); 8.00 (1H); 8.90 (1H).

### Example 37

### rac-N-(4-cyano-3-trifluoromethylphenyl)-2-hydroxy-2-phenyl-3-(3-methylphenyl)-propionamide

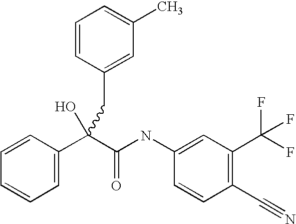

¹H NMR (ppm, CDCl₃, 400 MHz): 2.28 (3H); 2.80 (1H); 3.18 (1H); 3.90 (1H); 6.95 (2H); 7.09 (1H); 7.19 (1H); 7.30-7.45 (3H); 7.72 (3H); 7.88 (1H); 7.98 (1H); 8.90 (1H).

### Example 38

### rac-N-(4-cyano-3-trifluoromethylphenyl)-2-hydroxy-3-(3-methoxyphenyl)-2-phenylpropionamide

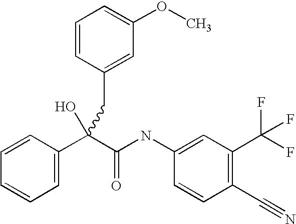

¹H NMR (ppm, CDCl₃, 400 MHz): 2.87 (1H); 3.21 (1H); 3.68 (3H); 3.89 (1H); 6.64 (1H); 6.74 (1H); 6.81 (1H); 7.22 (1H); 7.30-7.46 (3H); 7.73 (3H); 7.88 (1H); 8.00 (1H); 8.91 (1H).

### Example 39

### rac-6-[2-hydroxy-2-(4-phenylphenyl)-3-(4-methylphenyl)propionylamino]-4-methyl-2,3-benzoxazin-1-one

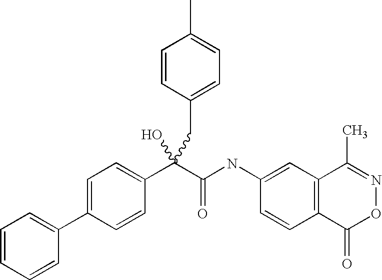

Compound 39 was prepared analogously to Example 5) from the substance described under 21a) and 4-methylbenzylmagnesium chloride.

¹H NMR (ppm, CDCl₃, 400 MHz): 2.31 (3H); 2.58 (3H), 2.91 (1H); 3.28 (1H); 3.97 (1H); 7.10 (4H); 7.36 (1H); 7.45 (2H); 7.60 (2H); 7.65 (3H); 7.81 (2H); 8.27 (2H); 9.04 (1H).

### Example 39a and 39b

### (+)-6-[2-hydroxy-2-(4-phenylphenyl)-3-(4-methylphenyl)propionylamino]-4-methyl-2,3-benzoxazin-1-one 39a and

### (−)-6-[2-hydroxy-2-(4-phenylphenyl)-3-(4-methylphenyl)propionylamino]-4-methyl-2,3-benzoxazin-1-one 39b

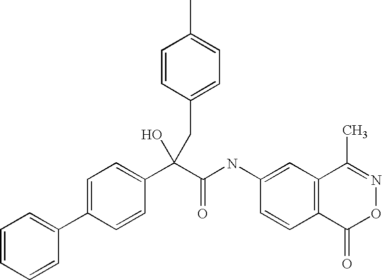

The racemic mixture obtained under Example 39 was separated by preparative chiral HPLC (column: Chiralpak AD 250×10 mm) into enantiomers 39a and 39b.

39a and 39b:

39a: [α]^{D}₂₀: +11.9° (CHCl₃, 10.2 mg/1 ml; λ=589 nM)

39b: [α]^{D}₂₀: −12.8° (CHCl₃, 10.3 mg/1 ml; λ=589 nM)

### Example 40

### rac-6-[2-hydroxy-2-(4-phenylphenyl)-3-(3-methylphenyl)propionylamino]-4-methyl-2,3-benzoxazin-1-one

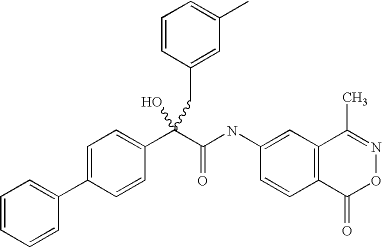

Compound 40 was prepared analogously to Example 5) from the substance described under 21a) and 3-methylbenzylmagnesium chloride.

¹H NMR (ppm, CDCl₃, 400 MHz): 2.28 (3H), 2.58 (3H); 2.93 (1H); 3.27 (1H); 3.97 (1H); 7.00 (2H); 7.11 (1H), 7.20 (1H), 7.36 (1H); 7.45 (2H); 7.60 (2H); 7.65 (3H); 7.81 (2H); 8.28 (2H); 9.03 (1H).

### Example 41

### rac-6-[2-hydroxy-2-(4-trifluoromethylphenyl)-3-(3-methylphenyl)propionylamino]-4-methyl-2,3-benzoxazin-1-one

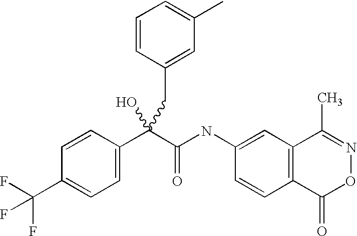

Compound 41 was prepared analogously to Example 5) from the substance described under 19a) and 3-methylbenzylmagnesium chloride.

¹H NMR (ppm, CDCl₃, 400 MHz): 2.28 (3H), 2.58 (3H); 3.01 (1H); 3.19 (1H); 3.96 (1H); 6.95 (2H), 7.12 (1H); 7.20 (1H), 7.66 (3H); 7.91 (2H); 8.27 (2H); 9.00 (1H).

### Example 42

### rac-6-[2-hydroxy-2-(4-trifluoromethylphenyl)-3-phenylpropionylamino]-4-methyl-2,3-benzoxazin-1-one

### a) ethyl (4-methoxyphenyl)glyoxate

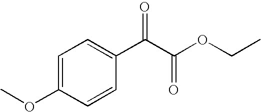

1,2-Dibromomethane (20 μl) and 4-bromoanisole (1.3 g) were added to a suspension of magnesium (170 mg) in THF (8 ml). This suspension was stirred at 40° C. for a further 1.5 hours. The reaction mixture was cooled to −70° C. A solution of diethyl oxalate (500 mg) in THF (4 ml) was then added dropwise. Subsequently, the mixture was stirred at −70° C. over a further 2.5 h. The reaction mixture was then poured onto ice-cold saturated ammonium chloride solution. It was extracted with ethyl acetate. The combined organic phases were washed with saturated sodium chloride solution and dried over sodium sulphate. The crude product was chromatographed on silica gel. 670 mg of product were obtained.

### b) 4-methoxyphenylglyoxylic acid

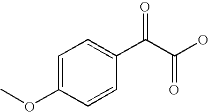

A solution of 0.5 g of sodium hydroxide in 8 ml of water was added to a solution of the compound described under 42a) in 9 ml of ethanol. The mixture was left to stir at 23° C. for a further 1.5 hours, then diluted with water and extracted with ethyl acetate. Subsequently, the aqueous phase was acidified with 2 normal hydrochloric acid (pH 4). This was followed by extraction with ethyl acetate and washing of the organic phase with saturated aqueous sodium chloride solution. It was then dried over sodium sulphate and concentrated under reduced pressure. The resulting crude product (0.4 g) was used in the next stage without purification.

### c) 6-[2-(4-methoxyphenyl)-2-oxoacetoylamino]-4-methyl-2,3-benzoxazin-1-one

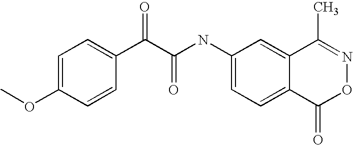

Compound 42c) was synthesized analogously to Example 1a) from compound 42b, thionyl chloride and 6-amino-4-methyl-2,3-benzoxazin-1-one in N,N-dimethylacetamide.

Compounds 42d) and 43-44 were prepared analogously to Example 5) from the substance described under 42c) and the particular benzyl Grignard reagent.

### d) rac-6-[2-hydroxy-2-(4-methoxyphenyl)-3-phenylpropionylamino]-4-methyl-2,3-benzoxazin-1-one

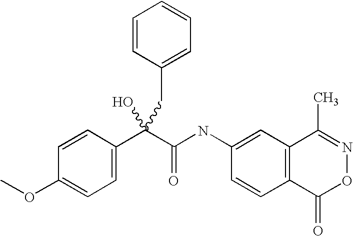

¹H NMR (ppm, CDCl₃, 400 MHz): 2.57 (3H); 2.80 (1H); 3.30 (1H); 3.83 (3H), 3.90 (1H); 6.95 (2H); 7.16 (2H), 7.28-7.32 (3H); 7.62-7.66 (3H); 8.26 (2H); 8.98 (1H).

### Example 42e and 42f

### (+)-6-[2-hydroxy-2-(4-methoxyphenyl)-3-phenylpropionylamino]-4-methyl-2,3-benzoxazin-1-one 42e and

### (−)-6-[2-hydroxy-2-(4-methoxyphenyl)-3-phenylpropionylamino]-4-methyl-2,3-benzoxazin-1-one 42f

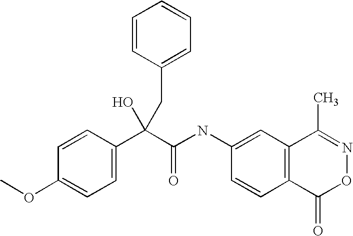

The racemic mixture obtained under Example 42d was separated by preparative chiral HPLC (column: Chiralpak AD 250×10 mm) into enantiomers 42e and 42f.

42e and 42f:

42e: [α]^{D}₂₀: +16.3° (CHCl₃, 10.3 mg/1 ml; λ=589 nM)

42f: [α]^{D}₂₀: −17.4° (CHCl₃, 10.3 mg/1 ml; λ=589 nM)

### Example 43

### rac-6-[2-hydroxy-2-(4-methoxyphenyl)-3-(4-methylphenyl)propionylamino]-4-methyl-2,3-benzoxazin-1-one

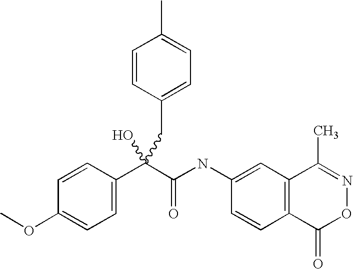

¹H NMR (ppm, CDCl₃, 400 MHz): 2.30 (3H), 2.57 (3H); 2.79 (1H); 3.23 (1H); 3.82 (3H), 3.87 (1H); 6.93 (2H); 7.04 (2H); 7.10 (2H), 7.62-7.66 (3H); 8.27 (2H); 8.98 (1H).

### Example 44

### rac-6-[2-hydroxy-2-(4-methoxyphenyl)-3-(3-methylphenyl)propionylamino]-4-methyl-2,3-benzoxazin-1-one

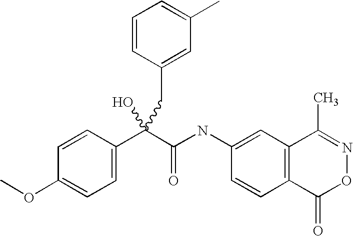

¹H NMR (ppm, CDCl₃, 400 MHz): 2.27 (3H), 2.57 (3H); 2.84 (1H); 3.22 (1H); 3.82 (3H), 3.88 (1H); 6.91-6.98 (4H), 7.09 (1H), 7.18 (1H); 7.62-7.66 (3H); 8.26 (2H); 8.99 (1H).

### Example 45

### rac-6-[2-hydroxy-2-(4-(1,1-dimethylethyl)phenyl)-3-phenylpropionylamino]-4-methyl-2,3-benzoxazin-1-one

### a) 4-(1,1-dimethylethyl)phenylglyoxylic acid

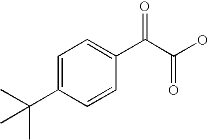

Compound 45a) was synthesized analogously to Example 42b) from ethyl 4-(1,1-dimethylethyl)phenylglyoxylate.

### b) 6-[2-(4-(1,1-dimethylethyl)phenyl)-2-oxoacetoylamino]-4-methyl-2,3-benzoxazin-1-one

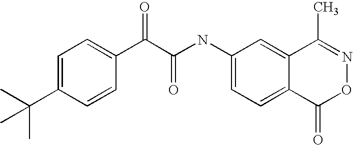

Compound 45b) was synthesized analogously to Example 1a) from compound 45a, thionyl chloride and 6-amino-4-methyl-2,3-benzoxazin-1-one in N,N-dimethylacetamide.

Compounds 45c and 46-47 were prepared analogously to Example 5) from the substance described under 45b) and the particular benzyl Grignard reagent.

### c) rac-6-[2-hydroxy-2-(4-(1,1-dimethylethyl)phenyl)-3-phenylpropionylamino]-4-methyl-2,3-benzoxazin-1-one

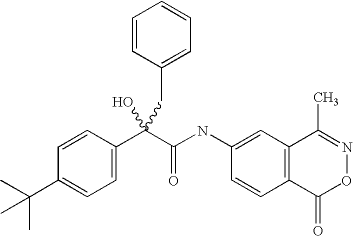

¹H NMR (ppm, CDCl₃, 400 MHz): 1.32 (9H), 2.57 (3H); 2.82 (1H); 3.23 (1H); 3.99 (1H); 7.20 (2H); 7.29 (3H); 7.44 (2H), 7.60-7.67 (3H); 8.26 (2H); 8.99 (1H).

### Example 45d and 45e

### (+)-6-[2-hydroxy-2-(4-(1,1-dimethylethyl)phenyl)-3-phenylpropionylamino]-4-methyl-2,3-benzoxazin-1-one 45d and

### (−)-6-[2-hydroxy-2-(4-(1,1-dimethylethyl)phenyl)-3-phenylpropionylamino]-4-methyl-2,3-benzoxazin-1-one 45e

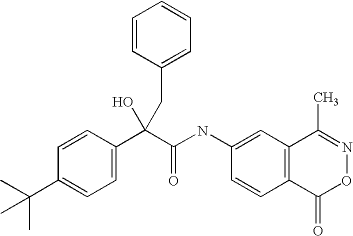

The racemic mixture obtained under Example 45c was separated by preparative chiral HPLC (column: Chiralpak AD 250×10 mm) into enantiomers 45d and 45e.

45d and 45e:

45d: [α]^{D}₂₀: +30.0° (CHCl₃, 9.4 mg/1 ml; λ=589 nM)

45e: [α]^{D}₂₀: −31.0° (CHCl₃, 9.4 mg/1 ml; λ=589 nM)

### Example 46

### rac-6-[2-hydroxy-2-(4-(1,1-dimethylethyl)phenyl)-3-(4-methylphenyl)propionyl-amino]-4-methyl-2,3-benzoxazin-1-one

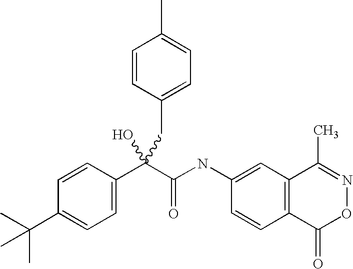

¹H NMR (ppm, CDCl₃, 400 MHz): 1.32 (9H), 2.30 (3H), 2.57 (3H); 2.84 (1H); 3.18 (1H); 3.97 (1H); 7.08 (4H); 7.43 (2H); 7.64 (3H); 8.26 (2H); 9.00 (1H).

### Example 47

### rac-6-[2-hydroxy-2-(4-(1,1-dimethylethyl)phenyl)-3-(3-methylphenyl)propionyl-amino]-4-methyl-2,3-benzoxazin-1-one

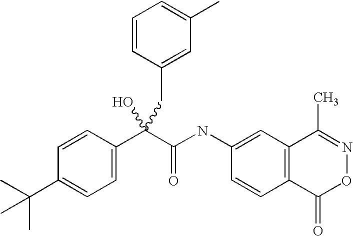

¹H NMR (ppm, CDCl₃, 400 MHz): 1.32 (9H), 2.26 (3H), 2.57 (3H); 2.84 (1H); 3.19 (1H); 3.95 (1H); 6.99 (2H); 7.09 (1H), 7.18 (1H), 7.43 (2H); 7.61-7.67 (3H); 8.26 (2H); 9.00 (1H).

### Example 48

### rac-6-[2-hydroxy-2-(4-dimethylaminophenyl)-3-phenylpropionylamino]-4-methyl-2,3-benzoxazin-1-one

### a) ethyl (4-dimethylaminophenyl)glyoxate

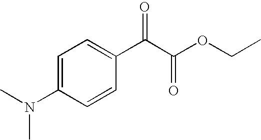

Compound 48a) was synthesized analogously to Example 42a) from 4-dimethylaminophenylmagnesium bromide and ethyl glyoxalate.

### b) 4-dimethylaminophenylglyoxylic acid

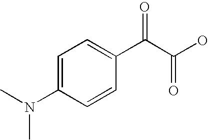

Compound 48b) was synthesized from 48a) analogously to Example 42b).

### c) 6-[2-(4-dimethylaminophenyl)-2-oxoacetoylamino]-4-methyl-2,3-benzoxazin-1-one

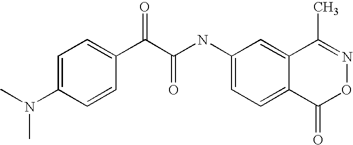

Compound 48c) was synthesized analogously to Example 1a) from compound 48b, thionyl chloride and 6-amino-4-methyl-2,3-benzoxazin-1-one in N,N-dimethylacetamide.

Compounds 48d) and 49-50 were prepared analogously to Example 5) from the substance described under 48c) and the particular benzyl Grignard reagent.

### d) rac-6-[2-hydroxy-2-(4-dimethylaminophenyl)-3-phenylpropionylamino]-4-methyl-2,3-benzoxazin-1-one

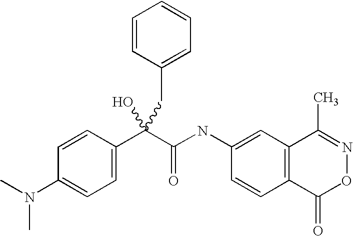

¹H NMR (ppm, CDCl₃, 400 MHz): 2.55 (3H); 2.76 (1H); 2.96 (6H), 3.29 (1H); 3.88 (1H); 6.75 (2H); 7.19 (2H); 7.25-7.30 (3H); 7.55 (2H); 7.61 (1H), 8.25 (2H); 8.97 (1H).

### Example 49

### rac-6-[2-hydroxy-2-(4-dimethylaminophenyl)-3-(4-methylphenyl)propionylamino]-4-methyl-2,3-benzoxazin-1-one

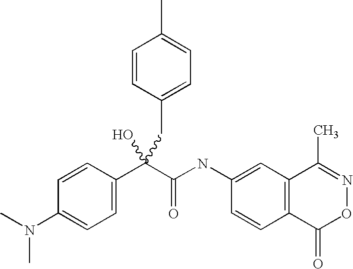

¹H NMR (ppm, CDCl₃, 400 MHz): 2.29 (3H), 2.56 (3H); 2.74 (1H); 2.96 (6H), 3.23 (1H); 3.86 (1H); 6.75 (2H); 7.08 (4H), 7.55 (2H); 7.61 (1H); 8.26 (2H); 8.98 (1H).

### Example 50

### rac-6-[2-hydroxy-2-(4-dimethylaminophenyl)-3-(3-methylphenyl)propionylamino]-4-methyl-2,3-benzoxazin-1-one

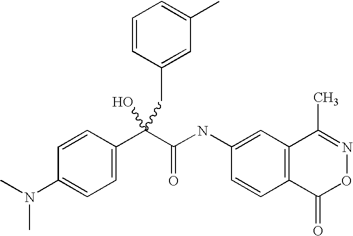

¹H NMR (ppm, CDCl₃, 400 MHz): 2.27 (3H), 2.56 (3H); 2.77 (1H); 2.96 (6H), 3.22 (1H); 3.88 (1H); 6.75 (2H); 6.99 (2H), 7.07 (1H), 7.17 (1H), 7.56 (2H); 7.62 (1H); 8.26 (2H); 8.97 (1H).

### Example 51

### rac-6-[2-hydroxy-2-(4-phenoxyphenyl)-3-phenylpropionylamino]-4-methyl-2,3-benzoxazin-1-one

### a) ethyl (4-phenoxyphenyl)glyoxate

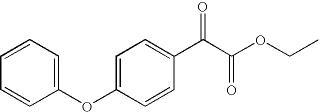

Compound 51a) was synthesized analogously to Example 42a) from 4-phenoxyphenyl bromide and ethyl glyoxalate.

### b) 4-phenoxyphenylglyoxylic acid

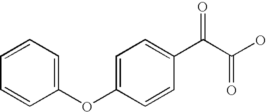

Compound 51b) was synthesized from 51a) analogously to Example 42b).

### c) 6-[2-(4-phenoxyphenyl)-2-oxoacetoylamino]-4-methyl-2,3-benzoxazin-1-one

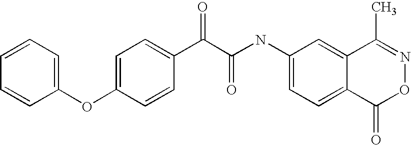

Compound 51c) was synthesized analogously to Example 1a) from compound 51b, thionyl chloride and 6-amino-4-methyl-2,3-benzoxazin-1-one in N,N-dimethylacetamide.

Compounds 51d) and 52-53 were prepared analogously to Example 5) from the substance described under 51c) and the particular benzyl Grignard reagent.

### d) rac-6-[2-hydroxy-2-(4-phenoxyphenyl)-3-phenylpropionylamino]-4-methyl-2,3-benzoxazin-1-one

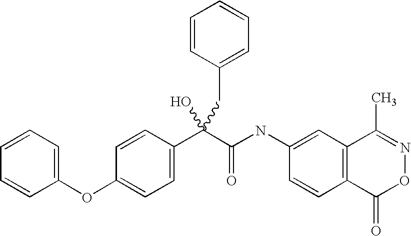

¹H NMR (ppm, CDCl₃, 400 MHz): 2.57 (3H); 2.91 (1H); 3.28 (1H); 3.93 (1H); 7.02 (4H); 7.10-7.20 (3H); 7.29-7.37 (5H); 7.63-7.70 (3H), 8.26 (2H); 9.01 (1H).

### Example 51e and 51f

### (+)-6-[2-hydroxy-2-(4-phenoxyphenyl)-3-phenylpropionylamino]-4-methyl-2,3-benzoxazin-1-one 51e and

### (−)-6-[2-hydroxy-2-(4-phenoxyphenyl)-3-phenylpropionylamino]-4-methyl-2,3-benzoxazin-1-one 51f

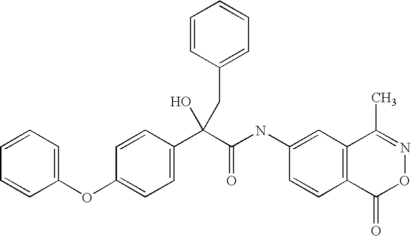

The racemic mixture obtained under Example 51d was separated by preparative chiral HPLC (column: Chiralpak AD 250×10 mm) into enantiomers 51e and 51f.

51e and 51f:

51e: [α]^{D}₂₀: +1.7° (CHCl₃, 12.4 mg/1 ml; λ=589 nM)

51f: [α]^{D}₂₀: −2.1° (CHCl₃, 12.6 mg/1 ml; λ=589 nM)

### Example 52

### rac-6-[2-hydroxy-2-(4-phenoxyphenyl)-3-(4-methylphenyl)propionylamino]-4-methyl-2,3-benzoxazin-1-one

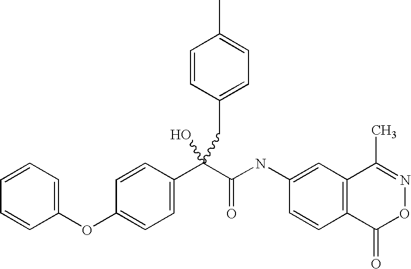

¹H NMR (ppm, CDCl₃, 400 MHz): 2.30 (3H), 2.58 (3H); 2.88 (1H); 3.22 (1H); 3.90 (1H); 7.01-7.15 (9H); 7.34 (2H); 7.64-7.70 (3H), 8.26 (2H); 9.02 (1H).

### Example 53

### rac-6-[2-hydroxy-2-(4-phenoxyphenyl)-3-(3-methylphenyl)propionylamino]-4-methyl-2,3-benzoxazin-1-one

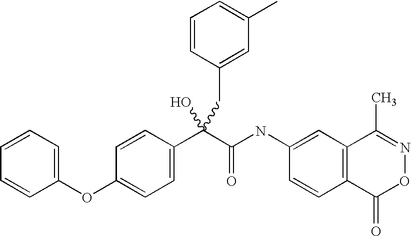

¹H NMR (ppm, CDCl₃, 400 MHz): 2.28 (3H), 2.58 (3H); 2.87 (1H); 3.23 (1H); 3.90 (1H); 6.95-7.05 (6H); 7.10-7.22 (3H); 7.34 (2H); 7.64-7.71 (3H); 8.28 (2H); 9.00 (1H).

### Example 54

### rac-6-[2-hydroxy-2-(4-hydroxyphenyl)-3-phenylpropionylamino]-4-methyl-2,3-benzoxazin-1-one

### a) 6-[2-(4-hydroxyphenyl)-2-oxoacetoylamino]-4-methyl-2,3-benzoxazin-1-one

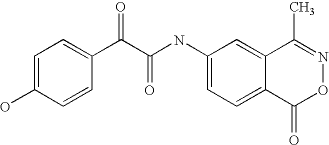

Boron tribromide (1M in dichloromethane, 4.4 ml) was added at −50° C. to a solution of the compound described under 42c (200 mg) in dichloromethane (6 ml). The reaction mixture was allowed to warm up to 230° C. over 3 hours and then left to stir for a further 24 hours. Thereafter, the reaction mixture was poured onto ice-cold saturated sodium hydrogencarbonate. It was extracted with ethyl acetate. The combined organic phases were washed with saturated sodium chloride solution and dried over sodium sulphate. The crude product was chromatographed on silica gel. 74 mg of product were obtained.

### b) 6-[2-(4-((1,1-dimethylethyl)dimethylsilyloxy)phenyl)-2-oxoacetoylamino]-4-methyl-2,3-benzoxazin-1-one

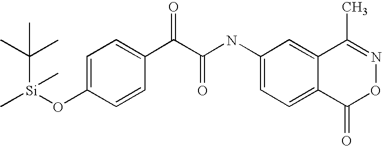

Imidazole (250 mg) and tert-butyldimethylsilyl chloride (350 mg) were added at 23° C. to a solution of the compound described under 54a) (150 mg) in absolute N,N-dimethyl-formamide (8 ml). The mixture was left to stir at 23° C. for a further 30 hours. Thereafter, the reaction mixture was poured onto saturated aqueous sodium hydrogencarbonate solution. The mixture was stirred for a further 10 minutes and then extracted with ethyl ether. The organic phase was washed with saturated aqueous sodium chloride solution, dried over sodium sulphate and concentrated under reduced pressure. The crude product was chromatographed on silica gel. 56 mg of product were obtained.

### c) rac-6-[2-hydroxy-2-(4-((1,1-dimethylethyl)dimethylsilyloxy)phenyl)-3-phenyl-propionylamino]-4-methyl-2,3-benzoxazin-1-one

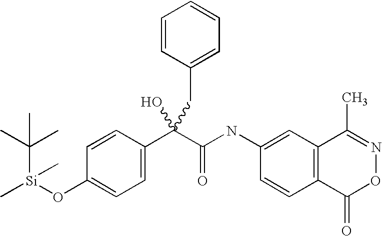

Compounds 54c) were prepared analogously to Example 5) from the substance described under 54b) and benzyl magnesium chloride.

### d) rac-6-[2-hydroxy-2-(4-hydroxyphenyl)-3-phenylpropionylamino]-4-methyl-2,3-benzoxazin-1-one

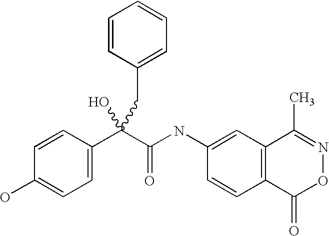

The product obtained under 54c) (50 mg) was dissolved in tetrahydrofuran. Tetrabutylammonium fluoride was added and the mixture was left to stir at 23° C. for a further 45 minutes. Thereafter, the reaction mixture was poured onto saturated aqueous sodium hydrogencarbonate solution. The mixture was stirred for a further 15 minutes and then extracted with ethyl acetate. The organic phase was washed with saturated aqueous sodium chloride solution, dried over sodium sulphate and concentrated under reduced pressure. The resulting crude product was chromatographed on silica gel. 30 mg of product were obtained.

¹H NMR (ppm, CDCl₃, 400 MHz): 2.56 (3H); 2.83 (1H); 3.28 (1H); 3.88 (1H); 5.27 (1H), 6.86 (2H); 7.16 (2H), 7.29 (3H); 7.58 (2H); 7.64 (1H), 8.26 (2H); 8.98 (1H).

### Example 55

### rac-6-[2-hydroxy-2-(4-acetylphenyl)-3-phenylpropionylamino]-4-methyl-2,3-benzoxazin-1-one

### a) 2,5,5-trimethyl-2-(4-bromo)phenyl-1,3-dioxane

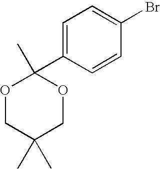

4-Bromoacetophenone (5 g) was dissolved in 50 ml of dichloromethane. 2,2-Dimethylpropylene glycol (8.3 g) and p-toluenesulphonic acid (50 mg) were added, and the mixture was left to stir at RT for 18 hours. Thereafter, the reaction mixture was poured onto saturated aqueous sodium hydrogencarbonate solution. It was extracted with ethyl acetate, then the organic phase was washed with saturated aqueous sodium chloride solution, dried over sodium sulphate and concentrated under reduced pressure. The resulting crude product was chromatographed on silica gel. 6.8 g of product was obtained.

### b) ethyl [4-(2,2,5-trimethyl-1,3-dioxolan-2-yl)phenyl]glyoxate

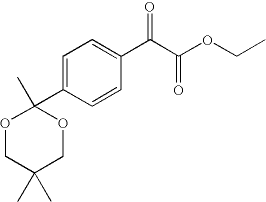

Compound 55b) was synthesized analogously to Example 42a) from 55a) and ethyl glyoxalate.

### c) [4-(2,2,5-trimethyl-1,3-dioxolan-2-yl)phenyl]glyoxylic acid

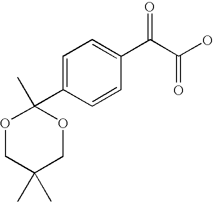

Compound 55c) was synthesized from 55b) analogously to Example 42b).

### d) 6-[2-(4-(2,2,5-trimethyl-1,3-dioxolan-2-yl)phenyl)-2-oxoacetoylamino]-4-methyl-2,3-benzoxazin-1-one

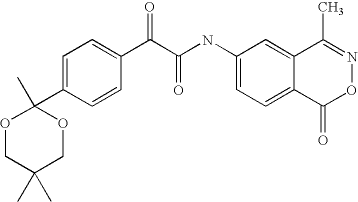

Compound 55d) was synthesized analogously to Example 1a) from compound 55c, thionyl chloride and 6-amino-4-methyl-2,3-benzoxazin-1-one in N,N-dimethylacetamide.

### e) rac-6-[2-hydroxy-2-(4-(2,2,5-trimethyl-1,3-dioxolan-2-yl)phenyl)-3-phenyl-propionylamino]-4-methyl-2,3-benzoxazin-1-one

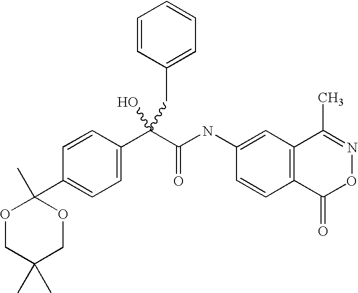

Compounds 55e) were prepared analogously to Example 5) from the substance described under 55d) and benzylmagnesium chloride.

### f) rac-6-[2-hydroxy-2-(4-acetylphenyl)-3-phenylpropionylamino]-4-methyl-2,3-benzoxazin-1-one

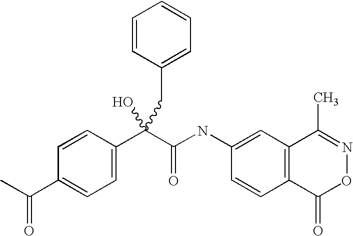

The product reaction obtained under 55e) (120 mg) was dissolved in 6 ml of acetone. 0.25 ml of 2 normal hydrochloric acid was added and the mixture was left to stir for a further 3 hours. Subsequently, the mixture was poured onto saturated aqueous sodium hydrogencarbonate solution and extracted with dichloromethane. The combined organic phases were washed with saturated aqueous sodium chloride solution, dried over sodium sulphate and concentrated under reduced pressure. The resulting crude product was chromatographed on silica gel. 75 mg of product were obtained.

¹H NMR (ppm, CDCl₃, 400 MHz): 2.57 (3H); 2.61 (3H), 3.04 (1H); 3.30 (1H); 3.95 (1H); 7.16 (2H); 7.30 (3H); 7.65 (1H); 7.87 (2H); 7.99 (2H), 8.26 (2H); 9.01 (1H).

### Example 55g and 55h

### (+)-6-[2-hydroxy-2-(4-acetylphenyl)-3-phenylpropionylamino]-4-methyl-2,3-benz-oxazin-1-one 55g and

### (−)-6-[2-hydroxy-2-(4-acetylphenyl)-3-phenylpropionylamino]-4-methyl-2,3-benz-oxazin-1-one 55h

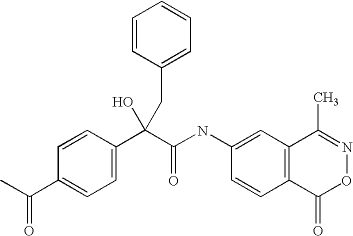

The racemic mixture obtained under Example 55f was separated by preparative chiral HPLC (column: Chiralpak AD 250×10 mm) into enantiomers 55g and 55h.

55g and 55h:

55g: [α]^{D}₂₀: +17.2° (CHCl₃, 5.4 mg/1 ml; λ=589 nM)

55h: [α]^{D}₂₀: −16.5° (CHCl₃, 5.3 mg/1 ml; λ=589 nM)

### Example 56

### rac-6-[2-hydroxy-2-(4-acetylphenyl)-3-(4-methylphenyl)propionylamino]-4-methyl-2,3-benzoxazin-1-one

### a) rac-6-[2-hydroxy-2-(4-(2,2,5-trimethyl-1,3-dioxolan-2-yl)phenyl)-3-(4-methyl-phenyl)propionylamino]-4-methyl-2,3-benzoxazin-1-one

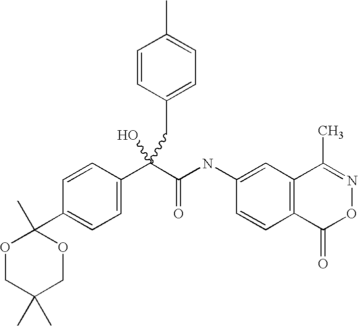

Compounds 56a) were prepared analogously to Example 5) from the substance described under 55d) and 4-methylbenzylmagnesium chloride.

### b) rac-6-[2-hydroxy-2-(4-acetylphenyl)-3-(4-methylphenyl)propionylamino]-4-methyl-2,3-benzoxazin-1-one

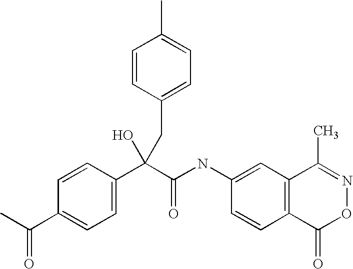

Compounds 56b) were prepared analogously to Example 55f) from the substance described under 56a).

¹H NMR (ppm, CDCl₃, 400 MHz): 2.30 (3H), 2.57 (3H); 2.61 (3H), 3.02 (1H); 3.24 (1H); 3.92 (1H); 7.03 (2H), 7.11 (2H); 7.66 (1H); 7.87 (2H); 7.99 (2H); 8.27 (2H); 9.02 (1H).

### Example 57

### rac-6-[2-hydroxy-2-(4-acetylphenyl)-3-(3-methylphenyl)propionylamino]-4-methyl-2,3-benzoxazin-1-one

### a) rac-6-[2-hydroxy-2-(4-(2,2,5-trimethyl-1,3-dioxolan-2-yl)phenyl)-3-(3-methyl-phenyl)propionylamino]-4-methyl-2,3-benzoxazin-1-one

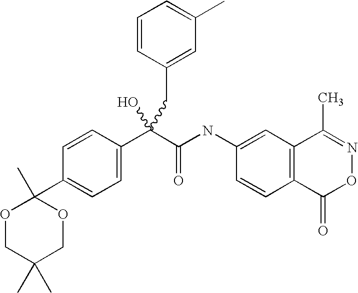

Compounds 57a) were prepared analogously to Example 5) from the substance described under 55d) and 4-methylbenzylmagnesium chloride.

### b) rac-6-[2-hydroxy-2-(4-acetylphenyl)-3-(3-methylphenyl)propionylamino]-4-methyl-2,3-benzoxazin-1-one

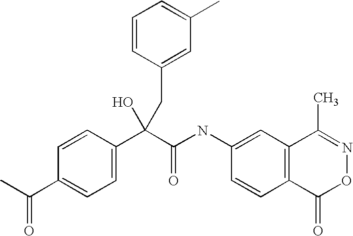

Compounds 57b) were prepared analogously to Example 55f) from the substance described under 57a).

¹H NMR (ppm, CDCl₃, 400 MHz): 2.27 (3H), 2.57 (3H); 2.61 (3H), 3.06 (1H); 3.23 (1H); 3.94 (1H); 6.95 (2H), 7.10 (1H); 7.19 (1H), 7.66 (1H); 7.87 (2H); 7.99 (2H); 8.26 (2H); 9.02 (1H).

### Example 58

### rac-6-[2-hydroxy-2-(3-acetylphenyl)-3-phenylpropionylamino]-4-methyl-2,3-benzoxazin-1-one

### a) 2,5,5-trimethyl-2-(3-bromo)phenyl-1,3-dioxane

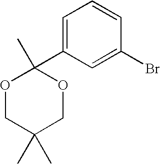

Compounds 58a) were prepared analogously to Example 55a) from 3-bromo-acetophenone.

### b) ethyl [3-(2,2,5-trimethyl-1,3-dioxolan-2-yl)phenyl]glyoxate

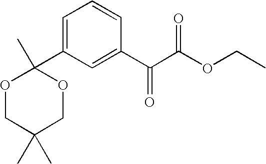

Compound 58b) was synthesized analogously to Example 42a) from 58a) and ethyl glyoxalate.

### c) [3-(2,2,5-trimethyl-1,3-dioxolan-2-yl)phenyl]glyoxylic acid

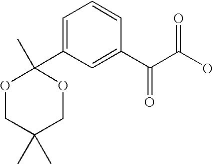

Compound 58c) was synthesized analogously to Example 42b) from 58b).

### d) 6-[2-(3-(2,2,5-trimethyl-1,3-dioxolan-2-yl)phenyl)-2-oxoacetoylamino]-4-methyl-2,3-benzoxazin-1-one

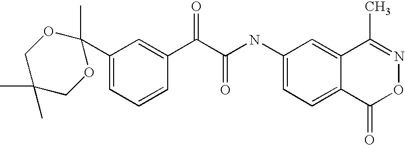

Compound 58d) was synthesized analogously to Example 1a) from compound 58c, thionyl chloride and 6-amino-4-methyl-2,3-benzoxazin-1-one in N,N-dimethylacetamide.

### e) rac-6-[2-hydroxy-2-(3-(2,2,5-trimethyl-1,3-dioxolan-2-yl)phenyl)-3-phenyl-propionylamino]-4-methyl-2,3-benzoxazin-1-one

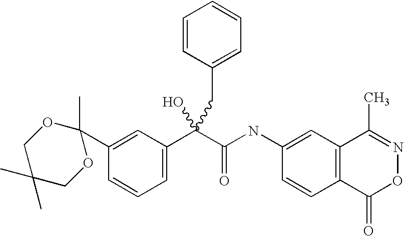

Compounds 58e) were prepared analogously to Example 5) from the substance described under 58d) and benzylmagnesium chloride.

### f) rac-6-[2-hydroxy-2-(3-acetylphenyl)-3-phenylpropionylamino]-4-methyl-2,3-benzoxazin-1-one

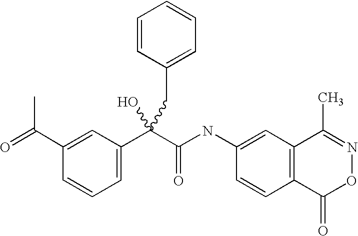

Compounds 58f) were prepared analogously to Example 55f) from the substance described under 58e).

¹H NMR (ppm, DMSO-d₆, 400 MHz): 2.41 (3H), 2.53 (3H); 3.27 (1H); 3.69 (1H); 6.87 (1H), 7.08-7.15 (5H); 7.49 (1H); 7.89 (2H); 8.12 (1H); 8.20 (2H); 8.33 (1H), 10.37 (1H).

### Example 59

### rac-6-[2-hydroxy-2-(2-methylphenyl)-3-phenylpropionylamino]-4-methyl-2,3-benzoxazin-1-one

### a) ethyl (2-methylphenyl)glyoxate

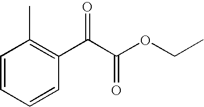

Compound 59a) was synthesized analogously to Example 42a) from 2-bromotoluene and ethyl glyoxalate.

### b) 2-methylphenylglyoxylic acid

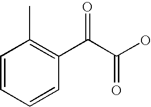

Compound 59b) was synthesized analogously to Example 42b) from 59a).

### c) 6-[2-(2-methylphenyl)-2-oxoacetoylamino]-4-methyl-2,3-benzoxazin-1-one

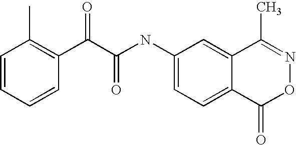

Compound 59c) was synthesized analogously to Example 1a) from compound 59b, thionyl chloride and 6-amino-4-methyl-2,3-benzoxazin-1-one in N,N-dimethylacetamide.

### d) rac-6-[2-hydroxy-2-(2-methylphenyl)-3-phenylpropionylamino]-4-methyl-2,3-benzoxazin-1-one

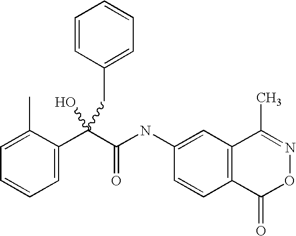

Compounds 59d) were prepared analogously to Example 5) from the substance described under 59c) and benzylmagnesium chloride.

¹H NMR (ppm, CDCl₃, 400 MHz): 2.47 (3H), 2.54 (3H); 2.97 (1H); 3.48 (1H) 3.87 (1H); 7.20-7.31 (8H); 7.40 (1H); 7.67 (1H); 8.14 (1H); 8.24 (1H); 8.43 (1H).

### Example 60

### rac-6-[2-hydroxy-2-(3-trifluoromethylphenyl)-3-phenylpropionylamino]-4-methyl-2,3-benzoxazin-1-one

### a) 3-trifluoromethylphenylglyoxylic acid

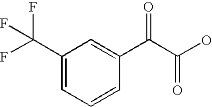

Compound 60a) was synthesized analogously to Example 42b) from ethyl 3-trifluoromethylphenylglyoxylate.

### b) 6-[2-(3-trifluoromethylphenyl)-2-oxoacetoylamino]-4-methyl-2,3-benzoxazin-1-one

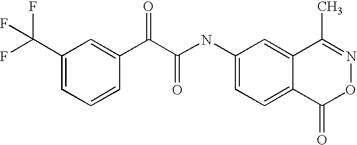

Compound 60b) was synthesized analogously to Example 1a) from compound 60a, thionyl chloride and 6-amino-4-methyl-2,3-benzoxazin-1-one in N,N-dimethylacetamide.

### c) rac-6-[2-hydroxy-2-(3-trifluoromethylphenyl)-3-phenylpropionylamino]-4-methyl-2,3-benzoxazin-1-one

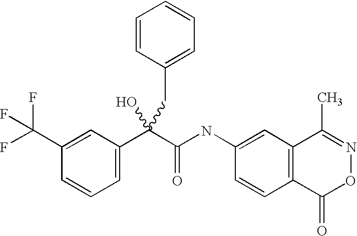

Compounds 60c) were prepared analogously to Example 5) from the substance described under 60b) and benzylmagnesium chloride.

¹H NMR (ppm, CDCl₃, 400 MHz): 2.58 (3H); 3.01 (1H); 3.23 (1H); 4.00 (1H); 7.18 (2H); 7.32 (3H); 7.54-7.69 (3H); 8.02 (2H); 8.26 (2H); 9.00 (1H).

### Example 60d and 60e

### (+)-6-[2-hydroxy-2-(3-trifluoromethylphenyl)-3-phenylpropionylamino]-4-methyl-2,3-benzoxazin-1-one 60d and

### (−)-6-[2-hydroxy-2-(3-trifluoromethylphenyl)-3-phenylpropionylamino]-4-methyl-2,3-benzoxazin-1-one 60e

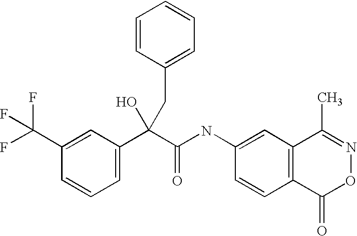

The racemic mixture obtained under example 60c was separated by preparative chiral HPLC (column: Chiralpak AD 250×10 mm) into enantiomers 60d and 60e.

60d and 60e:

60d: [α]^{D}₂₀: +29.1° (CHCl₃, 8.3 mg/1 ml; λ=589 nM)

60e: [α]^{D}₂₀: −28.7° (CHCl₃, 8.6 mg/1 ml; λ=589 nM)

### Example 61

### rac-6-[2-hydroxy-2-(3-methoxyphenyl)-3-phenylpropionylamino]-4-methyl-2,3-benzoxazin-1-one

### a) 3-methoxyphenylglyoxylic acid

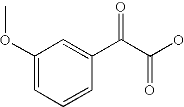

Compound 61a) was synthesized analogously to Example 42b) from ethyl 3-methoxyphenylglyoxylate.

### b) 6-[2-(3-methoxyphenyl)-2-oxoacetoylamino]-4-methyl-2,3-benzoxazin-1-one

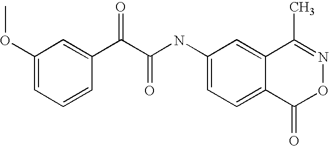

Compound 61b) was synthesized analogously to Example 1a) from compound 61a), thionyl chloride and 6-amino-4-methyl-2,3-benzoxazin-1-one in N,N-dimethylacetamide.

### c) rac-6-[2-hydroxy-2-(3-methoxyphenyl)-3-phenylpropionylamino]-4-methyl-2,3-benzoxazin-1-one

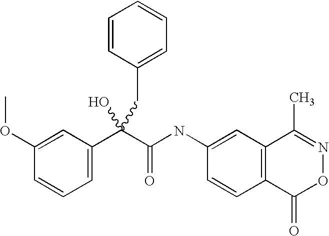

Compounds 61c) were prepared analogously to Example 5) from the substance described under 61b) and benzylmagnesium chloride.

¹H NMR (ppm, CDCl₃, 400 MHz): 2.57 (3H); 2.86 (1H); 3.29 (1H); 3.84 (3H), 3.93 (1H); 6.89 (1H), 7.18 (2H); 7.28-7.34 (6H); 7.64 (1H); 8.26 (2H); 8.96 (1H).

### Example 61d and 61e

### (+)-6-[2-hydroxy-2-(3-methoxyphenyl)-3-phenylpropionylamino]-4-methyl-2,3-benzoxazin-1-one 61d and

### (−)-6-[2-hydroxy-2-(3-methoxyphenyl)-3-phenylpropionylamino]-4-methyl-2,3-benzoxazin-1-one 60e

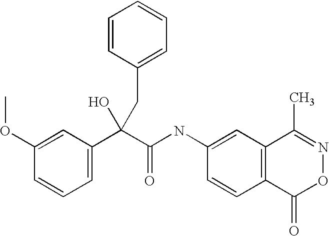

The racemic mixture obtained under Example 61c was separated by preparative chiral HPLC (column: Chiralpak AD 250×10 mm) into enantiomers 61d and 61e.

61d and 61e:

61d: [α]^{D}₂₀: +12.6° (CHCl₃, 10.3 mg/1 ml; λ=589 nM)

61e: [α]^{D}₂₀: −12.5° (CHCl₃, 11.7 mg/1 ml; λ=589 nM)

### Example 62

### rac-6-[2-hydroxy-2-(4-thiomethoxyphenyl)-3-phenylpropionylamino]-4-methyl-2,3-benzoxazin-1-one

### a) ethyl (4-thiomethoxyphenyl)glyoxate

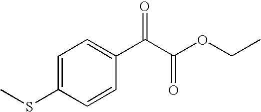

Compound 62a) was synthesized analogously to Example 42a) from 4-thiomethylphenylmagnesium bromide and ethyl glyoxalate.

### b) 4-thiomethoxyphenylglyoxylic acid

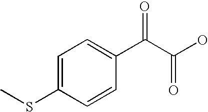

Compound 62b) was synthesized analogously to Example 42b) from 62a).

### c) 6-[2-(4-thiomethoxyphenyl)-2-oxoacetoylamino]-4-methyl-2,3-benzoxazin-1-one

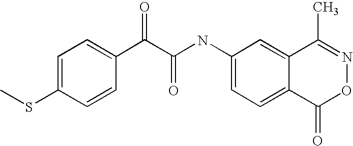

Compound 62c) was synthesized analogously to Example 1a) from compound 62b, thionyl chloride and 6-amino-4-methyl-2,3-benzoxazin-1-one in N,N-dimethylacetamide.

### d) rac-6-[2-hydroxy-2-(4-thiomethoxyphenyl)-3-phenylpropionylamino]-4-methyl-2,3-benzoxazin-1-one

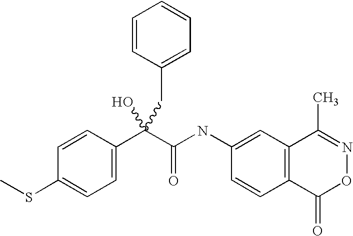

Compounds 62d) were prepared analogously to Example 5) from the substance described under 62c) and benzylmagnesium chloride.

¹H NMR (ppm, CDCl₃, 400 MHz): 2.48 (3H), 2.57 (3H); 2.84 (1H); 3.28 (1H); 3.90 (1H); 7.17 (2H); 7.29 (5H); 7.61-7.66 (3H); 8.26 (2H); 8.97 (1H).

### Example 63

### rac-6-[2-hydroxy-2-(4-hydroxymethylphenyl)-3-phenylpropionylamino]-4-methyl-2,3-benzoxazin-1-one

### a) tetrahydro-2-(benzyloxy)-2H-pyran none

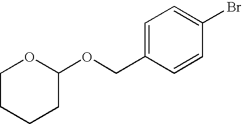

4-Bromophenylmethanol (4 g) was dissolved in 90 ml of dichloromethane. 3,4-Dihydro-2H-pyran (15 ml) and pyridinium tosylate (100 mg) were added, and the mixture was left to stir at 23° C. for 5 hours. Thereafter, the reaction mixture was poured onto saturated aqueous sodium hydrogencarbonate solution. It was extracted with ethyl acetate, then the organic phase was washed with saturated aqueous sodium chloride solution, dried over sodium sulphate and concentrated under reduced pressure. The resulting crude product was chromatographed on silica gel. 5.3 g of product were obtained.

### b) ethyl [4-(tetrahydropyran-2-yloxymethyl)phenyl]glyoxate

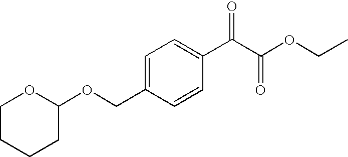

Compound 63b) was synthesized analogously to Example 42a) from 63a) and ethyl glyoxalate.

### c) [4-(tetrahydropyran-2-yloxymethyl)phenyl]glyoxylic acid

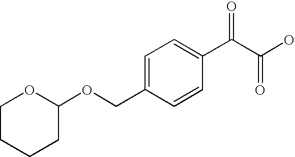

Compound 63c) was synthesized analogously to Example 42b) from 63b).

### d) 6-[2-(4-(tetrahydropyran-2-yloxymethyl)phenyl)-2-oxoacetoylamino]-4-methyl-2,3-benzoxazin-1-one

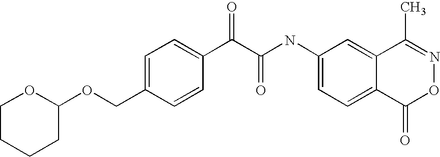

Compound 63d) was synthesized analogously to Example 1a) from compound 63c, thionyl chloride and 6-amino-4-methyl-2,3-benzoxazin-1-one in N,N-dimethylacetamide.

### e) rac-6-[2-hydroxy-2-(4-(tetrahydropyran-2-yloxymethyl)phenyl)-3-phenyl-propionylamino]-4-methyl-2,3-benzoxazin-1-one

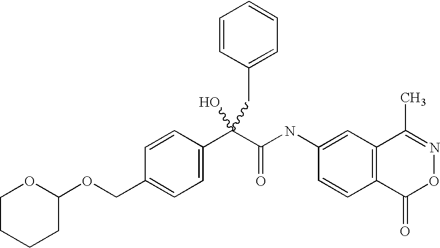

Compounds 63e) were prepared analogously to Example 5) from the substance described under 63d) and benzylmagnesium chloride.

### f) rac-6-[2-hydroxy-2-(4-hydroxymethylphenyl)-3-phenylpropionylamino]-4-methyl-2,3-benzoxazin-1-one

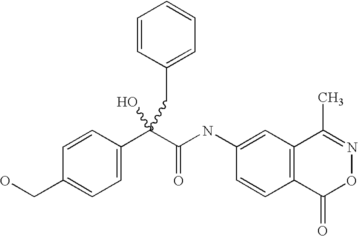

The product obtained under 63e) (130 mg) was dissolved in 5 ml of ethanol. 44 mg of p-toluenesulphonic acid were added and the reaction mixture was left to stir for a further 2 hours. Subsequently, the mixture was poured onto saturated aqueous sodium hydrogencarbonate solution and extracted with dichloromethane. The combined organic phases were washed with saturated aqueous sodium chloride solution, dried over sodium sulphate and concentrated under reduced pressure. The resulting crude product was chromatographed on silica gel. 56 mg of product were obtained.

¹H NMR (ppm, CDCl₃, 400 MHz): 2.17 (2H), 2.55 (3H); 3.29 (1H); 3.94 (1H); 4.71 (2H), 7.17 (2H); 7.28 (3H); 7.41 (2H), 7.63 (1H); 7.73 (2H); 8.23 (2H); 9.01 (1H).

### Example 64

### rac-6-[2-hydroxy-2-(thiophen-2-yl)-3-phenylpropionylamino]-4-methyl-2,3-benzoxazin-1-one

### a) 6-[2-(thiophen-2-yl)-2-oxoacetoylamino]-4-methyl-2,3-benzoxazin-1-one

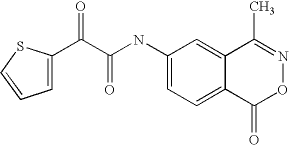

Compound 64a) was synthesized analogously to Example 1a) from thiophen-2-yl-glyoxylic acid, thionyl chloride and 6-amino-4-methyl-2,3-benzoxazin-1-one in N,N-dimethylacetamide.

### b) rac-6-[2-hydroxy-2-(thiophen-2-yl)-3-phenylpropionylamino]-4-methyl-2,3-benzoxazin-1-one

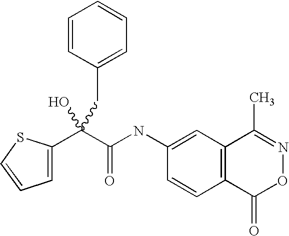

Compounds 64b) were prepared analogously to Example 5) from the substance described under 64a) and benzylmagnesium chloride.

¹H NMR (ppm, CDCl₃, 400 MHz): 2.58 (3H); 3.17 (1H); 3.41 (1H); 3.83 (1H); 7.04 (1H); 7.19 (2H), 7.24 (1H); 7.29-7.33 (4H); 7.66 (1H), 8.28 (2H); 8.96 (1H).

### Example 64c and 64d

### (+)-6-[2-hydroxy-2-(thiophen-2-yl)-3-phenylpropionylamino]-4-methyl-2,3-benzoxazin-1-one 20a and

### 6-[2-hydroxy-2-(thiophen-2-yl)-3-phenylpropionylamino]-4-methyl-2,3-benzoxazin-1-one 64d

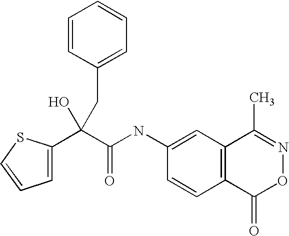

The racemic mixture obtained under Example 64b was separated by preparative chiral HPLC (column: Chiralpak AD 250×10 mm) into enantiomers 64c and 64d.

64c and 64d:

64c: [α]^{D}₂₀: +30.1° (CHCl₃, 10.2 mg/1 ml; λ=589 nM)

64d: [α]^{D}₂₀: −31.0° (CHCl₃, 10.3 mg/1 ml; λ=589 nM)

### Example 65

### rac-6-[2-hydroxy-2-(N-methylindol-3-yl)-3-phenylpropionylamino]-4-methyl-2,3-benzoxazin-1-one

### a) 6-[2-(N-methylindol-3-yl)-2-oxoacetoylamino]-4-methyl-2,3-benzoxazin-1-one

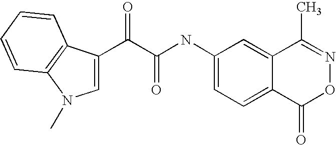

Compound 65a) was synthesized analogously to Example 1a) from N-methylindol-3-yl-glyoxylic acid, thionyl chloride and 6-amino-4-methyl-2,3-benzoxazin-1-one in N,N-dimethylacetamide.

### b) rac-6-[2-hydroxy-2-(N-methylindol-3-yl)-3-phenylpropionylamino]-4-methyl-2,3-benzoxazin-1-one

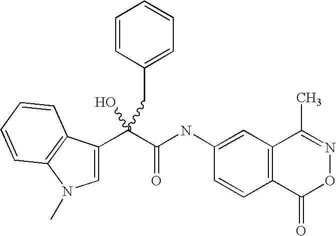

Compounds 65b) were prepared analogously to Example 5) from the substance described under 65a) and benzylmagnesium chloride.

¹H NMR (ppm, CDCl₃, 400 MHz): 2.55 (3H); 2.94 (1H); 3.56 (1H); 3.80 (3H), 3.90 (1H); 7.18 (1H); 7.00-7.29 (5H); 7.34 (2H), 7.59 (1H); 7.94 (1H); 8.24 (2H); 9.03 (1H).

### Example 66

### rac-6-[2-hydroxy-2-(furan-2-yl)-3-phenylpropionylamino]-4-methyl-2,3-benzoxazin-1-one

### a) 6-[2-(furan-2-yl)-2-oxoacetoylamino]-4-methyl-2,3-benzoxazin-1-one

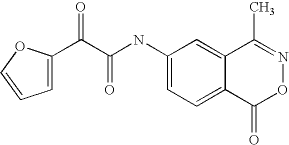

Compound 66a) was synthesized analogously to Example 1a) from furan-2-ylglyoxylic acid, thionyl chloride and 6-amino-4-methyl-2,3-benzoxazin-1-one in N,N-dimethylacetamide.

### b) rac-6-[2-hydroxy-2-(furan-2-yl)-3-phenylpropionylamino]-4-methyl-2,3-benzoxazin-1-one

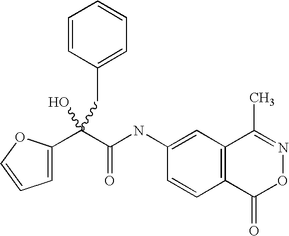

Compounds 66b) were prepared analogously to Example 5) from the substance described under 66a) and benzylmagnesium chloride.

¹H NMR (ppm, CDCl₃, 400 MHz): 2.57 (3H); 3.21 (1H); 3.57 (2H); 6.42 (1H), 6.50 (1H), 7.16 (2H); 7.27 (3H); 7.48 (1H); 7.61 (1H); 8.29 (2H); 8.93 (1H).

### Example 67

### rac-6-[2-hydroxy-2-(pyridin-3-yl)-3-phenylpropionylamino]-4-methyl-2,3-benzoxazin-1-one

### a) 6-[2-(pyridin-3-yl)-2-oxoacetoylamino]-4-methyl-2,3-benzoxazin-1-one

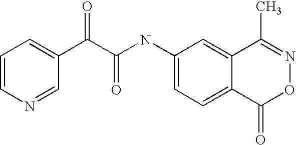

Compound 67a) was synthesized analogously to Example 1a) from pyrid-3-ylglyoxylic acid, thionyl chloride and 6-amino-4-methyl-2,3-benzoxazin-1-one in N,N-dimethylacetamide.

### b) rac-6-[2-hydroxy-2-(pyridin-3-yl)-3-phenylpropionylamino]-4-methyl-2,3-benzoxazin-1-one

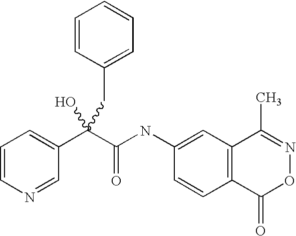

Compounds 67b) were prepared analogously to Example 5) from the substance described under 67a) and benzylmagnesium chloride.

¹H NMR (ppm, DMSO-d₆, 400 MHz): 2.46 (3H); 3.38 (1H); 3.70 (1H); 7.13-7.19 (5H); 7.69 (1H); 8.17 (1H); 8.24 (1H); 8.34 (2H); 8.65 (1H), 8.92 (1H), 10.48 (1H).

Analogously to Example 5, compounds 68-73 were prepared from the substance described under 1a) and the particular benzyl Grignard reagent.

### Example 68

### rac-6-[2-hydroxy-2-phenyl-3-(2-bromophenyl)propionylamino]-4-methyl-2,3-benzoxazin-1-one

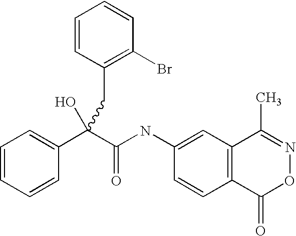

¹H NMR (ppm, CDCl₃, 400 MHz): 2.57 (3H); 3.35 (1H); 3.66 (1H); 4.09 (1H); 7.10-7.18 (3H); 7.37 (1H); 7.44 (2H); 7.62 (2H); 7.74 (2H); 8.26 (2H); 9.01 (1H).

### Example 68a and 68b

### (+)-6-[2-hydroxy-2-phenyl-3-(2-bromophenyl)propionylamino]-4-methyl-2,3-benzoxazin-1-one 68a and

### (−)-6-[2-hydroxy-2-phenyl-3-(2-bromophenyl)propionylamino]-4-methyl-2,3-benzoxazin-1-one 68b

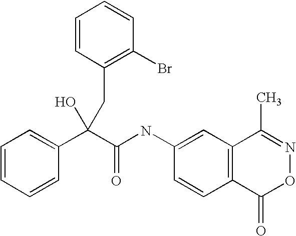

The racemic mixture obtained under example 68 was separated by preparative chiral HPLC (column: Chiralpak AD 250×10 mm) into enantiomers 68a and 68b.

20 68a and 68b:

68a: [α]^{D}₂₀: +99.8° (CHCl₃, 7.1 mg/1 ml; λ=589 nM)

68b: [α]^{D}₂₀: −101.2° (CHCl₃, 7.5 mg/1 ml; λ=589 nM)

### Example 69

### rac-6-[2-hydroxy-2-phenyl-3-(3-bromophenyl)propionylamino]-4-methyl-2,3-benzoxazin-1-one

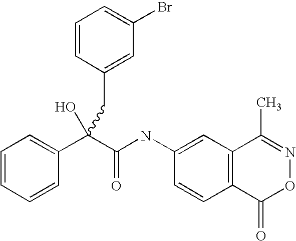

¹H NMR (ppm, CDCl₃, 400 MHz): 2.58 (3H); 2.88 (1H); 3.30 (1H); 3.88 (1H); 7.13 (2H); 7.37-7.47 (5H); 7.65 (1H); 7.72 (2H); 8.26 (2H); 8.96 (1H).

### Example 69a and 69b

### (+)-6-[2-hydroxy-2-phenyl-3-(3-bromophenyl)propionylamino]-4-methyl-2,3-benzoxazin-1-one 69a and

### (−)-6-[2-hydroxy-2-phenyl-3-(3-bromophenyl)propionylamino]-4-methyl-2,3-benzoxazin-1-one 69b

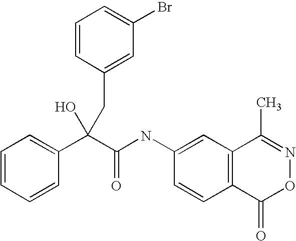

The racemic mixture obtained under Example 69 was separated by preparative chiral HPLC (column: Chiralpak AD 250×10 mm) into enantiomers 69a and 69b.

69a and 69b:

69a: [α]^{D}₂₀: +60.4° (CHCl₃, 8.9 mg/1 ml; λ=589 nM)

69b: [α]^{D}₂₀: −73.7° (CHCl₃, 9.5 mg/1 ml; λ=589 nM)

### Example 70

### rac-6-[2-hydroxy-2-phenyl-3-(naphth-1-yl)propionylamino]-4-methyl-2,3-benzoxazin-1-one

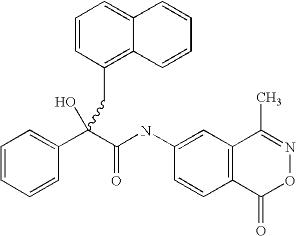

¹H NMR (ppm, CDCl₃, 400 MHz): 2.55 (3H); 2.97 (1H); 3.45 (1H); 4.12 (1H); 7.24 (1H); 7.34-7.49 (5H); 7.65 (2H); 7.75-7.80 (5H); 8.23 (2H); 8.98(1H).

### Example 70a and 70b

### (+)-6-[2-hydroxy-2-phenyl-3-(naphth-1-yl)propionylamino]-4-methyl-2,3-benzoxazin-1-one 70a and

### (−)-6-[2-hydroxy-2-phenyl-3-(naphth-1-yl)propionylamino]-4-methyl-2,3-benzoxazin-1-one 70b

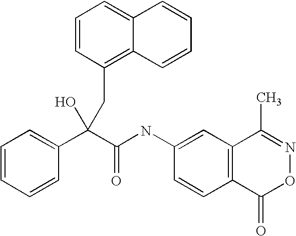

The racemic mixture obtained under Example 70 was separated by preparative chiral HPLC (column: Chiralpak AD 250×10 mm) into enantiomers 70a and 70b.

70a and 70b:

70a: [α]^{D}₂₀: +84.8° (CHCl₃, 10.2 mg/1 ml; λ=589 nM)

70b: [α]^{D}₂₀: −73.3° (CHCl₃, 10.0 mg/1 ml; λ=589 nM)

### Example 71

### rac-6-[2-hydroxy-2-phenyl-3-(2-methylphenyl)propionylamino]-4-methyl-2,3-benzoxazin-1-one

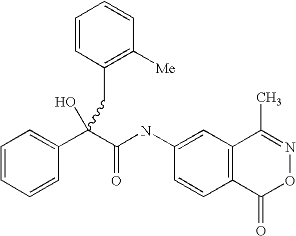

¹H NMR (ppm, CDCl₃, 400 MHz): 2.29 (3H); 2.57 (3H); 2.87 (1H); 3.44 (1H); 3.97 (1H); 7.10 (2H); 7.18 (2H); 7.3-7.45 (3H); 7.65 (1H); 7.73 (2H); 8.26 (2H); 9.03 (1H).

### Example 72

### rac-6-[2-hydroxy-2-phenyl-3-(4-(1,1-dimethylethyl)phenyl)propionylamino]-4-methyl-2,3-benzoxazin-1-one

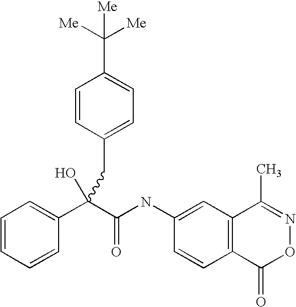

¹H NMR (ppm, CDCl₃, 400 MHz): 1.28 (9H); 2.58 (3H); 2.89 (1H); 3.25 (1H); 3.95 (1H); 7.12 (2H); 7.33 (2H); 7.36-7.46 (3H); 7.64 (1H); 7.76 (2H); 8.28 (2H); 9.02 (1H).

### Example 73

### rac-6-[2-hydroxy-2-phenyl-3-(4-phenylphenyl)propionylamino]-4-methyl-2,3-benzoxazin-1-one

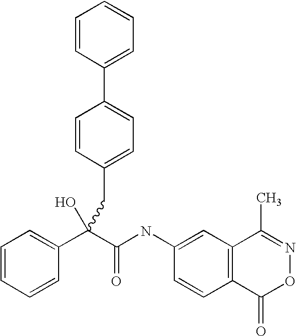

¹H NMR (ppm, CDCl₃, 400 MHz): 2.56 (3H); 2.90 (1H); 3.34 (1H); 4.00 (1H); 7.24 (2H); 7.35-7.46 (6H); 7.53 (4H); 7.66 (1H); 7.77 (2H); 8.26 (2H); 9.02 (lH).

### Example 74

### rac-6-[2-hydroxy-2-(4-iodophenyl)-3-phenylpropionylamino]-4-methyl-2,3-benzoxazin-1-one

### a) ethyl (4-iodophenyl)glyoxate

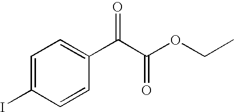

Ethyloxalyl chloride (6.7 ml) was added dropwise at RT to a solution of iodobenzene (11 g) and aluminium chloride (8 g) in carbon disulphide (100 ml) within 15 minutes. The mixture was left to stir at 23° C. for 3 hours. The reaction mixture was then poured onto ice-cold 3N hydrochloric acid. The mixture was then stirred for a further 10 minutes and then extracted with dichloromethane. The organic phase was washed with 1 N hydrochloric acid and saturated aqueous sodium chloride solution, dried over sodium sulphate and concentrated under reduced pressure. The crude product was chromatographed on silica gel. 5.3 g of product were obtained.

### b) 4-iodophenylglyoxylic acid

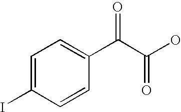

Compound 74b) was synthesized analogously to Example 42b) from 74a).

### c) 6-[2-(4-iodophenyl)-2-oxoacetoylamino]-4-methyl-2,3-benzoxazin-1-one

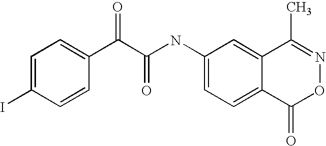

Compound 74c) was synthesized analogously to Example 1a) from compound 74b, thionyl chloride and 6-amino-4-methyl-2,3-benzoxazin-1-one in N,N-dimethylacetamide.

### d) rac-6-[2-hydroxy-2-(4-iodophenyl)-3-phenylpropionylamino]-4-methyl-2,3-benzoxazin-1-one

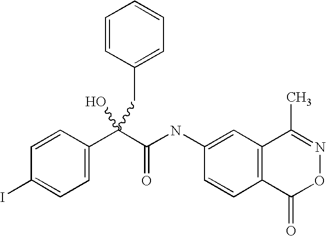

Compounds 74d) were prepared analogously to Example 5) from the substance described under 74c) and benzylmagnesium chloride.

¹H NMR (ppm, CDCl₃, 400 MHz): 2.58 (3H); 2.93 (1H); 3.26 (1H); 3.91 (1H); 7.17 (2H); 7.32 (3H); 7.51 (2H); 7.65 (1H), 7.75 (2H); 8.26 (2H); 8.98 (1H).

### Example 75

### rac-6-[2-hydroxy-2-(4-thiophen-2-ylphenyl)-3-phenylpropionylamino]-4-methyl-2,3-benzoxazin-1-one

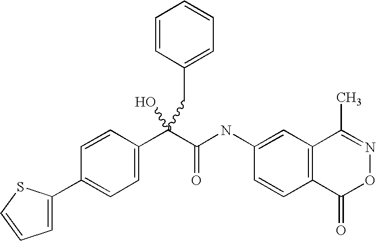

Tetrakistriphenylphosphinepalladium (22 mg), thiophene-2-boronic acid (130 mg), lithium chloride (16 mg) and 2M aqueous sodium carbonate solution (0.2 ml) were added to a solution of the compound described under 74) (100 mg) in ethanol/toluene (1 ml/2.5 ml), and the mixture was left to stir at 95° C. for 2 hours. Subsequently, the reaction mixture was poured onto water. It was extracted with ethyl acetate. The combined organic phases were washed with saturated sodium chloride solution, dried over sodium sulphate and concentrated. The crude product was chromatographed on silica gel and then chromatographed with HPLC. 22 mg of product are obtained.

¹H NMR (ppm, CDCl₃, 400 MHz): 2.58 (3H); 2.90 (1H); 3.34 (1H); 3.95 (1H); 7.10 (1H); 7.20 (2H); 7.30-7.35 (5H); 7.66 (3H), 7.76 (2H); 8.28 (2H); 9.00 (1H).

### Example 75

### rac-N-(3-chloro-4-cyanophenyl)-2-hydroxy-2-(4-nitrophenyl)-3-phenylpropionamide

### a) N-(3-chloro-4-cyanophenyl)-2-oxo-2-(4-nitrophenyl)propionamide

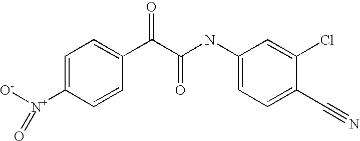

Compound 75a) was synthesized analogously to Example 1a) from 4-nitrophenylglyoxylic acid, thionyl chloride and 3-chloro-4-cyanoaniline in N,N-dimethylacetamide.

### b) rac-N-(3-chloro-4-cyanophenyl)-2-hydroxy-2-(4-nitrophenyl)-3-phenylpropion-amide

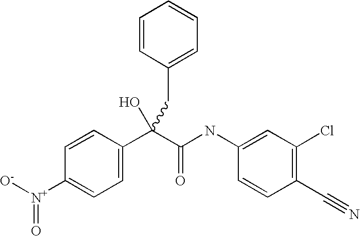

Compounds 75b) were prepared analogously to Example 5) from the substance described under 75a) and benzylmagnesium chloride.

¹H NMR (ppm, CDCl₃, 300 MHz): 2.99 (1H); 3.21 (1H); 3.98 (1H); 7.16 (2H); 7.34 (3H); 7.45 (1H); 7.59 (1H), 7.91 (1H), 7.96 (2H); 8.26 (2H); 8.78 (1H).

Compounds 76-82 were prepared analogously to Example 1 from the substance described under 29a) and the particular lithium arylacetylide.

### Example 76

### rac-N-(3-chloro-4-cyanophenyl)-2-hydroxy-2,3-diphenylbut-3-ynamide

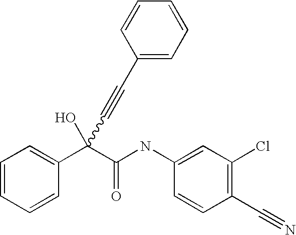

¹H NMR (ppm, CDCl₃, 400 MHz): 4.10 (1H); 7.33-7.50 (6H); 7.55 (3H); 7.61 (1H); 7.80 (2H); 7.91 (1H); 8.59 (1H).

### Example 76a and 76b

### (+)-N-(3-chloro-4-cyanophenyl)-2-hydroxy-2,3-diphenylbut-3-ynamide 76a

### and (−)-N-(3-chloro-4-cyanophenyl)-2-hydroxy-2,3-diphenylbut-3-ynamide 76b

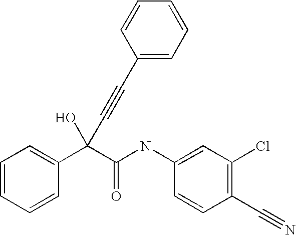

The racemic mixture obtained under Example 76 was separated by preparative chiral HPLC (column: Chiralpak AD 250×10 mm) into enantiomers 76a and 76b.

76a and 76b:

76a: [α]^{D}₂₀: +17.5° (CHCl₃, 10.6 mg/1 ml; λ=589 nM)

76b: [α]^{D}₂₀: −17.4° (CHCl₃, 9.8 mg/1 ml; λ=589 nM)

### Example 77

### rac-N-(3-chloro-4-cyanophenyl)-2-hydroxy-2-phenyl-3-(2-methylphenyl)but-3-ynamide

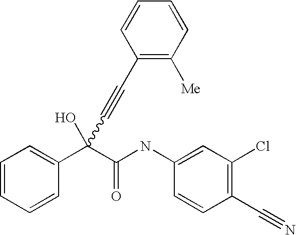

¹H NMR (ppm, CDCl₃, 400 MHz): 2.48 (3H); 4.09 (1H); 7.15-7.29 (3H); 7.42-7.52 (5H); 7.60 (1H); 7.81 (2H); 7.90 (1H); 8.58 (1H).

### Example 78

### rac-N-(3-chloro-4-cyanophenyl)-2-hydroxy-2-phenyl-3-(4-methylphenyl)but-3-ynamide

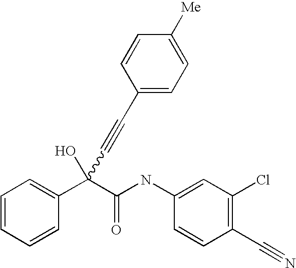

¹H NMR (ppm, CDCl₃, 400 MHz): 2.38 (3H); 4.09 (1H); 7.17 (2H); 7.41-7.53 (6H); 7.61 (1H); 7.80 (2H); 7.91 (1H), 8.58 (1H).

### Example 79

### rac-N-(3-chloro-4-cyanophenyl)-2-hydroxy-2-phenyl-3-(4-trifluoromethyl-phenyl)but-3-ynamide

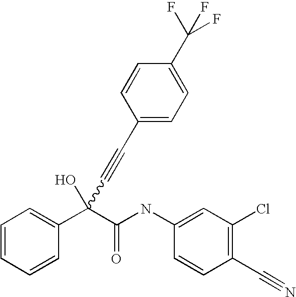

¹H NMR (ppm, CDCl₃, 400 MHz): 4.07 (1H); 7.42-7.54 (4H); 7.60-7.67 (5H); 7.78 (2H); 7.92 (1H); 8.60 (1H).

### Example 80

### rac-N-(3-chloro-4-cyanophenyl)-2-hydroxy-2-phenyl-3-(4-methoxyphenyl)but-3-ynamide

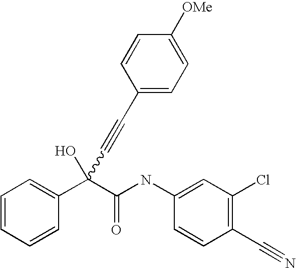

¹H NMR (ppm, CDCl₃, 400 MHz): 3.83 (3H); 4.10 (1H); 6.87 (2H); 7.40-7.52 (6H); 7.60 (1H); 7.78 (2H); 7.90 (1H); 8.59 (1H).

### Example 80a and 80b

### (+)-N-(3-chloro-4-cyanophenyl)-2-hydroxy-2-phenyl-3-(4-methoxyphenyl)but-3-ynamide 80a and

### (−)-N-(3-chloro-4-cyanophenyl)-2-hydroxy-2-phenyl-3-(4-methoxyphenyl)but-3-ynamide 80b

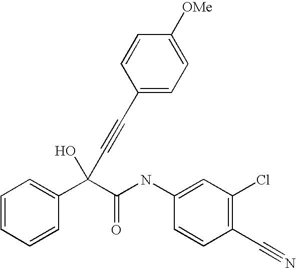

The racemic mixture obtained under Example 80 was separated by preparative chiral HPLC (column: Chiralpak AD 250×10 mm) into enantiomers 80a and 80b.

80a and 80b:

80a: [α]^{D}₂₀: +60.5° (CHCl₃, 10.4 mg/1 ml; λ=589 nM)

80b: [α]^{D}₂₀: −61.3° (CHCl₃, 10.1 mg/1 ml; λ=589 nM)

### Example 81

### rac-N-(3-chloro-4-cyanophenyl)-2-hydroxy-2-phenyl-3-(3-methylphenyl)but-3-ynamide

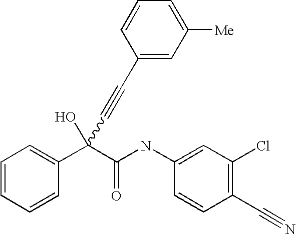

¹H NMR (ppm, CDCl₃, 400 MHz): 2.35 (3H); 4.09 (1H); 7.20-7.26 (2H); 7.36 (2H); 7.42-7.53 (4H); 7.61 (1H); 7.80 (2H); 7.91 (1H); 8.58 (1H).

### Example 82

### rac-N-(3-chloro-4-cyanophenyl)-2-hydroxy-2-phenyl-3-(4-carbomethoxyphenyl)-but-3-ynamide

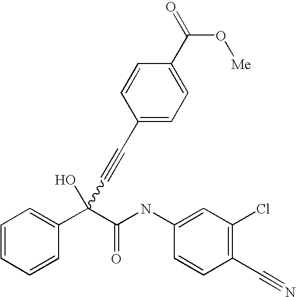

¹H NMR (ppm, DMSO-d₆, 400 MHz): 3.83 (3H); 7.39 (3H); 7.65 (2H); 7.75 (2H); 7.87 (1H); 7.94 (3H); 8.20 (1H); 10.68 (1H).

### Example 83

### rac-N-(3-chloro-4-cyanophenyl)-2-hydroxy-2-phenyl-3-(4-carboxyphenyl)but-3-ynamide

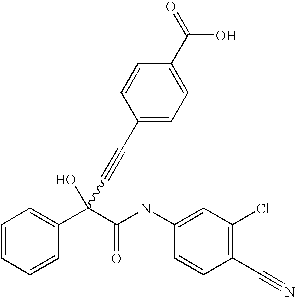

Compound 83) was synthesized analogously to Example 42b) from the substance described under 82).

¹H NMR (ppm, DMSO-d₆, 400 MHz): 7.32-7.43 (3H); 7.62 (2H); 7.75 (2H); 7.87 (1H); 7.94 (3H); 8.20 (1H); 10.68 (1H).

### Example 84

### rac-N-(3-chloro-4-cyanophenyl)-2-hydroxy-2-methylphenyl-4-phenylbut-3-ynamide

### a) methyl N-(3-chloro-4-cyanophenyl)oxalamide

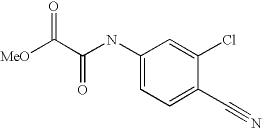

Compound 84a) was synthesized analogously to Example 1a) from methyloxalyl chloride and 3-chloro-4-cyanoaniline in N,N-dimethylacetamide.

### b) N-(3-chloro-4-cyanophenyl)-2-oxo-4-phenylbut-3-ynamide

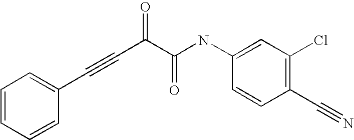

Compound 84b) was synthesized analogously to Example 1b) from the substance described under 84a) and lithium phenylacetylide.

### c) rac-N-(3-chloro-4-cyanophenyl)-2-hydroxy-2-methylphenyl-4-phenylbut-3-ynamide

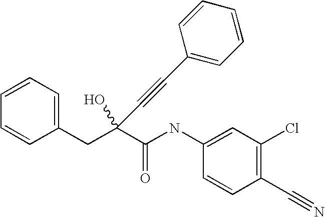

Compound 84c) was synthesized analogously to Example 5) from the substance described under 84b) and benzylmagnesium chloride.

¹H NMR (ppm, CDCl₃, 300 MHz): 3.18 (1H); 3.34 (1H); 3.47 (1H); 7.30-7.37 (8H); 7.43 (2H); 7.47 (1H); 7.61 (1H); 7.90 (1H); 8.61 (1H).

### Example 85

### N-(3-chloro-4-cyanophenyl)-2-hydroxy-2-methylphenyl-3-phenylpropionamide

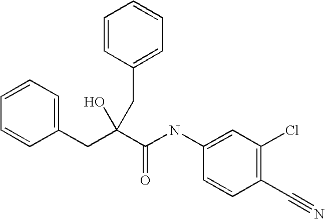

Compound 85) was synthesized analogously to Example 5) from the substance described under 84a) and benzylmagnesium chloride.

¹H NMR (ppm, CDCl₃, 300 MHz): 2.34 (1H); 2.98 (2H); 3.54 (2H); 7.24-7.33 (11H); 7.56 (1H); 7.71 (1H); 8.25 (1H).

### Example 86

### rac-N-(3-chloro-4-cyanophenyl)-2-hydroxy-3-(3-iodophenyl)-2-phenyl-propionamide

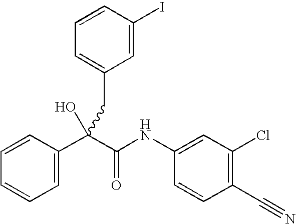

39.3 ml of 3-iodobenzylzinc bromide (0.5 M solution in THF) were initially charged in 35 ml of THF, and 2.8 g of N-(3-chloro-4-cyanophenyl)-2-oxo-2-phenylacetamide (see Ex. 30) dissolved in 35 ml of THF were added dropwise at −70° C. After 30 min at −70° C., the mixture was allowed to come to 0° C. The reaction was added to 500 ml of sat. ammonium chloride solution and extracted with ethyl acetate, and the organic phases were washed with sat. sodium chloride solution and dried with sodium sulphate. After filtration and removal of the solvent, the crude product was chromatographed on silica gel. 4.65 g of product were obtained as yellowish foam. ¹H NMR (ppm, CDCl₃, 400 MHz): 2.72 (s, 1H), 3.20 (d, 1H), 3.81 (d, 1H), 7.00 (m, 1H), 7.11 (m, 1H), 7.40 (m, 4H), 7.55 (m, 3H), 7.68 (dd, 2H), 7.88 (d, 1H), 8.70 (s, 1H).

### Example 87

### rac-N-(3-chloro-4-cyanophenyl)-2-hydroxy-3-(4-iodophenyl)-2-phenyl-propionamide

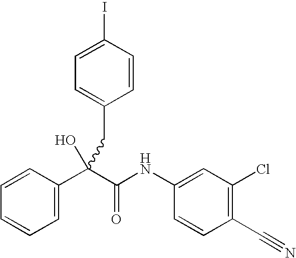

The compound was prepared analogously to Example 86 using 4-iodobenzylzinc bromide. 1.3 g of keto amide afforded 1.43 g of the desired product as a pale yellow foam. ¹H NMR (ppm, CDCl₃, 400 MHz): 2.68 (s, 1H), 3.20 (d, 1H), 3.81 (d, 1H), 6.90 (d, 2H), 7.40 (m, 4H), 7.62 (m, 5H), 7.89 (dd, 1H), 8.71 (s, 1H).

### Example 88

### rac-N-(3-chloro-4-cyanophenyl)-2-hydroxy-3-(4-fluorophenyl)-2-phenyl-propionamide

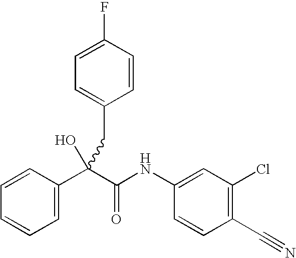

The compound was prepared analogously to Example 86 using 4-fluorobenzylzinc bromide. 1H NMR (ppm, CDCl3, 400 MHz): 2.78 (s, 1H), 3.23 (d, 1H), 3.86 (d, 1H), 6.97 (dd, 2H), 7.12 (dd, 2H), 7.32-7.45 (m, 4H), 7.54 (d, 1H), 7.68 (dd, 2H), 7.89 (dd, 1H), 8.74 (s, 1H).

### Example 89

### rac-N-(3-chloro-4-cyanophenyl)-2-hydroxy-3-(2-iodophenyl)-2-phenyl-propionamide

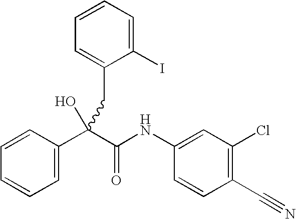

The compound was prepared analogously to Example 86 using 2-iodobenzylzinc bromide. 3 g of keto amide afforded 4.11 g of the desired product as a yellow foam. ¹H NMR (ppm, CDCl₃, 400 MHz): 3.09 (s, 1H), 3.58 (d, 1H), 4.15 (d, 1H), 6.96 (m, 1H), 7.19 (m, 2H), 7.41 (m, 4H), 7.56 (d, 1H), 7.72 (d, 2H), 7.87 (dd, 1H), 7.92 (d, 1H), 8.78 (s, 1H).

### Example 90

### rac-3-[2-(3-chloro-4-cyanophenylcarbamoyl)-2-hydroxy-2-phenylethyl]benzoic acid methyl ester

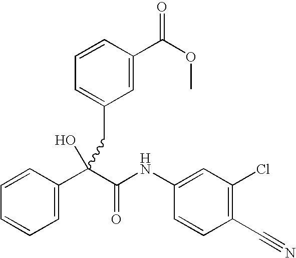

3.65 g of rac-N-(3-chloro-4-cyanophenyl)-2-hydroxy-3-(3-iodophenyl)-2-phenyl-propionamide, 510 mg of bis(triphenylphosphino)palladium dichloride and 2.21 ml of triethylamine were dissolved in a pressure vessel in 6.5 ml of DMSO and 30 ml of methanol. The mixture was degassed and saturated with carbon monoxide, and a carbon monoxide atmosphere was created in the reaction vessel with the aid of a CO balloon. The reaction vessel was sealed and the mixture was stirred at 100-110° C. for 18 h. After cooling, the reaction vessel was opened and purged with argon, the mixture was transferred to a round-bottomed flask, diatomaceous earth was added and the mixture was concentrated to dryness on a rotary evaporator. It was chromatographed on silica gel. The product fractions were admixed with ice-water and the desired product was crystallized. A total of 2.1 g of the desired product were obtained as a colourless solid. ¹H NMR (ppm, DMSO-D₆, 400 MHz): 3.25 (d, 1H), 3.70 (d, 1H), 3.76 (s, 3H), 6.73 (s, 1H), 7.30 (m, 5H), 7.60 (dd, 2H), 7.70 (dd, 1H), 7.80 (m, 3H), 8.09 (s, 1H), 10.15 (s, 1H).

### Example 91

### rac-4-[2-(3-chloro-4-cyanophenylcarbamoyl)-2-hydroxy-2-phenylethyl]benzoic acid methyl ester

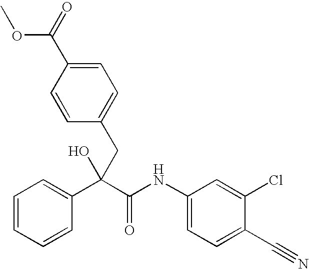

The compound was prepared analogously to Example 90 using 2.9 g of rac-N-(3-chloro-4-cyanophenyl)-2-hydroxy-3-(4-iodophenyl)-2-phenylpropionamide. 1.29 g of product were obtained. ¹H NMR (ppm, DMSO-D₆, 400 MHz): 3.29 (d, 1H), 3.70 (d, 1H), 6.76 (s, 1H), 7.24 (m, 3H), 7.31 (m, 2H), 7.59 (d, 2H), 7.72 (d, 2H), 7.81 (m, 2H), 8.10 (d, 1H), 10.20 (s, 1H).

### Example 92

### rac-3-[2-(3-chloro-4-cyanophenylcarbamoyl)-2-hydroxy-2-phenylethyl]benzoic acid

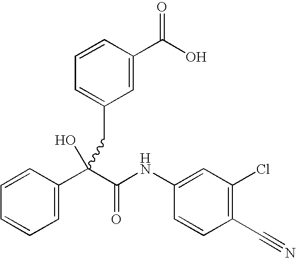

5 790 mg of rac-3-[2-(3-chloro-4-cyanophenylcarbamoyl)-2-hydroxy-2-phenylethyl]-benzoic acid methyl ether were initially charged in THF, admixed with 435 mg of lithium hydroxide, heated to 90° C. and then stirred at room temperature for 18 h. The mixture was diluted with 10 ml of water, adjusted to pH 4 with 2 M hydrochloric acid and extracted with ethyl acetate, and the organic phases were washed with water and sat. NaCl solution and dried over sodium sulphate. After removal of the solvents and chromatographic purification, 591 mg of the desired product were obtained. ¹H NMR (ppm, DMSO-D₆, 400 MHz): 3.79 (d, 1H), 3.74 (d, 1H), 6.75 (s, 1H), 7.28 (m, 2H), 7.37 (m, 3H), 7.65 (d, 2H), 7.73 (m, 2H), 7.35 (m, 3H), 8.12 (d, 1H), 10.21 (s, 1H), 12.75 (s, 1H).

### Example 93

### rac-4-[2-(3-chloro-4-cyanophenylcarbamoyl)-2-hydroxy-2-phenylethyl]benzoic acid

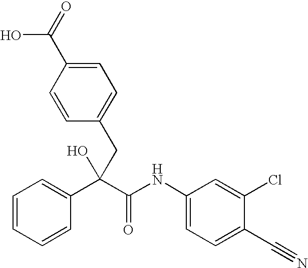

The compound was prepared analogously to Example 92 using 700 mg of rac-4-[2-(3-chloro-4-cyanophenylcarbamoyl)-2-hydroxy-2-phenylethyl]benzoic acid methyl ester. After chromatography, 366 mg of product were obtained. ¹H NMR (ppm, DMSO-D₆, 400 MHz): 3.27 (d, 1H), 3.70 (d, 1H), 6.74 (s, 1H), 7.28 (m, 5H), 7.60 (d, 2H), 7.70 (d, 2H), 7.81 (m, 2H), 8.12 (d, 1H), 10.21 (s, 1H), 12.70 (s, 1H).

### Example 94

### rac-N-(3-chloro-4-cyanophenyl)-2-hydroxy-3-(3-hydroxymethylphenyl)-2-phenyl-propionamide

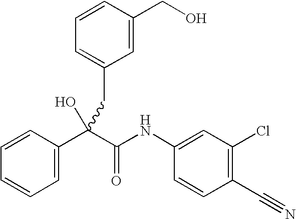

60 mg of rac-3-[2-(3-chloro-4-cyanophenylcarbamoyl)-2-hydroxy-2-phenylethyl]benzoic acid were initially charged in 3 ml of THF, and 0.43 ml of borane-THF complex solution (3 eq.) was added dropwise at 0° C. After stirring at room temperature for 18 h, a further 0.43 ml of borane-THF complex was added. After a further 2.5 h, 1 ml of sat. sodium carbonate solution was added and the mixture was stirred for a further 30 minutes. The reaction mixture was concentrated to dryness, the residue was partitioned between water and ethyl acetate, the phases were separated, extraction was effected with ethyl acetate, and the combined organic phases were washed with sat. NaCl solution and dried over sodium sulphate. After chromatography on silica gel, 28.8 mg of product were obtained as white foam. ¹H NMR (ppm, DMSO-D₆, 400 MHz): 3.15 (d, 1H), 3.64 (d, 1H), 4.31 (d, 2H), 5.01 (t, 1H), 6.60 (s, 1H), 7.04 (m, 4H), 7.80 (m, 3H), 7.60 (dd, 2H), 7.69 (m, 2H), 8.10 (d, 1H), 10.15 (s, 1H).

### Example 95

### rac-N-(3-chloro-4-cyanophenyl)-2-hydroxy-3-(4-hydroxymethylphenyl)-2-phenyl-propionamide

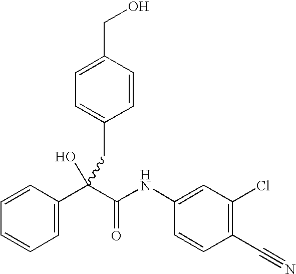

150 mg of rac-4-[2-(3-chloro-4-cyanophenylcarbamoyl)-2-hydroxy-2-phenylethyl]-benzoic acid methyl ester were initially charged in 7 ml of THF, and 1 equivalent of lithium aluminium hydride was added at room temperature. After 2 h, one further equivalent of lithium aluminium hydride was added and the mixture was kept at 4° C. for 18 h. The mixture was admixed with water and extracted with ethyl acetate, and the combined organic phases were washed with water and sat. NaCl solution and dried over sodium sulphate. After chromatography on silica gel, 65.3 mg of product were obtained as yellowish foam. ¹H NMR (ppm, DMSO-D₆, 400 MHz): 3.23 (d, 1H), 3.68 (d, 1H), 4.40 (d, 2H), 5.06 (t, 1H), 6.63 (s, 1H), 6.8-7.5 (m, 7H), 7.65 (d, 2H), 7.86 (m, 2H), 8.17 (d, 1H), 10.22 (s, 1H).

### Example 96

### rac-N-(3-chloro-4-cyanophenyl)-2-hydroxy-3-(2-hydroxymethylphenyl)-2-phenyl-propionamide

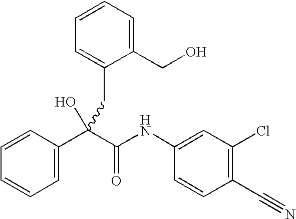

Conversion of 3.1 g of rac-N-(3-chloro-4-cyanophenyl)-2-hydroxy-3-(2-iodophenyl)-2-phenylpropionamide analogously to Example 90 led to 1.4 g of N-(3-chloro-4-cyanophenyl)-1-oxo-3-phenylisochroman-3-carboxamide. 500 mg of this intermediate were reacted with lithium aluminium hydride analogously to Example 95. 464 mg of the desired product were obtained as a yellowish foam. ¹H NMR (ppm, DMSO-D₆, 400 MHz): 3.30 (d, 1H), 3.72 (d, 1H), 4.47 (s, 2H), 6.01 (s, br, 1H), 7.04 (m, 3H), 7.10 (m, 2H), 7.26 (m, 2H), 7.32 (m, 2H), 7.61 (d, 2H), 7.84 (m, 2H), 8.17 (d, 1H), 10.21 (s, 1H).

### Example 97

### rac-N-3-(3-chloro-4-cyanophenyl)-3-(3-formylphenyl)-2-hydroxy-2-phenyl-propionamide

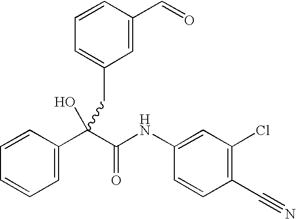

359 mg of rac-N-(3-chloro-4-cyanophenyl)-2-hydroxy-3-(3-hydroxymethylphenyl)-2-phenylpropionamide were initially charged in 7 ml of dichloromethane and admixed at 0° C. with 560 mg of Dess-Martin periodinane. After stirring at room temperature for 18 h, the mixture was added to an sat. sodium hydrogencarbonate/sodium thiosulphate solution (1:1). The mixture was extracted with ethyl acetate, washed with sat. NaCl solution and dried over sodium sulphate, and the solvents were removed. 343 mg of product were obtained as white foam. ¹H NMR (ppm, CDCl₃, 400 MHz): 3.40 (d, 1H), 3.92 (d, 1H), 7.3-7.5 (m, 6H), 7.56 (d, 1H), 7.69 (m, 3H), 7.79 (m, 1H), 7.88 (d, 1H), 8.72 (s, 1H), 9.95 (s, 1H).

### Example 98

### rac-N-3-(3-chloro-4-cyanophenyl)-3-(4-formylphenyl)-2-hydroxy-2-phenyl-propionamide

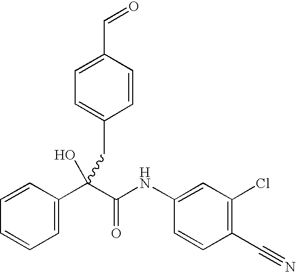

221 mg of rac-N-(3-chloro-4-cyanophenyl)-2-hydroxy-3-(4-hydroxymethylphenyl)-2-phenylpropionamide were converted analogously to Example 97. 108 mg of product were obtained. ¹H NMR (ppm, CDCl₃, 400 MHz): 3.44 (d, 1H), 4.00 (d, 1H), 7.34-7.53 (m, 6H), 7.61 (d, 1H), 7.72 (m, 2H), 7.83 (d, 2H), 7.94 (d, 1H), 8.75 (s, 1H), 10.00(s, 1H).

### Example 99

### rac-N-(3-chloro-4-cyanophenyl)-3-(4′-cyanobiphenyl-3-yl)-2-hydroxy-2-phenyl-propionamide

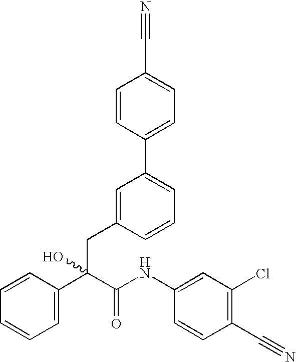

100 mg of rac-N-(3-chloro-4-cyanophenyl)-2-hydroxy-3-(3-iodophenyl)-2-phenyl-propionamide and 46 mg of 3-cyanophenylboronic acid were initially charged in 2 ml of toluene and 2 ml of ethanol, and 0.4 ml of sat. sodium carbonate solution and 23 mg of tetrakis(triphenylphosphine)palladium were added. The mixture was converted at 120° C. in a microwave for 20 min. The mixture was then filtered through Celite, rinsed through with ethyl acetate, washed with sat. NaCl solution and dried over sodium sulphate. After chromatographic purification of the crude product, 23 mg of target product were obtained as white foam. ¹H NMR (ppm, CDCl₃, 400 MHz): 2.92 (s, 1H), 3.40 (d, 1H), 3.90 (d, 1H), 7.1-7.8 (m, 15H), 7.90 (d, 1H), 8.77 (s, 1H).

Analogously to Example 99, Examples 100-123 were prepared by reacting the corresponding aryl iodides from Example 86, 87 or 89 with the arylboronic acids required in each case.

### Example 100

### rac-N-(3-chloro-4-cyanophenyl)-3-(4′-cyanobiphenyl-4-yl)-2-hydroxy-2-phenyl-propionamide

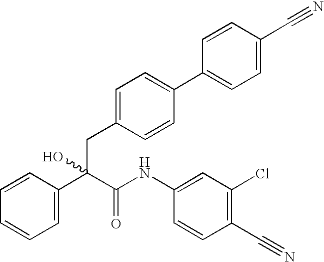

¹H NMR (ppm, CDCl₃, 400 MHz): 2.81 (s, 1H), 3.36 (d, 1H), 4.00 (d, 1H), 7.32 (d, 2H), 7.4-7.8 (m, 1 3H), 7.97 (d, 1H), 8.80 (s, 1H).

### Example 101

### rac-N-(3-chloro-4-cyanophenyl)-3-(4′-cyanobiphenyl-2-yl)-2-hydroxy-2-phenyl-propionamide

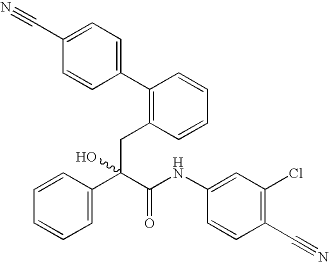

¹H NMR (ppm, CDCl₃, 400 MHz): 2.51 (s, 1H), 3.51 (d, 1H), 3.83 (d, 1H), 7.20 (d, 2H), 7.25-7.45 (m, 11H), 7.55 (d, 1H), 7.64 (d, 2H), 7.83 (d, 1H), 8.61 (s, 1H),

### Example 102

### rac-N-(3-chloro-4-cyanophenyl)-2-hydroxy-2-phenyl-3-(3-pyridin-4-ylphenyl)-propionamide

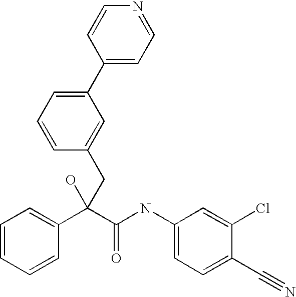

¹H NMR (ppm, DMSO-D₆, 400 MHz): 3.36 (d, 1H), 3.71 (d, 1H), 6.72 (s, 1H), 7.29 (m, 5H), 7.48 (m, 4H), 7.55 (d, 1H), 7.63 (d, 2H), 7.80 (m, 2H), 8.12 (d, 1H), 8.55 (d, 2H), 10.21 (s, 1H).

### Example 103

### rac-N-(3-chloro-4-cyanophenyl)-2-hydroxy-2-phenyl-3-(4-pyridin-4-ylphenyl)-propionamide

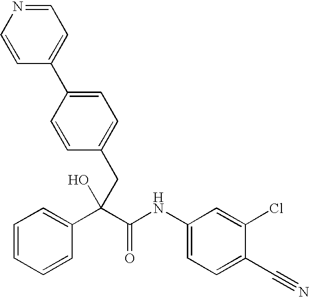

¹H NMR (ppm, DMSO-D₆, 400 MHz): 3.39 (d, 1H), 3.71 (d, 1H), 6.73 (s, 1H), 7.29 (m, 4H), 7.58 (m, 7H), 7.82 (m, 2H), 8.13 (d, 1H), 8.55 (d, 2H), 10.24 (s, 1H).

### Example 104

### rac-N-(3-chloro-4-cyanophenyl)-2-hydroxy-2-phenyl-3-(2-pyridin-4-ylphenyl)-propionamide

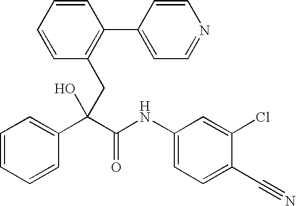

¹H NMR (ppm, DMSO-D₆, 400 MHz): 3.49 (m, 2H), 6.63 (s, 1H), 6.9-7.6 (m, 10H), 7.56 (d, 1H), 7.80 (m, 2H), 8.10 (s, 1H), 8.50 (d, 2H), 10.27 (s, 1H).

### Example 105

### rac-N-(3-chloro-4-cyanophenyl)-2-hydroxy-2-phenyl-3-(3-pyridin-3-ylphenyl)-propionamide

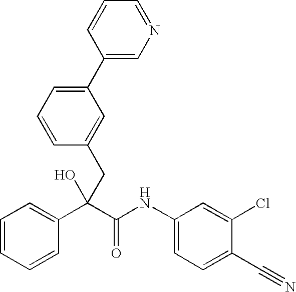

¹H NMR (ppm, DMSO-D₆, 400 MHz): 3.29 (d, 1H), 3.70 (d, 1H), 6.69 (s, 1H), 7.18 (d, 1H), 7.28 (m, 2H), 7.34 (m, 2H), 7.40 (m, 2H), 7.45 (m, 1H), 7.63 (d, 2H), 7.81 (m, 3H), 8.12 (d, 1H), 8.50 (dd, 1H), 8.65 (d, 1H), 10.22 (s, 1H).

### Example 106

### rac-N-(3-chloro-4-cyanophenyl)-2-hydroxy-2-phenyl-3-(4-pyridin-3-ylphenyl)-propionamide

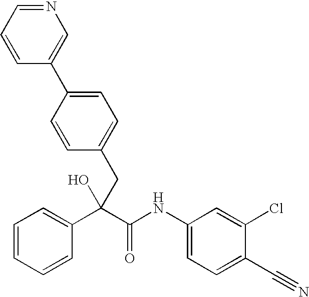

¹H NMR (ppm, DMSO-D₆, 400 MHz): 3.26 (d, 1H), 3.70 (d, 1H), 6.70 (s, 1H), 7.21-7.43 (m, 6H), 7.52 (d, 2H), 7.63 (dd, 2H), 7.82 (m, 2H), 7.97 (m, 1H), 8.13 (d, 1H), 8.49 (dd, 2H), 8.80 (d, 1H), 10.21 (s, 1H).

### Example 107

### rac-N-(3-chloro-4-cyanophenyl)-2-hydroxy-2-phenyl-3-(2-pyridin-3-ylphenyl)-propionamide

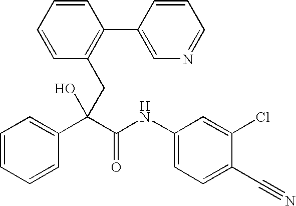

¹H NMR (ppm, DMSO-D₆, 400 MHz): 3.47 (s, 2H), 6.64 (s, 1H), 7.01 (dd, 1H), 7.18 (m, 7H), 7.36 (dd, 1H), 7.46 (d, 1H), 7.56 (d, 1H), 7.79 (m, 2H), 8.10 (s, 1H), 8.22 (d, 1H), 8.50 (dd, 1H), 10.27 (s, 1H).

### Example 108

### rac-N-(3-chloro-4-cyanophenyl)-2-hydroxy-2-phenyl-3-(4′-trifluoromethylbiphenyl-3-yl)propionamide

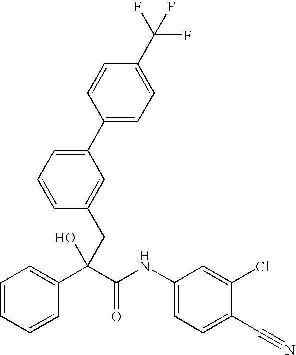

¹H NMR (ppm, DMSO-D₆, 400 MHz): 3.28 (d, 1H), 3.71 (d, 1H), 6.71 (s, 1H), 7.19 (d, 1H), 7.28 (m, 2H), 7.34 (m, 2H), 7.42 (s, 1H), 7.46 (d, 1H), 7.64 (m, 4H), 7.73 (d, 2H), 7.80 (m, 2H), 8.11 (d, 1H), 10.21 (s, 1H).

### Example 109

### rac-N-(3-chloro-4-cyanophenyl)-2-hydroxy-2-phenyl-3-(4′-trifluoromethylbiphenyl-4-yl)propionamide

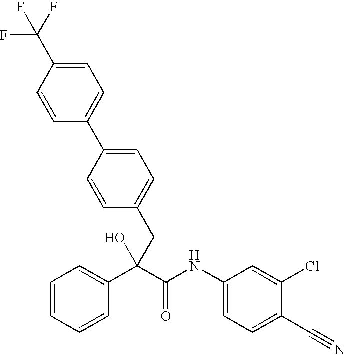

¹H NMR (ppm, CDCl₃, 400 MHz): 3.30 (d, 1H), 3.98 (d, 1H), 7.27 (d, 2H), 7.40 (m, 4H), 7.54 (m, 3H), 7.66 (m, 4H), 7.72 (m, 2H), 7.92 (d, 1H), 8.77 (s, 1H).

### Example 110

### rac-N-(3-chloro-4-cyanophenyl)-2-hydroxy-2-phenyl-3-(4′-trifluoromethylbiphenyl-2-yl)propionamide

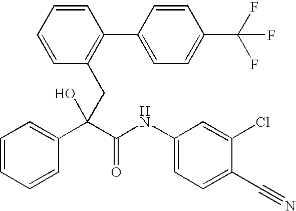

¹H NMR (ppm, DMSO-D₆, 400 MHz): 3.49 (dd, 2H), 6.62 (s, 1H), 7.00 (dd, 1H), 7.18 (m, 9H), 7.57 (d, 1H), 7.66 (d, 2H), 7.79 (m, 2H), 8.10 (s, 1H), 10.26 (s, 1H).

### Example 111

### rac-N-(3-chloro-4-cyanophenyl)-2-hydroxy-3-(4′-methanesulphonylbiphenyl-3-yl)-2-phenylpropionamide

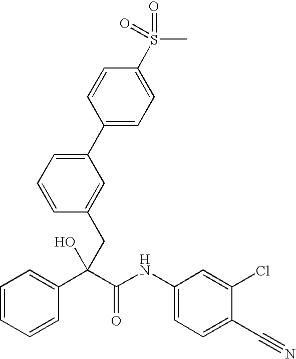

¹H NMR (ppm, DMSO-D₆, 400 MHz): 3.21 (s, 3H), 3.32 (d, 1H), 3.71 (d, 1H), 6.72 (s, 1H), 7.15-7.75 (m, 11H), 7.81 (m, 2H), 7.92 (d, 2H), 8.12 (d, 1H), 10.23 (s, 1H).

### Example 112

### rac-N-(3-chloro-4-cyanophenyl)-2-hydroxy-3-(4′-methanesulphonylbiphenyl-4-yl)-2-phenylpropionamide

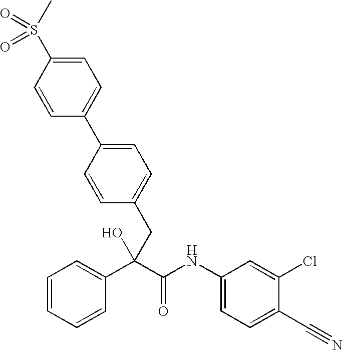

¹H NMR (ppm, DMSO-D₆, 400 MHz): 3.20 (s, 3H), 3.30 (d, 1H), 3.71 (d, 1H), 6.72 (s, 1H), 7.27 (m, 3H), 7.34 (m, 2H), 7.45-7.65 (m, 4H), 7.83 (m, 4H), 7.91 (d, 2H), 8.14 (d, 1H), 10.23 (s, 1H).

### Example 113

### rac-N-(3-chloro-4-cyanophenyl)-2-hydroxy-3-(4′-methanesulphonylbiphenyl-2-yl)-2-phenylpropionamide

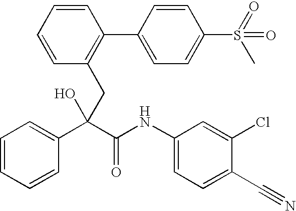

¹H NMR (ppm, DMSO-D₆, 400 MHz): 3.24 (s, 3H), 3.50 (dd, 2H), 6.62 (s, 1H), 6.99 (dd, 1H), 7.1-7.3 (m, 6H), 7.55 (m, 4H), 7.80 (d, 2H), 7.85 (d, 2H), 8.11 (m, 1H), 10.27 (s, 1H).

### Example 114

### rac-N-(3-chloro-4-cyanophenyl)-2-hydroxy-3-(4′-hydroxymethyl biphenyl-3-yl)-2-phenylpropionamide

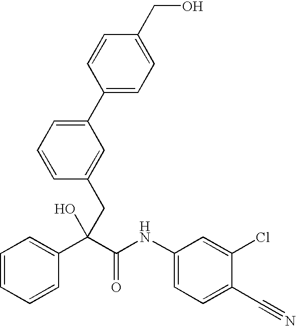

¹H NMR (ppm, DMSO-D₆, 400 MHz): 3.26 (d, 1H), 3.70 (d, 1H), 4.49 (d, 2H), 5.16 (t, 1H), 6.67 (s, 1H), 7.10 (d, 1H), 7.18-7.42 (m, 10H), 7.64 (m, 2H), 7.82 (m, 2H), 8.14 (d, 1H), 10.22 (s, 1H).

### Example 115

### rac-N-(3-chloro-4-cyanophenyl)-2-hydroxy-3-(4′-hydroxymethyl biphenyl-4-yl)-2-phenylpropionamide

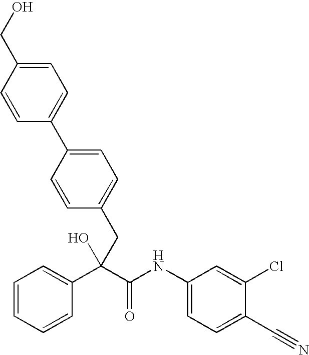

¹H NMR (ppm, DMSO-D₆, 400 MHz): 3.24 (d, 1H), 3.68 (d, 1H), 4.47 (d, 2H), 5.15 (t, 1H), 6.68 (s, 1H), 7.20 (d, 2H), 7.25 (m, 1H), 7.32 (m, 4H), 7.43 (d, 2H), 7.52 (d, 2H), 7.63 (d, 2H), 7.82 (m, 2H), 8.14 (d, 1H), 10.22 (s, 1H).

### Example 116

### rac-N-(3-chloro-4-cyanophenyl)-2-hydroxy-3-(4′-hydroxymethyl biphenyl-2-yl)-2-phenylpropionamide

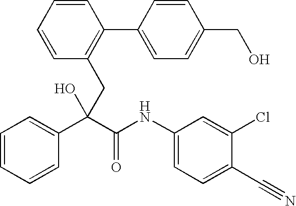

¹H NMR (ppm, DMSO-D₆, 400 MHz): 3.54 (dd, 2H), 4.56 (d, 2H), 5.23 (t, 1H), 6.72 (s, 1H), 7.02 (m, 1H), 7.09 (d, 2H), 7.19 (m, 5H), 7.32 (m, 4H), 7.61 (dd, 1H), 7.85 (d, 2H), 8.17 (s, 1H), 10.37 (s, 1H).

### Example 117

### rac-3-(4′-acetylbiphenyl-3-yl)-N-(3-chloro-4-cyanophenyl)-2-hydroxy-2-phenyl-propionamide

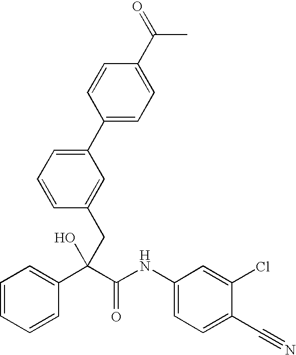

¹H NMR (ppm, DMSO-D₆, 400 MHz): 2.61 (s, 3H), 3.34 (d, 1H), 3.75 (d, 1H), 6.76 (s, 1H), 7.23 (d, 1H), 7.30-7.54 (m, 4H), 7.50 (m, 2H), 7.63 (d, 2H), 7.68 (d, 2H), 7.86 (m, 2H), 7.99 (d, 2H), 8.17 (d, 1H), 10.27 (s, 1H).

### Example 118

### rac-3-(4′-acetylbiphenyl-4-yl)-N-(3-chloro-4-cyanophenyl)-2-hydroxy-2-phenyl-propionamide

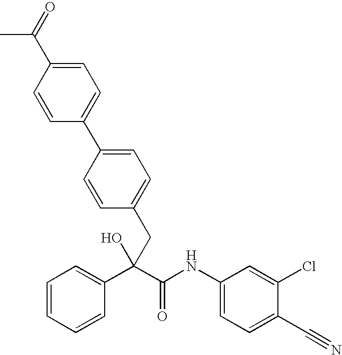

¹H NMR (ppm, CDCl₃, 400 MHz): 2.62 (s, 3H), 2.88 (s, 1H), 3.30 (d, 1H), 3.96 (d, 1H), 7.27 (d, 2H), 7.35-7.45 (m, 4H), 7.52-7.59 (m, 3H), 7.63 (d, 2H), 7.73 (dd, 2H), 7.91 (d, 1H), 8.00 (d, 2H), 8.78 (s, 1H).

### Example 119

### rac-3-(4′-acetylbiphenyl-2-yl)-N-(3-chloro-4-cyanophenyl)-2-hydroxy-2-phenyl-propionamide

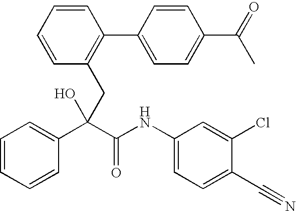

¹H NMR (ppm, DMSO-D₆, 400 MHz): 2.59 (s, 3H), 3.50 (dd, 2H), 6.65 (s, 1H), 6.99 (dd, 1H), 7.18 (m, 9H), 7.58 (dd, 1H), 7.79 (s, 2H), 7.91 (d, 2H), 8.10 (s, 1H), 10.28 (s, 1H).

### Example 120

### rac-N-(3-chloro-4-cyanophenyl)-2-hydroxy-3-(4′-methoxybiphenyl-3-yl)-2-phenyl-propionamide

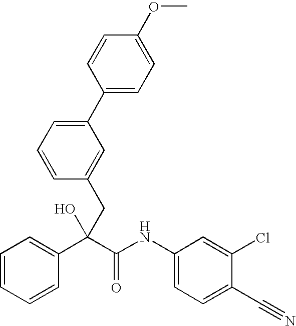

¹H NMR (ppm, CDCl₃, 400 MHz): 2.84 (s, 3H), 3.30 (d, 1H), 3.85 (s, 3H), 3.96 (d, 1H), 6.93 (d, 2H), 7.09 (d, 1H), 7.28-7.50 (m, 9H), 7.54 (d, 1H), 7.73 (d, 2H), 7.92 (d, 1H), 8.78 (s, 1H).

### Example 121

### rac-N-(3-chloro-4-cyanophenyl)-2-hydroxy-3-(4′-methoxybiphenyl-4-yl)-2-phenyl-propionamide

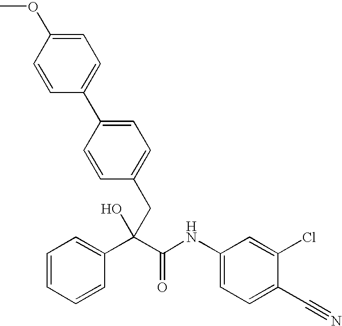

¹H NMR (ppm, CDCl₃, 400 MHz): 2.84 (s, 3H), 3.30 (d, 1H), 3.85 (s, 3H), 3.96 (d, 1H), 6.93 (d, 2H), 7.09 (d, 1H), 7.28-7.50 (m, 9H), 7.54 (d, 1H), 7.73 (d, 2H), 7.92 (d, 1H), 8.78 (s, 1H).

### Example 122

### rac-N-(3-chloro-4-cyanophenyl)-2-hydroxy-3-(4′-methoxybiphenyl-2-yl)-2-phenyl-propionamide

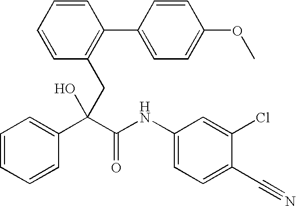

¹H NMR (ppm, CDCl₃, 400 MHz): 2.51 (s, 1H), 3.57 (d, 1H), 3.85 (s, 3H), 3.92 (d, 1H), 6.94 (d, 2H), 7.18 (dd, 2H), 7.20-7.33 (m, 7H), 7.36 (dd, 1H), 7.48 (dd, 2H), 7.53 (d, 1H), 7.84 (d, 1H), 8.63 (s, 1H).

### Example 123

### rac-4′-[2-(3-chloro-4-cyanophenylcarbamoyl)-2-hydroxy-2-phenylethyl]biphenyl-4-carboxylic acid

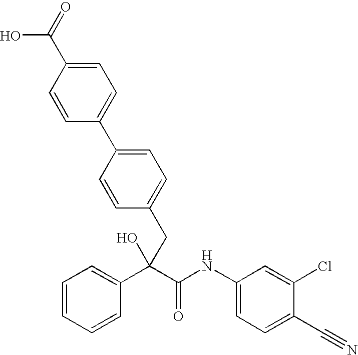

¹H NMR (ppm, DMSO-D₆, 400 MHz): 3.26 (d, 1H), 3.70 (d, 1H), 6.71 (s, 1H), 7.25 (m, 2H), 7.33 (m, 2H), 7.50-7.65 (m, 5H), 7.70 (d, 2H), 7.82 (m, 2H), 7.93 (d, 2H), 8.14 (d, 1H), 10.23 (s, 1H).

### Example 124

### rac-N-(3-chloro-4-cyanophenyl)-3-[4′-(1,2-dihydroxyethyl)biphenyl-2-yl]-2-hydroxy-2-phenylpropionamide

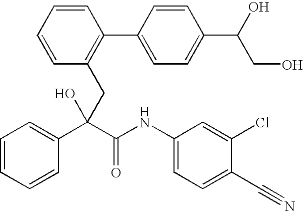

Analogously to Example 99, N-(3-chloro-4-cyanophenyl)-2-hydroxy-2-phenyl-3-(4′-vinyl-biphenyl-2-yl)propionamide was prepared by means of a Suzuki reaction with 4-vinylphenylboronic acid. 100 mg of this intermediate were initially charged in 5 ml of acetone and 0.7 ml of water, and 25 mg of N-methylmorpholine N-oxide and 26 μl of osmium tetroxide solution were added at 0° C. After stirring at room temperature for 24 h, the solvents were removed, the residue was partitioned between water and ethyl acetate and the phases were separated. The aqueous phase was extracted with ethyl acetate, and the combined organic phases were washed with sat. NaCl solution and dried over sodium sulphate. The crude product obtained after removal of the solvents was purified by chromatography on silica gel. 40 mg of the desired product were obtained as a colourless foam. ¹H NMR (ppm, DMSO-D₆, 400 MHz): 3.45-3.65 (m, 4H), 4.60 (m, 1H), 4.76 (t, 1H), 5.27 (d, 1H), 6.70 (s, 1H), 7.02 (m, 1H), 7.09 (d, 2H), 7.15-7.24 (m, 5H), 7.30 (dd, 2H), 7.36 (d, 2H), 7.61 (dd, 1H), 7.85 (s, 2H), 8.17 (s, 1H), 10.37 (s, 1H).

### Example 125

### rac-N-(3-chloro-4-cyanophenyl)-2-hydroxy-2-phenyl-3-[4-(piperidine-1-carbonyl)-phenyl]propionamide

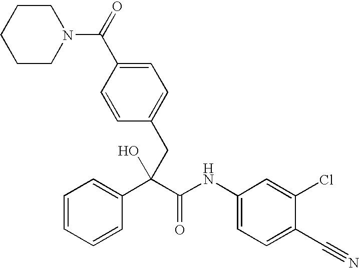

50 mg of rac-4-[2-(3-chloro-4-cyanophenylcarbamoyl)-2-hydroxy-2-phenylethyl]benzoic acid (Example 93) were initially charged in 3 ml of DMF, and 55 mg of HATU and then 50 μl of triethylamine were added. Finally, 18 μl of piperidine were added dropwise. After one hour at room temperature, the mixture was admixed with sat. ammonium chloride solution and stirred for a further 30 min. The resulting precipitate was filtered off and dried. 47 mg of the desired product were obtained as a colourless solid. ¹H NMR (ppm, CDCl₃, 400 MHz): 1.2-1.6 (m, 6H), 3.0-3.2 (m, 2H), 3.23 (d, 1H), 3.4-3.6 (m, 2H), 3.65 (d, 1H), 6.72 (s, 1H), 7.11 (d, 2H), 7.16 (m, 2H), 7.25 (m, 1H), 7.32 (dd, 2H), 7.60 (d, 2H), 7.80 (dd, 2H), 8.10 (d, 1H), 10.18 (s, 1H).

Analogously to Example 125, Examples 126-133 were prepared by reacting the corresponding carboxylic acids from Example 92 or 93 with the amines required in each case.

### Example 126

### rac-N-(3-chloro-4-cyanophenyl)-2-hydroxy-3-[4-(morpholine-1-carbonyl)phenyl]-2-phenylpropionamide

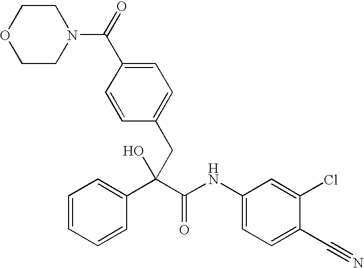

¹H NMR (ppm, DMSO-D₆, 400 MHz): 3.1-3.6 (m, 8H), 3.25 (d, 1H), 3.65 (d, 1H), 6.71 (s, 1H), 7.17 (m, 4H), 7.25 (m, 1H), 7.32 (m, 2H), 7.60 (m, 2H), 7.80 (s, 2H), 8.11 (s, 1H), 10.17(s, 1H).

### Example 127

### rac-4-[2-(3-chloro-4-cyanophenylcarbamoyl)-2-hydroxy-2-phenylethyl]-N-(2-dimethylaminoethyl)benzamide

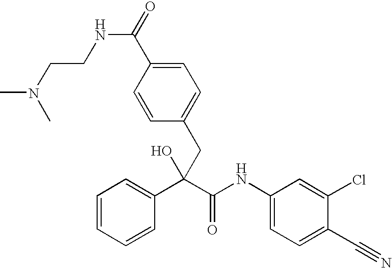

¹H NMR (ppm, DMSO-D₆, 400 MHz): 2.64 (s, 6H), 3.00 (s br, 2H), 3.26 (d, 1H), 3.45 (m, 2H), 3.69 (d, 1H), 6.72 (s, 1H), 7.23 (m, 3H), 7.32 (dd, 2H), 7.60 (m, 4H), 7.82 (m, 2H), 8.13 (d, 1H), 8.43 (m, 1H), 10.22 (s, 1H).

### Example 128

### rac-4-[2-(3-chloro-4-cyanophenylcarbamoyl)-2-hydroxy-2-phenylethyl]-N-(2-dimethylaminoethyl)-N-methylbenzamide

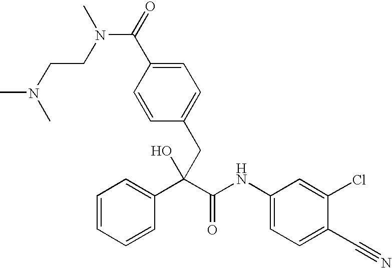

¹H NMR (ppm, DMSO-D₆, 400 MHz): 1.65-3.50 (br, 13H), 3.24 (d, 1H), 3.64 (d, 1H), 6.70 (s, 1H), 7.13 (m, 4H), 7.24 (m, 1H), 7.31 (m, 2H), 7.59 (d, 2H), 7.81 (m, 2H), 8.10 (d, 1H), 10.18(s, 1H).

### Example 129

### rac-N-(3-chloro-4-cyanophenyl)-2-hydroxy-2-phenyl-3-[3-(piperidine-1-carbonyl)-phenyl]propionamide

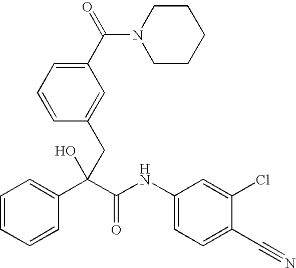

¹H NMR (ppm, DMSO-D₆, 400 MHz): 1.1-1.6 and 2.2-3.6 (br, 10H), 3.23 (d, 1H), 3.65 (d, 1H), 6.73 (s, 1H), 7.08 (m, 2H), 7.21 (m, 3H), 7.31 (dd, 2H), 7.59 (d, 2H), 7.81 (m, 2H), 8.14 (d, 1H), 10.22 (s, 1H).

### Example 130

### rac-N-(3-chloro-4-cyanophenyl)-2-hydroxy-3-[3-(morpholine-1-carbonyl)phenyl]-2-phenylpropionamide

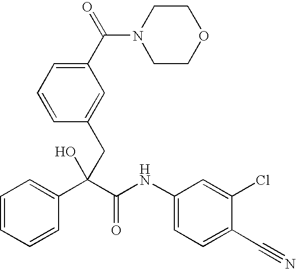

¹H NMR (ppm, DMSO-D₆, 400 MHz): 2.90-3.65 (br, 8H), 3.26 (d, 1H), 3.63 (d, 1H), 6.72 (s, 1H), 7.08 (s, 1H), 7.13 (m, 1H), 7.23 (m, 3H), 7.31 (dd, 2H), 7.58 (dd, 2H), 7.81 (s, 2H), 8.12 (s, 1H), 10.23 (s, 1H).

### Example 131

### rac-3-[2-(3-chloro-4-cyanophenylcarbamoyl)-2-hydroxy-2-phenylethyl]-N-(2-dimethylaminoethyl)benzamide

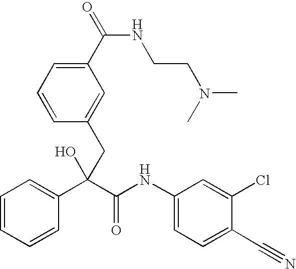

¹H NMR (ppm, DMSO-D₆, 400 MHz): 2.55-2.75 (br, 6H), 2.93 (s br, 2H), 3.22 (d, 1H), 3.44 (m, 2H), 3.70 (d, 1H), 6.70 (s, 1H), 7.28 (m, 5H), 7.59 (m, 3H), 7.68 (s, 1H), 7.79 (s, 2H), 8.10 (s, 1H), 8.39 (m, 1H), 10.16 (s, 1H).

### Example 132

### rac-3-[2-(3-chloro-4-cyanophenylcarbamoyl)-2-hydroxy-2-phenylethyl]-N-(2-dimethylaminoethyl)-N-methylbenzamide

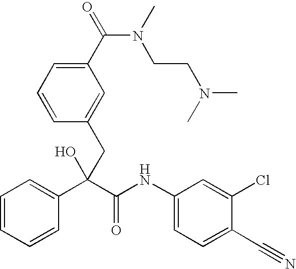

¹H NMR (ppm, DMSO-D₆, 400 MHz): 2.05 (s, 3H), 2.65 (s, 3H), 2.69 (s, 3H), 2.6-3.3 (br, 4H), 3.24 (d, 1H), 3.65 (d, 1H), 6.73 (s, 1H), 7.12-7.28 (m, 5H), 7.32 (dd, 2H), 7.59 (d, 2H), 7.81 (s, 2H), 8.11 (s, 1H), 10.20 (s, 1H).

### Example 133

### rac-3-[2-(3-chloro-4-cyanophenylcarbamoyl)-2-hydroxy-2-phenylethyl]-N-(2-piperidin-1-ylethyl)benzamide

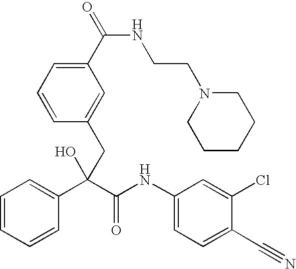

¹H NMR (ppm, DMSO-D₆, 400 MHz): 1.2-1.9 and 2.6-3.6 (br, 14H), 3.24 (d, 1H), 3.70 (d, 1H), 6.71 (s, 1H), 7.22-7.35 (m, 5H), 7.59 (m, 3H), 7.69 (s, 1H), 7.79 (m, 2H), 8.10 (s, 1H), 8.50 (s, 1H), 10.16 (s, 1H).

### Example 134

### rac-N-(3-chloro-4-cyanophenyl)-2-hydroxy-3-(4-morpholin-4-ylmethylphenyl)-2-phenylpropionamide

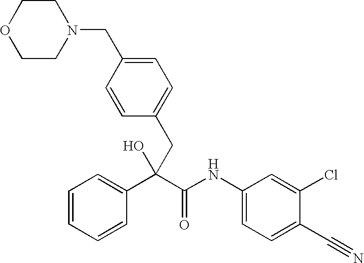

35 mg of rac-N-3-(3-chloro-4-cyanophenyl)-3-(4-formylphenyl)-2-hydroxy-2-phenyl-propionamide 10 μl of morpholine were added. After stirring for 15 min, 37 mg of sodium trisacetoxyborohydride were added. On completion of conversion (TLC monitoring), sat. sodium hydrogencarbonate was added, the mixture was partitioned between water and ethyl acetate, extraction was effected with ethyl acetate, and the combined organic phases were washed with sat. NaCl solution and dried over sodium sulphate. After chromatographic purification of the crude product, 18.7 mg of the desired product were obtained as a pale yellowish foam. ¹H NMR (ppm, DMSO-D₆, 400 MHz): 2.25 (m, 4H), 3.20 (d, 1H), 3.35 (m, 2H), 3.52 (m, 4H), 3.65 (d, 1H), 6.66 (s, 1H), 7.11 (dd, 4H), 7.33 (m, 3H), 7.64 (dd, 2H), 7.84 (m, 2H), 8.13 (s, 1H), 10.15 (s, 1H).

Analogously to Example 134, Examples 135-139 were prepared by reacting the corresponding aldehydes from Example 98 or 97 with the amines required in each case by reductive amination.

### Example 135

### rac-N-(3-chloro-4-cyanophenyl)-2-hydroxy-3-(3-morpholin-4-ylmethylphenyl)-2-phenylpropionamide

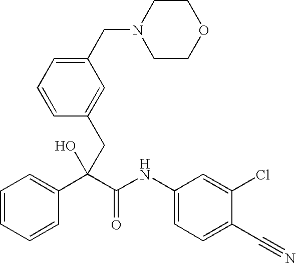

¹H NMR (ppm, DMSO-D₆, 400 MHz): 2.16 and 2.4-3.6 (br, 10H), 3.22 (d, 1H), 3.67 (d, 1H), 6.66 (s, 1H), 7.05 (m, 2H), 7.13 (m, 2H), 7.33 (m, 3H), 7.63 (dd, 2H), 7.85 (s, 2H), 8.18 (s, 1H), 10.22 (s, 1H).

### Example 136

### rac-N-(3-chloro-4-cyanophenyl)-2-hydroxy-2-phenyl-3-(3-piperidin-1-ylmethyl-phenyl)propionamide

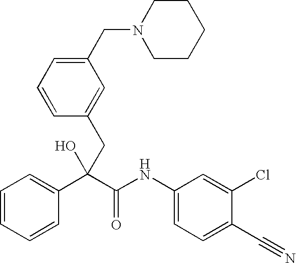

¹H NMR (ppm, DMSO-D₆, 400 MHz): 1.29 (br, 6H), 2.08 (br, 4H), 3.20 (d, 1H), 3.20 (m, 2H), 3.65 (d, 1H), 6.60 (s, 1H), 7.00 (m, 2H), 7.06 (m, 2H), 7.29 (m, 3H), 7.60 (dd, 2H), 7.80 (d, 2H), 8.14 (d, 1H), 10.13 (s, 1H).

### Example 137

### rac-N-(3-chloro-4-cyanophenyl)-3-{3-[(2-dimethylaminoethylamino)methyl]-phenyl}-2-hydroxy-2-phenylpropionamide

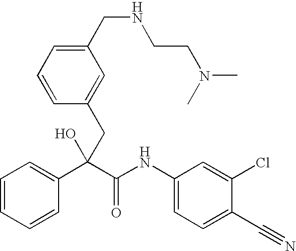

¹H NMR (ppm, DMSO-D₆, 400 MHz): 2.01 (s, 6H), 2.14 (t, 2H), 2.33 (t, 2H), 3.14 (d, 1H), 3.51 (dd, 2H), 3.64 (d, 1H), 6.59 (s, 1H), 7.05 (m, 4H), 7.29 (m, 3H), 7.60 (d, 2H), 7.80 (dd, 2H), 8.12 (s, 1H), 10.13 (s, 1H).

### Example 138

### rac-N-(3-chloro-4-cyanophenyl)-3-{3-[(2-dimethylaminoethyl)methylamino]-methyl}phenyl)-2-hydroxy-2-phenylpropionamide

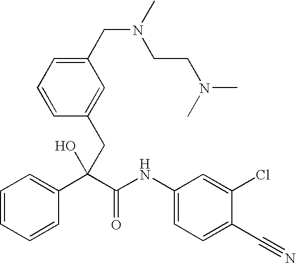

¹H NMR (ppm, DMSO-D₆, 400 MHz): 1.90 (s, 3H), 2.06 (s, 6H), 2.25 (m, 4H), 3.14 (d, 1H), 3.27 (s, 4H), 3.65 (d, 1H), 6.60 (s, 1H), 7.05 (m, 4H), 7.29 (m, 3H), 7.60 (dd, 2H), 7.79 (s, 2H), 8.13 (s, 1H), 10.13 (s, 1H).

### Example 139

### rac-N-(3-chloro-4-cyanophenyl)-2-hydroxy-2-phenyl-3-{3-[(2-piperidin-1-ylethyl-amino)methyl]phenyl}propionamide

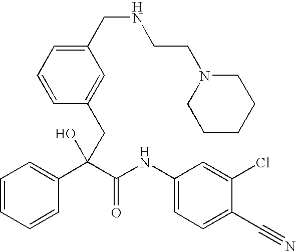

¹H NMR (ppm, DMSO-D₆, 400 MHz): 1.20-1.45 (m br, 6H), 2.18 (m, 6H), 2.35 (t, 2H), 3.14 (d, 1H), 3.53 (dd, 2H), 3.65 (d, 1H), 6.59 (s, 1H), 7.05 (m, 4H), 7.29 (m, 3H), 7.60 (d, 2H), 7.80 (dd, 2H), 8.12 (d, 1H), 10.13 (s, 1H).

Analogously to the examples based on conversions of N-(3-chloro-4-cyanophenyl)-2-oxo-2-phenylacetamide, it is possible to prepare corresponding examples based on N-(4-cyano-3-trifluoromethylphenyl)-2-oxo-2-phenylacetamide. Some representative compounds are described below:

### Example 140

### rac-3-(4-cyanophenyl)-N-(4-cyano-3-trifluoromethylphenyl)-2-hydroxy-2-phenyl-propionamide

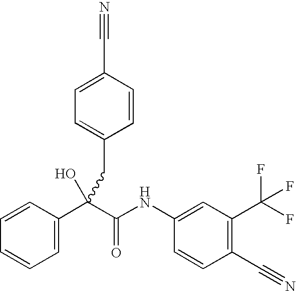

382 mg of magnesium turnings were initially charged in 15 ml of THF, a little iodine was added and then 3.08 g of 4-cyanobenzyl bromide dissolved in 15 ml of THF were added dropwise slowly and in a controlled manner. The reaction mixture was stirred at 70° C. for 5 h, then cooled to −70° C., and 500 mg of N-(4-cyano-3-trifluoromethylphenyl)-2-oxo-2-phenylacetamide (Example 35.1)), dissolved in 10 ml of THF, were added dropwise. The mixture was left to thaw overnight, the reaction was ended by adding sat. ammonium chloride solution and extraction was effected with ethyl acetate. The combined organic phases were washed with sat. NaCl solution and dried over sodium sulphate. The crude product was purified by chromatography. 596 mg of the desired product were obtained. ¹H NMR (ppm, CDCl₃, 400 MHz): 2.73 (s, 1H), 3.44 (d, 1H), 3.94 (d, 1H), 7.26 (m, 1H), 7.34 (m, 1H), 7.46 (m, 3H), 7.60 (m, 2H), 7.70 (dd, 2H), 7.80 (d, 1H), 7.89 (dd, 1H), 8.03 (d, 1H), 8.85 (s, 1H).

### Example 141

### rac-N-(4-cyano-3-trifluoromethylphenyl)-2-hydroxy-3-(3-iodophenyl)-2-phenyl-propionamide

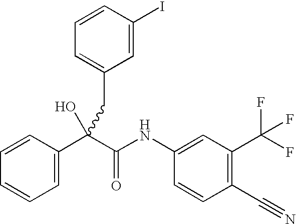

The compound was prepared analogously to Example 86.

¹H NMR (ppm, CDCl₃, 400 MHz): 2.76(s, 1H), 3.25(d, 1H), 3.86(d, 1H), 7.05(m, 1H), 7.14(m, 1H), 7.45 (m, 3H), 7.58 (s, 1H), 7.68 (m, 3H), 7.80 (d, 1H), 7.91 (dd, 1H), 8.02 (d, 1H), 8.89 (s, 1H).

### Example 142

### rac-2-(4′-acetylbiphenyl-2-yl)-N-(4-cyano-3-trifluoromethylphenyl)-2-hydroxy-2-phenylpropionamide

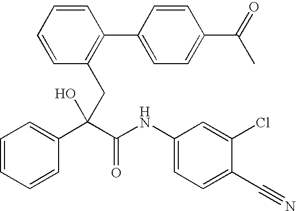

The compound was prepared analogously to Example 119 from the corresponding 2-iodophenyl compound. ¹H NMR (ppm, DMSO-D₆, 400 MHz): 2.59 (s, 3H), 3.52 (dd, 2H), 6.66 (s, 1H), 7.00 (dd, 1H), 7.18 (m, 9H), 7.58 (d, 1H), 7.91 (d, 2H), 7.99 (d, 1H), 8.13 (dd, 1H), 8.37 (d, 1H), 10.48 (s, 1H).

Analogously to Example 5, Examples 143-147 were prepared by reacting N-(3-chloro-4-cyano-2-methylphenyl)-2-oxo-2-phenylacetamide (prepared analogously to Example 1.1) from phenylglyoxylic acid, thionyl chloride and 4-amino-2-chloro-3-methylbenzonitrile, ¹H NMR (ppm, CDCl₃, 400 MHz): 2.50 (s, 3H), 7.55 (dd, 2H), 7.62 (d, 1H), 7.71 (dd, 1H), 8.36 (d, 1H), 8.44 (dd, 1H), 9.27 (s, 1H)) with the Grignard reagents required in each case.

### Example 143

### rac-N-(3-chloro-4-cyano-2-methylphenyl)-2-hydroxy-2,3-diphenylpropionamide

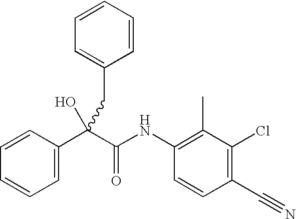

¹H NMR (ppm, CDCl₃, 400 MHz): 2.15 (s, 3H), 2.84 (s, 1H), 3.25 (d, 1H), 3.94 (d, 1H), 7.18 (dd, 2H), 7.30 (m, 3H), 7.40 (m, 3H), 7.50 (d, 1H), 7.73 (d, 2H), 8.15 (d, 1H), 8.77 (s, 1H).

### Example 144

### rac-N-(3-chloro-4-cyano-2-methylphenyl)-2-hydroxy-2-phenyl-3-p-tolyl-propionamide

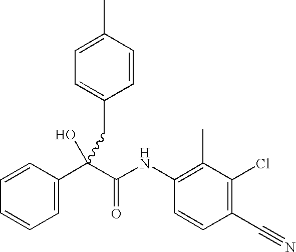

¹H NMR (ppm, CDCl₃, 400 MHz): 2.16 (s, 3H), 2.31 (s, 3H), 2.83 (s, 1H), 3.20 (d, 1H), 3.91 (d, 1H), 7.08 (dd, 4H), 7.38 (m, 3H), 7.50 (d, 1H), 7.50 (d, 1H), 7.73 (dd, 2H), 8.15 (d, 1H), 8.78 (s, 1H).

### Example 145

### rac-N-(3-chloro-4-cyano-2-methylphenyl)-2-hydroxy-2-phenyl-3-m-tolyl-propionamide

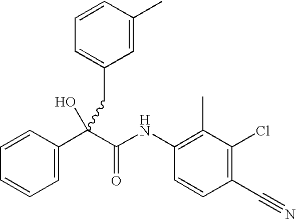

¹H NMR (ppm, CDCl₃, 400 MHz): 2.16 (s, 3H), 2.28 (s, 3H), 2.84 (s, 1H), 3.18 (d, 1H), 3.92 (d, 1H), 6.98 (m, 2H), 7.11 (m, 1H), 7.19 (m, 1H), 7.39 (m, 3H), 7.51 (d, 1H), 7.73 (dd, 2H), 8.15 (d, 1H), 8.78 (s, 1H).

### Example 146

### rac-N-(3-chloro-4-cyano-2-methylphenyl)-2-hydroxy-3-(4-methoxyphenyl)-2-phenylpropionamide

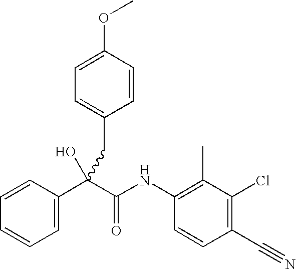

¹H NMR (ppm, CDCl₃, 400 MHz): 2.18 (s, 3H), 2.82 (s, 3H), 3.18 (d, 1H), 3.77 (s, 3H), 3.88 (d, 1H), 6.83 (d, 2H), 7.09 (d, 2H), 7.38 (m, 3H), 7.50 (d, 1H), 7.72 (dd, 2H), 8.16 (d, 1H), 8.79 (s, 1H).

### Example 147

### rac-N-(3-chloro-4-cyano-2-methylphenyl)-2-hydroxy-3-(3-methoxyphenyl)-2-phenylpropionamide

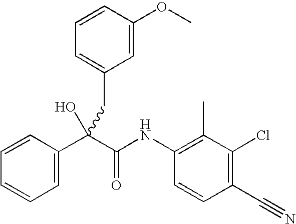

¹H NMR (ppm, CDCl₃, 400 MHz): 2.16 (s, 3H), 2.88 (s, 1H), 3.23 (d, 1H), 3.69 (s, 3H), 3.90 (d, 1H), 6.67 (m, 1H), 6.76 (d, 1H), 6.83 (dd, 1H), 7.22 (dd, 1H), 7.35 (m, 1H), 7.41 (dd, 2H), 7.50 (d, 1H), 7.73 (dd, 2H), 8.18 (d, 1H), 8.81 (s, 1H).

### Example 148

### rac-N-(3-chloro-4-cyanophenyl)-2-hydroxy-3-phenyl-2-thiophen-2-ylpropionamide

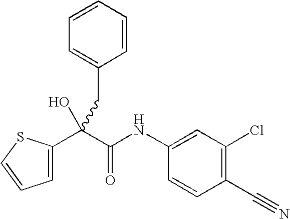

was prepared analogously to Example 5 by reacting N-(3-chloro-4-cyanophenyl)-2-oxo-2-thiophen-2-ylacetamide (prepared analogously to Example 1.1) from oxothiophen-2-ylacetic acid, thionyl chloride and 4-amino-2-chlorobenzonitrile, ¹H NMR (ppm, DMSO-D₆, 400 MHz): 7.35 (m, 1H), 7.99 (m, 2H), 8.27 (m, 3H), 11.41 (s, 1H)) with benzylmagnesium chloride. ¹H NMR (ppm, CDCl₃, 400 MHz): 3.06 (s, 1H), 3.40 (d, 1H), 3.85 (d, 1H), 7.06 (dd, 1H), 7.21 (dd, 2H), 7.26 (dd, 1H), 7.34 (m, 4H), 7.48 (dd, 1H), 7.61 (d, 1H), 7.95 (d, 1H), 8.78 (s, 1H).

### Example 149

### rac-N-(4-cyano-3-trifluoromethylphenyl)-2-hydroxy-3-phenyl-2-thiophen-2-yl-propionamide

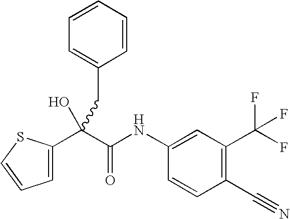

was prepared analogously to Example 5 by reacting N-(4-cyano-3-trifluoromethyl-phenyl)-2-oxo-2-thiophen-2-ylacetamide (prepared analogously to Example 1.1) from oxothiophen-2-ylacetic acid, thionyl chloride and 4-amino-2-trifluoromethylbenzonitrile) with benzylmagnesium chloride. ¹H NMR (ppm, CDCl₃, 400 MHz): 3.11 (s, 1H), 3.42 (d, 1H), 3.85 (d, 1H), 7.07 (dd, 1H), 7.22 (dd, 2H), 7.26 (dd, 1H), 7.34 (m, 2H), 7.42 (d, 2H), 7.81 (d, 1H), 7.92 (d, 1H), 8.92 (s, 1H)

### Example 150

### rac-N-(4-cyano-3-trifluoromethylphenyl)-2-hydroxy-2-thiophen-2-yl-3-p-tolyl-propionamide

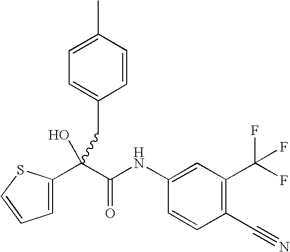

was prepared analogously to Example 149 with 4-methylbenzylmagnesium chloride.

¹H NMR (ppm, DMSO-D₆, 400 MHz): 2.16 (s, 3H), 3.16 (d, 1H), 3.58 (d, 1H), 6.96 (s, 1H), 6.97 (m, 2H), 7.06 (m, 3H), 7.18 (dd, 1H), 7.41 (dd, 1H), 8.00 (d, 1H), 8.18 (dd, 1H), 8.35 (d, 1H), 10.35 (s, 1H).

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2006 061 912.9, filed Dec. 21, 2006, U.S. Provisional Application Ser. No. 60/880,707, filed Jan. 17, 2007 and U.S. Provisional Application Ser. No. 60/979,208, filed Oct. 11, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula I 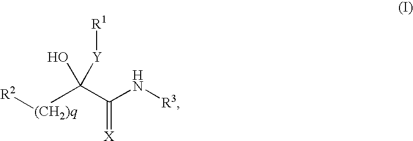
in which
q is 0 or 1,
R¹ is a mono- or bicyclic C₆-C₁₂-aryl, 5-12-membered heteroaryl, C₃-C₁₀-cycloalkyl or a 3-10-membered heterocycloalkyl radical which may in each case be unsubstituted or optionally substituted by up to 3 radicals, where the substituents each independently of one another have the following meaning:
C₁-C₈-alkyl, C₂-C₈-alkenyl, C₂-C₈-alkynyl, partly or fully fluorinated C₁-C₆-alkyl, partly or fully fluorinated C₁-C₆-alkoxy, C₁-C₆-alkoxy-C₁-C₆-alkyl, C₁-C₆-alkoxy-C₁-C₆-alkoxy, (CH₂)ₚ-C₃-C₁₀-cycloalkyl, (CH₂)ₚ-heterocycloalkyl, (CH₂)ₚCN, (CH₂)ₚHal, (CH₂)ₚNO₂, an optionally Z-substituted (CH₂)ₚ-C₆-C₁₂-aryl, an optionally Z-substituted (CH₂)ₚ-heteroaryl,
—(CH₂)ₚPO₃(R^{b})₂, —(CH₂)ₚNR^{c}R^{d}, —(CH₂)ₚNR^{e}COR^{b},
—(CH₂)ₚNR^{e}CSR^{b}, —(CH₂)ₚNR^{e}S(O)R^{b}, —(CH₂)ₚNR^{e}S(O)₂R^{b},
—(CH₂)ₚNR^{e}CONR^{c}R^{d}, —(CH₂)ₚNR^{e}COOR^{b},
—(CH₂)ₚNR^{e}C(NH)NR^{c}R^{d}, —(CH₂)ₚNR^{e}CSNR^{c}R^{d},
—(CH₂)ₚNR^{e}S(O)NR^{c}R^{d}, —(CH₂)ₚNR^{e}S(O)₂NR^{c}R^{d}, —(CH₂)ₚCOR^{b},
—(CH₂)ₚCSR^{b}, —(CH₂)ₚ S(O)R^{b}, —(CH₂)ₚS(O)(NH)R^{b}, —(CH₂)ₚS(O)₂R^{b},
—(CH₂)ₚS(O)₂NR^{c}R^{d}, —(CH₂)ₚSO₂OR^{b}, —(CH₂)ₚCO₂R^{b},
—(CH₂)ₚCONR^{c}R^{d}, —(CH₂)ₚCSNR^{c}R^{d}, —(CH₂)ₚOR^{b}, —(CH₂)ₚSR^{b},
—(CH₂)ₚCR^{b}(OH)—R^{b}, —(CH₂)ₚ—C═NOR^{b},
—O—(CH₂)ₙ—O—, —O—(CH₂)ₙ—CH₂—, —O—CH═CH— or —CH₂)ₙ₊₂—,
where n is 1 or 2 and the terminal oxygen atoms and/or carbon atoms are linked to directly adjacent ring carbon atoms, and p is 0, 1, 2, 3, 4, 5 or 6, and
Z is a cyano, halogen, nitro, —(CH₂)ₚOR^{b}, —(CH₂)ₚS(O)₂R^{b}, —C(O)R^{b}, CO₂R^{b}, —O—R^{b}, —S—R^{b}, SO₂NR^{c}R^{d}, —C(O)—NR^{c}R^{d}, —OC(O)—NR^{c}R^{d}, —C═NOR^{b}—NR^{c}R^{d}, partly or fully fluorinated C₁-C₆-alkyl or partly or fully fluorinated C₁-C₆-alkoxy,
R^{b} is a hydrogen or a C₁-C₆-alkyl, hydroxy-C₁-C₃-alkyl, C₁-C₃-alkoxy-C₁-C₃-alkyl, C₂-C₈-alkenyl, C₂-C₈-alkynyl, C₃-C₁₀-cycloalkyl, C₆-C₁₂-aryl or a partly or completely fluorinated C₁-C₃-alkyl and
R^{c} and R^{d} are independently of one another a hydrogen, an optionally W-substituted C₁-C₆-alkyl, C₂-C₈-alkenyl, C₂-C₈-alkynyl, C₃-C₁₀-cycloalkyl, C₆-C₁₂-aryl, a 5- to 12-membered heteroaryl radical, C(O)R^{b} or a hydroxy group and
w is —NR^{f}R^{g} where
R^{f} is hydrogen or C₁-C₃-alkyl and
R^{g} is hydrogen or C₁-C₃-alkyl or
R^{f} and R^{g}, with inclusion of the nitrogen, form a 3- to 7-membered ring which is optionally extended by O, S or NR^{h} where
R^{h} is hydrogen, C₁-C₃-alkyl, C₁-C₃-alkanoyl, C₁-C₃-alkylsulphonyl or C₁-C₃-alkoxycarbonyl,
where if
R^{c} is a hydroxy group, R^{d} can only be a hydrogen, a C₁-C₆-alkyl, C₂-C₈-alkenyl, C₂-C₈-alkynyl, C₃-C₁₀-cycloalkyl or C₆-C₁₂-aryl and vice versa, and
R^{e} is a hydrogen, C₁-C₆-alkyl, C₂-C₈-alkenyl, C₂-C₈-alkynyl, C₃-C₁₀-cycloalkyl or C₆-C₁₂-aryl,
R² is a mono- or bicyclic C₆-C₁₂-aryl or 5-12-membered heteroaryl radical which may be unsubstituted or optionally substituted by up to 3 of the radicals mentioned under R¹,
R³ has the meaning indicated for R² or is one of the following groups mentioned under A or B:
A: 6-Membered/6Membered Ring Systems 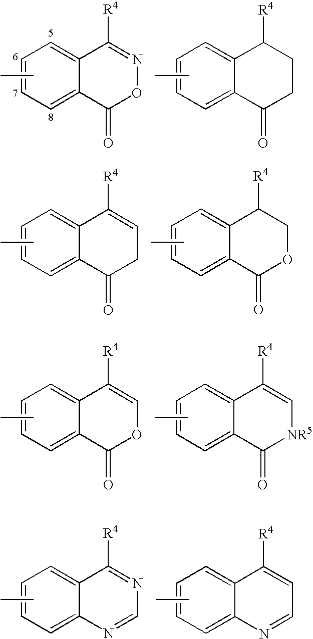
Linkage to the Basic Structure at Position 5, 6, 7 or 8
B: 6-Membered/5-Membered Ring Systems 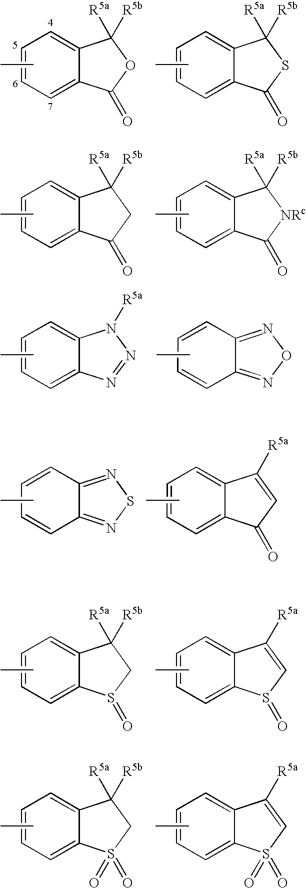
Linkage to the Basic Structure at Position 4, 5, 6 or 7
where
R⁴ is hydrogen or C₁-C₄-alkyl or partly or fully fluorinated C₁-C₄-alkyl,
R^{5a} and R^{5b} are independently of one another hydrogen, C₁-C₄-alkyl or partly or fully fluorinated C₁-C₄-alkyl, or together with the ring carbon atom form a 3- to 6-membered ring,
and
X is oxygen or two hydrogen atoms,
Y is (CH₂)ₘ, —C≡C— or —CH═CH—, and
m is 0 or 1,
and the pharmaceutically acceptable salts thereof.

2. Compounds according to claim 1, in which R¹ is a mono- or bicyclic C₆-C₁₂-aryl or 5-12-membered heteroaryl radical, each of which may be unsubstituted or optionally substituted by up to 2 radicals, where the substituents each independently of one another have the meanings mentioned under R¹.

3. Compounds according to claim 2, in which R¹ is a monocyclic C₆-C₁₂-aryl or 5-12-membered heteroaryl radical which is unsubstituted or substituted by up to 2 of the groups appropriately mentioned under R¹.

4. Compounds according to claim 3, in which R¹ is a 5- or 6-membered aromatic or heteroaromatic ring, preferably an aromatic ring, particularly preferably a phenyl radical.

5. Compounds according to claim 1, in which R² and R³ are a mono- or bicyclic C₆-C₁₂-aryl or 5-12-membered heteroaryl radical which may be unsubstituted or optionally substituted by up to 3 of the radicals mentioned under R¹.

6. Compounds according to claim 1, in which R²is an optionally substituted phenyl or naphthyl radical or an optionally substituted mono- or bicyclic 5-10-membered heteroaryl radical.

7. Compounds according to claim 6, in which the mono- or bicyclic 5-10-membered heteroaryl radicals bear preferably 1 to 3 nitrogen atoms and/or 1 sulphur atom and/or 1 oxygen atom.

8. Compounds according to claim 7, wherein furanyl, thiophenyl, oxazolyl, thiazolyl, pyridinyl, pyrimidinyl and quinolinyl and quinazolyl are particularly preferred.

9. Compounds according to claim 6, wherein the phenyl ring substituted by nitro, cyano, trifluoromethyl, phenyl, tert-butyl, methoxy, dimethylamino, methylsulphonyl, phenoxy, acetyl, hydroxy, acetoxy, thiomethyl, hydroxymethyl, fluorine, chlorine, bromine is particularly preferred.

10. Compounds according to claim 1, in which R³ is a phenyl ring substituted by up to 2 of the radicals mentioned under R¹.

11. Compounds according to claim 10, in which the phenyl ring is substituted by chlorine, by a cyano radical and/or a trifluoromethyl radical.

12. Compounds according to claim 1, in which R³ has one of the following definitions:

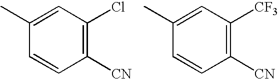

13. Compounds according to claim 1, in which R³ is a radical from the group B mentioned under R³, where the linkage to the NH group which is adjacent according to general formula (I) takes place at position 4 or 5 of the ring systems mentioned under B.

14. Compounds according to claim 1, in which R³ has one of the following meanings:

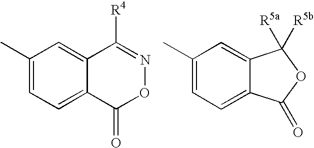

15. Compounds according to claim 1, in which R⁴ is a methyl or an ethyl radical.

16. Compounds according to claim 1, in which R^{5a} and R^{5b} are both simultaneously hydrogen.

17. Compounds according to claim 1, in which q is equal to 0.

18. Compounds according to claim 1, in which p is a number 0, 1 or 2.

19. Compounds according to claim 1, specifically

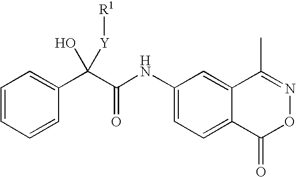

| | | Racemic or | |
|---|---|---|---|
| No. | Example | enantiomer | —Y—R¹ |
| 123 | 1 | rac+− | 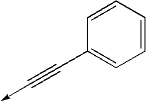 |
| 456 | 2 | rac+− | 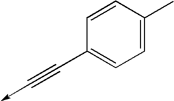 |
| 789 | 4 | rac+− | 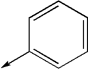 |
| 101112 | 5 6a 6b | rac+− | 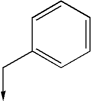 |
| 131415 | 7 | rac+− | 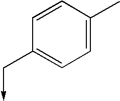 |
| 161718 | 8 | rac+− | 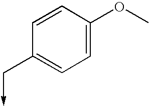 |
| 192021 | 9 | rac+− | 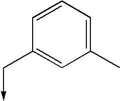 |
| 222324 | 10 | rac+− | 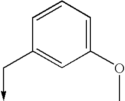 |

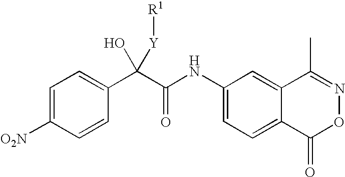
| | | Racemic or | |
|---|---|---|---|
| No. | Example | enantiomer | —Y—R¹ |
| 252627 | | rac+− | 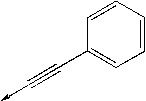 |
| 282930 | | rac+− | 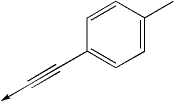 |
| 313233 | | rac+− | 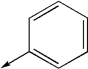 |
| 343536 | 11 | rac+− | 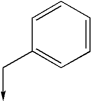 |
| 373839 | 12 | rac+− | 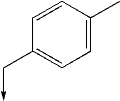 |
| 404142 | 13 | rac+− | 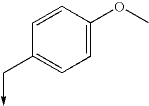 |
| 434445 | 14 | rac+− | 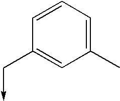 |
| 464748 | 15 | rac+− | 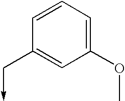 |

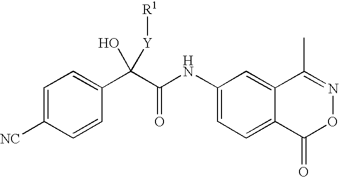
| | | Racemic or | |
|---|---|---|---|
| No. | Example | enantiomer | —Y—R¹ |
| 495051 | | rac+− | 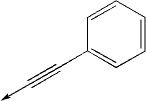 |
| 525354 | | rac+− | 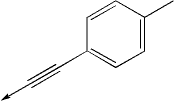 |
| 555657 | 16 | rac+− | 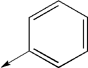 |
| 585960 | 17 | rac+− | 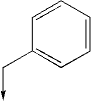 |
| 616263 | 19 | rac+− | 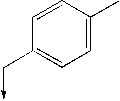 |
| 646566 | | rac+− | 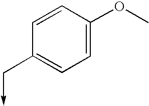 |
| 676869 | 18 | rac+− | 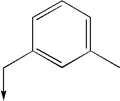 |
| 707172 | | rac+− | 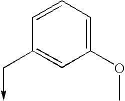 |

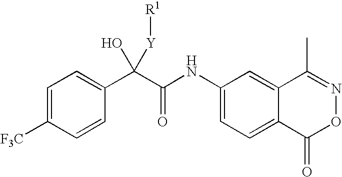
| | | Racemic or | |
|---|---|---|---|
| No. | Example | enantiomer | —Y—R¹ |
| 737475 | | rac+− | 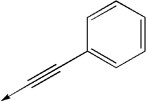 |
| 767778 | | rac+− | 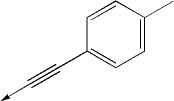 |
| 798081 | | rac+− | 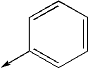 |
| 828384 | 20 | rac+− | 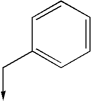 |
| 858687 | 21 | rac+− | 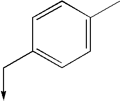 |
| 888990 | | rac+− | 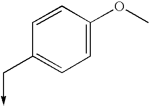 |
| 919293 | | rac+− | 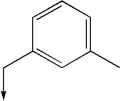 |
| 949596 | | rac+− | 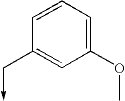 |

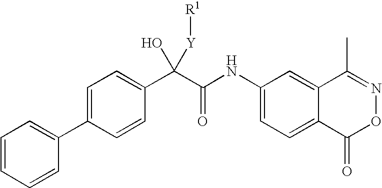
| | | Racemic or | |
|---|---|---|---|
| No. | Example | enantiomer | —Y—R¹ |
| 97 98 99 | | rac+− | 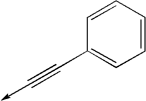 |
| 100101102 | | rac+− | 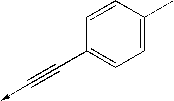 |
| 103104105 | | rac+− | 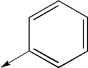 |
| 106107108 | 22 | rac+− | 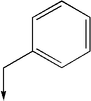 |
| 109110111 | | rac+− | 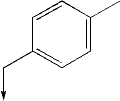 |
| 112113114 | | rac+− | 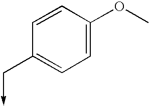 |
| 115116117 | | rac+− | 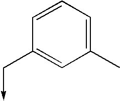 |
| 118119120 | | rac+− | 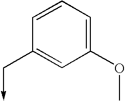 |

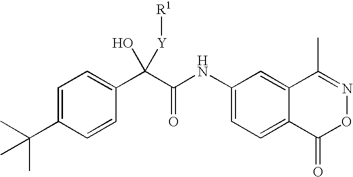
| | | Racemic or | |
|---|---|---|---|
| No. | Example | enantiomer | —Y—R¹ |
| 121122123 | | rac+− | 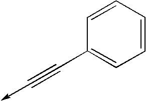 |
| 124125126 | | rac+− | 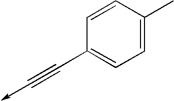 |
| 127128129 | | rac+− | 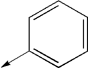 |
| 130131132 | 22 | rac+− | 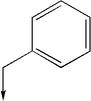 |
| 133134135 | | rac+− | 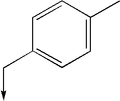 |
| 136137138 | | rac+− | 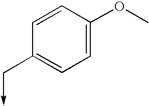 |
| 139140141 | | rac+− | 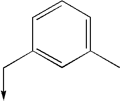 |
| 142143144 | | rac+− | 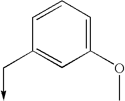 |

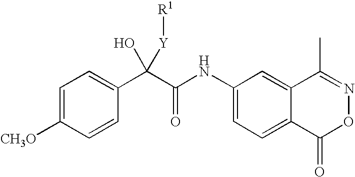
| | | Racemic or | |
|---|---|---|---|
| No. | Example | enantiomer | —Y—R¹ |
| 145146147 | | rac+− | 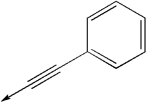 |
| 148149150 | | rac+− | 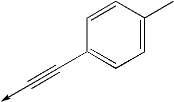 |
| 151152153 | | rac+− | 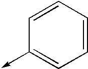 |
| 154155156 | | rac+− | 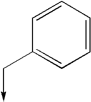 |
| 157158159 | | rac+− | 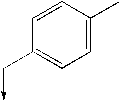 |
| 160161162 | | rac+− | 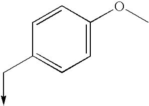 |
| 163164165 | | rac+− | 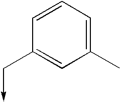 |
| 166167168 | | rac+− | 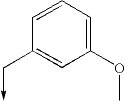 |

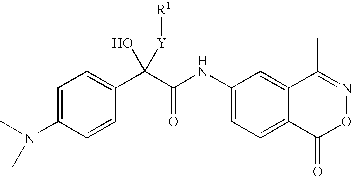
| | | Racemic or | |
|---|---|---|---|
| No. | Example | enantiomer | —Y—R¹ |
| 169170171 | | rac+− | 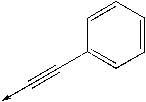 |
| 172173174 | | rac+− | 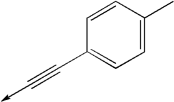 |
| 175176177 | | rac+− | 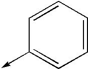 |
| 178179180 | | rac+− | 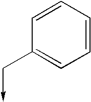 |
| 181182183 | | rac+− | 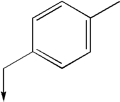 |
| 184185186 | | rac+− | 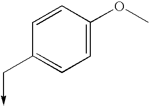 |
| 187188189 | | rac+− | 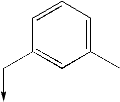 |
| 190191192 | | rac+− | 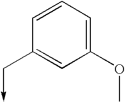 |

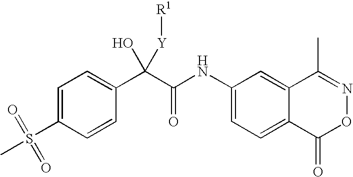
| | | Racemic or | |
|---|---|---|---|
| No. | Example | enantiomer | —Y—R¹ |
| 193194195 | | rac+− | 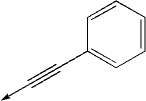 |
| 196197198 | | rac+− | 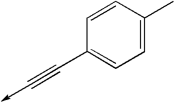 |
| 199200201 | | rac+− | 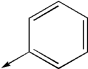 |
| 202203204 | | rac+− | 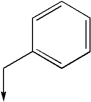 |
| 205206207 | | rac+− | 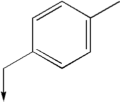 |
| 208209210 | | rac+− | 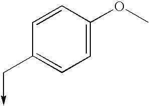 |
| 211212213 | | rac+− | 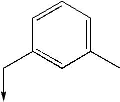 |
| 214215216 | | rac+− | 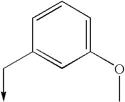 |

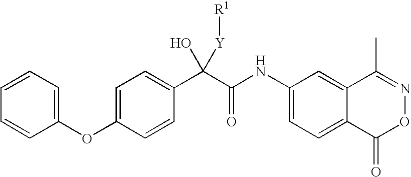
| | | Racemic or | |
|---|---|---|---|
| No. | Example | enantiomer | —Y—R¹ |
| 217218219 | | rac+− | 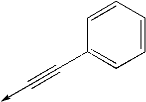 |
| 220221222 | | rac+− | 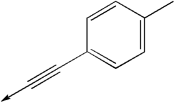 |
| 223224225 | | rac+− | 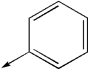 |
| 226227228 | | rac+− | 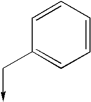 |
| 229230231 | | rac+− | 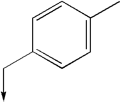 |
| 232233234 | | rac+− | 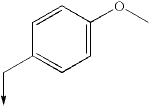 |
| 235236237 | | rac+− | 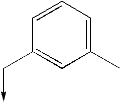 |
| 238239240 | | rac+− | 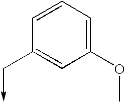 |

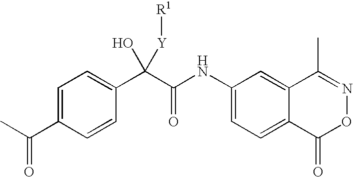
| | | Racemic or | |
|---|---|---|---|
| No. | Example | enantiomer | —Y—R¹ |
| 241242243 | | rac+− | 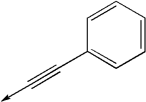 |
| 244245246 | | rac+− | 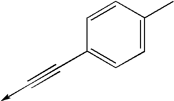 |
| 247248249 | | rac+− | 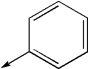 |
| 250251252 | | rac+− | 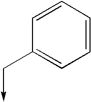 |
| 253254255 | | rac+− | 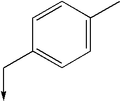 |
| 256257258 | | rac+− | 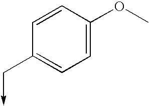 |
| 259260261 | | rac+− | 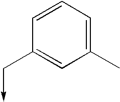 |
| 262263264 | | rac+− | 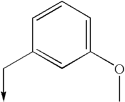 |

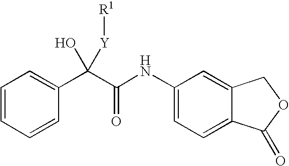
| | | Racemic or | |
|---|---|---|---|
| No. | Example | enantiomer | —Y—R¹ |
| 265266267 | 23 | rac+− | 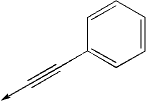 |
| 268269270 | | rac+− | 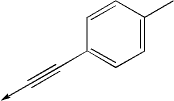 |
| 271272273 | 24 | rac+− | 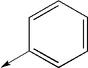 |
| 274275276 | 25 | rac+− | 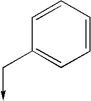 |
| 277278279 | | rac+− | 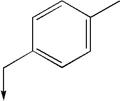 |
| 280281282 | | rac+− | 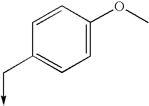 |
| 283284285 | | rac+− | 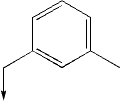 |
| 286287288 | | rac+− | 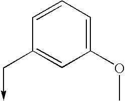 |

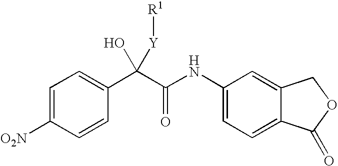
| | | Racemic or | |
|---|---|---|---|
| No. | Example | enantiomer | —Y—R¹ |
| 289290291 | 26 | rac+− | 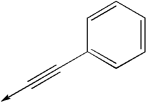 |
| 292293294 | | rac+− | 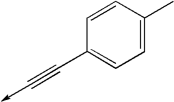 |
| 295296297 | 27 | rac+− | 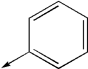 |
| 298299300 | 28 | rac+− | 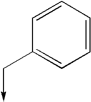 |
| 301302303 | | rac+− | 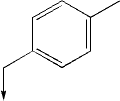 |
| 304305306 | | rac+− | 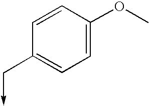 |
| 307308309 | | rac+− | 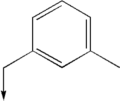 |
| 310311312 | | rac+− | 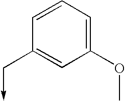 |

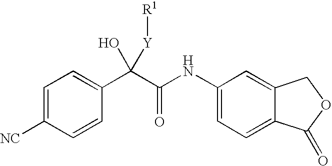
| | | Racemic or | |
|---|---|---|---|
| No. | Example | enantiomer | —Y—R¹ |
| 313314315 | 26 | rac+− | 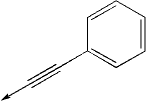 |
| 316317318 | | rac+− | 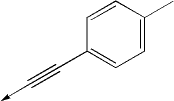 |
| 319320321 | | rac+− | 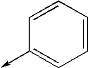 |
| 322323324 | 29 | rac+− | 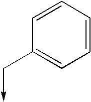 |
| 325326327 | | rac+− | 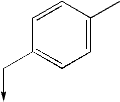 |
| 328329330 | | rac+− | 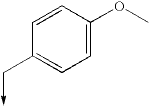 |
| 331332333 | | rac+− | 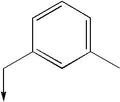 |
| 334335336 | | rac+− | 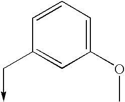 |

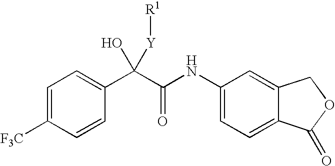
| | | Racemic or | |
|---|---|---|---|
| No. | Example | enantiomer | —Y—R¹ |
| 337338339 | | rac+− | 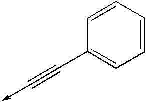 |
| 340341342 | | rac+− | 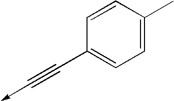 |
| 343344345 | | rac+− | 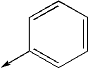 |
| 346347348 | | rac+− | 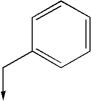 |
| 349350351 | | rac+− | 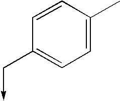 |
| 352353354 | | rac+− | 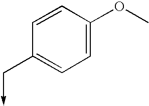 |
| 355356357 | | rac+− | 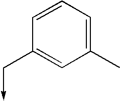 |
| 358359360 | | rac+− | 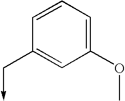 |

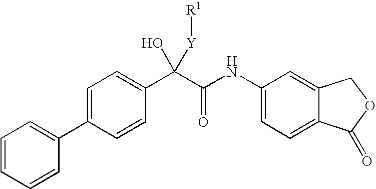
| | | Racemic or | |
|---|---|---|---|
| No. | Example | enantiomer | —Y—R¹ |
| 361362363 | | rac+− | 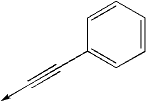 |
| 364365366 | | rac+− | 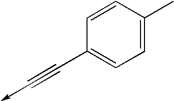 |
| 367368369 | | rac+− | 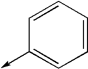 |
| 370371372 | | rac+− | 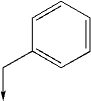 |
| 373374375 | | rac+− | 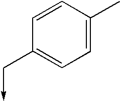 |
| 376377378 | | rac+− | 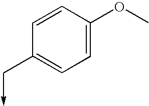 |
| 379380381 | | rac+− | 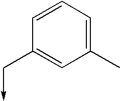 |
| 382383384 | | rac+− | 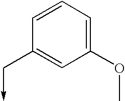 |

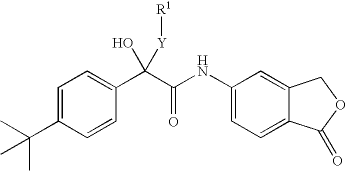
| | | Racemic or | |
|---|---|---|---|
| No. | Example | enantiomer | —Y—R¹ |
| 385386387 | | rac+− | 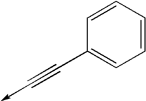 |
| 388389390 | | rac+− | 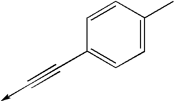 |
| 391392393 | | rac+− | 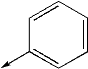 |
| 394395396 | | rac+− | 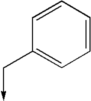 |
| 397398399 | | rac+− | 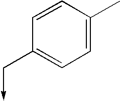 |
| 400401402 | | rac+− | 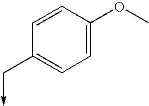 |
| 403404405 | | rac+− | 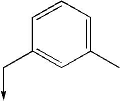 |
| 406407408 | | rac+− | 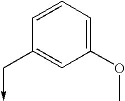 |

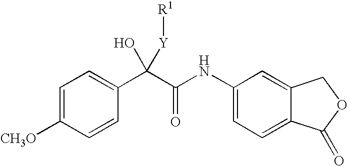
| | | Racemic or | |
|---|---|---|---|
| No. | Example | enantiomer | —Y—R¹ |
| 409410411 | | rac+− | 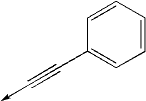 |
| 412413414 | | rac+− | 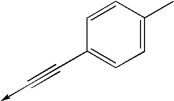 |
| 415416417 | | rac+− | 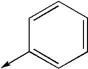 |
| 418419420 | | rac+− | 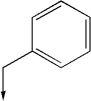 |
| 421422423 | | rac+− | 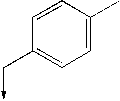 |
| 424425426 | | rac+− | 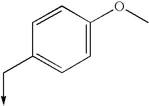 |
| 427428429 | | rac+− | 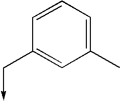 |
| 430431432 | | rac+− | 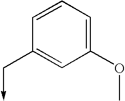 |

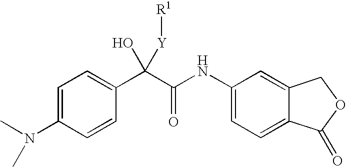
| | | Racemic or | |
|---|---|---|---|
| No. | Example | enantiomer | —Y—R¹ |
| 433434435 | | rac+− | 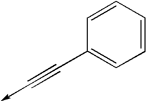 |
| 436437438 | | rac+− | 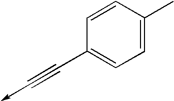 |
| 439440441 | | rac+− | 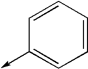 |
| 442443444 | | rac+− | 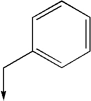 |
| 445446447 | | rac+− | 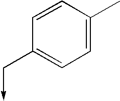 |
| 448449450 | | rac+− | 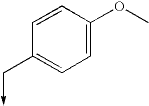 |
| 451452453 | | rac+− | 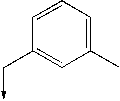 |
| 454455456 | | rac+− | 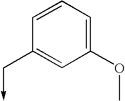 |

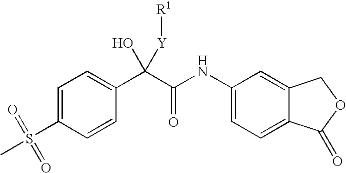
| | | Racemic or | |
|---|---|---|---|
| No. | Example | enantiomer | —Y—R¹ |
| 457458459 | | rac+− | 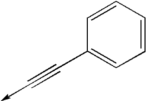 |
| 460461462 | | rac+− | 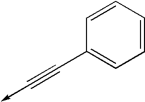 |
| 463464465 | | rac+− | 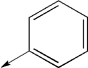 |
| 466467468 | | rac+− | 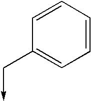 |
| 469470471 | | rac+− | 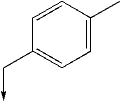 |
| 472473474 | | rac+− | 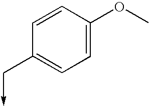 |
| 475476477 | | rac+− | 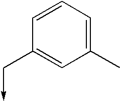 |
| 478479480 | | rac+− | 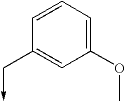 |

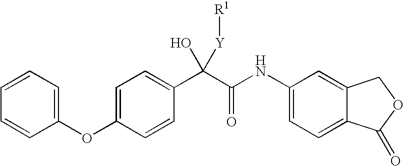
| | | Racemic or | |
|---|---|---|---|
| No. | Example | enantiomer | —Y—R¹ |
| 481482483 | | rac+− | 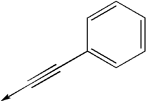 |
| 484485486 | | rac+− | 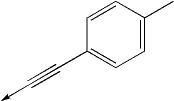 |
| 487488489 | | rac+− | 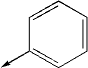 |
| 490491492 | | rac+− | 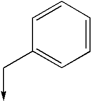 |
| 493494495 | | rac+− | 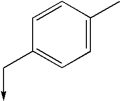 |
| 496497498 | | rac+− | 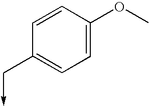 |
| 499500501 | | rac+− | 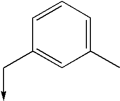 |
| 502503504 | | rac+− | 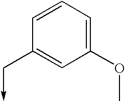 |

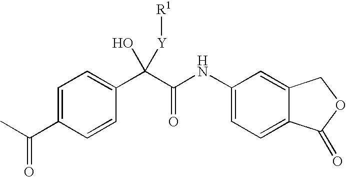
| | | Racemic or | |
|---|---|---|---|
| No. | Example | enantiomer | —Y —R¹ |
| 505506507 | | rac+− | 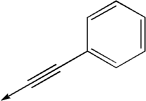 |
| 508509510 | | rac+− | 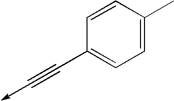 |
| 511512513 | | rac+− | 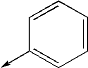 |
| 514515516 | | rac+− | 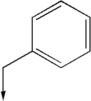 |
| 517518519 | | rac+− | 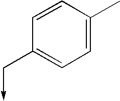 |
| 520521522 | | rac+− | 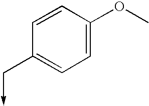 |
| 523524525 | | rac+− | 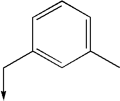 |
| 526527528 | | rac+− | 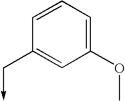 |

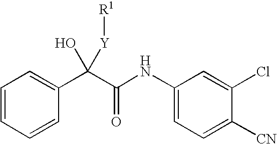
| | | Racemic or | |
|---|---|---|---|
| No. | Example | enantiomer | —Y—R¹ |
| 529530531 | | rac+− | 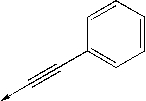 |
| 532533534 | | rac+− | 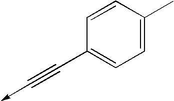 |
| 535536537 | | rac+− | 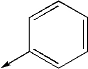 |
| 538539540 | 30 | rac+− | 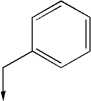 |
| 541542543 | 31 | rac+− | 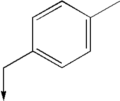 |
| 544545546 | 32 | rac+− | 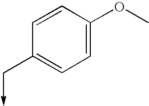 |
| 547548549 | 33 | rac+− | 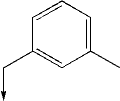 |
| 550551552 | 34 | rac+− | 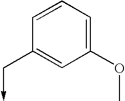 |

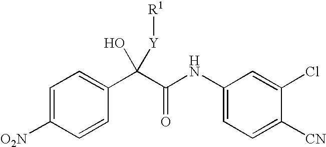
| | | racemisch oder | |
|---|---|---|---|
| Nr | Beispiel | Enantiomer | —Y —R¹ |
| 553554555 | | rac+− | 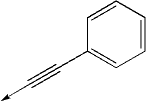 |
| 556557558 | | rac+− | 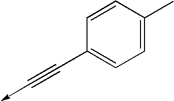 |
| 559560561 | | rac+− | 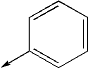 |
| 562563564 | | rac+− | 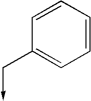 |
| 565566567 | | rac+− | 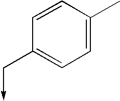 |
| 568569570 | | rac+− | 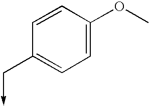 |
| 571572573 | | rac+− | 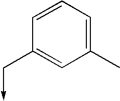 |
| 574575576 | | rac+− | 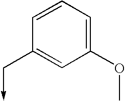 |

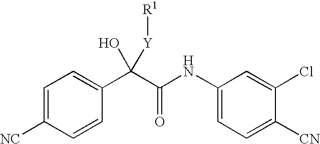
| | | Racemic or | |
|---|---|---|---|
| No. | Example | enantiomer | —Y —R¹ |
| 577578579 | | rac+− | 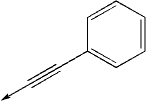 |
| 580581582 | | rac+− | 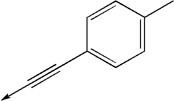 |
| 583584585 | | rac+− | 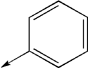 |
| 586587588 | | rac+− | 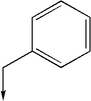 |
| 589590591 | | rac+− | 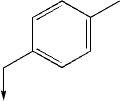 |
| 592593594 | | rac+− | 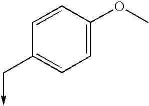 |
| 595596597 | | rac+− | 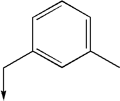 |
| 598599600 | | rac+− | 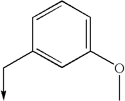 |

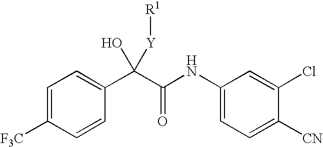
| | | Racemic or | |
|---|---|---|---|
| No. | Example | enantiomer | —Y —R¹ |
| 601602603 | | rac+− | 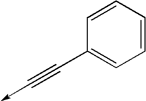 |
| 604605606 | | rac+− | 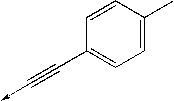 |
| 607608609 | | rac+− | 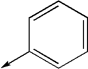 |
| 610611612 | | rac+− | 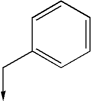 |
| 613614615 | | rac+− | 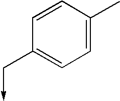 |
| 616617618 | | rac+− | 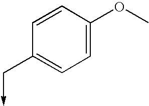 |
| 619620621 | | rac+− | 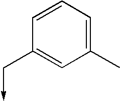 |
| 622623624 | | rac+− | 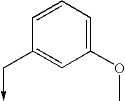 |

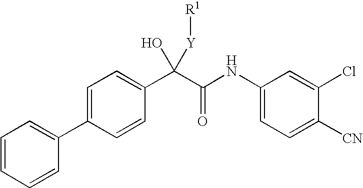
| | | Racemic or | |
|---|---|---|---|
| No. | Example | enantiomer | —Y —R¹ |
| 625626627 | | rac+− | 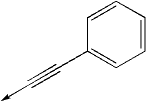 |
| 628629630 | | rac+− | 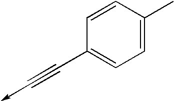 |
| 631632633 | | rac+− | 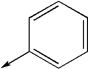 |
| 634635636 | | rac+− | 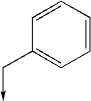 |
| 637638639 | | rac+− | 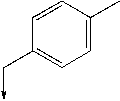 |
| 640641642 | | rac+− | 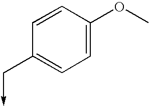 |
| 643644645 | | rac+− | 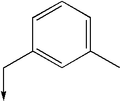 |
| 646647648 | | rac+− | 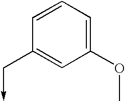 |

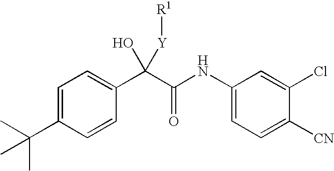
| | | Racemic or | |
|---|---|---|---|
| No. | Example | enantiomer | —Y —R¹ |
| 649650651 | | rac+− | 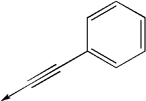 |
| 652653654 | | rac+− | 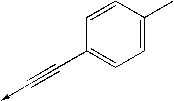 |
| 655656657 | | rac+− | 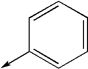 |
| 658659660 | | rac+− | 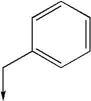 |
| 661662663 | | rac+− | 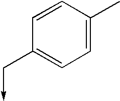 |
| 664665666 | | rac+− | 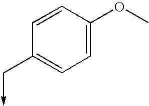 |
| 667668669 | | rac+− | 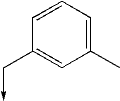 |
| 670671672 | | rac+− | 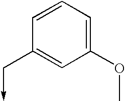 |

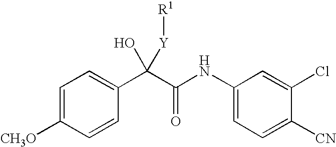
| | | Racemic or | |
|---|---|---|---|
| No. | Example | enantiomer | —Y —R¹ |
| 673674675 | | rac+− | 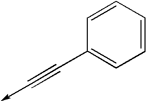 |
| 676677678 | | rac+− | 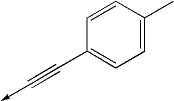 |
| 679680681 | | rac+− | 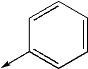 |
| 682683684 | | rac+− | 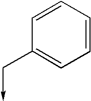 |
| 685686687 | | rac+− | 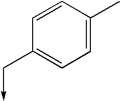 |
| 688689690 | | rac+− | 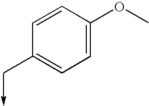 |
| 691692693 | | rac+− | 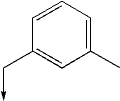 |
| 694695696 | | rac+− | 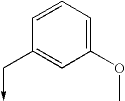 |

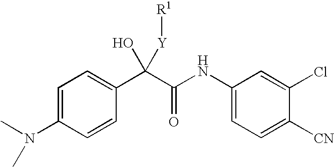
| | | Racemic or | |
|---|---|---|---|
| No. | Example | enantiomer | —Y —R¹ |
| 697698699 | | rac+− | 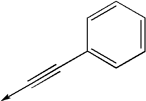 |
| 700701702 | | rac+− | 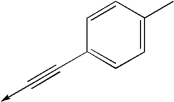 |
| 703704705 | | rac+− | 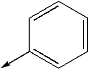 |
| 706707708 | | rac+− | 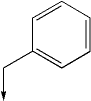 |
| 709710711 | | rac+− | 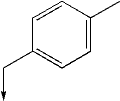 |
| 712713714 | | rac+− | 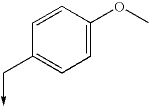 |
| 715716717 | | rac+− | 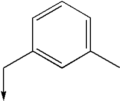 |
| 718719720 | | rac+− | 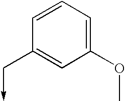 |

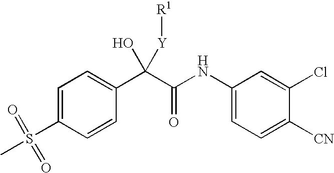
| | | Racemic or | |
|---|---|---|---|
| No. | Example | enantiomer | —Y —R¹ |
| 721722723 | | rac+− | 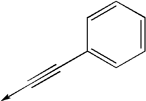 |
| 724725726 | | rac+− | 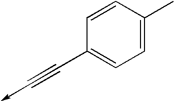 |
| 727728729 | | rac+− | 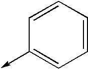 |
| 730731732 | | rac+− | 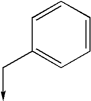 |
| 733734735 | | rac+− | 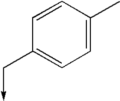 |
| 736737738 | | rac+− | 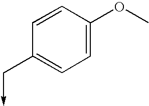 |
| 739740741 | | rac+− | 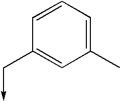 |
| 742743744 | | rac+− | 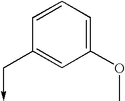 |

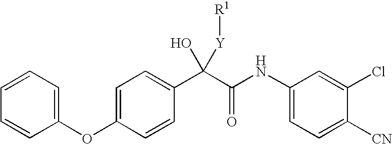
| | | Racemic or | |
|---|---|---|---|
| No. | Example | enantiomer | —Y —R¹ |
| 745746747 | | rac+− | 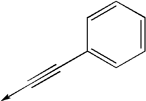 |
| 748749750 | | rac+− | 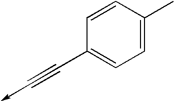 |
| 751752753 | | rac+− | 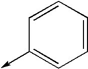 |
| 754755756 | | rac+− | 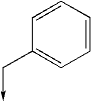 |
| 757758759 | | rac+− | 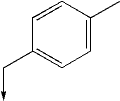 |
| 760761762 | | rac+− | 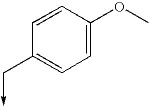 |
| 763764765 | | rac+− | 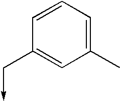 |
| 766767768 | | rac+− | 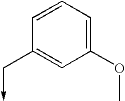 |

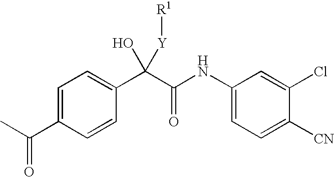
| | | Racemic or | |
|---|---|---|---|
| No. | Example | enantiomer | —Y —R¹ |
| 769770771 | | rac+− | 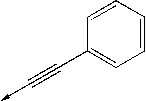 |
| 772773774 | | rac+− | 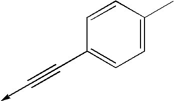 |
| 775776777 | | rac+− | 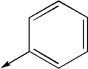 |
| 778779780 | | rac+− | 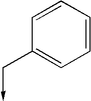 |
| 781782783 | | rac+− | 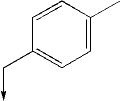 |
| 784785786 | | rac+− | 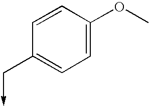 |
| 787788789 | | rac+− | 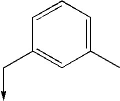 |
| 790791792 | | rac+− | 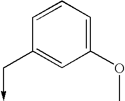 |

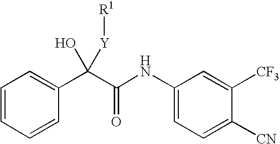
| | | Racemic or | |
|---|---|---|---|
| No. | Example | enantiomer | —Y —R¹ |
| 793794795 | | rac+− | 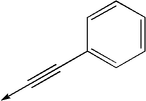 |
| 796797798 | | rac+− | 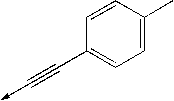 |
| 799800801 | | rac+− | 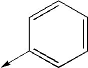 |
| 802803804 | | rac+− | 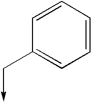 |
| 805806807 | | rac+− | 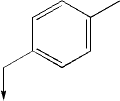 |
| 808809810 | | rac+− | 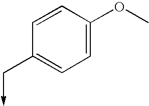 |
| 811812813 | | rac+− | 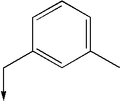 |
| 814815816 | | rac+− | 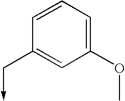 |

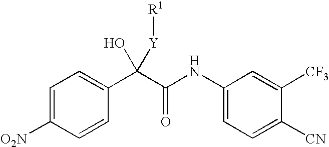
| | | Racemic or | |
|---|---|---|---|
| No. | Example | enantiomer | —Y —R¹ |
| 817818819 | | rac+− | 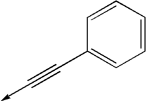 |
| 820821822 | | rac+− | 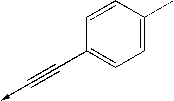 |
| 823824825 | | rac+− | 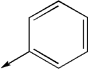 |
| 826827828 | | rac+− | 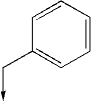 |
| 829830831 | | rac+− | 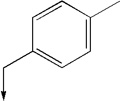 |
| 832833834 | | rac+− | 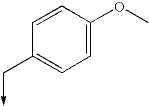 |
| 835836837 | | rac+− | 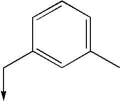 |
| 838839840 | | rac+− | 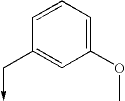 |

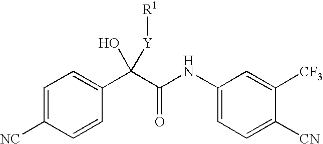
| | | Racemic or | |
|---|---|---|---|
| No. | Example | enantiomer | —Y —R¹ |
| 841842843 | | rac+− | 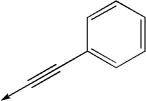 |
| 844845846 | | rac+− | 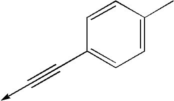 |
| 847848849 | | rac+− | 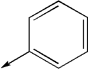 |
| 850851852 | | rac+− | 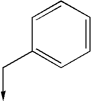 |
| 853854855 | | rac+− | 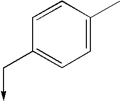 |
| 856857858 | | rac+− | 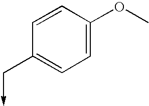 |
| 859860861 | | rac+− | 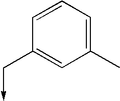 |
| 862863864 | | rac+− | 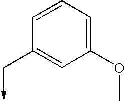 |

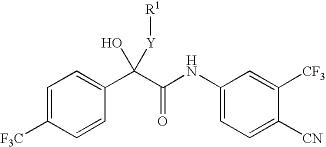
| | | Racemic or | |
|---|---|---|---|
| No. | Example | enantiomer | —Y —R¹ |
| 865866867 | | rac+− | 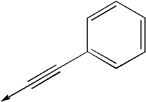 |
| 868869870 | | rac+− | 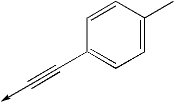 |
| 871872873 | | rac+− | 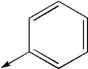 |
| 874875876 | | rac+− | 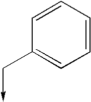 |
| 877878879 | | rac+− | 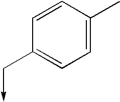 |
| 880881882 | | rac+− | 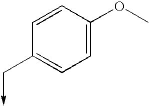 |
| 883884885 | | rac+− | 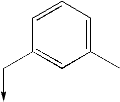 |
| 886887888 | | rac+− | 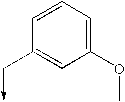 |

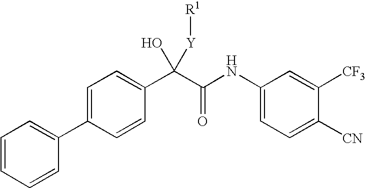
| | | racemisch oder | |
|---|---|---|---|
| Nr | Beispiel | Enantiomer | —Y —R¹ |
| 889890891 | | rac+− | 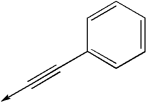 |
| 892893894 | | rac+− | 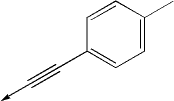 |
| 895896897 | | rac+− | 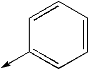 |
| 898899900 | | rac+− | 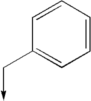 |
| 901902903 | | rac+− | 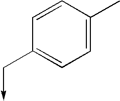 |
| 904905906 | | rac+− | 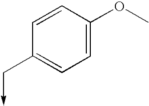 |
| 907908909 | | rac+− | 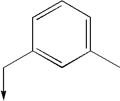 |
| 910911912 | | rac+− | 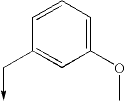 |

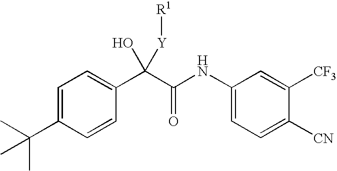
| | | Racemic or | |
|---|---|---|---|
| No. | Example | enantiomer | —Y —R¹ |
| 913914915 | | rac+− | 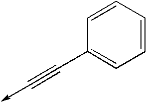 |
| 916917918 | | rac+− | 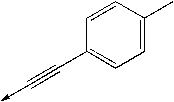 |
| 919920921 | | rac+− | 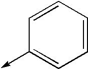 |
| 922923924 | | rac+− | 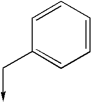 |
| 925926927 | | rac+− | 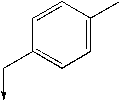 |
| 928929930 | | rac+− | 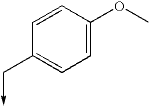 |
| 931932933 | | rac+− | 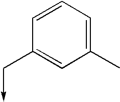 |
| 934935936 | | rac+− | 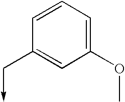 |

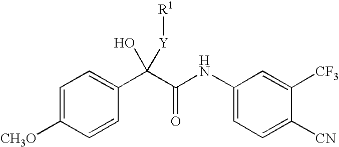
| | | racemisch oder | |
|---|---|---|---|
| Nr | Beispiel | Enantiomer | —Y —R¹ |
| 937938939 | | rac+− | 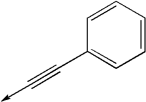 |
| 940941942 | | rac+− | 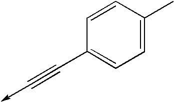 |
| 943944945 | | rac+− | 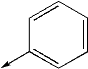 |
| 946947948 | | rac+− | 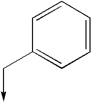 |
| 949950951 | | rac+− | 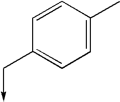 |
| 952953954 | | rac+− | 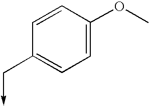 |
| 955956957 | | rac+− | 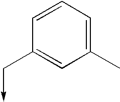 |
| 958959960 | | rac+− | 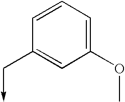 |

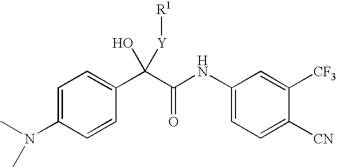
| | | Racemic or | |
|---|---|---|---|
| No. | Example | enantiomer | —Y—R¹ |
| 961962963 | | rac+− | 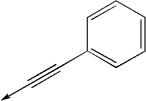 |
| 964965966 | | rac+− | 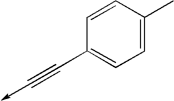 |
| 967968969 | | rac+− | 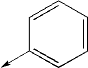 |
| 970971972 | | rac+− | 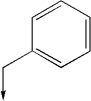 |
| 973974975 | | rac+− | 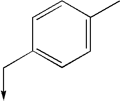 |
| 976977978 | | rac+− | 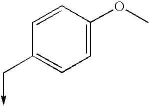 |
| 979980981 | | rac+− | 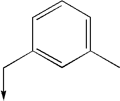 |
| 982983984 | | rac+− | 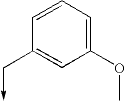 |

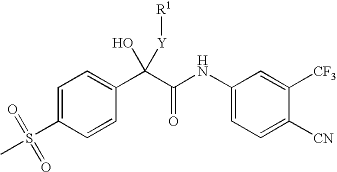
| | | Racemic or | |
|---|---|---|---|
| No. | Example | enantiomer | —Y—R¹ |
| 985 986 987 | | rac+− | 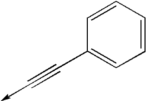 |
| 988 989 990 | | rac+− | 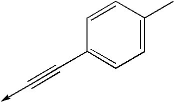 |
| 991 992 993 | | rac+− | 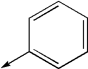 |
| 994 995 996 | | rac+− | 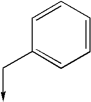 |
| 997 998 999 | | rac+− | 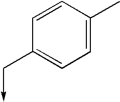 |
| 100010011002 | | rac+− | 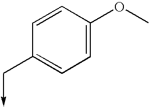 |
| 100310041005 | | rac+− | 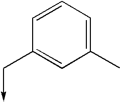 |
| 100610071008 | | rac+− | 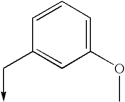 |

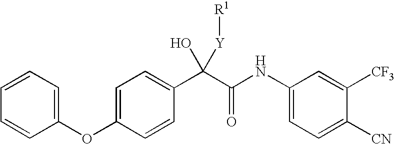
| | | Racemic or | |
|---|---|---|---|
| No. | Example | enantiomer | —Y—R¹ |
| 100910101011 | | rac+− | 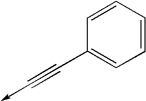 |
| 101210131014 | | rac+− | 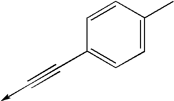 |
| 101510161017 | | rac+− | 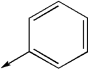 |
| 101810191020 | | rac+− | 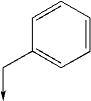 |
| 102110221023 | | rac+− | 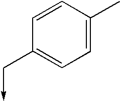 |
| 102410251026 | | rac+− | 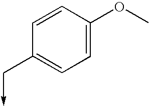 |
| 102710281029 | | rac+− | 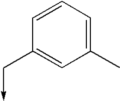 |
| 103010311032 | | rac+− | 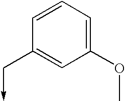 |

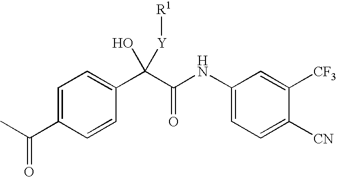
| | | Racemic or | |
|---|---|---|---|
| No. | Example | enantiomer | —Y—R¹ |
| 103310341035 | | rac+− | 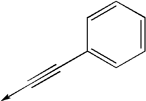 |
| 103610371038 | | rac+− | 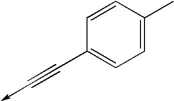 |
| 103910401041 | | rac+− | 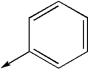 |
| 104210431044 | | rac+− | 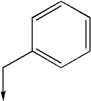 |
| 104510461047 | | rac+− | 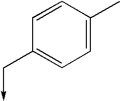 |
| 104810491050 | | rac+− | 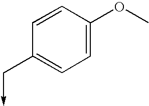 |
| 105110521053 | | rac+− | 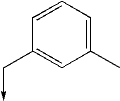 |
| 105410551056 | | rac+− | 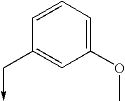 |

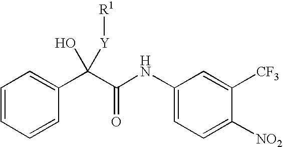
| | | Racemic or | |
|---|---|---|---|
| No. | Example | enantiomer | —Y—R¹ |
| 105710581059 | | rac+− | 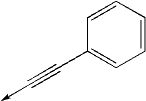 |
| 106010611062 | | rac+− | 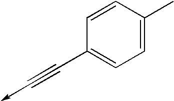 |
| 106310641065 | | rac+− | 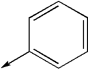 |
| 106610671068 | | rac+− | 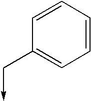 |
| 106910701071 | | rac+− | 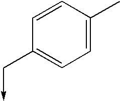 |
| 107210731074 | | rac+− | 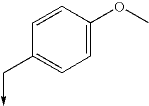 |
| 107510761077 | | rac+− | 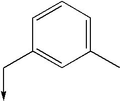 |
| 107810791080 | | rac+− | 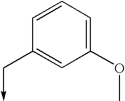 |

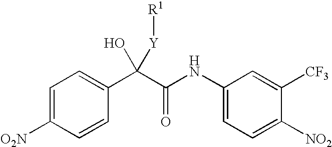
| | | Racemic or | |
|---|---|---|---|
| No. | Example | enantiomer | —Y—R¹ |
| 108110821083 | | rac+− | 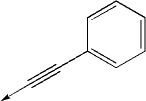 |
| 108410851086 | | rac+− | 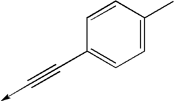 |
| 108710881089 | | rac+− | 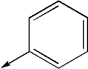 |
| 109010911092 | | rac+− | 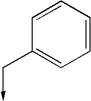 |
| 109310941095 | | rac+− | 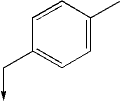 |
| 109610971098 | | rac+− | 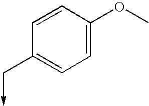 |
| 109911001101 | | rac+− | 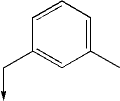 |
| 110211031104 | | rac+− | 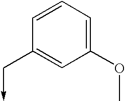 |

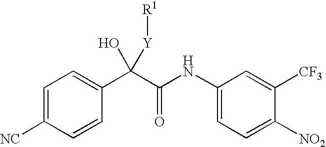
| | | Racemic or | |
|---|---|---|---|
| No. | Example | enantiomer | —Y—R¹ |
| 110511061107 | | rac+− | 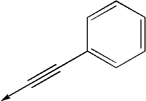 |
| 110811091110 | | rac+− | 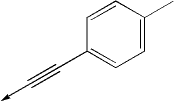 |
| 111111121113 | | rac+− | 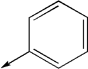 |
| 111411151116 | | rac+− | 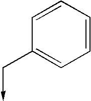 |
| 111711181119 | | rac+− | 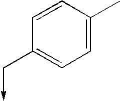 |
| 112011211122 | | rac+− | 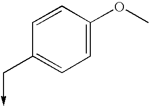 |
| 112311241125 | | rac+− | 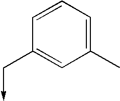 |
| 112611271128 | | rac+− | 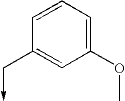 |

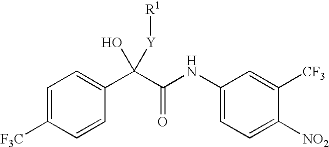
| | | Racemic or | |
|---|---|---|---|
| No. | Example | enantiomer | —Y—R¹ |
| 112911301131 | | rac+− | 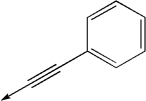 |
| 113211331134 | | rac+− | 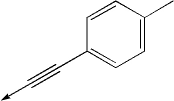 |
| 113511361137 | | rac+− | 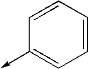 |
| 113811391140 | | rac+− | 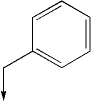 |
| 114111421143 | | rac+− | 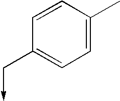 |
| 114411451146 | | rac+− | 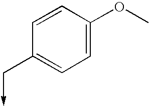 |
| 114711481149 | | rac+− | 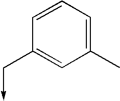 |
| 115011511152 | | rac+− | 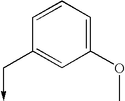 |

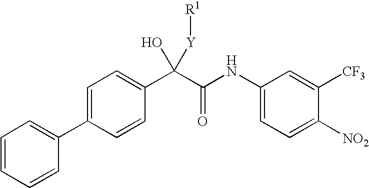
| | | Racemic or | |
|---|---|---|---|
| No. | Example | enantiomer | —Y—R¹ |
| 115311541155 | | rac+− | 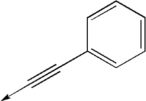 |
| 115611571158 | | rac+− | 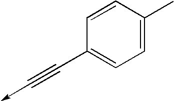 |
| 115911601161 | | rac+− | 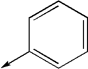 |
| 116211631164 | | rac+− | 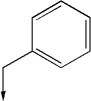 |
| 116511661167 | | rac+− | 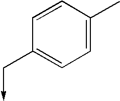 |
| 116811691170 | | rac+− | 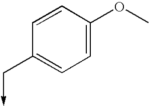 |
| 117111721173 | | rac+− | 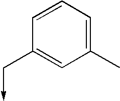 |
| 117411751176 | | rac+− | 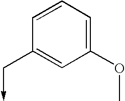 |

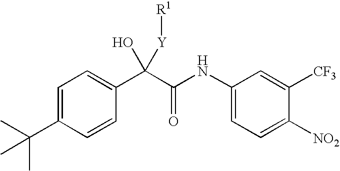
| | | Racemic or | |
|---|---|---|---|
| No. | Example | enantiomer | —Y—R¹ |
| 117711781179 | | rac+− | 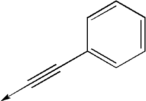 |
| 118011811182 | | rac+− | 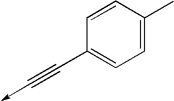 |
| 118311841185 | | rac+− | 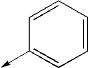 |
| 118611871188 | | rac+− | 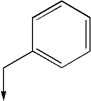 |
| 118911901191 | | rac+− | 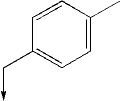 |
| 119211931194 | | rac+− | 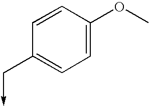 |
| 119511961197 | | rac+− | 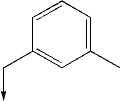 |
| 119811991200 | | rac+− | 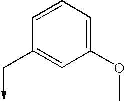 |

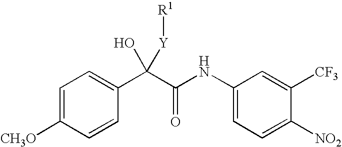
| | | Racemic or | |
|---|---|---|---|
| No. | Example | enantiomer | —Y—R¹ |
| 120112031203 | | rac+− | 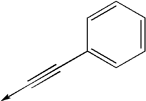 |
| 120412051206 | | rac+− | 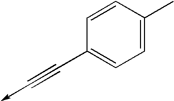 |
| 120712081209 | | rac+− | 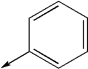 |
| 121012111212 | | rac+− | 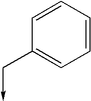 |
| 121312141215 | | rac+− | 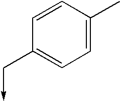 |
| 121612171218 | | rac+− | 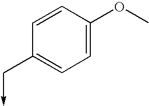 |
| 121912201221 | | rac+− | 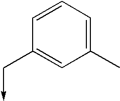 |
| 122212231224 | | rac+− | 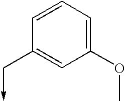 |

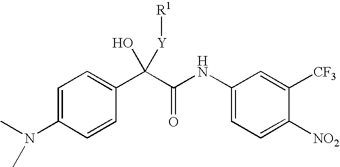
| | | Racemic or | |
|---|---|---|---|
| No. | Example | enantiomer | —Y—R¹ |
| 122512261227 | | rac+− | 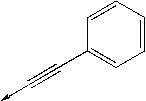 |
| 122812291230 | | rac+− | 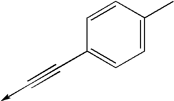 |
| 123112321233 | | rac+− | 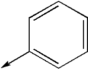 |
| 123412351236 | | rac+− | 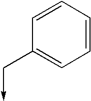 |
| 123712381239 | | rac+− | 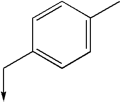 |
| 124012411242 | | rac+− | 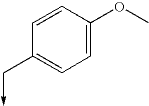 |
| 124312441245 | | rac+− | 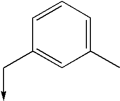 |
| 124612471248 | | rac+− | 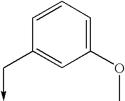 |

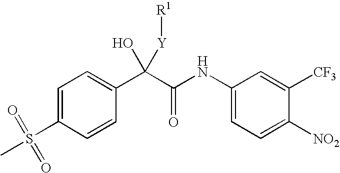
| | | Racemic or | |
|---|---|---|---|
| No. | Example | enantiomer | —Y—R¹ |
| 124912501251 | | rac+− | 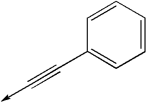 |
| 125212531254 | | rac+− | 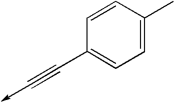 |
| 125512561257 | | rac+− | 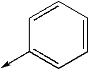 |
| 125812591260 | | rac+− | 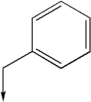 |
| 126112621263 | | rac+− | 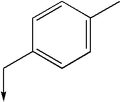 |
| 126412651266 | | rac+− | 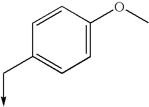 |
| 126712681269 | | rac+− | 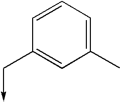 |
| 127012711272 | | rac+− | 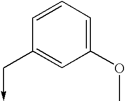 |

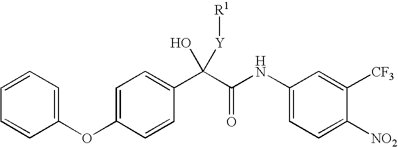
| | | Racemic or | |
|---|---|---|---|
| No. | Example | enantiomer | —Y—R¹ |
| 127312741275 | | rac+− | 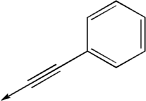 |
| 127612771278 | | rac+− | 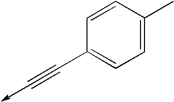 |
| 127912801281 | | rac+− | 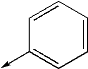 |
| 128212831284 | | rac+− | 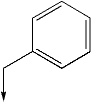 |
| 128512861287 | | rac+− | 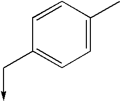 |
| 128812891290 | | rac+− | 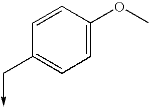 |
| 129112921293 | | rac+− | 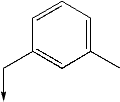 |
| 129412951296 | | rac+− | 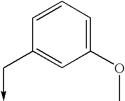 |

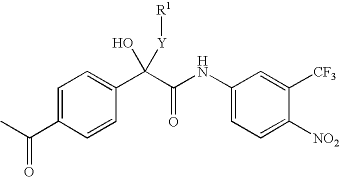
| | | Racemic or | |
|---|---|---|---|
| No. | Example | enantiomer | —Y—R¹ |
| 129712981299 | | rac+− | 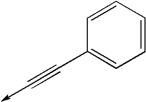 |
| 130013011302 | | rac+− | 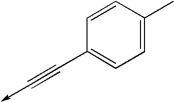 |
| 130313041305 | | rac+− | 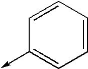 |
| 130613071308 | | rac+− | 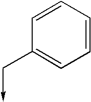 |
| 130913101311 | | rac+− | 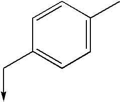 |
| 131213131314 | | rac+− | 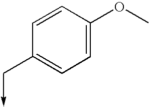 |
| 131513161317 | | rac+− | 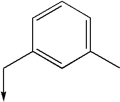 |
| 131813191320 | | rac+− | 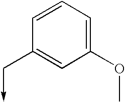 |

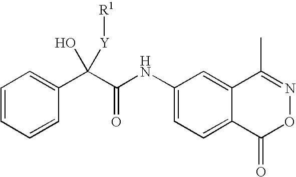
| | | Racemic or | |
|---|---|---|---|
| No. | Example | enantiomer | —Y—R¹ |
| 132113221323 | | rac+− | 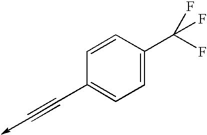 |
| 132413251326 | | rac+− | 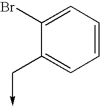 |
| 132713281329 | | rac+− | 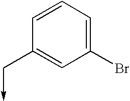 |
| 133013311332 | | rac+− | 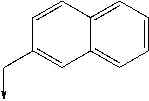 |
| 133313341335 | | rac+− | 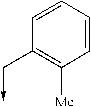 |
| 133613371338 | | rac+− | 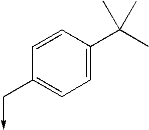 |
| 133913401341 | | rac+− | 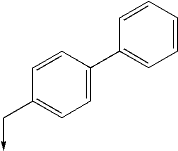 |

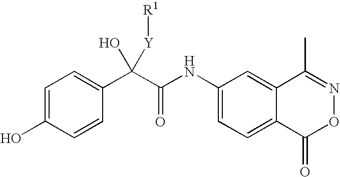
| | | Racemic or | |
|---|---|---|---|
| No. | Example | enantiomer | —Y—R¹ |
| 134213431344 | | rac+− | 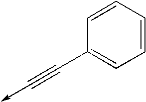 |
| 134513461347 | | rac+− | 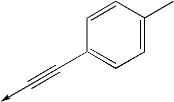 |
| 134813491350 | | rac+− | 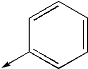 |
| 135113521353 | | rac+− | 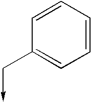 |
| 135413551356 | | rac+− | 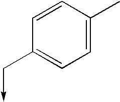 |
| 135713581359 | | rac+− | 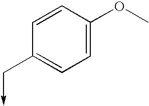 |
| 136013611362 | | rac+− | 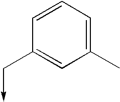 |
| 136313641365 | | rac+− | 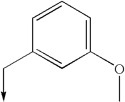 |

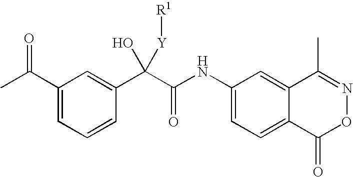
| | | Racemic or | |
|---|---|---|---|
| No. | Example | enantiomer | —Y—R¹ |
| 136613671368 | | rac+− | 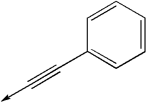 |
| 136913701371 | | rac+− | 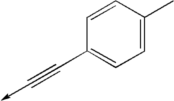 |
| 137213731374 | | rac+− | 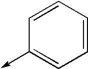 |
| 137513761377 | | rac+− | 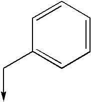 |
| 137813791380 | | rac+− | 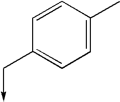 |
| 138113821383 | | rac+− | 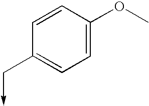 |
| 138413851386 | | rac+− | 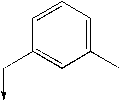 |
| 138713881389 | | rac+− | 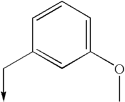 |

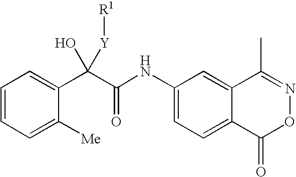
| | | Racemic or | |
|---|---|---|---|
| No. | Example | enantiomer | —Y—R¹ |
| 139013911392 | | rac+− | 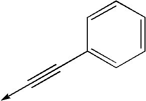 |
| 139313941395 | | rac+− | 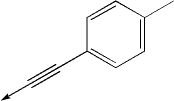 |
| 139613971398 | | rac+− | 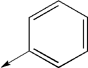 |
| 139914001401 | | rac+− | 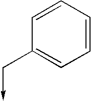 |
| 140214031404 | | rac+− | 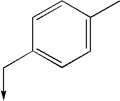 |
| 140514061407 | | rac+− | 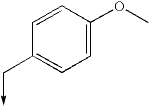 |
| 140814091410 | | rac+− | 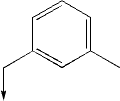 |
| 141114121413 | | rac+− | 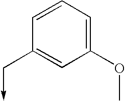 |

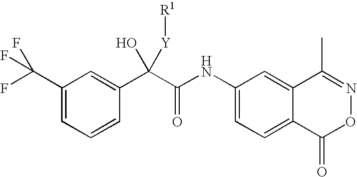
| | | Racemic or | |
|---|---|---|---|
| No. | Example | enantiomer | —Y—R¹ |
| 141414151416 | | rac+− | 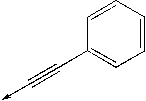 |
| 141714181419 | | rac+− | 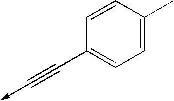 |
| 142014211422 | | rac+− | 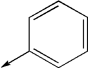 |
| 142314241425 | | rac+− | 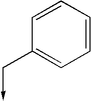 |
| 142614271428 | | rac+− | 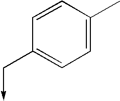 |
| 142914301431 | | rac+− | 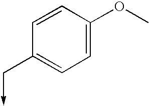 |
| 143214331434 | | rac+− | 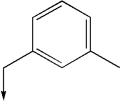 |
| 143514361437 | | rac+− | 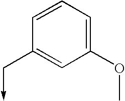 |

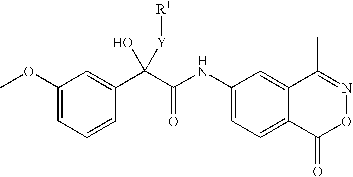
| | | Racemic or | |
|---|---|---|---|
| No. | Example | enantiomer | —Y—R¹ |
| 143814391440 | | rac+− | 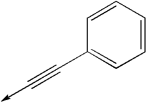 |
| 144114421443 | | rac+− | 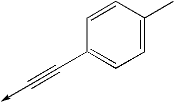 |
| 144414451446 | | rac+− | 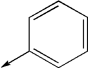 |
| 144714481449 | | rac+− | 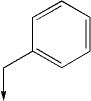 |
| 145014511452 | | rac+− | 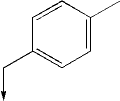 |
| 145314541455 | | rac+− | 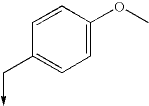 |
| 145614571458 | | rac+− | 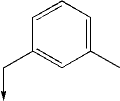 |
| 145914601461 | | rac+− | 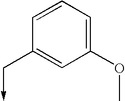 |

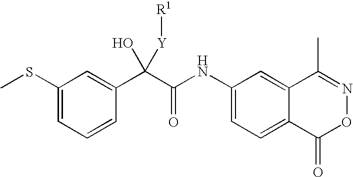
| | | Racemic or | |
|---|---|---|---|
| No. | Example | enantiomer | —Y—R¹ |
| 146214631464 | | rac+− | 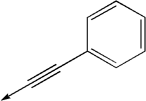 |
| 146514661467 | | rac+− | 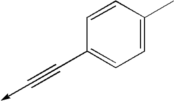 |
| 146814691470 | | rac+− | 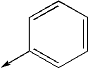 |
| 147114721473 | | rac+− | 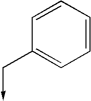 |
| 147414751476 | | rac+− | 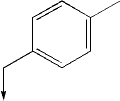 |
| 147714781479 | | rac+− | 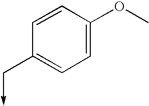 |
| 148014811482 | | rac+− | 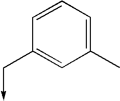 |
| 148314841485 | | rac+− | 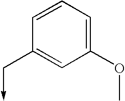 |

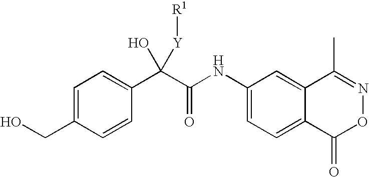
| | | Racemic or | |
|---|---|---|---|
| No. | Example | enantiomer | —Y—R¹ |
| 148614871488 | | rac+− | 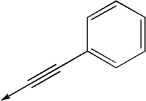 |
| 148914901491 | | rac+− | 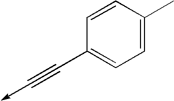 |
| 149214931494 | | rac+− | 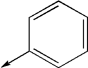 |
| 149514961497 | | rac+− | 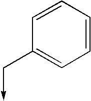 |
| 149814991500 | | rac+− | 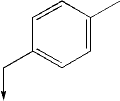 |
| 150115021503 | | rac+− | 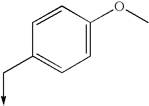 |
| 150415051506 | | rac+− | 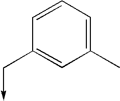 |
| 150715081509 | | rac+− | 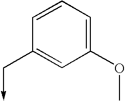 |

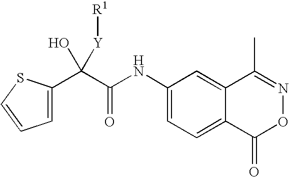
| | | Racemic or | |
|---|---|---|---|
| No. | Example | enantiomer | —Y—R¹ |
| 151015111512 | | rac+− | 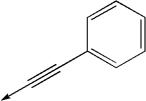 |
| 151315141515 | | rac+− | 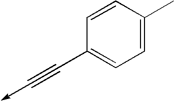 |
| 151615171518 | | rac+− | 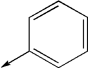 |
| 151915201521 | | rac+− | 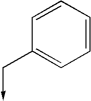 |
| 152215231524 | | rac+− | 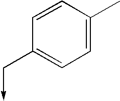 |
| 152515261527 | | rac+− | 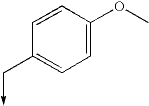 |
| 152815291530 | | rac+− | 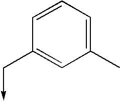 |
| 153115321533 | | rac+− | 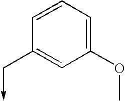 |

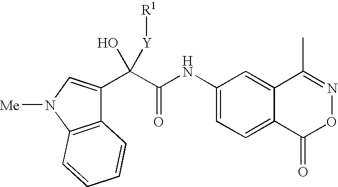
| | | Racemic or | |
|---|---|---|---|
| No. | Example | enantiomer | —Y—R¹ |
| 153415351536 | | rac+− | 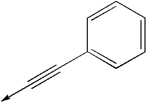 |
| 153715381539 | | rac+− | 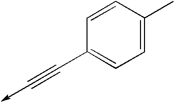 |
| 154015411542 | | rac+− | 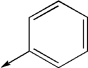 |
| 154315441545 | | rac+− | 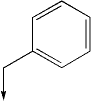 |
| 154615471548 | | rac+− | 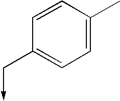 |
| 154915501551 | | rac+− | 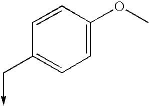 |
| 155215531554 | | rac+− | 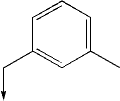 |
| 155515561557 | | rac+− | 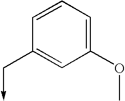 |

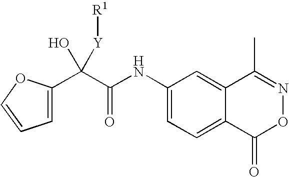
| | | Racemic or | |
|---|---|---|---|
| No. | Example | enantiomer | —Y—R¹ |
| 155815591560 | | rac+− | 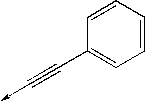 |
| 156115621563 | | rac+− | 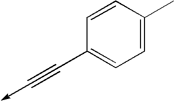 |
| 156415651566 | | rac+− | 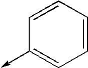 |
| 156715681569 | | rac+− | 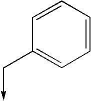 |
| 157015711572 | | rac+− | 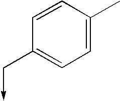 |
| 157315741575 | | rac+− | 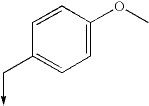 |
| 157615771578 | | rac+− | 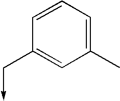 |
| 157915801581 | | rac+− | 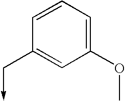 |

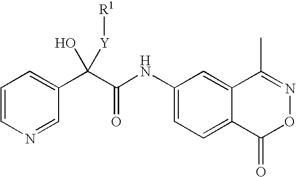
| | | Racemic or | |
|---|---|---|---|
| No. | Example | enantiomer | —Y—R¹ |
| 158215831584 | | rac+− | 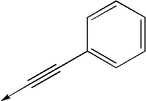 |
| 158515861587 | | rac+− | 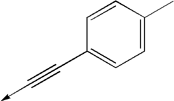 |
| 158815891590 | | rac+− | 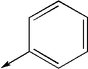 |
| 159115921593 | | rac+− | 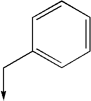 |
| 159415951596 | | rac+− | 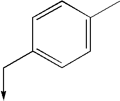 |
| 159715981599 | | rac+− | 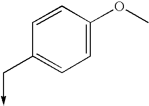 |
| 160016011602 | | rac+− | 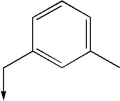 |
| 160316041605 | | rac+− | 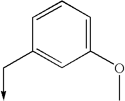 |

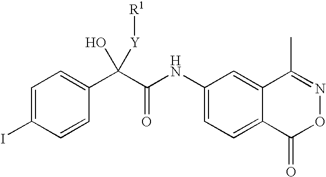
| | | Racemic or | |
|---|---|---|---|
| No. | Example | enantiomer | —Y—R¹ |
| 160616071608 | | rac+− | 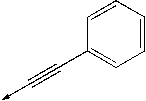 |
| 160916101611 | | rac+− | 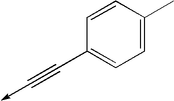 |
| 161216131614 | | rac+− | 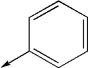 |
| 161516161617 | | rac+− | 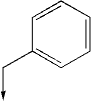 |
| 161816191620 | | rac+− | 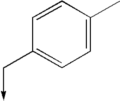 |
| 162116221623 | | rac+− | 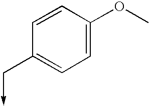 |
| 162416251626 | | rac+− | 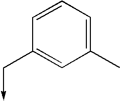 |
| 162716281629 | | rac+− | 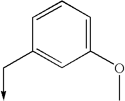 |

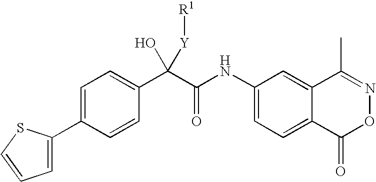
| | | Racemic or | |
|---|---|---|---|
| No. | Example | enantiomer | —Y—R¹ |
| 163016311632 | | rac+− | 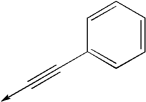 |
| 163316341635 | | rac+− | 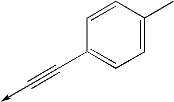 |
| 163616371638 | | rac+− | 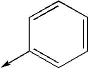 |
| 163916401641 | | rac+− | 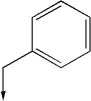 |
| 164216431644 | | rac+− | 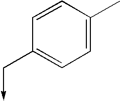 |
| 164516461647 | | rac+− | 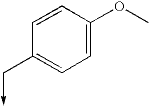 |
| 164816491650 | | rac+− | 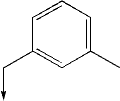 |
| 165116521653 | | rac+− | 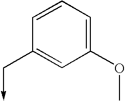 |

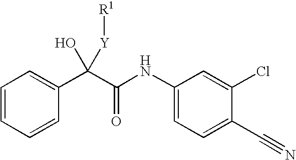
| | | Racemic or | |
|---|---|---|---|
| No. | Example | enantiomer | —Y—R¹ |
| 165416551656 | | rac+− | 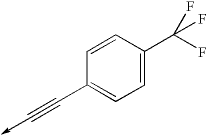 |
| 165716581659 | | rac+− | 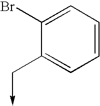 |
| 166016611662 | | rac+− | 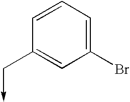 |
| 166316641665 | | rac+− | 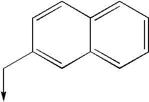 |
| 166616671668 | | rac+− | 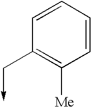 |
| 166916701671 | | rac+− | 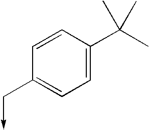 |
| 167216731674 | | rac+− | 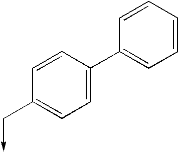 |
| 167516761677 | | rac+− | 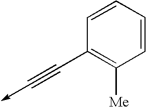 |
| 167816791680 | | rac+− | 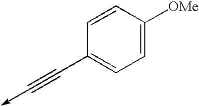 |
| 168116821683 | | rac+− | 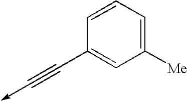 |
| 168416851686 | | rac+− | 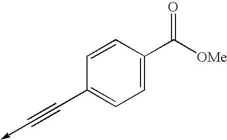 |
| 168716881689 | | rac+− | 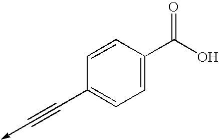 |

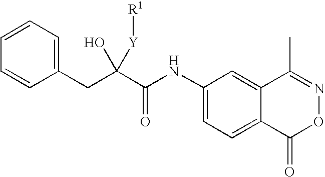
| | | Racemic or | |
|---|---|---|---|
| No. | Example | enantiomer | —Y—R¹ |
| 169016911692 | | rac+− | 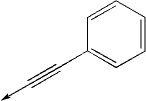 |
| 169316941695 | | rac+− | 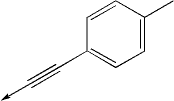 |
| 169616971698 | | rac+− | 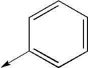 |
| 169917001701 | | rac+− | 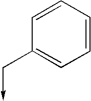 |
| 170217031704 | | rac+− | 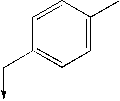 |
| 170517061707 | | rac+− | 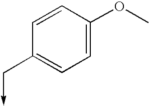 |
| 170817091710 | | rac+− | 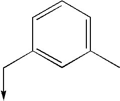 |
| 171117121713 | | rac+− | 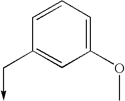 |

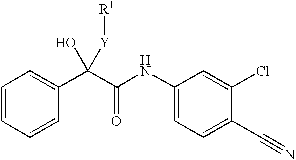
| | Ex- | | |
|---|---|---|---|
| | am- | Racemic or | |
| No. | ple | enantiomer | —Y—R¹ |
| 171417151716 | 86 | rac+− | 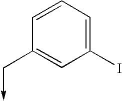 |
| 171717181719 | 97 | rac+− | 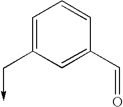 |
| 172017211722 | 90 | rac+− | 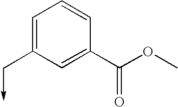 |
| 172317241725 | 92 | rac+− | 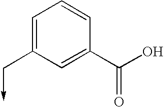 |
| 172617271728 | 94 | rac+− | 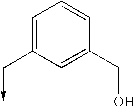 |
| 172917301731 | 87 | rac+− | 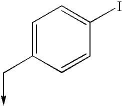 |
| 173217331734 | 88 | rac+− | 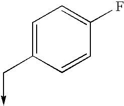 |
| 173517361737 | 93 | rac+− | 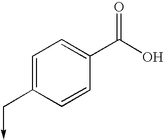 |
| 173817391740 | 98 | rac+− | 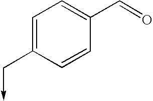 |
| 174117421743 | 91 | rac+− | 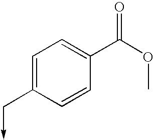 |
| 174417451746 | 100 | rac+− | 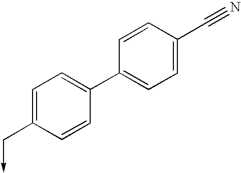 |
| 174717481749 | 103 | rac+− | 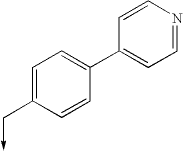 |
| 175017521753 | 109 | rac+− | 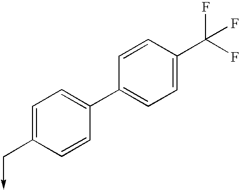 |
| 175417551756 | 112 | rac+− | 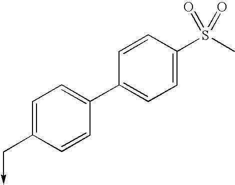 |
| 175717581759 | 115 | rac+− | 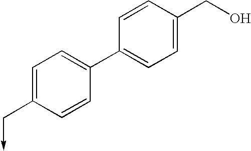 |
| 176017611762 | 118 | rac+− | 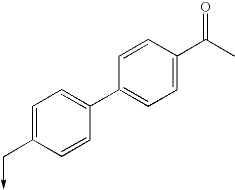 |
| 176317641765 | 106 | rac+− | 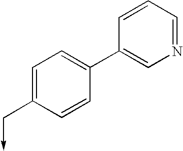 |
| 176617671768 | 121 | rac+− | 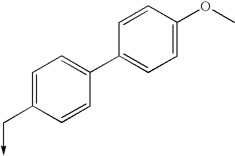 |
| 176917701771 | 123 | rac+− | 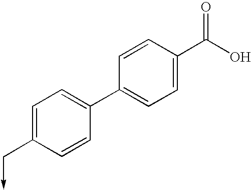 |
| 177217731774 | 99 | rac+− | 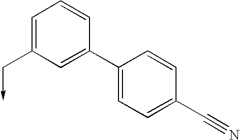 |
| 177517761777 | 120 | rac+− | 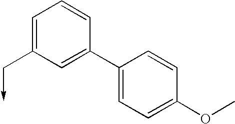 |
| 177817791780 | 105 | rac+− | 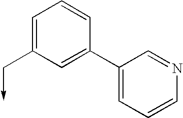 |
| 178117821783 | 111 | rac+− | 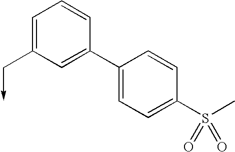 |
| 178417851787 | 114 | rac+− | 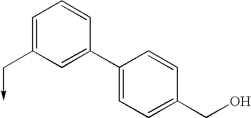 |
| 178817891790 | 102 | rac+− | 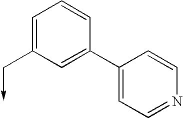 |
| 179117921793 | 117 | rac+− | 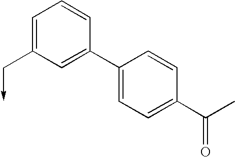 |
| 179417951796 | 108 | rac+− | 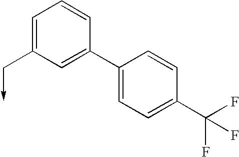 |
| 179717981799 | 101 | rac+− | 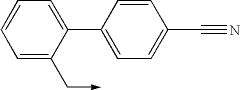 |
| 180018011802 | 122 | rac+− | 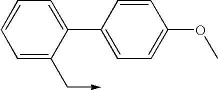 |
| 180318041805 | 119 | rac+− | 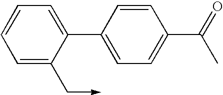 |
| 180618071808 | 124 | rac+− | 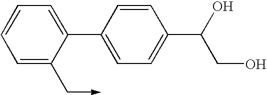 |
| 180918101811 | 107 | rac+− | 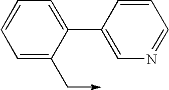 |
| 181218131814 | 110 | rac+− | 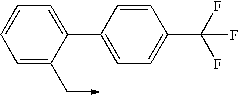 |
| 181518161817 | 113 | rac+− | 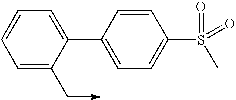 |
| 181818191820 | 116 | rac+− | 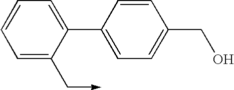 |
| 182118221823 | 104 | rac+− | 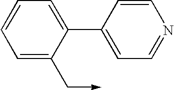 |
| 182418251826 | 125 | rac+− | 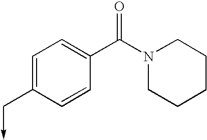 |
| 182718281829 | 127 | rac+− | 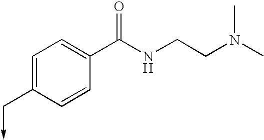 |
| 183018311832 | 128 | rac+− | 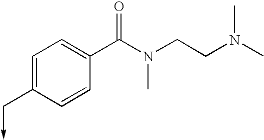 |
| 183318341835 | 126 | rac+− | 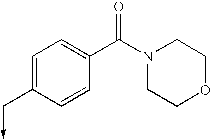 |
| 183618371838 | 134 | rac+− | 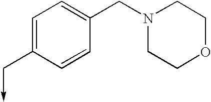 |
| 183918401841 | 131 | rac+− | 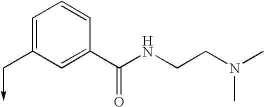 |
| 184218431844 | 133 | rac+− | 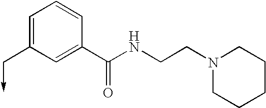 |
| 184518461847 | 132 | rac+− | 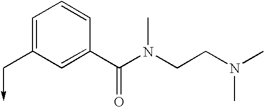 |
| 184818491850 | 130 | rac+− | 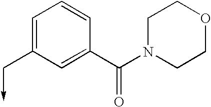 |
| 185118521853 | 129 | rac+− | 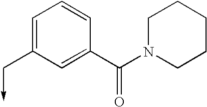 |
| 185418551856 | 135 | rac+− | 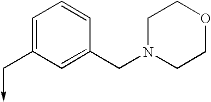 |
| 185718581859 | 137 | rac+− | 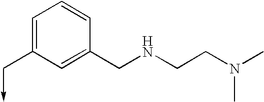 |
| 186018611862 | 136 | rac+− | 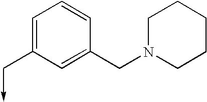 |
| 186318641865 | 138 | rac+− | 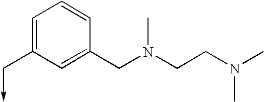 |
| 186618671868 | 139 | rac+− | 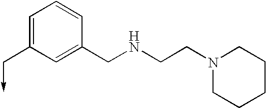 |
| 186918701871 | 95 | rac+− | 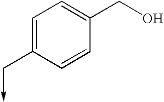 |
| 187218731874 | 96 | rac+− | 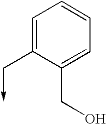 |
| 187518761877 | 89 | rac+− | 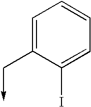 |

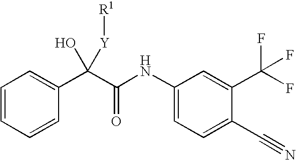
| | | Racemic or | |
|---|---|---|---|
| No. | Example | enantiomer | —Y—R¹ |
| 187818791880 | 140 | rac+− | 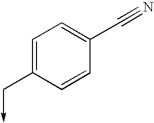 |
| 188118821883 | 141 | rac+− | 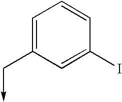 |
| 188418851886 | 142 | rac+− | 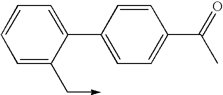 |

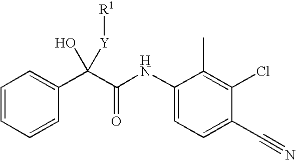
| | | Racemic or | |
|---|---|---|---|
| No. | Example | enantiomer | —Y—R¹ |
| 188718881889 | 144 | rac+− | 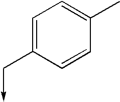 |
| 189018911892 | 145 | rac+− | 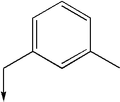 |
| 189318941895 | 146 | rac+− | 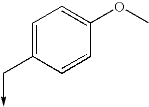 |
| 189618971898 | 143 | rac+− | 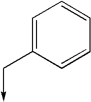 |
| 189919001901 | 147 | rac+− | 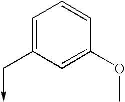 |

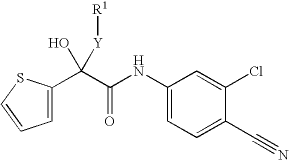
| | | Racemic or | |
|---|---|---|---|
| No. | Example | enantiomer | —Y—R¹ |
| 190219031904 | 148 | rac+− | 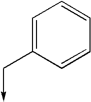 |

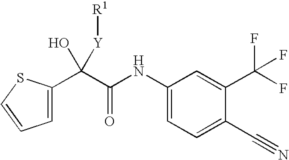
| | | Racemic or | |
|---|---|---|---|
| No. | Example | enantiomer | —Y—R¹ |
| 190519061907 | 150 | rac+− | 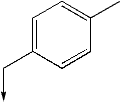 |
| 190819091910 | 149 | rac+− | 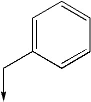 |

20. Pharmaceutical composition comprising at least one compound of the general formula I according to claim 1 and, where appropriate, at least one further active ingredient together with pharmaceutically suitable excipients and/or carriers.

21. Pharmaceutical composition according to claim 20, where the further active ingredient is a SERM (selective estrogen receptor modulator), an aromatase inhibitor, antiestrogen or a prostaglandin.

22. Pharmaceutical composition according to claim 21, where the further active ingredients may be tamoxifen, 5-(4-{5-[(RS)-(4,4,5,5,5-pentafluoropentyl)sulphinyl]-pentyloxy}phenyl)-6-phenyl-8,9-dihydro-7H-benzocyclohepten-2-ol, ICI 182 780 (7alpha-[9-(4,4,5,5-pentafluoropentylsulphinyl)nonyl]estra-1,3,5(10)-triene-3,17beta-diol), 11beta-fluoro-7alpha-[5-(methyl {3-[(4,4,5,5,5-pentafluoropentyl)sulphanyl]-propyl}amino)pentyl]estra-1,3,5(10)-triene-3,17beta-diol, 11beta-fluoro-7alpha-{5-[methyl(7,7,8,8,9,9,10,10,10-nonafluorodecyl)amino]pentyl}estra-1,3,5(10)-triene-3,17beta-diol, 11beta-fluoro-17alpha-methyl-7alpha-{5-[methyl(8,8,9,9,9-pentafluoronanyl)amino]pentyl}estra-1,3,5(10)-triene-3,17beta-diol, clomifene, raloxifene, fadrozole, formestane, letrozole, anastrozole or atamestane.

23. Compounds according to claim 1 for the manufacture of a medicament.

24. Use of compounds according to claim 1 for the manufacture of a medicament for the therapy and/or prophylaxis of gynaecological disorders such as endometriosis, leiomyomas of the uterus, dysfunctional bleeding and dysmenorrhoea.

25. Use of compounds according to claim 1 for the manufacture of a medicament for the therapy and/or prophylaxis of hormone-dependent tumours.

26. Use of compounds according to claim 1 for the manufacture of a medicament for the therapy and/or prophylaxis of breast carcinomas.

27. Use of compounds according to claim 1 for the manufacture of a medicament for the therapy and/or prophylaxis of endometrial carcinoma.

28. Use of compounds according to claim 1 for the manufacture of a medicament for the therapy and/or prophylaxis of ovarian carcinomas.

29. Use of compounds according to claim 1 for the manufacture of a medicament for the therapy and/or prophylaxis of prostate carcinomas.

30. Use of compounds according to claim 1 for the manufacture of a medicament for female hormone replacement therapy.

31. Use of compounds according to claim 1 for female fertility control.
